SEQUENCE LISTING (695 sequences)

SEQ ID NO: 1 - DNA, 21 bp, synthetic construct
  source 1..21: mol_type = other DNA
caacaagatgaagagcaccaa

SEQ ID NO: 2 - DNA, 21 bp, synthetic construct
  source 1..21: mol_type = other DNA
cttgagaagttcgtggacgat

SEQ ID NO: 3 - DNA, 21 bp, synthetic construct
  source 1..21: mol_type = other DNA
cctcaccattgagcggctcaa

SEQ ID NO: 4 - DNA, 21 bp, synthetic construct
  source 1..21: mol_type = other DNA
cattgggcatctctgcaagat

SEQ ID NO: 5 - DNA, 21 bp, synthetic construct
  source 1..21: mol_type = other DNA
ctgggaggatgtgcccatcat

SEQ ID NO: 6 - DNA, 21 bp, synthetic construct
  source 1..21: mol_type = other DNA
tgtctgttcttcagctggcaa

SEQ ID NO: 7 - protein, 9 aa, unidentified
LAGLIDADG

SEQ ID NO: 8 - protein, 7 aa, synthetic construct
DRGDLTR

SEQ ID NO: 9 - protein, 7 aa, synthetic construct
TSGALVR

SEQ ID NO: 10 - protein, 7 aa, synthetic construct
RSDNLAR

SEQ ID NO: 11 - protein, 7 aa, synthetic construct
RSDALAR

SEQ ID NO: 12 - protein, 7 aa, synthetic construct
RSDNRTN

SEQ ID NO: 13 - protein, 7 aa, synthetic construct
DRSNLSR

SEQ ID NO: 14 - protein, 7 aa, synthetic construct
RSDNLTR

SEQ ID NO: 15 - protein, 7 aa, synthetic construct
QSSDLSR

SEQ ID NO: 16 - protein, 7 aa, synthetic construct
RSDHLSR

SEQ ID NO: 17 - protein, 7 aa, synthetic construct
QSGHRKT

SEQ ID NO: 18 - protein, 7 aa, synthetic construct
RSDSLTR

SEQ ID NO: 19 - protein, 7 aa, synthetic construct
QSGDLTR

SEQ ID NO: 20 - protein, 7 aa, synthetic construct
TSGDLSR

SEQ ID NO: 21 - protein, 7 aa, synthetic construct
RSDSLAR

SEQ ID NO: 22 - protein, 7 aa, synthetic construct
DRSALTR

SEQ ID NO: 23 - protein, 7 aa, synthetic construct
QSSTLAI

SEQ ID NO: 24 - protein, 7 aa, synthetic construct
RSSALST

SEQ ID NO: 25 - protein, 7 aa, synthetic construct
DRSNLTR

SEQ ID NO: 26 - protein, 7 aa, synthetic construct
RSDHLSE

SEQ ID NO: 27 - protein, 7 aa, synthetic construct
DSRDRTK

SEQ ID NO: 28 - protein, 7 aa, synthetic construct
RSDHLAR

SEQ ID NO: 29 - protein, 7 aa, synthetic construct
QSSDLRR

SEQ ID NO: 30 - protein, 7 aa, synthetic construct
RNDNRKT

SEQ ID NO: 31 - protein, 7 aa, synthetic construct
QSAHLAR

SEQ ID NO: 32 - protein, 7 aa, synthetic construct
QSGDLRT

SEQ ID NO: 33 - protein, 7 aa, synthetic construct
DRSSRTR

SEQ ID NO: 34 - protein, 7 aa, synthetic construct
RSDHLSQ

SEQ ID NO: 35 - protein, 7 aa, synthetic construct
TSATRTT

SEQ ID NO: 36 - protein, 7 aa, synthetic construct
RSDHLSA

SEQ ID NO: 37 - protein, 7 aa, synthetic construct
RNAHRKR

SEQ ID NO: 38 - protein, 7 aa, synthetic construct
RSAHLSR

SEQ ID NO: 39 - protein, 7 aa, synthetic construct
QSGDLTT

SEQ ID NO: 40 - protein, 7 aa, synthetic construct
RSDHLTT

SEQ ID NO: 41 - protein, 7 aa, synthetic construct
TSSNLTR

SEQ ID NO: 42 - protein, 7 aa, synthetic construct
RSDHLTR

SEQ ID NO: 43 - protein, 7 aa, synthetic construct
RSDNLSA

SEQ ID NO: 44 - protein, 7 aa, synthetic construct
QSTHRIK

SEQ ID NO: 45 - protein, 7 aa, synthetic construct
QSGHRKA

SEQ ID NO: 46 - protein, 7 aa, synthetic construct
RSHHLAR

SEQ ID NO: 47 - protein, 7 aa, synthetic construct
QSGSLTR

SEQ ID NO: 48 - protein, 7 aa, synthetic construct
RNASRTR

SEQ ID NO: 49 - protein, 7 aa, synthetic construct
DSSTRKK

SEQ ID NO: 50 - protein, 7 aa, synthetic construct
QSAHLSR

SEQ ID NO: 51 - protein, 7 aa, synthetic construct
RSDNLSR

SEQ ID NO: 52 - protein, 7 aa, synthetic construct
DSSTRKT

SEQ ID NO: 53 - protein, 7 aa, synthetic construct
RSDHLST

SEQ ID NO: 54 - protein, 7 aa, synthetic construct
TSGHLSR

SEQ ID NO: 55 - protein, 7 aa, synthetic construct
QSGDRKT

SEQ ID NO: 56 - protein, 7 aa, synthetic construct
DPSSLVR

SEQ ID NO: 57 - protein, 7 aa, synthetic construct
RSDHRTK

SEQ ID NO: 58 - protein, 7 aa, synthetic construct
QSGHLIR

SEQ ID NO: 59 - protein, 7 aa, synthetic construct
RSSALSR

SEQ ID NO: 60 - protein, 7 aa, synthetic construct
RSGHLTR

SEQ ID NO: 61 - protein, 7 aa, synthetic construct
DRSALSR

SEQ ID NO: 62 - protein, 7 aa, synthetic construct
RSDSLSR

SEQ ID NO: 63 - protein, 7 aa, synthetic construct
RSDNLST

SEQ ID NO: 64 - protein, 7 aa, synthetic construct
DRSNRTK

SEQ ID NO: 65 - protein, 7 aa, synthetic construct
RSDVLSE

SEQ ID NO: 66 - protein, 7 aa, synthetic construct
QSANLAR

SEQ ID NO: 67 - protein, 7 aa, synthetic construct
QSGNLAR

SEQ ID NO: 68 - protein, 7 aa, synthetic construct
RSDSLTM

SEQ ID NO: 69 - protein, 7 aa, synthetic construct
QNATRKK

SEQ ID NO: 70 - protein, 7 aa, synthetic construct
QSGDLRK

SEQ ID NO: 71 - protein, 7 aa, synthetic construct
RSANLSR

SEQ ID NO: 72 - protein, 7 aa, synthetic construct
QLGDLST

SEQ ID NO: 73 - protein, 7 aa, synthetic construct
RSANLTR

SEQ ID NO: 74 - protein, 7 aa, synthetic construct
QSGHLTR

SEQ ID NO: 75 - protein, 7 aa, synthetic construct
QSGNLSR

SEQ ID NO: 76 - protein, 7 aa, synthetic construct
RKGDRKN

SEQ ID NO: 77 - protein, 7 aa, synthetic construct
QSANRNR

SEQ ID NO: 78 - protein, 7 aa, synthetic construct
QSGHLAR

SEQ ID NO: 79 - protein, 7 aa, synthetic construct
QSGHLSR

SEQ ID NO: 80 - protein, 7 aa, synthetic construct
DRGHLVR

SEQ ID NO: 81 - protein, 7 aa, synthetic construct
RSGNLSR

SEQ ID NO: 82 - protein, 7 aa, synthetic construct
RSDNRIT

SEQ ID NO: 83 - protein, 7 aa, synthetic construct
TSSNLAR

SEQ ID NO: 84 - protein, 7 aa, synthetic construct
RSDALSR

SEQ ID NO: 85 - protein, 7 aa, synthetic construct
RLDARKS

SEQ ID NO: 86 - protein, 7 aa, synthetic construct
RSDARTK

SEQ ID NO: 87 - protein, 7 aa, synthetic construct
DSSNRKK

SEQ ID NO: 88 - protein, 7 aa, synthetic construct
QSGALAR

SEQ ID NO: 89 - protein, 7 aa, synthetic construct
RSDALTR

SEQ ID NO: 90 - protein, 7 aa, synthetic construct
QSGNRKR

SEQ ID NO: 91 - protein, 7 aa, synthetic construct
DRSDLSR

SEQ ID NO: 92 - protein, 7 aa, synthetic construct
QSGALSR

SEQ ID NO: 93 - protein, 7 aa, synthetic construct
QSGNLRR

SEQ ID NO: 94 - protein, 7 aa, synthetic construct
QSATRKT

SEQ ID NO: 95 - protein, 7 aa, synthetic construct
ASSTRKK

SEQ ID NO: 96 - protein, 7 aa, synthetic construct
HRANLTR

SEQ ID NO: 97 - protein, 7 aa, synthetic construct
TSSTRTS

SEQ ID NO: 98 - protein, 7 aa, synthetic construct
QSSALSR

SEQ ID NO: 99 - protein, 7 aa, synthetic construct
LKSNLAR

SEQ ID NO: 100 - protein, 7 aa, synthetic construct
RSDNLTE

SEQ ID NO: 101 - protein, 7 aa, synthetic construct
TSSNRKK

SEQ ID NO: 102 - protein, 7 aa, synthetic construct
ERSDLRE

SEQ ID NO: 103 - protein, 7 aa, synthetic construct
QSDDLAR

SEQ ID NO: 104 - protein, 7 aa, synthetic construct
RSDALSV

SEQ ID NO: 105 - protein, 7 aa, synthetic construct
DRSHLTR

SEQ ID NO: 106 - protein, 7 aa, synthetic construct
LRSNRKS

SEQ ID NO: 107 - protein, 7 aa, synthetic construct
TSGHLTR

SEQ ID NO: 108 - protein, 7 aa, synthetic construct
TSHHLTR

SEQ ID NO: 109 - protein, 7 aa, synthetic construct
DRSNLRR

SEQ ID NO: 110 - protein, 7 aa, synthetic construct
QSGNLTR

SEQ ID NO: 111 - protein, 7 aa, synthetic construct
QSAHLTR

SEQ ID NO: 112 - protein, 7 aa, synthetic construct
RSDHLSK

SEQ ID NO: 113 - protein, 7 aa, synthetic construct
DSSNRTT

SEQ ID NO: 114 - protein, 7 aa, synthetic construct
RSDNLSV

SEQ ID NO: 115 - protein, 7 aa, synthetic construct
DRSDRTR

SEQ ID NO: 116 - protein, 7 aa, synthetic construct
QSSNLSR

SEQ ID NO: 117 - protein, 7 aa, synthetic construct
DRSDLTR

SEQ ID NO: 118 - protein, 7 aa, synthetic construct
QSSTLLR

SEQ ID NO: 119 - protein, 7 aa, synthetic construct
QSGTLTR

SEQ ID NO: 120 - protein, 7 aa, synthetic construct
RSDHLNQ

SEQ ID NO: 121 - protein, 7 aa, synthetic construct
RSGHLSR

SEQ ID NO: 122 - protein, 7 aa, synthetic construct
RSHHRKT

SEQ ID NO: 123 - protein, 7 aa, synthetic construct
RSDTLTV

SEQ ID NO: 124 - protein, 7 aa, synthetic construct
DRSHLAR

SEQ ID NO: 125 - protein, 7 aa, synthetic construct
RSDARKV

SEQ ID NO: 126 - protein, 7 aa, synthetic construct
RNDHRKT

SEQ ID NO: 127 - protein, 7 aa, synthetic construct
RSSDLSE

SEQ ID NO: 128 - protein, 7 aa, synthetic construct
RNASLSR

SEQ ID NO: 129 - protein, 7 aa, synthetic construct
QSAHLSE

SEQ ID NO: 130 - protein, 7 aa, synthetic construct
TSQVLRR

SEQ ID NO: 131 - protein, 7 aa, synthetic construct
RSDSLLR

SEQ ID NO: 132 - protein, 7 aa, synthetic construct
RLDNRTA

SEQ ID NO: 133 - protein, 7 aa, synthetic construct
RSDNLSE

SEQ ID NO: 134 - protein, 7 aa, synthetic construct
QNSTRTK

SEQ ID NO: 135 - protein, 7 aa, synthetic construct
RSDDLSK

SEQ ID NO: 136 - protein, 7 aa, synthetic construct
DSATRTK

SEQ ID NO: 137 - protein, 7 aa, synthetic construct
QSGDLTA

SEQ ID NO: 138 - protein, 7 aa, synthetic construct
RSGSLTR

SEQ ID NO: 139 - protein, 7 aa, synthetic construct
TSGNLRT

SEQ ID NO: 140 - protein, 7 aa, synthetic construct
RSANLRT

SEQ ID NO: 141 - protein, 7 aa, synthetic construct
RSDNRTR

SEQ ID NO: 142 - protein, 7 aa, synthetic construct
RNDNRKK

SEQ ID NO: 143 - protein, 7 aa, synthetic construct
QSGDLSV

SEQ ID NO: 144 - protein, 7 aa, synthetic construct
RSDVRKV

SEQ ID NO: 145 - protein, 7 aa, synthetic construct
DRSHLSR

SEQ ID NO: 146 - protein, 7 aa, synthetic construct
RSSHLSR

SEQ ID NO: 147 - protein, 7 aa, synthetic construct
QSSNLTR

SEQ ID NO: 148 - protein, 7 aa, synthetic construct
RSGHLVR

SEQ ID NO: 149 - protein, 7 aa, synthetic construct
TSGDLVR

SEQ ID NO: 150 - protein, 7 aa, synthetic construct
QSSTRTK

SEQ ID NO: 151 - protein, 7 aa, synthetic construct
DRSSLAR

SEQ ID NO: 152 - protein, 7 aa, synthetic construct
QKGTLGE

SEQ ID NO: 153 - protein, 7 aa, synthetic construct
QSSDRKR

SEQ ID NO: 154 - protein, 7 aa, synthetic construct
RLDNLPM

SEQ ID NO: 155 - protein, 7 aa, synthetic construct
DRGTLTR

SEQ ID NO: 156 - protein, 7 aa, synthetic construct
TSGHLAR

SEQ ID NO: 157 - protein, 7 aa, synthetic construct
TSGNLAR

SEQ ID NO: 158 - protein, 7 aa, synthetic construct
DRSDRIT

SEQ ID NO: 159 - protein, 7 aa, synthetic construct
TSSTRKK

SEQ ID NO: 160 - protein, 7 aa, synthetic construct
DRDHLSE

SEQ ID NO: 161 - protein, 7 aa, synthetic construct
DRSHRIR

SEQ ID NO: 162 - protein, 7 aa, synthetic construct
RLDNRKA

SEQ ID NO: 163 - protein, 7 aa, synthetic construct
DRDHLAQ

SEQ ID NO: 164 - protein, 7 aa, synthetic construct
QSGNRKT

SEQ ID NO: 165 - protein, 7 aa, synthetic construct
RSDHRKT

SEQ ID NO: 166 - protein, 7 aa, synthetic construct
RNHHLSR

SEQ ID NO: 167 - protein, 7 aa, synthetic construct
RSSNLAR

SEQ ID NO: 168 - protein, 7 aa, synthetic construct
RSANLAR

SEQ ID NO: 169 - protein, 7 aa, synthetic construct
RSDNRKK

SEQ ID NO: 170 - protein, 7 aa, synthetic construct
QNGNLTR

SEQ ID NO: 171 - protein, 7 aa, synthetic construct
QSGDLRR

SEQ ID NO: 172 - protein, 7 aa, synthetic construct
RSGDLTR

SEQ ID NO: 173 - protein, 7 aa, synthetic construct
DSSNLSR

SEQ ID NO: 174 - protein, 7 aa, synthetic construct
DRGDLTE

SEQ ID NO: 175 - protein, 7 aa, synthetic construct
QSDNLAA

SEQ ID NO: 176 - protein, 7 aa, synthetic construct
QNGDLTT

SEQ ID NO: 177 - protein, 7 aa, synthetic construct
RSASLTR

SEQ ID NO: 178 - protein, 7 aa, synthetic construct
TSSNLSR

SEQ ID NO: 179 - protein, 7 aa, synthetic construct
QSATLAR

SEQ ID NO: 180 - protein, 7 aa, synthetic construct
RSDALRS

SEQ ID NO: 181 - protein, 7 aa, synthetic construct
TSSHLSR

SEQ ID NO: 182 - protein, 7 aa, synthetic construct
LKSYRIT

SEQ ID NO: 183 - protein, 7 aa, synthetic construct
DSATRKK

SEQ ID NO: 184 - protein, 7 aa, synthetic construct
RSDSLST

SEQ ID NO: 185 - protein, 7 aa, synthetic construct
DRATRKT

SEQ ID NO: 186 - protein, 7 aa, synthetic construct
QKSTRKK

SEQ ID NO: 187 - protein, 7 aa, synthetic construct
DRGNLSR

SEQ ID NO: 188 - protein, 7 aa, synthetic construct
QSGSLSR

SEQ ID NO: 189 - protein, 7 aa, synthetic construct
LKQHLRT

SEQ ID NO: 190 - protein, 7 aa, synthetic construct
ERGTLAR

SEQ ID NO: 191 - protein, 7 aa, synthetic construct
DRSDLAR

SEQ ID NO: 192 - protein, 7 aa, synthetic construct
LKQHLRS

SEQ ID NO: 193 - protein, 7 aa, synthetic construct
RSDNRKA

SEQ ID NO: 194 - protein, 7 aa, synthetic construct
RSDTLSE

SEQ ID NO: 195 - protein, 7 aa, synthetic construct
QSATRTT

SEQ ID NO: 196 - protein, 7 aa, synthetic construct
HSGHLSR

SEQ ID NO: 197 - protein, 7 aa, synthetic construct
RSDVLSQ

SEQ ID NO: 198 - protein, 7 aa, synthetic construct
DNHHRKT

SEQ ID NO: 199 - protein, 7 aa, synthetic construct
DSSNLTR

SEQ ID NO: 200 - protein, 7 aa, synthetic construct
TSSSLAK

SEQ ID NO: 201 - protein, 7 aa, synthetic construct
RSDALSQ

SEQ ID NO: 202 - protein, 7 aa, synthetic construct
DRSSLTR

SEQ ID NO: 203 - protein, 7 aa, synthetic construct
QSTSLRK

SEQ ID NO: 204 - protein, 7 aa, synthetic construct
TPSALSR

SEQ ID NO: 205 - protein, 7 aa, synthetic construct
DRATRTK

SEQ ID NO: 206 - protein, 7 aa, synthetic construct
QNGDLTR

SEQ ID NO: 207 - protein, 7 aa, synthetic construct
TSSTRIK

SEQ ID NO: 208 - protein, 7 aa, synthetic construct
RSDNLKD

SEQ ID NO: 209 - protein, 7 aa, synthetic construct
RSDSLSV

SEQ ID NO: 210 - protein, 7 aa, synthetic construct
DRSTRKR

SEQ ID NO: 211 - protein, 7 aa, synthetic construct
RSGTLTR

SEQ ID NO: 212 - protein, 7 aa, synthetic construct
QSQDRKT

SEQ ID NO: 213 - protein, 7 aa, synthetic construct
TSGNLVR

SEQ ID NO: 214 - protein, 7 aa, synthetic construct
RSDALRV

SEQ ID NO: 215 - protein, 7 aa, synthetic construct
DRGNLTR

SEQ ID NO: 216 - protein, 7 aa, synthetic construct
TSSHLAR

SEQ ID NO: 217 - protein, 7 aa, synthetic construct
TSGHLVR

SEQ ID NO: 218 - protein, 7 aa, synthetic construct
RRDALTT

SEQ ID NO: 219 - protein, 7 aa, synthetic construct
RLGNLSE

SEQ ID NO: 220 - protein, 7 aa, synthetic construct
QNANRIR

SEQ ID NO: 221 - protein, 7 aa, synthetic construct
RSHHRIN

SEQ ID NO: 222 - protein, 7 aa, synthetic construct
RSDDRKE

SEQ ID NO: 223 - protein, 7 aa, synthetic construct
RNSNRIK

SEQ ID NO: 224 - protein, 7 aa, synthetic construct
RSGHLAR

SEQ ID NO: 225 - protein, 7 aa, synthetic construct
QNANRTK

SEQ ID NO: 226 - protein, 7 aa, synthetic construct
LKSNRTR

SEQ ID NO: 227 - protein, 7 aa, synthetic construct
RSDSLSQ

SEQ ID NO: 228 - protein, 7 aa, synthetic construct
TSQHLSK

SEQ ID NO: 229 - protein, 7 aa, synthetic construct
QSANLTR

SEQ ID NO: 230 - protein, 7 aa, synthetic construct
RSHHLAN

SEQ ID NO: 231 - protein, 7 aa, synthetic construct
QSSNRTK

SEQ ID NO: 232 - protein, 7 aa, synthetic construct
QKGDRKS

SEQ ID NO: 233 - protein, 7 aa, synthetic construct
QAGNLSK

SEQ ID NO: 234 - protein, 7 aa, synthetic construct
RSDHLAE

SEQ ID NO: 235 - protein, 7 aa, synthetic construct
QSSTRNK

SEQ ID NO: 236 - protein, 7 aa, synthetic construct
TSGSLAR

SEQ ID NO: 237 - protein, 7 aa, synthetic construct
RSDALRA

SEQ ID NO: 238 - protein, 7 aa, synthetic construct
TSGTLTR

SEQ ID NO: 239 - protein, 7 aa, synthetic construct
RSDHLAA

SEQ ID NO: 240 - protein, 7 aa, synthetic construct
QNDNLAR

SEQ ID NO: 241 - protein, 7 aa, synthetic construct
RSAHLAR

SEQ ID NO: 242 - protein, 7 aa, synthetic construct
RSDHRKK

SEQ ID NO: 243 - protein, 7 aa, synthetic construct
QSSHLAR

SEQ ID NO: 244 - protein, 7 aa, synthetic construct
QSAHRKT

SEQ ID NO: 245 - protein, 7 aa, synthetic construct
RSDTLST

SEQ ID NO: 246 - protein, 7 aa, synthetic construct
QNANRTR

SEQ ID NO: 247 - protein, 7 aa, synthetic construct
RNQNRTN

SEQ ID NO: 248 - protein, 7 aa, synthetic construct
TSDHRKT

SEQ ID NO: 249 - protein, 7 aa, synthetic construct
QSSNLRT

SEQ ID NO: 250 - protein, 7 aa, synthetic construct
RSDNLRA

SEQ ID NO: 251 - protein, 7 aa, synthetic construct
TSSNRTK

SEQ ID NO: 252 - protein, 7 aa, synthetic construct
RSDTLSV

SEQ ID NO: 253 - protein, 7 aa, synthetic construct
QSDVLSQ

SEQ ID NO: 254 - protein, 7 aa, synthetic construct
TSSNRTT

SEQ ID NO: 255 - protein, 7 aa, synthetic construct
RSDVLTA

SEQ ID NO: 256 - protein, 7 aa, synthetic construct
QNATRTK

SEQ ID NO: 257 - protein, 7 aa, synthetic construct
RSDTLSK

SEQ ID NO: 258 - protein, 7 aa, synthetic construct
TSSNRKQ

SEQ ID NO: 259 - protein, 7 aa, synthetic construct
QSATRTK

SEQ ID NO: 260 - protein, 7 aa, synthetic construct
TSQNLTK

SEQ ID NO: 261 - protein, 7 aa, synthetic construct
TSSALSR

SEQ ID NO: 262 - protein, 7 aa, synthetic construct
HRSYLTD

SEQ ID NO: 263 - protein, 7 aa, synthetic construct
QSGDLSQ

SEQ ID NO: 264 - protein, 7 aa, synthetic construct
RSGHLSE

SEQ ID NO: 265 - protein, 7 aa, synthetic construct
DSGHLTR

SEQ ID NO: 266 - protein, 7 aa, synthetic construct
QSGHLSE

SEQ ID NO: 267 - protein, 7 aa, synthetic construct
RNDARTK

SEQ ID NO: 268 - protein, 7 aa, synthetic construct
DRDTLSA

SEQ ID NO: 269 - protein, 7 aa, synthetic construct
TSGNLTR

SEQ ID NO: 270 - protein, 7 aa, synthetic construct
DRDTLSQ

SEQ ID NO: 271 - protein, 7 aa, synthetic construct
QSANRTK

SEQ ID NO: 272 - protein, 7 aa, synthetic construct
TSHNRTH

SEQ ID NO: 273 - protein, 7 aa, synthetic construct
RSSNLSR

SEQ ID NO: 274 - protein, 7 aa, synthetic construct
TSANRIN

SEQ ID NO: 275 - protein, 7 aa, synthetic construct
QSSNRTT

SEQ ID NO: 276 - protein, 7 aa, synthetic construct
QSSNLAR

SEQ ID NO: 277 - protein, 7 aa, synthetic construct
LKANLRK

SEQ ID NO: 278 - protein, 7 aa, synthetic construct
RSDALRQ

SEQ ID NO: 279 - protein, 7 aa, synthetic construct
DRSNLRK

SEQ ID NO: 280 - protein, 7 aa, synthetic construct
TSGNLAA

SEQ ID NO: 281 - protein, 7 aa, synthetic construct
DRSNRTR

SEQ ID NO: 282 - protein, 7 aa, synthetic construct
QSSNRKT

SEQ ID NO: 283 - protein, 7 aa, synthetic construct
TSSNRKT

SEQ ID NO: 284 - protein, 7 aa, synthetic construct
RNGNLTR

SEQ ID NO: 285 - protein, 7 aa, synthetic construct
RSDALST

SEQ ID NO: 286 - protein, 7 aa, synthetic construct
RNDNRIT

SEQ ID NO: 287 - protein, 7 aa, synthetic construct
TSGDLTR

SEQ ID NO: 288 - protein, 7 aa, synthetic construct
RSGNLVR

SEQ ID NO: 289 - protein, 7 aa, synthetic construct
RSGNLTR

SEQ ID NO: 290 - protein, 7 aa, synthetic construct
NSSTRIK

SEQ ID NO: 291 - protein, 7 aa, synthetic construct
QNGNLSK

SEQ ID NO: 292 - protein, 7 aa, synthetic construct
DSDVLNE

SEQ ID NO: 293 - protein, 7 aa, synthetic construct
TSAHRIT

SEQ ID NO: 294 - protein, 7 aa, synthetic construct
RNSSRKN

SEQ ID NO: 295 - protein, 7 aa, synthetic construct
DRGTLSR

SEQ ID NO: 296 - protein, 7 aa, synthetic construct
RSDDLSR

SEQ ID NO: 297 - protein, 7 aa, synthetic construct
DNQHLTS

SEQ ID NO: 298 - protein, 7 aa, synthetic construct
QSGNLRA

SEQ ID NO: 299 - protein, 7 aa, synthetic construct
RNDALTS

SEQ ID NO: 300 - protein, 7 aa, synthetic construct
TSDTLSE

SEQ ID NO: 301 - protein, 7 aa, synthetic construct
RNRDRTK

SEQ ID NO: 302 - protein, 7 aa, synthetic construct
DNSNRTK

SEQ ID NO: 303 - protein, 7 aa, synthetic construct
DRANRKK

SEQ ID NO: 304 - protein, 7 aa, synthetic construct
DRSYLSR

SEQ ID NO: 305 - protein, 7 aa, synthetic construct
HRSYLTR

SEQ ID NO: 306 - protein, 7 aa, synthetic construct
RSDNLRE

SEQ ID NO: 307 - protein, 7 aa, synthetic construct
DRSSRTK

SEQ ID NO: 308 - protein, 7 aa, synthetic construct
RSDTLSA

SEQ ID NO: 309 - protein, 7 aa, synthetic construct
DNATRTK

SEQ ID NO: 310 - protein, 7 aa, synthetic construct
DRSALRR

SEQ ID NO: 311 - protein, 7 aa, synthetic construct
DRQHLSR

SEQ ID NO: 312 - protein, 7 aa, synthetic construct
QSGDLAR

SEQ ID NO: 313 - protein, 7 aa, synthetic construct
RSDALSA

SEQ ID NO: 314 - protein, 7 aa, synthetic construct
DRRDRTK

SEQ ID NO: 315 - protein, 7 aa, synthetic construct
DNSTRIK

SEQ ID NO: 316 - protein, 7 aa, synthetic construct
RSDSLSE

SEQ ID NO: 317 - protein, 7 aa, synthetic construct
RSASRIN

SEQ ID NO: 318 - protein, 7 aa, synthetic construct
RSDDLSA

SEQ ID NO: 319 - protein, 7 aa, synthetic construct
DRSHRTK

SEQ ID NO: 320 - protein, 7 aa, synthetic construct
RSDARKA

SEQ ID NO: 321 - protein, 7 aa, synthetic construct
QSGNRKA

SEQ ID NO: 322 - DNA, 20 bp, synthetic construct
  source 1..20: mol_type = other DNA
ggaagcagatcacttacccg

SEQ ID NO: 323 - DNA, 20 bp, synthetic construct
  source 1..20: mol_type = other DNA
gtagatagcccacgaagaga

SEQ ID NO: 324 - DNA, 20 bp, synthetic construct
  source 1..20: mol_type = other DNA
gcccaggaccaagtagcccc

SEQ ID NO: 325 - DNA, 20 bp, synthetic construct
  source 1..20: mol_type = other DNA
gccctagcgaagaacaaccc

SEQ ID NO: 326 - DNA, 20 bp, synthetic construct
  source 1..20: mol_type = other DNA
agatagcccacgaagagagg

SEQ ID NO: 327 - DNA, 20 bp, synthetic construct
  source 1..20: mol_type = other DNA
acgaaactgaggacagaaag

SEQ ID NO: 328 - DNA, 20 bp, synthetic construct
  source 1..20: mol_type = other DNA
ggggaatgtagacccaaccc

SEQ ID NO: 329 - DNA, 20 bp, synthetic construct
  source 1..20: mol_type = other DNA
tagatagcccacgaagagag

SEQ ID NO: 330 - DNA, 20 bp, synthetic construct
  source 1..20: mol_type = other DNA
ctcagtggcctcaagccagg

SEQ ID NO: 331 - DNA, 20 bp, synthetic construct
  source 1..20: mol_type = other DNA
aactccacataccagatgag

SEQ ID NO: 332 - DNA, 20 bp, synthetic construct
  source 1..20: mol_type = other DNA
agtagatagcccacgaagag

SEQ ID NO: 333 - DNA, 20 bp, synthetic construct
  source 1..20: mol_type = other DNA
gtgccagaggaataaagccc

SEQ ID NO: 334 - DNA, 20 bp, synthetic construct
  source 1..20: mol_type = other DNA
ctcacccaccaccttgactg

SEQ ID NO: 335 - DNA, 20 bp, synthetic construct
  source 1..20: mol_type = other DNA
agtcagagccaagccagcga

SEQ ID NO: 336 - DNA, 20 bp, synthetic construct
  source 1..20: mol_type = other DNA
agtcttctgaatggtcccag

SEQ ID NO: 337 - DNA, 20 bp, synthetic construct
  source 1..20: mol_type = other DNA
gccgcccactgacagcgcga

SEQ ID NO: 338 - DNA, 20 bp, synthetic construct
  source 1..20: mol_type = other DNA
gagtcagagccaagccagcg

SEQ ID NO: 339 - DNA, 20 bp, synthetic construct
  source 1..20: mol_type = other DNA
ggccactgaggctgtcagaa

SEQ ID NO: 340 - DNA, 20 bp, synthetic construct
  source 1..20: mol_type = other DNA
actgacagcgcgatggcccc

SEQ ID NO: 341 - DNA, 20 bp, synthetic construct
  source 1..20: mol_type = other DNA
agagccaagccagcgaggga

SEQ ID NO: 342 - DNA, 20 bp, synthetic construct
  source 1..20: mol_type = other DNA
cctgccaggacccacccccc

SEQ ID NO: 343 - DNA, 20 bp, synthetic construct
  source 1..20: mol_type = other DNA
gtccatcttgaggtagacgc

SEQ ID NO: 344 - DNA, 20 bp, synthetic construct
  source 1..20: mol_type = other DNA
gctagcctgtggtttcccag

SEQ ID NO: 345 - DNA, 20 bp, synthetic construct
  source 1..20: mol_type = other DNA
aggccactgaggctgtcaga

SEQ ID NO: 346 - DNA, 20 bp, synthetic construct
  source 1..20: mol_type = other DNA
ggcagcgggcaacgcaggca

SEQ ID NO: 347 - DNA, 20 bp, synthetic construct
  source 1..20: mol_type = other DNA
caaaggtgatgggtctgagc

SEQ ID NO: 348 - DNA, 20 bp, synthetic construct
  source 1..20: mol_type = other DNA
gcctcagtggcctcaagcca

SEQ ID NO: 349 - DNA, 20 bp, synthetic construct
  source 1..20: mol_type = other DNA
tgcagaagtgcccaatgccc

SEQ ID NO: 350 - DNA, 20 bp, synthetic construct
  source 1..20: mol_type = other DNA
ggctagcctgtggtttccca

SEQ ID NO: 351 - DNA, 20 bp, synthetic construct
  source 1..20: mol_type = other DNA
gcacctctgggcagccaatg

SEQ ID NO: 352 - DNA, 20 bp, synthetic construct
  source 1..20: mol_type = other DNA
cagggagacaagtttgcctg

SEQ ID NO: 353 - DNA, 20 bp, synthetic construct
  source 1..20: mol_type = other DNA
tcagttgatccctccctcgc

SEQ ID NO: 354 - DNA, 20 bp, synthetic construct
  source 1..20: mol_type = other DNA
ggacccacccccctggcttg

SEQ ID NO: 355 - DNA, 20 bp, synthetic construct
  source 1..20: mol_type = other DNA
attggatgaagccttcgagc

SEQ ID NO: 356 - DNA, 20 bp, synthetic construct
  source 1..20: mol_type = other DNA
cagtcttctgaatggtccca

SEQ ID NO: 357 - DNA, 20 bp, synthetic construct
  source 1..20: mol_type = other DNA
tgaggctgtcagaagggagg

SEQ ID NO: 358 - DNA, 20 bp, synthetic construct
  source 1..20: mol_type = other DNA
tagcgaagaacaacccgggt

SEQ ID NO: 359 - DNA, 20 bp, synthetic construct
  source 1..20: mol_type = other DNA
agaaagagtctgtcaaggct

SEQ ID NO: 360 - DNA, 20 bp, synthetic construct
  source 1..20: mol_type = other DNA
tcagtggcctcaagccaggg

SEQ ID NO: 361 - DNA, 20 bp, synthetic construct
  source 1..20: mol_type = other DNA
tgaactccacataccagatg

SEQ ID NO: 362 - DNA, 20 bp, synthetic construct
  source 1..20: mol_type = other DNA
gacatagaaagagtctgtca

SEQ ID NO: 363 - DNA, 20 bp, synthetic construct
  source 1..20: mol_type = other DNA
agcctcagtggcctcaagcc

SEQ ID NO: 364 - DNA, 20 bp, synthetic construct
  source 1..20: mol_type = other DNA
gccatcgcgctgtcagtggg

SEQ ID NO: 365 - DNA, 20 bp, synthetic construct
  source 1..20: mol_type = other DNA
acagtcttctgaatggtccc

SEQ ID NO: 366 - DNA, 20 bp, synthetic construct
  source 1..20: mol_type = other DNA
cctcagtggcctcaagccag

SEQ ID NO: 367 - DNA, 20 bp, synthetic construct
  source 1..20: mol_type = other DNA
accctggcagcgggcaacgc

SEQ ID NO: 368 - DNA, 20 bp, synthetic construct
  source 1..20: mol_type = other DNA
cgggtggacagtcttctgaa

SEQ ID NO: 369 - DNA, 20 bp, synthetic construct
  source 1..20: mol_type = other DNA
gtgggtcctggcaggaggga

SEQ ID NO: 370 - DNA, 20 bp, synthetic construct
  source 1..20: mol_type = other DNA
ggggccatcgcgctgtcagt

SEQ ID NO: 371 - DNA, 20 bp, synthetic construct
  source 1..20: mol_type = other DNA
tctatgtctccctcttcccc

SEQ ID NO: 372 - DNA, 20 bp, synthetic construct
  source 1..20: mol_type = other DNA
cactgaggctgtcagaaggg

SEQ ID NO: 373 - DNA, 20 bp, synthetic construct
  source 1..20: mol_type = other DNA
tccggctccttcaagatccg

SEQ ID NO: 374 - DNA, 20 bp, synthetic construct
  source 1..20: mol_type = other DNA
cctcagccccctctcttcgt

SEQ ID NO: 375 - DNA, 20 bp, synthetic construct
  source 1..20: mol_type = other DNA
cccctggcttgaggccactg

SEQ ID NO: 376 - DNA, 20 bp, synthetic construct
  source 1..20: mol_type = other DNA
actccacataccagatgagg

SEQ ID NO: 377 - DNA, 20 bp, synthetic construct
  source 1..20: mol_type = other DNA
tgtggtttcccaggggaaga

SEQ ID NO: 378 - DNA, 20 bp, synthetic construct
  source 1..20: mol_type = other DNA
tgggttgtgggaaaggcagg

SEQ ID NO: 379 - DNA, 20 bp, synthetic construct
  source 1..20: mol_type = other DNA
gctctttcacgatggaagcg

SEQ ID NO: 380 - DNA, 20 bp, synthetic construct
  source 1..20: mol_type = other DNA
tcctcagccccctctcttcg

SEQ ID NO: 381 - DNA, 20 bp, synthetic construct
  source 1..20: mol_type = other DNA
ctcccttctgacagcctcag

SEQ ID NO: 382 - DNA, 20 bp, synthetic construct
  source 1..20: mol_type = other DNA
tgctgtgtggagtggtccag

SEQ ID NO: 383 - DNA, 20 bp, synthetic construct
  source 1..20: mol_type = other DNA
gagagggggctgaggacggg

SEQ ID NO: 384 - DNA, 20 bp, synthetic construct
  source 1..20: mol_type = other DNA
gtcagaagggaggtggaatc

SEQ ID NO: 385 - DNA, 20 bp, synthetic construct
  source 1..20: mol_type = other DNA
ggccctagcgaagaacaacc

SEQ ID NO: 386 - DNA, 20 bp, synthetic construct
  source 1..20: mol_type = other DNA
cagagccaagccagcgaggg

SEQ ID NO: 387 - DNA, 20 bp, synthetic construct
  source 1..20: mol_type = other DNA
agaaaggggaagtggaaaga

SEQ ID NO: 388 - DNA, 20 bp, synthetic construct
  source 1..20: mol_type = other DNA
agacactgtgggaaaagccg

SEQ ID NO: 389 - DNA, 20 bp, synthetic construct
  source 1..20: mol_type = other DNA
gaagagagggggctgaggac

SEQ ID NO: 390 - DNA, 20 bp, synthetic construct
  source 1..20: mol_type = other DNA
gggtcctggcaggagggagg

SEQ ID NO: 391 - DNA, 20 bp, synthetic construct
  source 1..20: mol_type = other DNA
aaagtctccatggcgatgac

SEQ ID NO: 392 - DNA, 20 bp, synthetic construct
  source 1..20: mol_type = other DNA
cgaagagagggggctgagga

SEQ ID NO: 393 - DNA, 20 bp, synthetic construct
  source 1..20: mol_type = other DNA
cagaaaggggaagtggaaag

SEQ ID NO: 394 - DNA, 20 bp, synthetic construct
  source 1..20: mol_type = other DNA
ggtgctgggcaccacgatgg

SEQ ID NO: 395 - DNA, 20 bp, synthetic construct
  source 1..20: mol_type = other DNA
cccacgaagagagggggctg

SEQ ID NO: 396 - DNA, 20 bp, synthetic construct
  source 1..20: mol_type = other DNA
aaggggaagtggaaagaggg

SEQ ID NO: 397 - DNA, 20 bp, synthetic construct
  source 1..20: mol_type = other DNA
acagacagcacgatagcccc

SEQ ID NO: 398 - DNA, 20 bp, synthetic construct
  source 1..20: mol_type = other DNA
tctagatcaggatgtcgctg

SEQ ID NO: 399 - DNA, 20 bp, synthetic construct
  source 1..20: mol_type = other DNA
actacgtgtccagcaactgc

SEQ ID NO: 400 - DNA, 20 bp, synthetic construct
  source 1..20: mol_type = other DNA
ggtgttgggcacaacgatag

SEQ ID NO: 401 - DNA, 20 bp, synthetic construct
  source 1..20: mol_type = other DNA
gatgtcgctggggtggcatg

SEQ ID NO: 402 - DNA, 20 bp, synthetic construct
  source 1..20: mol_type = other DNA
gttattctgagactgacccc

SEQ ID NO: 403 - DNA, 20 bp, synthetic construct
  source 1..20: mol_type = other DNA
gctccaacttaccagatgag

SEQ ID NO: 404 - DNA, 20 bp, synthetic construct
  source 1..20: mol_type = other DNA
gtccctgactgttaaccttg

SEQ ID NO: 405 - DNA, 20 bp, synthetic construct
  source 1..20: mol_type = other DNA
ttattctgagactgaccccc

SEQ ID NO: 406 - DNA, 20 bp, synthetic construct
  source 1..20: mol_type = other DNA
gggaaatgtacacccaaccc

SEQ ID NO: 407 - DNA, 20 bp, synthetic construct
  source 1..20: mol_type = other DNA
ttctagatcaggatgtcgct

SEQ ID NO: 408 - DNA, 20 bp, synthetic construct
  source 1..20: mol_type = other DNA
gtcctgggagagtacaaagt

SEQ ID NO: 409 - DNA, 20 bp, synthetic construct
  source 1..20: mol_type = other DNA
ctctcccaccacttcaactg

SEQ ID NO: 410 - DNA, 20 bp, synthetic construct
  source 1..20: mol_type = other DNA
agatcaggatgtcgctgggg

SEQ ID NO: 411 - DNA, 20 bp, synthetic construct
  source 1..20: mol_type = other DNA
tatcgctaagaaacagccac

SEQ ID NO: 412 - DNA, 20 bp, synthetic construct
  source 1..20: mol_type = other DNA
tgggcacaacgatagtggct

SEQ ID NO: 413 - DNA, 20 bp, synthetic construct
  source 1..20: mol_type = other DNA
gtaaaatgggacgaagtggt

SEQ ID NO: 414 - DNA, 20 bp, synthetic construct
  source 1..20: mol_type = other DNA
cagggggcggagcttgcttg

SEQ ID NO: 415 - DNA, 20 bp, synthetic construct
  source 1..20: mol_type = other DNA
tcaggctccttcaagatccg

SEQ ID NO: 416 - DNA, 20 bp, synthetic construct
  source 1..20: mol_type = other DNA
ctgtccctgactgttaacct

SEQ ID NO: 417 - DNA, 20 bp, synthetic construct
  source 1..20: mol_type = other DNA
gggcggagcttgcttggggt

SEQ ID NO: 418 - DNA, 20 bp, synthetic construct
  source 1..20: mol_type = other DNA
tccactgacagctggcaacg

SEQ ID NO: 419 - DNA, 20 bp, synthetic construct
  source 1..20: mol_type = other DNA
tgccaactttgtactctccc

SEQ ID NO: 420 - DNA, 20 bp, synthetic construct
  source 1..20: mol_type = other DNA
gggcctcattggctacccgg

SEQ ID NO: 421 - DNA, 20 bp, synthetic construct
  source 1..20: mol_type = other DNA
attgagcggctcaagaatga

SEQ ID NO: 422 - DNA, 20 bp, synthetic construct
  source 1..20: mol_type = other DNA
ggccacatacccggcacagg

SEQ ID NO: 423 - DNA, 20 bp, synthetic construct
  source 1..20: mol_type = other DNA
aagagcccttgcctggaccc

SEQ ID NO: 424 - DNA, 20 bp, synthetic construct
  source 1..20: mol_type = other DNA
cgaccgcctacgctgccagg

SEQ ID NO: 425 - DNA, 20 bp, synthetic construct
  source 1..20: mol_type = other DNA
tgactgttaaccttggggag

SEQ ID NO: 426 - DNA, 20 bp, synthetic construct
  source 1..20: mol_type = other DNA
cagccctgagaataaagctc

SEQ ID NO: 427 - DNA, 20 bp, synthetic construct
  source 1..20: mol_type = other DNA
cacagacagcacgatagccc

SEQ ID NO: 428 - DNA, 20 bp, synthetic construct
  source 1..20: mol_type = other DNA
tgtccctgactgttaacctt

SEQ ID NO: 429 - DNA, 20 bp, synthetic construct
  source 1..20: mol_type = other DNA
gtgccgcatgccctcctccg

SEQ ID NO: 430 - DNA, 20 bp, synthetic construct
  source 1..20: mol_type = other DNA
gatgagagggtcatcaaagg

SEQ ID NO: 431 - DNA, 20 bp, synthetic construct
  source 1..20: mol_type = other DNA
ccagcttcagcctctcccca

SEQ ID NO: 432 - DNA, 20 bp, synthetic construct
  source 1..20: mol_type = other DNA
gagcccttgcctggacccgg

SEQ ID NO: 433 - DNA, 20 bp, synthetic construct
  source 1..20: mol_type = other DNA
aggcgccacagttgaagtgg

SEQ ID NO: 434 - DNA, 20 bp, synthetic construct
  source 1..20: mol_type = other DNA
gggggccacatacccggcac

SEQ ID NO: 435 - DNA, 20 bp, synthetic construct
  source 1..20: mol_type = other DNA
gagcctccgggtagccaatg

SEQ ID NO: 436 - DNA, 20 bp, synthetic construct
  source 1..20: mol_type = other DNA
gcccagcctcctggcagcgt

SEQ ID NO: 437 - DNA, 20 bp, synthetic construct
  source 1..20: mol_type = other DNA
agctggacagagcgagcgag

SEQ ID NO: 438 - DNA, 20 bp, synthetic construct
  source 1..20: mol_type = other DNA
tgacccccgggtccaggcaa

SEQ ID NO: 439 - DNA, 20 bp, synthetic construct
  source 1..20: mol_type = other DNA
cacctgccctcaccattgag

SEQ ID NO: 440 - DNA, 20 bp, synthetic construct
  source 1..20: mol_type = other DNA
tggggagaggctgaagctgg

SEQ ID NO: 441 - DNA, 20 bp, synthetic construct
  source 1..20: mol_type = other DNA
cggccacggaggagggcatg

SEQ ID NO: 442 - DNA, 20 bp, synthetic construct
  source 1..20: mol_type = other DNA
gacgctgagcgccaagcccg

SEQ ID NO: 443 - DNA, 20 bp, synthetic construct
  source 1..20: mol_type = other DNA
ttggggagaggctgaagctg

SEQ ID NO: 444 - DNA, 20 bp, synthetic construct
  source 1..20: mol_type = other DNA
cagccccggcctggaccagg

SEQ ID NO: 445 - DNA, 20 bp, synthetic construct
  source 1..20: mol_type = other DNA
tgctgtgcggagtggtccag

SEQ ID NO: 446 - DNA, 20 bp, synthetic construct
  source 1..20: mol_type = other DNA
gaagctgggggccacatacc

SEQ ID NO: 447 - DNA, 20 bp, synthetic construct
  source 1..20: mol_type = other DNA
ggctgggcggccacggagga

SEQ ID NO: 448 - DNA, 20 bp, synthetic construct
  source 1..20: mol_type = other DNA
tgaaggcgccacagttgaag

SEQ ID NO: 449 - DNA, 20 bp, synthetic construct
  source 1..20: mol_type = other DNA
gtagattgtgtctgtgtcct

SEQ ID NO: 450 - DNA, 20 bp, synthetic construct
  source 1..20: mol_type = other DNA
gaagagcccttgcctggacc

SEQ ID NO: 451 - DNA, 20 bp, synthetic construct
  source 1..20: mol_type = other DNA
cagcctcctggcagcgtagg

SEQ ID NO: 452 - DNA, 20 bp, synthetic construct
  source 1..20: mol_type = other DNA
atacccggcacaggaggagc

SEQ ID NO: 453 - DNA, 20 bp, synthetic construct
  source 1..20: mol_type = other DNA
tgggagagtacaaagttggc

SEQ ID NO: 454 - DNA, 20 bp, synthetic construct
  source 1..20: mol_type = other DNA
gctatcgtgctgtctgtggg

SEQ ID NO: 455 - DNA, 20 bp, synthetic construct
  source 1..20: mol_type = other DNA
ctcctcctgtgccgggtatg

SEQ ID NO: 456 - DNA, 20 bp, synthetic construct
  source 1..20: mol_type = other DNA
agagcccttgcctggacccg

SEQ ID NO: 457 - DNA, 20 bp, synthetic construct
  source 1..20: mol_type = other DNA
ggggctatcgtgctgtctgt

SEQ ID NO: 458 - DNA, 20 bp, synthetic construct
  source 1..20: mol_type = other DNA
ctgtccagctcctcctgtgc

SEQ ID NO: 459 - DNA, 20 bp, synthetic construct
  source 1..20: mol_type = other DNA
ccagccccggcctggaccag

SEQ ID NO: 460 - DNA, 20 bp, synthetic construct
  source 1..20: mol_type = other DNA
ggcgccacagttgaagtggt

SEQ ID NO: 461 - DNA, 20 bp, synthetic construct
  source 1..20: mol_type = other DNA
tgtccagctcctcctgtgcc

SEQ ID NO: 462 - DNA, 20 bp, synthetic construct
  source 1..20: mol_type = other DNA
cgggtagccaatgaggcccc

SEQ ID NO: 463 - DNA, 20 bp, synthetic construct
  source 1..20: mol_type = other DNA
agatgagagggtcatcaaag

SEQ ID NO: 464 - DNA, 20 bp, synthetic construct
  source 1..20: mol_type = other DNA
cttggggagaggctgaagct

SEQ ID NO: 465 - DNA, 20 bp, synthetic construct
  source 1..20: mol_type = other DNA
gctgccatgtggctagcctg

SEQ ID NO: 466 - DNA, 20 bp, synthetic construct
  source 1..20: mol_type = other DNA
gctctttcacaagggaagtg

SEQ ID NO: 467 - DNA, 20 bp, synthetic construct
  source 1..20: mol_type = other DNA
agtagattgtgtctgtgtcc

SEQ ID NO: 468 - DNA, 20 bp, synthetic construct
  source 1..20: mol_type = other DNA
caagctccgccccctggtcc

SEQ ID NO: 469 - DNA, 20 bp, synthetic construct
  source 1..20: mol_type = other DNA
aaggtctccatggcgatgat

SEQ ID NO: 470 - DNA, 20 bp, synthetic construct
  source 1..20: mol_type = other DNA
ccttggggagaggctgaagc

SEQ ID NO: 471 - DNA, 20 bp, synthetic construct
  source 1..20: mol_type = other DNA
tggtccaggccggggctggg

SEQ ID NO: 472 - DNA, 20 bp, synthetic construct
  source 1..20: mol_type = other DNA
gggagatgtacacccaacct

SEQ ID NO: 473 - DNA, 20 bp, synthetic construct
  source 1..20: mol_type = other DNA
gagggccacatacccggcac

SEQ ID NO: 474 - DNA, 20 bp, synthetic construct
  source 1..20: mol_type = other DNA
aggcctataggactcgtggg

SEQ ID NO: 475 - DNA, 20 bp, synthetic construct
  source 1..20: mol_type = other DNA
ggtgcttggcacaacaatag

SEQ ID NO: 476 - DNA, 20 bp, synthetic construct
  source 1..20: mol_type = other DNA
agatcaggacgtcgccgggg

SEQ ID NO: 477 - DNA, 20 bp, synthetic construct
  source 1..20: mol_type = other DNA
agtggtgcagcatggaccag

SEQ ID NO: 478 - DNA, 20 bp, synthetic construct
  source 1..20: mol_type = other DNA
agatgagagggtcatcgaag

SEQ ID NO: 479 - DNA, 20 bp, synthetic construct
  source 1..20: mol_type = other DNA
ggccaagtcacacgccaccc

SEQ ID NO: 480 - DNA, 20 bp, synthetic construct
  source 1..20: mol_type = other DNA
gcccggagaataaagccctg

SEQ ID NO: 481 - DNA, 20 bp, synthetic construct
  source 1..20: mol_type = other DNA
tctagatcaggacgtcgccg

SEQ ID NO: 482 - DNA, 20 bp, synthetic construct
  source 1..20: mol_type = other DNA
aagtggtgcagcatggacca

SEQ ID NO: 483 - DNA, 20 bp, synthetic construct
  source 1..20: mol_type = other DNA
gatgagagggtcatcgaagg

SEQ ID NO: 484 - DNA, 20 bp, synthetic construct
  source 1..20: mol_type = other DNA
ccggcgacgtcctgatctag

SEQ ID NO: 485 - DNA, 20 bp, synthetic construct
  source 1..20: mol_type = other DNA
aaagttggcaggacacccca

SEQ ID NO: 486 - DNA, 20 bp, synthetic construct
  source 1..20: mol_type = other DNA
cctctagatcaggacgtcgc

SEQ ID NO: 487 - DNA, 20 bp, synthetic construct
  source 1..20: mol_type = other DNA
gtggtgcagcatggaccagg

SEQ ID NO: 488 - DNA, 20 bp, synthetic construct
  source 1..20: mol_type = other DNA
tccccacagaaggcaaaaca

SEQ ID NO: 489 - DNA, 20 bp, synthetic construct
  source 1..20: mol_type = other DNA
atgataggaccctctagatc

SEQ ID NO: 490 - DNA, 20 bp, synthetic construct
  source 1..20: mol_type = other DNA
cgaaaggcctataggactcg

SEQ ID NO: 491 - DNA, 20 bp, synthetic construct
  source 1..20: mol_type = other DNA
attgagcggctgaagaacga

SEQ ID NO: 492 - DNA, 20 bp, synthetic construct
  source 1..20: mol_type = other DNA
cggcgacgtcctgatctaga

SEQ ID NO: 493 - DNA, 20 bp, synthetic construct
  source 1..20: mol_type = other DNA
gaaaggcctataggactcgt

SEQ ID NO: 494 - DNA, 20 bp, synthetic construct
  source 1..20: mol_type = other DNA
tccactgacagcgggcaacg

SEQ ID NO: 495 - DNA, 20 bp, synthetic construct
  source 1..20: mol_type = other DNA
tgctctggctgtcaatcttg

SEQ ID NO: 496 - DNA, 20 bp, synthetic construct
  source 1..20: mol_type = other DNA
caagtggtgcagcatggacc

SEQ ID NO: 497 - DNA, 20 bp, synthetic construct
  source 1..20: mol_type = other DNA
tgacggcagcgtggaagctg

SEQ ID NO: 498 - DNA, 20 bp, synthetic construct
  source 1..20: mol_type = other DNA
ctggacagagcgagcgagaa

SEQ ID NO: 499 - DNA, 20 bp, synthetic construct
  source 1..20: mol_type = other DNA
agcccggagaataaagccct

SEQ ID NO: 500 - DNA, 20 bp, synthetic construct
  source 1..20: mol_type = other DNA
tctggctccttcaagatccg

SEQ ID NO: 501 - DNA, 20 bp, synthetic construct
  source 1..20: mol_type = other DNA
ctctagatcaggacgtcgcc

SEQ ID NO: 502 - DNA, 20 bp, synthetic construct
  source 1..20: mol_type = other DNA
gaaagttggcaggacacccc

SEQ ID NO: 503 - DNA, 20 bp, synthetic construct
  source 1..20: mol_type = other DNA
gctctggaaaagaacaaccc

SEQ ID NO: 504 - DNA, 20 bp, synthetic construct
  source 1..20: mol_type = other DNA
agagaaggtgaggcggacct

SEQ ID NO: 505 - DNA, 20 bp, synthetic construct
  source 1..20: mol_type = other DNA
tggggtgagactgaggctga

SEQ ID NO: 506 - DNA, 20 bp, synthetic construct
  source 1..20: mol_type = other DNA
gcagaacaagtggtgcagca

SEQ ID NO: 507 - DNA, 20 bp, synthetic construct
  source 1..20: mol_type = other DNA
gacactgagcgccaagcccg

SEQ ID NO: 508 - DNA, 20 bp, synthetic construct
  source 1..20: mol_type = other DNA
tcaatcttggggtgagactg

SEQ ID NO: 509 - DNA, 20 bp, synthetic construct
  source 1..20: mol_type = other DNA
aaggtgaggcggaccttgga

SEQ ID NO: 510 - DNA, 20 bp, synthetic construct
  source 1..20: mol_type = other DNA
gctggacagagcgagcgaga

SEQ ID NO: 511 - DNA, 20 bp, synthetic construct
  source 1..20: mol_type = other DNA
gagcctccgggcagccaatg

SEQ ID NO: 512 - DNA, 20 bp, synthetic construct
  source 1..20: mol_type = other DNA
cttaaggaacacactagtgc

SEQ ID NO: 513 - DNA, 20 bp, synthetic construct
  source 1..20: mol_type = other DNA
cctgctctggctgtcaatct

SEQ ID NO: 514 - DNA, 20 bp, synthetic construct
  source 1..20: mol_type = other DNA
ggccctcattggctgcccgg

SEQ ID NO: 515 - DNA, 20 bp, synthetic construct
  source 1..20: mol_type = other DNA
ggaaaagaacaacccaggtt

SEQ ID NO: 516 - DNA, 20 bp, synthetic construct
  source 1..20: mol_type = other DNA
atttgactcatgcgagcact

SEQ ID NO: 517 - DNA, 20 bp, synthetic construct
  source 1..20: mol_type = other DNA
cagggggcggggatttctgg

SEQ ID NO: 518 - DNA, 20 bp, synthetic construct
  source 1..20: mol_type = other DNA
ttggcacaacaatagtggct

SEQ ID NO: 519 - DNA, 20 bp, synthetic construct
  source 1..20: mol_type = other DNA
ctgctctggctgtcaatctt

SEQ ID NO: 520 - DNA, 20 bp, synthetic construct
  source 1..20: mol_type = other DNA
accacttgttctgccctcca

SEQ ID NO: 521 - DNA, 20 bp, synthetic construct
  source 1..20: mol_type = other DNA
tgagcggctgaagaacgaag

SEQ ID NO: 522 - DNA, 20 bp, synthetic construct
  source 1..20: mol_type = other DNA
ccagggggcggggatttctg

SEQ ID NO: 523 - DNA, 20 bp, synthetic construct
  source 1..20: mol_type = other DNA
cttcagccgctcaatggtga

SEQ ID NO: 524 - DNA, 20 bp, synthetic construct
  source 1..20: mol_type = other DNA
ccaagattgacagccagagc

SEQ ID NO: 525 - DNA, 20 bp, synthetic construct
  source 1..20: mol_type = other DNA
accttggagggcagaacaag

SEQ ID NO: 526 - DNA, 20 bp, synthetic construct
  source 1..20: mol_type = other DNA
gacggcagcgtggaagctgt

SEQ ID NO: 527 - DNA, 20 bp, synthetic construct
  source 1..20: mol_type = other DNA
ccccagaaatccccgccccc

SEQ ID NO: 528 - DNA, 20 bp, synthetic construct
  source 1..20: mol_type = other DNA
aggccacacttcccttgtga

SEQ ID NO: 529 - DNA, 20 bp, synthetic construct
  source 1..20: mol_type = other DNA
cagcccggagaataaagccc

SEQ ID NO: 530 - DNA, 20 bp, synthetic construct
  source 1..20: mol_type = other DNA
agacactgagcgccaagccc

SEQ ID NO: 531 - DNA, 20 bp, synthetic construct
  source 1..20: mol_type = other DNA
gaggctgagggccacatacc

SEQ ID NO: 532 - DNA, 20 bp, synthetic construct
  source 1..20: mol_type = other DNA
gtcctgggagagtggaaagt

SEQ ID NO: 533 - DNA, 20 bp, synthetic construct
  source 1..20: mol_type = other DNA
cagatgagagggtcatcgaa

SEQ ID NO: 534 - DNA, 20 bp, synthetic construct
  source 1..20: mol_type = other DNA
aagaacggggagagaaggtg

SEQ ID NO: 535 - DNA, 20 bp, synthetic construct
  source 1..20: mol_type = other DNA
ttccacgctgccgtcaagga

SEQ ID NO: 536 - DNA, 20 bp, synthetic construct
  source 1..20: mol_type = other DNA
ttggggtgagactgaggctg

SEQ ID NO: 537 - DNA, 20 bp, synthetic construct
  source 1..20: mol_type = other DNA
agcctccgggcagccaatga

SEQ ID NO: 538 - DNA, 20 bp, synthetic construct
  source 1..20: mol_type = other DNA
gtgttcctgaaacagcttca

SEQ ID NO: 539 - DNA, 20 bp, synthetic construct
  source 1..20: mol_type = other DNA
ttggacacctctgcaagatg

SEQ ID NO: 540 - DNA, 20 bp, synthetic construct
  source 1..20: mol_type = other DNA
gctgccattgagaagttcgt

SEQ ID NO: 541 - DNA, 20 bp, synthetic construct
  source 1..20: mol_type = other DNA
tggacacctctgcaagatgt

SEQ ID NO: 542 - DNA, 20 bp, synthetic construct
  source 1..20: mol_type = other DNA
catacccacgaacttctcaa

SEQ ID NO: 543 - DNA, 20 bp, synthetic construct
  source 1..20: mol_type = other DNA
atggacagtgcccaaccttc

SEQ ID NO: 544 - DNA, 20 bp, synthetic construct
  source 1..20: mol_type = other DNA
caccacgcggctgtacacgg

SEQ ID NO: 545 - DNA, 20 bp, synthetic construct
  source 1..20: mol_type = other DNA
atagatggtcctctcacgga

SEQ ID NO: 546 - DNA, 20 bp, synthetic construct
  source 1..20: mol_type = other DNA
cccgcagacaatgaccacga

SEQ ID NO: 547 - DNA, 20 bp, synthetic construct
  source 1..20: mol_type = other DNA
ctgaccaaggcagtgaaggt

SEQ ID NO: 548 - DNA, 20 bp, synthetic construct
  source 1..20: mol_type = other DNA
ggtggcagcgtggaagctgt

SEQ ID NO: 549 - DNA, 20 bp, synthetic construct
  source 1..20: mol_type = other DNA
ctggatagatggtcctctca

SEQ ID NO: 550 - DNA, 20 bp, synthetic construct
  source 1..20: mol_type = other DNA
tgccggcagtggtggcagcg

SEQ ID NO: 551 - DNA, 20 bp, synthetic construct
  source 1..20: mol_type = other DNA
atgtctgctggatcgtccca

SEQ ID NO: 552 - DNA, 20 bp, synthetic construct
  source 1..20: mol_type = other DNA
gaccagcttgccggcagtgg

SEQ ID NO: 553 - DNA, 20 bp, synthetic construct
  source 1..20: mol_type = other DNA
ggtggtgctgggcacaacga

SEQ ID NO: 554 - DNA, 20 bp, synthetic construct
  source 1..20: mol_type = other DNA
tctccctgtcccaggttacc

SEQ ID NO: 555 - DNA, 20 bp, synthetic construct
  source 1..20: mol_type = other DNA
tgagggtgccatagtcaagg

SEQ ID NO: 556 - DNA, 20 bp, synthetic construct
  source 1..20: mol_type = other DNA
ctctgctcacctggtaacct

SEQ ID NO: 557 - DNA, 20 bp, synthetic construct
  source 1..20: mol_type = other DNA
gctgaccaaggcagtgaagg

SEQ ID NO: 558 - DNA, 20 bp, synthetic construct
  source 1..20: mol_type = other DNA
cctgtgcgtgaagactgtgg

SEQ ID NO: 559 - DNA, 20 bp, synthetic construct
  source 1..20: mol_type = other DNA
gggtgccatagtcaaggtgg

SEQ ID NO: 560 - DNA, 20 bp, synthetic construct
  source 1..20: mol_type = other DNA
agatgacttcagagaagatg

SEQ ID NO: 561 - DNA, 20 bp, synthetic construct
  source 1..20: mol_type = other DNA
ggtgccatagtcaaggtggt

SEQ ID NO: 562 - DNA, 20 bp, synthetic construct
  source 1..20: mol_type = other DNA
ctgcaccacgcggctgtaca

SEQ ID NO: 563 - DNA, 20 bp, synthetic construct
  source 1..20: mol_type = other DNA
agcgacatgctgtcacggat

SEQ ID NO: 564 - DNA, 20 bp, synthetic construct
  source 1..20: mol_type = other DNA
gcgtggtgcagaagctgcaa

SEQ ID NO: 565 - DNA, 20 bp, synthetic construct
  source 1..20: mol_type = other DNA
caccagcgtctacctcaaga

SEQ ID NO: 566 - DNA, 20 bp, synthetic construct
  source 1..20: mol_type = other DNA
gaccacgagggaggacagcg

SEQ ID NO: 567 - DNA, 20 bp, synthetic construct
  source 1..20: mol_type = other DNA
gaatgagggtgccatagtca

SEQ ID NO: 568 - DNA, 20 bp, synthetic construct
  source 1..20: mol_type = other DNA
atgaccacgagggaggacag

SEQ ID NO: 569 - DNA, 20 bp, synthetic construct
  source 1..20: mol_type = other DNA
ggtgctgggcacaacgatgg

SEQ ID NO: 570 - DNA, 20 bp, synthetic construct
  source 1..20: mol_type = other DNA
catgcccagctgttccttga

SEQ ID NO: 571 - DNA, 20 bp, synthetic construct
  source 1..20: mol_type = other DNA
tgcagaggtgtccaatgccc

SEQ ID NO: 572 - DNA, 20 bp, synthetic construct
  source 1..20: mol_type = other DNA
gtgggctccggcggtctcca

SEQ ID NO: 573 - DNA, 20 bp, synthetic construct
  source 1..20: mol_type = other DNA
ctgggcacaacgatggtggc

SEQ ID NO: 574 - DNA, 20 bp, synthetic construct
  source 1..20: mol_type = other DNA
gtggaagctgtgggctccgg

SEQ ID NO: 575 - DNA, 20 bp, synthetic construct
  source 1..20: mol_type = other DNA
cgacatgctgtcacggatgg

SEQ ID NO: 576 - DNA, 20 bp, synthetic construct
  source 1..20: mol_type = other DNA
tcacctggtaacctgggaca

SEQ ID NO: 577 - DNA, 20 bp, synthetic construct
  source 1..20: mol_type = other DNA
ggccctgtgcgtgaagactg

SEQ ID NO: 578 - DNA, 20 bp, synthetic construct
  source 1..20: mol_type = other DNA
cttcaggctccttcaagatc

SEQ ID NO: 579 - DNA, 20 bp, synthetic construct
  source 1..20: mol_type = other DNA
gccctgtgcgtgaagactgt

SEQ ID NO: 580 - DNA, 20 bp, synthetic construct
  source 1..20: mol_type = other DNA
gatggtcctctcacggatgg

SEQ ID NO: 581 - DNA, 20 bp, synthetic construct
  source 1..20: mol_type = other DNA
ccctgtgcgtgaagactgtg

SEQ ID NO: 582 - DNA, 20 bp, synthetic construct
  source 1..20: mol_type = other DNA
cttgaaggagcctgaaggtt

SEQ ID NO: 583 - DNA, 20 bp, synthetic construct
  source 1..20: mol_type = other DNA
gagatgacttcagagaagat

SEQ ID NO: 584 - DNA, 20 bp, synthetic construct
  source 1..20: mol_type = other DNA
cattgagcggctcaagaatg

SEQ ID NO: 585 - DNA, 20 bp, synthetic construct
  source 1..20: mol_type = other DNA
cgagatgacttcagagaaga

SEQ ID NO: 586 - DNA, 20 bp, synthetic construct
  source 1..20: mol_type = other DNA
tcttgaaggagcctgaaggt

SEQ ID NO: 587 - DNA, 20 bp, synthetic construct
  source 1..20: mol_type = other DNA
atctgcccagtctatccatc

SEQ ID NO: 588 - DNA, 20 bp, synthetic construct
  source 1..20: mol_type = other DNA
gtgatcaccatcgtaagtct

SEQ ID NO: 589 - DNA, 20 bp, synthetic construct
  source 1..20: mol_type = other DNA
agatggatagactgggcaga

SEQ ID NO: 590 - DNA, 20 bp, synthetic construct
  source 1..20: mol_type = other DNA
catggatgcaacaagccagg

SEQ ID NO: 591 - DNA, 20 bp, synthetic construct
  source 1..20: mol_type = other DNA
atggtccagatggatagact

SEQ ID NO: 592 - DNA, 20 bp, synthetic construct
  source 1..20: mol_type = other DNA
cctggcaagattgatttcat

SEQ ID NO: 593 - DNA, 20 bp, synthetic construct
  source 1..20: mol_type = other DNA
gatggtccagatggatagac

SEQ ID NO: 594 - DNA, 20 bp, synthetic construct
  source 1..20: mol_type = other DNA
aaaagattttgttactatgt

SEQ ID NO: 595 - DNA, 20 bp, synthetic construct
  source 1..20: mol_type = other DNA
atgactggagatggtccaga

SEQ ID NO: 596 - DNA, 20 bp, synthetic construct
  source 1..20: mol_type = other DNA
agacatggatgcaacaagcc

SEQ ID NO: 597 - DNA, 20 bp, synthetic construct
  source 1..20: mol_type = other DNA
catctccagtcatccctcct

SEQ ID NO: 598 - DNA, 20 bp, synthetic construct
  source 1..20: mol_type = other DNA
gtacacttgaaactagtata

SEQ ID NO: 599 - DNA, 20 bp, synthetic construct
  source 1..20: mol_type = other DNA
caaggagggatgactggaga

SEQ ID NO: 600 - DNA, 20 bp, synthetic construct
  source 1..20: mol_type = other DNA
catgacatttatttgataac

SEQ ID NO: 601 - DNA, 20 bp, synthetic construct
  source 1..20: mol_type = other DNA
gtaagccaaggagggatgac

SEQ ID NO: 602 - DNA, 20 bp, synthetic construct
  source 1..20: mol_type = other DNA
agatgttcctagacttacga

SEQ ID NO: 603 - DNA, 20 bp, synthetic construct
  source 1..20: mol_type = other DNA
tagagcgagtaagccaagga

SEQ ID NO: 604 - DNA, 20 bp, synthetic construct
  source 1..20: mol_type = other DNA
aaagaattctttgcttttag

SEQ ID NO: 605 - DNA, 20 bp, synthetic construct
  source 1..20: mol_type = other DNA
gtagagcgagtaagccaagg

SEQ ID NO: 606 - DNA, 20 bp, synthetic construct
  source 1..20: mol_type = other DNA
aaagattttgttactatgta

SEQ ID NO: 607 - DNA, 20 bp, synthetic construct
  source 1..20: mol_type = other DNA
tgggtagagcgagtaagcca

SEQ ID NO: 608 - DNA, 20 bp, synthetic construct
  source 1..20: mol_type = other DNA
acaggaggaaaatttaataa

SEQ ID NO: 609 - DNA, 20 bp, synthetic construct
  source 1..20: mol_type = other DNA
tgctggcaaagcgatggagc

SEQ ID NO: 610 - DNA, 20 bp, synthetic construct
  source 1..20: mol_type = other DNA
gcaaagaattctttttttcc

SEQ ID NO: 611 - DNA, 20 bp, synthetic construct
  source 1..20: mol_type = other DNA
ttgctggcaaagcgatggag

SEQ ID NO: 612 - DNA, 20 bp, synthetic construct
  source 1..20: mol_type = other DNA
gtctaaaaagagctctttgt

SEQ ID NO: 613 - DNA, 20 bp, synthetic construct
  source 1..20: mol_type = other DNA
cgtcattgctggcaaagcga

SEQ ID NO: 614 - DNA, 20 bp, synthetic construct
  source 1..20: mol_type = other DNA
atttacagatgaaataatac

SEQ ID NO: 615 - DNA, 20 bp, synthetic construct
  source 1..20: mol_type = other DNA
ggctctgtggacgtcattgc

SEQ ID NO: 616 - DNA, 20 bp, synthetic construct
  source 1..20: mol_type = other DNA
gttatcaaataaatgtcatg

SEQ ID NO: 617 - DNA, 20 bp, synthetic construct
  source 1..20: mol_type = other DNA
accggggtctgcacgtgcag

SEQ ID NO: 618 - DNA, 20 bp, synthetic construct
  source 1..20: mol_type = other DNA
tacaggaggaaaatttaata

SEQ ID NO: 619 - DNA, 20 bp, synthetic construct
  source 1..20: mol_type = other DNA
gaccggggtctgcacgtgca

SEQ ID NO: 620 - DNA, 20 bp, synthetic construct
  source 1..20: mol_type = other DNA
agttatcaaataaatgtcat

SEQ ID NO: 621 - DNA, 20 bp, synthetic construct
  source 1..20: mol_type = other DNA
tgcacgtgcagaccccggtc

SEQ ID NO: 622 - DNA, 20 bp, synthetic construct
  source 1..20: mol_type = other DNA
aaaagaattctttgctttta

SEQ ID NO: 623 - DNA, 20 bp, synthetic construct
  source 1..20: mol_type = other DNA
ggaccggggtctgcacgtgc

SEQ ID NO: 624 - DNA, 20 bp, synthetic construct
  source 1..20: mol_type = other DNA
ccaatgaaatcaatcttgcc

SEQ ID NO: 625 - DNA, 20 bp, synthetic construct
  source 1..20: mol_type = other DNA
gcacgtgcagaccccggtcc

SEQ ID NO: 626 - DNA, 20 bp, synthetic construct
  source 1..20: mol_type = other DNA
cagttatcaaataaatgtca

SEQ ID NO: 627 - DNA, 20 bp, synthetic construct
  source 1..20: mol_type = other DNA
cagcccgtccctgtccaaag

SEQ ID NO: 628 - DNA, 20 bp, synthetic construct
  source 1..20: mol_type = other DNA
aaaaagaattctttgctttt

SEQ ID NO: 629 - DNA, 20 bp, synthetic construct
  source 1..20: mol_type = other DNA
ccgtccctgtccaaagtggc

SEQ ID NO: 630 - DNA, 20 bp, synthetic construct
  source 1..20: mol_type = other DNA
ggaggaaaatttaataaggg

SEQ ID NO: 631 - DNA, 20 bp, synthetic construct
  source 1..20: mol_type = other DNA
cggccactttggacagggac

SEQ ID NO: 632 - DNA, 20 bp, synthetic construct
  source 1..20: mol_type = other DNA
aagggaggaaaaaataacag

SEQ ID NO: 633 - DNA, 20 bp, synthetic construct
  source 1..20: mol_type = other DNA
ccggccactttggacaggga

SEQ ID NO: 634 - DNA, 20 bp, synthetic construct
  source 1..20: mol_type = other DNA
aggaaaaaataacagaggaa

SEQ ID NO: 635 - DNA, 20 bp, synthetic construct
  source 1..20: mol_type = other DNA
ggtgccggccactttggaca

SEQ ID NO: 636 - DNA, 20 bp, synthetic construct
  source 1..20: mol_type = other DNA
gaggaaaaaataacagagga

SEQ ID NO: 637 - DNA, 20 bp, synthetic construct
  source 1..20: mol_type = other DNA
ctggagagcccctgcacgtg

SEQ ID NO: 638 - DNA, 20 bp, synthetic construct
  source 1..20: mol_type = other DNA
catgtccctgtccaaagtgg

SEQ ID NO: 639 - DNA, 20 bp, synthetic construct
  source 1..20: mol_type = other DNA
ggacatgctgtcacggacgg

SEQ ID NO: 640 - DNA, 20 bp, synthetic construct
  source 1..20: mol_type = other DNA
ttccatcgtggaagaactga

SEQ ID NO: 641 - DNA, 20 bp, synthetic construct
  source 1..20: mol_type = other DNA
gggacatgctgtcacggacg

SEQ ID NO: 642 - DNA, 20 bp, synthetic construct
  source 1..20: mol_type = other DNA
caaggtggtgggtgagatgt

SEQ ID NO: 643 - DNA, 20 bp, synthetic construct
  source 1..20: mol_type = other DNA
agggacatgctgtcacggac

SEQ ID NO: 644 - DNA, 20 bp, synthetic construct
  source 1..20: mol_type = other DNA
agacgctgagtgcaaagccg

SEQ ID NO: 645 - DNA, 20 bp, synthetic construct
  source 1..20: mol_type = other DNA
cagggacatgctgtcacgga

SEQ ID NO: 646 - DNA, 20 bp, synthetic construct
  source 1..20: mol_type = other DNA
gacgctgagtgcaaagccgg

SEQ ID NO: 647 - DNA, 20 bp, synthetic construct
  source 1..20: mol_type = other DNA
cagcatgtccctgtccaaag

SEQ ID NO: 648 - DNA, 20 bp, synthetic construct
  source 1..20: mol_type = other DNA
gttggatgaggcctttgagc

SEQ ID NO: 649 - DNA, 20 bp, synthetic construct
  source 1..20: mol_type = other DNA
atggacagcgtccaaccttc

SEQ ID NO: 650 - DNA, 20 bp, synthetic construct
  source 1..20: mol_type = other DNA
atgtccctgtccaaagtggc

SEQ ID NO: 651 - DNA, 20 bp, synthetic construct
  source 1..20: mol_type = other DNA
ctccttcagttcttccacga

SEQ ID NO: 652 - DNA, 20 bp, synthetic construct
  source 1..20: mol_type = other DNA
ggtgcccgccactttggaca

SEQ ID NO: 653 - DNA, 20 bp, synthetic construct
  source 1..20: mol_type = other DNA
ctttgagctggccaaggccc

SEQ ID NO: 654 - DNA, 20 bp, synthetic construct
  source 1..20: mol_type = other DNA
tggtgcccgccactttggac

SEQ ID NO: 655 - DNA, 20 bp, synthetic construct
  source 1..20: mol_type = other DNA
cacctgccctcaccatcgag

SEQ ID NO: 656 - DNA, 20 bp, synthetic construct
  source 1..20: mol_type = other DNA
caccagcgtctatctcaaga

SEQ ID NO: 657 - DNA, 20 bp, synthetic construct
  source 1..20: mol_type = other DNA
gagacgctgagtgcaaagcc

SEQ ID NO: 658 - DNA, 20 bp, synthetic construct
  source 1..20: mol_type = other DNA
gtccatcttgagatagacgc

SEQ ID NO: 659 - DNA, 20 bp, synthetic construct
  source 1..20: mol_type = other DNA
ggagacgctgagtgcaaagc

SEQ ID NO: 660 - DNA, 20 bp, synthetic construct
  source 1..20: mol_type = other DNA
cctttgacgaccccctcatc

SEQ ID NO: 661 - DNA, 20 bp, synthetic construct
  source 1..20: mol_type = other DNA
ttcaggctccttcaagatcc

SEQ ID NO: 662 - DNA, 20 bp, synthetic construct
  source 1..20: mol_type = other DNA
gacgaccccctcatctggga

SEQ ID NO: 663 - DNA, 20 bp, synthetic construct
  source 1..20: mol_type = other DNA
cggatcttgaaggagcctga

SEQ ID NO: 664 - DNA, 20 bp, synthetic construct
  source 1..20: mol_type = other DNA
ctttgacgaccccctcatct

SEQ ID NO: 665 - DNA, 20 bp, synthetic construct
  source 1..20: mol_type = other DNA
tgtggccttcccagatgagg

SEQ ID NO: 666 - DNA, 20 bp, synthetic construct
  source 1..20: mol_type = other DNA
tccttcaagatccggggcat

SEQ ID NO: 667 - DNA, 20 bp, synthetic construct
  source 1..20: mol_type = other DNA
ggggcatcggacatctctgc

SEQ ID NO: 668 - DNA, 20 bp, synthetic construct
  source 1..20: mol_type = other DNA
tcggacatctctgcaggatg

SEQ ID NO: 669 - DNA, 20 bp, synthetic construct
  source 1..20: mol_type = other DNA
tgaacatttcgtctgctcct

SEQ ID NO: 670 - DNA, 20 bp, synthetic construct
  source 1..20: mol_type = other DNA
acatttcgtctgctcctcgg

SEQ ID NO: 671 - DNA, 20 bp, synthetic construct
  source 1..20: mol_type = other DNA
catttcgtctgctcctcggc

SEQ ID NO: 672 - DNA, 20 bp, synthetic construct
  source 1..20: mol_type = other DNA
ggaaggccacacttccatcg

SEQ ID NO: 673 - DNA, 20 bp, synthetic construct
  source 1..20: mol_type = other DNA
agggatgcccagcttcctgg

SEQ ID NO: 674 - DNA, 20 bp, synthetic construct
  source 1..20: mol_type = other DNA
gaggaatgtagacccagcct

SEQ ID NO: 675 - DNA, 21 bp, synthetic construct
  source 1..21: mol_type = other DNA
caagcaaggctgtgcacattt

SEQ ID NO: 676 - DNA, 21 bp, synthetic construct
  source 1..21: mol_type = other DNA
tgcaggttaacttcttgttat

SEQ ID NO: 677 - DNA, 21 bp, synthetic construct
  source 1..21: mol_type = other DNA
ccacgcttccatcgtgaaaga

SEQ ID NO: 678 - DNA, 21 bp, synthetic construct
  source 1..21: mol_type = other DNA
cgaggctatgaattggacctt

SEQ ID NO: 679 - DNA, 21 bp, synthetic construct
  source 1..21: mol_type = other DNA
tcaggccctgaagctgtttca

SEQ ID NO: 680 - DNA, 21 bp, synthetic construct
  source 1..21: mol_type = other DNA
ctctgaagttatctcggacca

SEQ ID NO: 681 - DNA, 21 bp, synthetic construct
  source 1..21: mol_type = other DNA
cattgggcacttctgcaagag

SEQ ID NO: 682 - DNA, 21 bp, synthetic construct
  source 1..21: mol_type = other DNA
cgctgtctatagccacgtgat

SEQ ID NO: 683 - DNA, 21 bp, synthetic construct
  source 1..21: mol_type = other DNA
gctgggcatgacaaataggtt

SEQ ID NO: 684 - DNA, 21 bp, synthetic construct
  source 1..21: mol_type = other DNA
caagatcaccagtgttgccaa

SEQ ID NO: 685 - DNA, 21 bp, synthetic construct
  source 1..21: mol_type = other DNA
gccattgagaagttcgtggat

SEQ ID NO: 686 - DNA, 21 bp, synthetic construct
  source 1..21: mol_type = other DNA
tgagttattggatgaagcctt

SEQ ID NO: 687 - DNA, 21 bp, synthetic construct
  source 1..21: mol_type = other DNA
caccgcaggcaaacttgtctc

SEQ ID NO: 688 - DNA, 21 bp, synthetic construct
  source 1..21: mol_type = other DNA
agcatggcgttgtccaaattg

SEQ ID NO: 689 - DNA, 21 bp, synthetic construct
  source 1..21: mol_type = other DNA
gccacacttcccttgtgaaag

SEQ ID NO: 690 - DNA, 11943 bp, Homo sapiens
  source 1..11943: mol_type = genomic DNA
ccacacccggctgatttttgtatttttagtagagacgggatttcaccatgttggccaggctggtttcgaattcctgacctcaggtgatccacccgcctcggcctcccaaaatgcggagccaatatttaacctttcatctgaagctatctgcttaggaactaaaggaaaggcagtttcttacatgactcagcttttagcttaattctttttcttttggcatagtgagttgggggtcccaattttttattttcctttcacgaaactgaggacagaaaggggaagtggaaagagggaggaggagtgggttgtgggaaaggcaggaggaagctgccaaagaagaaagcttggccagattccacctcccttctgacagcctcagtggcctcaagccaggggggtgggtcctggcaggagggaggggggggtgtagccagccccagggccccattggctgcccagaggtgccagaggaataaagcccaggaccaagtagcccctgggaccattcagaagactgtccacccgtcctcagccccctctcttcgtgggctatctactcagttgatccctccctcgctggcttggctctgactcctgctcagacccatcacctttgccgggtaagtggccccatcctcagtcccacctcaggactggagtgtcccgggctagcctgtggtttcccaggggaagagggagacatagaaagagtctgtcaaggctgggcatggtggctgatgcctgtaatcccaacactttggaaggctgaggtgggagggttgcttgaggccacaagttcaagactgagctgggcaacagagtgagaccccccccaatctctacaaaaaaaattttttttttttaagacagaggtctcactctgtcacccaggctgaagtgcaatggcacaatctgggctcactgcaacctccacctcctgggttcaagcgattctcctgcctcagcctcccgagtagcttggactacaggcgtgtgccaccacgcccggctaatttttgtatttttagtagagatggggtttcaccatgttggccaggctggtctcgaactcctgacctaaaaaaagaatttaattagtatagtgtggtggtgcacacctgtagtcccagctactgggaaggctgagttgggaggatcgctggagcccaagaggtcgagtctgcagtgagcaattattgcgcccctgcactccagcctgggtgacacagcaagaccttgtctcaaatttttaaaaaacagccatttgggcctaaacactgagtttgtgtccagttctgtgcctcagcttccacatctgtagaatggggatgagcatagcgccttcctcctcaggtggttgtgacggtgaaaggatgacctgcacttaaggtgtccagcccagatttcataatagttcatatcgactgagcacttactgtgaaccaagtgaaagggaaggtggcttccgactgccggagtgggcagacagcggcttcagggctccctccctcctcctccccgggcacaacctcttctgaagtcttgcgatccttggggcagggatccttggggcaggctggatgcagttaatggatgctcacttcacggaaagccgcggcagagggagccctgtccttttacctgaaggcatcccatggcaccgcctgggacaaacggcgagagaggcgccccaaacccacctgaaaagggtcttcgtggccggctcctctgactcccaccccgattgtccctgaggccaggccttggtgacctacaccggcctttaatgtcaagtcagtcggtgtggtggctcacgcctgtaatcccagcactttgggaggccaaggcaggcagatgacttgaggtcaggagttcgagaccagcctgcccaatatggtgaaaccttgtctccactaaaaatgcaaaaattattcgggcgtggtggtgcacgtctgtgatcccagctacttgggaggctgaggcaggaaaatcgcttgaacccagaaggcggaggttgcagtgagccgagatcatgccgctgcactccagcctgggtgacagagggaggttctctaaaaataactaaataaaaaccgggatcccattgtttggggattaacctccaggagctgaaagttcatgccgatttggggatgtcctgttttgaagtttgccccagtctctgatttttattcgagagatccagcggagagagggtgtctgttcaagttcccctcccaggccccgaatgtcagttaattcagatagacaaggggtgacctatttaggggatgctaagtctggcggggcagttggaatccccacccctcattctccctctgaccaagggatgtctggctcattctgggagcgggggatggatgtccaagctaggagtgcccaggacagggggctggacaaggtggcattggagatgggtctctggctagccacccttgcctggccttctgggatggtggagggagggggaaccagagagccccttggccatcgtcagctgatctcttctggcccggcccaagttgaggagaatcaaatcctccctgagactctcagcagaaggcaggggagatgggaaggctgggtgggtggacgtggctgtggctagaccccgggccaccccagctgcttcatcccccagagcccatccagaagcaggagcctgagccacacccatgggtcgcagagagagcctgagccctggagccagctgcccgagttcaaatcctgcctccctgcctcactcacagcgtgactttgggcaaattatgttaatattccccttcctgttgctgtgtaccaaactaccccaaaatgtgacttaaaacaacagcaagccaggtgctgtggctcatgcctgtaatcccagcagtttgggaggctgaggtgggcagaacgcttgaggccaggagttggagaccagcctggccaacatagcaaaaccccatctctactggaatgaatgaatgaatgaatgaatgaatgaatgaataaaatttaaaagctgtgattagtcactgctataataattatgacaaaattaataattatgaaagagcttttcctaactgaatattgggcttcccaaaagttaggttattttgtttttttttaaataaatatttattgaacatctactatgagtcaggcattttgctgggtcctgggatcgtccatttaagccaagatacttggaactgcatgtcagaagagccagctgccaggttgtttttgtttgtttgtttgtttgtttgagacagagtctcgatctgtagacgaggctggagtgcagtggcgcgatctcggctcactacaagctccgcctcccgggttcacgccattctcctgcctcagcctcccaagtagcttgtatttttagtagagacggggtttcaccatgttagccaggatggtctcgatctcctgacctcgtgatccgcccgcttcggcctcccaaagtgctgggattacaggtgtgagtcaccgcgcccagcccaggttttttttaaatgtatgcatttaagtaagtaatagaggcacatggtaaaacatcaaactgtgcaaaaggtgggtgctatgatcccgagggccagctctcctcagaggccccaccctggccagcctgtcatgcatgtttcctgaaatgctctgtgcacatggaaacatgtggaacaaaacaaaaaaaaattcaaataaaaaaattaaaaaaatatataaaagaaacatgtggactatatgtgtgtgtggatggattgtgcgtgtgtgtggatggattgtgtgtgggagtggatcgtgtgtgtgtggatggagtatgtgtgtgtggatggattatctgtgtgtgtgggtgtccctgaatttttcacatagatagatagactattcttttttttttttgagacacagtctcactctgttgcccaggctggagtgcagtggctcgatcttggcccattacaacctccacctcctcggttcaagtgatgctcctccctcagcctcctgagtagctgagattacaggtgcctgccaccatgcctggctaattctctttcttcttcttttttttttttttctcaagacagagtcttgctctgtcacccaggcaggagtgcaatggcgcgatctcgctcactgcaacctccgcctcctgggttcaagtgattctcctgcctcagcctcctaagtagctggggttacaggcgtgcgccaccatgcccagctaattttttgtatttttagtagagacggggtttcaccatgttggccaggctggtctcgaacttctgaccttgtgatccaccagccttggcctcccaaagtgctggtattacatgcataagccactgtgcccggcctcacctcatgcattttgaaagctccaaggtttgagtgttctctcttcagcctcccagcaaatgtgtgagaggtcaggagtggagggtgatccctgatagagctgaggccaaggcgtgattacctgtctgatgtcacatggtgagtaggtggcccacttgcgattagagcccaggtctccaggatgctctctcgtctgaggccttgcttggcttgctgggaagcgaaggaagggatcactctggcagtgctggcaccggggctagctctcctaaccctctgggtttccttcctttctcccaggaatgatgtctggagaacccctgcacgtgaagacccccatccgtgacagcatggccctgtccaaaatggccggcaccagcgtctacctcaagatggacagtgcccagccctccggctccttcaagatccggggcattgggcacttctgcaagagggtacgggaccgggtcagcattatctgggcaggggtggcaggtcctccttcctcaacagggcagggccagtgctgtgtgaaattaaaggcgttgtaggataagcctactgactccccgcaaggactgagcgtccaccattagggcttcctctgtttgaggggtgcccttccaggagggttccttgggactgatcccagagccatcagtcgcctattttcctccaccctttcctgagccagtctctttggagatgggaggtgcagtctcagacaagggtagaacaaattctgagtgaaggaaacgtccgtgtccactggaatcctggcaggtattccaggtggggaggaagagcacaggcaatgactgggaggtgggcctgagtgcctctcctgttgtctccacagtgggccaagcaaggctgtgcacattttgtctgctcctcgggtaagtgatctgcttccccatcctgtcctctgctgtgtgtcctgggatcagacactctgcttctctgagccctggtttccccagcaatgtgccactgaaaacaatggcagcagccaccaatttggaattccagggggaggccagcagtcgcccagggtggggaggtgaggggggaactccagagcccccagtagagggtacgcagtcctgcctgggctcccagatgctcccgcactgacacgctctccacccccgaccctggcagcgggcaacgcaggcatggcggctgcatatgcggccaggcaactcggcgtccccgccaccatcgtggtgcccagcaccacacctgctctcaccattgagcgcctcaagaatgaaggtgccacagtcaaggtggtgggtgaggtgagtgccgacccagggcaaggaagagagagggcgcctggtggctggacagggtgccccctgtgcctcaccccctcctatctcccctgtggctttgcagttattggatgaagccttcgagctggccaaggccctagcgaagaacaacccgggttgggtctacattcccccctttgatgaccccctcatctggtatgtggagttcaaggtcacttggtgggggtggcagccactgccctatatcactgggcaggcattgctatgatgcacatcatcacgtcctcatgcacagcagctgcacatgtgtgcgcacaccaccatgtagaagcacacttaggcggggcacagtggctcacgcctgtaatcccagcactttgggaggccaaggtgggaggatcacttgaggtcaggtgtttgagaccagtctggccaacatggggaaaccccatctctactaaaaatacaaaaattacctgtacttggtgacacacgcctgtaatcccagctcctagggagactaaggcaggagaatcgcttgaacccaggaggtggaagttgcagtgagccatgatcacgccactgcactccagcctgggcaagagagtgagactccatctcaaaaaaaaaaacaaaaaaaaaaaaaaagaagaaacacacgttacacccaggtgcagcagcaagtgcacacatgtcaccaaatgcccattatccacggctgcacatgcaacacacccaaggctgaacatgcaagcacacagatgcccaagtgtctgcagttgagtcctcctggaagcagacttggggacaaaggtttcagggaaaggagtttatttgggaggtgattcagtgggggagtggggaagtaagccagggaaggaagggggctttgctgtttcttttttctttttctatcatccagtccaaaatgtcaggaagggggctttcaaaggcaggtggccgctgtgggtggctagagcttaatccctcggggaccctgggagccagtgcagaaggcgcacagcagtttcccgccccctgggagaaggagcaggggagtttgtacaccgactcgtagccattggttgagggctgccttaaggggctgccatctcctgactctgtttgcaggtagctcaggtggccagagaaggtcctcaggcagagacacagatgctggcagctagaagccaaggttgggggtggggagggaactagctccaggccccatcagctgggataaggccggtgcaaacctgagctggtgtctccaggaagcctagccctgacctgccaccccctctccccaccctccagggaaggccacgcttccatcgtgaaagagctgaaggagacactgtgggaaaagccgggggccatcgcgctgtcagtgggcggcgggggcctgctgtgtggagtggtccaggggctgcaggaggtgggctggggggacgtgcctgtcatcgccatggagacttttggtgcccacagcttccacgctgccaccaccgcaggcaaacttgtctccctgcccaagatcaccaggtgagcagctggggtgcctccctcgggtgtccagcaagcactggggagtcccccttagggctggctttgagatagatcctgatgtcagcctgtttcacagatgagacaatggaggctttgatcgcttgcccagggccacgtccagtgagtggtacagcttggactcaaacccaagtctgtgtgactccaacatgtcacacttcctcacgtgaaaagcctcatatgaggctgggcacagtgacttgtgcctgtaatcccagcactttgggtggctgaggcaggaagatcacttgaggccaggagttcaagaccagcctgagcaacatagagagattcccacctctaaaaaaagttttttgttttttttttaaattaactgggcgtggtagcacacacgcatagtcccaggctaaggtgggaaggattgcttaagcccaggagactgaggctgtagtgagccatgatcatgccagtgcactccagcctgggtgacagagcaagaccctgtctagaaacaaagaaaaaggaaggaagggagagagggagggagggaggaagggagggaaggaaggaaggaaggaaggaaggaaggaaggaaggaaggaaggaaggaaggaagggagagagggagggagggagggaaagaaggaaggaaggagaaaggaagaggaaagaaagagagagaaagaaagaaaaaaggagaaagagaaagagagaaagaaaaagaaagagaaagaaagagagaaagaaagaaaaaagagaaagaaaggaagaaagaaagaaaaagagagaaagagaaagaaaggaaaggaaaggaaaggaaaaagggagggagggaggaaggaaggaaggaaggaaggtctcataggaatcatttactgagtcctctgtggcaaggttttacatacctcattactttgccaaccatttactgagcccctattgtgtgtaagacactggctgatctggggagagagtggtgcataaacacaagtcttgctcctaaggagttcatatccaagccccaaacctttgatagtttgtttgtttttgtctttgttttttgagacagagtttcgctcttgttgcccaagctggagtgcaatggtacaatctcagctcaccgcaacctctgcctcccaagttcaagcgattctcctgcctcagcctcaggagtaggcgtctgccccaacacttggctaattttgtatttttagtagaaatggggttttaccgtgttagccaggctagtctcgaactcctcacctcaggtgatctgcccacctcagcctcccaaagtgctgggattacaggcgtgagccaccgcacccggctggtagatagtatttaattaggattaggttttgaccccattacacagaaggcccaaataacagtgacttaaatgagattgaactttaccatgaaaagtcagggcgccaggtcccttgagtttgtttccccattatccttagcatgtagtgtttcagtccaaaatggttgcctgagtgccagctatcacacccacattccatttggcagggatggggaagcaagggcagaaggctatgctccaaccttcaaagacagacacaacacttccacttacctttcgttggcctgaacttagtcacatggccacatccagctgcaagggaggctgggaaatgtctttacaccaggtgcccataagtgcagctagaaaagacaccggggcagacaatagtctcccctacagatagggaagactgagcccagccaggtaagaacttcttccagattcctgagcctggcatctggagctgctgtgtcccacactgtagggactgaagacgtcatggctgagcgcctactgtaaaccacgtgctgctgagggctgaggtggctcaggccctcaagatgctcctggggaagggagagggggcagctgagctgggaatagagctagtggtcaccccaggaagctggggacagcgtcaacacattggttaccagtgcaggtcttgaagttatacagatcctgcttgcagccccagctccagcactccctaggtgtgtgaccttgggcaagtgactgtacactctctgcctcagtttcccctttccaaaatcaaaccaattaacaacagcagaattcttgtgtgaattcaataatgcttgtgaagtgcttggcacctaacactttctcaagaagtactcagggcgcttgttactagcagggaggactatttgagaattccacatagggccgtgggactcagtactctcccaggaggactccttggaggaggtggccttaatcctgagccttgaaggacacagcaggatgtgggcaggcagagtggtgctaagcttcatgagggttaaccaggtagcggttgtaacccggagcaaaagcagggagtcctagcagggaacaacaagggtcctcttaggcatttgctcctgacttagtgttgatcagaatatgacgctgtgccgaggcaggaggatcacttgagcccaggagttcaagaccagcctggggtcgggcatggtggctcatgcctgtaatcccagcactttggaaggccgaggggggtggatcacctaaggtcaggaattcgaggccagcctggtcaacatagtgaaaccccatctctactaaaaatacaaaaattagccgggtgtggtggtgggcacctgtagtcccagctactcggtaggctgaggctggagaattgcttgaacccgggaggcagagattgcagtgagctgagatcgcaccactgcactccagcctgggcaacagagcaagactccgtctaaaaaaaaaaaaaaacaaaaaaacaacaaaaaaacaaacaaaacaaaaacccgcctgggcaacatagtgacactcagtctcaaaaaaattaaaaaaatgatactaaagaatatactatatgatgtcaattcaaacactctgcaccttattagaagaccccacttggtttggggatccctgaggcttttcagggttttacagctgcagaggtgatacctgtccccctgcccccagatgcattgccttctctgtctcacatccatctccttctctccctccctgcccccatctctctcctgtttcccccactcccatcctcttctgggttcccacttctctcagtgttgccaaggccctgggcgtgaagactgtgggggctcaggccctgaagctgtttcaggaacaccccattttctctgaagttatctcggaccaggaggctgtggccgccattgagaagttcgtgggtatgtgccaggtcctcccatgacctgacctgctgactcaggcgctaagtggctgtccacttggtatgccactggggatcccactcaccccaagtttccttcacagttctgtcttccacctctgcctagaattttccattaatagaagataatacaatgccgtgtagaaatagaaaagcaaaagccacacataatggaaaagtaaatagaaaagtaagcctgataaccatagcagcagaaaacaataagtgaatatttaaaatgttgcttctgagtttcctggcagccagggtaaaaaaaggaaaacatggggctcagagtttgattcattcagaggtgagtctgtaactaggctggactctttctgggttccgtctagggcagggatggcaaaaaagagagaaaaaaatagtaccatttatgcttctgtacctgctgcacacttggctaatcaaccatggaactcttgctcagatgatgcctcagaatccttctcaacacagtgctcacgcaaccacactcattctatcagtgttagcactcatgatgaaacccaggccctggctcaggcttggaagggccaattcaggccgatgacgggacgtttggttcaggacctgcggctgcagccagggctgagagctgattggctcagtattcccaggtcctgccaatggcctgtcagctcctgtgcaccctgggaaactcaggccacgcccctgtcacgccccttctctggcagatgatgagaagatcctggtggagcccgcctgcggggcagccctggccgctgtctatagccacgtgatccagaagctccaactggaggggaatctccgaaccccgctgccatccctcgtggtcatcgtctgcgggggcagcaacatcagcctggcccagctgcgggcgctcaaggaacagctgggcatgacaaataggttgcccaagtgaggacggaccccttaccgatctgtgctctcctagcccaagagacccctggaggggctggagttttatccagcgcctcgtcgtatgtttggctgagcacctgtggcctgggtgcaggttaacttcttgttatcaggagcccactatgcagaggccaaaggtcggcagccagcgaggctatgaattggacctttttggtatctgtgtgactgctctgtgcccatccttagccaacttgctggcgtgacaagtgcccacaagtaacacaccaggtacccagagcagggtggacaggagagacctgaatcacagcagtgaggaattcctcaaagctgtgacctcgcccctgaacttgttcccagtgaaggtcaacagggccaccaaccagggttgaggaaccatctcagccatcctcccaggaagcccacccctgacccttactaatttttctaatgtgcaaactttttcattgaaaaataaaatatatttatgaaacaaa

SEQ ID NO: 691 - DNA, 7753 bp, Mus musculus
  source 1..7753: mol_type = genomic DNA
ctgtgcctcaaccccaccccccgcaagtcccgcctctcactatctctgagagcggtgcttcatttccactctgtacagacccattgtatggtctgacactaatttgggtttaggtggctgagcctaagcccaatactccccaccccttttctttctattttaagacaggatctcactccttagttcaggctcaaacttgtagtcctgcttcagcttccactgtgcggggatcacaggtgtgagctgccatgtggctagcctgtggctgtttcttagcgatatactacgtgtccagcaactgcaggcttttttgttattctgagactgacccccgggtccaggcaagggctcttcagtgccgcatgccctcctccgtggccgcccagccccggcctggaccagggggcggagcttgcttggggtggggggtggcctgcctggggcctcattggctacccggaggctcagccctgagaataaagctctggagtgtcctgccaactttgtactctcccaggacacagacacaatctactcctctcgctcgctctgtccagctcctcctgtgccgggtatgtggcccccagcttcagcctctccccaaggttaacagtcagggacagccagagcagggtacccgggaagtggagaggaactgggactcatccagttcaagtctccgtgttcagatctgtaaaatgggacgaagtggtaggaccttctagatcaggatgtcgctggggtggcatgtggcaggctatctttgctgaatgctcgcatgagccaaatgacaaagaagggacttctgttctggaaccgatgtcgcactagccacctttgccaagacccccagtgctttttttctctcttgcaggtcatactatttatggtgccagatgggaccggcctctggggtgggaatcagaacagagtaccctgcccatttccccaaaggttcccaatgctgcagcctgggacagggactggtggggctccccaaagccacctggaaggggtttgcgtggtggaagctatccactagccagaggctgcaacagctgggccttttaggtccagagaagacccactataccaggatgggactagctaggacaaagcaaacacatttggaggtactcttgatgtgtgccattcttcccggcagctgtctttcaagaggtcttctagaaagaggagtctggtgggctggcctgttatctcagcacttgagagactgagagcacttgagagactgaggcagggggaacacaaggagtttgagtctctctctctctctctctctctctctctctctctctctctctctctctctctctctctctctctcccctaaggcaggaggatcacaaggagtttgagtctctcctacactcacacacatgcccttgaatacatgtggatgctcggaagagacaccatctgcctggatttgatcccacttggctctgaatctctctcagggcttctcacagggccccgaggtgacagtagctgagccagtgttagcccaaaggcttcctcactgccatggctaccgaagggaccttggggtgtcattagccagacttgaacttgtcatgcagtctctcctgtgtccaggttccctctcagtgtggcagctgtgtgatggggtgggccagggttctctgttgcctcttccccatgccagaaaaaaaaatctataactctatctttttaaaagaacaagggcctttcagagctcgatactggacttccaggtcttttgggtttttttttgtttttgaaacagggtcctacgtagcccgggatagcctcaaactccctacatagccaaggttatgaatgactaattctcttgccttcgcctgccaagtgatgggattacaggcctgccccaaaccccaaaggctcttggtttttgggtttttttcccccgaaatatgtatttgttgaacgccagacatagcagggaacacctgtgatcccagcaattgggagactgagatagaaggacccaacgttcaaaactagcctaggtcacatggtaagaacgcactgagttttttcctttttaaaagcatttgtgggtttgggatgtggctccgtctgcgaagggcttcccatgaaagtgcgaggacctatgttcatattcccagaatggagcacataaaaatgctgagctcagcaataaatgcctgccatccattcactagggagcaagagacaggaggacctcagggaacctctggaggacctcagggaacctctggaggacctcagggaacctctggggacctcaggggacctctggaggacctcagggaacctctggggacctcaggggacctccagggagcctccagaaaaatttaggggacctccagagatctcagtgtaccttgggagagctcagtgaacctcaggggacctcaagggacctcagggagtctctaaggttccctggtcagccagttgaactgaatcagggagctcccagctcaatgcaagaccctgtctcaaaaaataaactggagggcaactgagggagatactgacctatgttgacctctggcctctgcacacatacacacacatatgcgcacacacacacacgtgcacactgctgagcatggagggatgctgtggacccgagaaccactcttcacaggtactccacttggcccatttcttttgtgtctgctgacgtgctctgagctcacgggaataggtcagctggggttggttcctggggactttgcaggaacagaccacagctgactctgaccttgcttggtccccatcttgtggctcattctgagacagtttaaacaagccccttcccctgaacttgcccaaggatgtttttgacctcgtgaccttctaactttgggggtcaaagttgagggcttgacctcctatttggccactaggtagtcagagctgagatttgattcggtgtcctttctttatcattatgggtatgaaagaggagtcatagagcccaggagggcagcctctcttgaccctctgtatctcctccctgcccccagccatggctgcccaggagtccctgcatgtgaagaccccacttcgtgacagcatggcgttgtccaaattggccggcaccagcgtgttcctaaaaatggacagctcccagccctcaggctccttcaagatccgaggcattgggcatctctgcaagatggtacaaggcagggtggcatcctggcaggaggggcagtccctcccacctgacagttgtagaggcaggtgtgcagacagacacccaggattccaaaccgagtcaggaaggtagaaagtgaacggtatcccagagtgtgtagcaagaccctgtctctacggaaataataattttagggcctggggtttagacagctcagcgcttaagagcaccggctgcttttgcagaggacctgagctcagttcccagcacccacatgggggctcacaaccacctataactccagttccaggagatctgacaccctcttctgagttccaggagcaactgcacttccgtgtatataatccaaaataagtaaacattgggctggggagatagctcagctgttaggaatgcttggggctctttcagaagaaccatgctcaccctcagcatccaagtcagtgagtggctccagcccaaagatttatttagtaagcacatacattttatgtgctgtctttagacatcccagaagagggcgccagatctcattatgggtggtttgaactcaggacctttggaagaacaatcagtgctcctacccgctgagccatctcaccagctcaatgaaacaaatctcttttattattattatttcttatatatatgatgagtacactgtcactgtcttcagacgcaccagaagaaggcattacagaaggttgtgagccaccacgtgggtgccaggaattgaactcaggacacctggaagagcagtcagtgctcttaactgctgagccgtctctccagccccaacaaatcttaaataaaaaaaaattttttaagaaaatcattttagaataggttacacatatcagggctctctctggctaaggggtgtccacccatacaggtccctagagactgacacctgcctccttgccccagttaatctccttggggaagatatacctgagagagaatggttcagatactaagtgggtaagctcagcttaagatagaagatgttccagtgggagagatgaacctaggctgggactggggggtggcctgaccctccctcttgtctccacagaaagcaaaacaaggctgtagacattttgtctgttcttcaggtaagtgcccagttagctttctttcccttcctaccctggtctgtgcgtcctgtggcccagtaccaggcactgtttctctctgggccccgggactccgcaccagtgtatccattcgtcatcacccagatttgaggttccagaatgaagcagaattccctctggagatgagcagtcccacctggggtcccagaccctcctccctgacacgccctctccactgacagctggcaacgcgggcatggcgaccgcctacgctgccaggaggctgggcatcccagccactatcgttgtgcccaacaccacacctgccctcaccattgagcggctcaagaatgaaggcgccacagttgaagtggtgggagaggtgagtgcggatccctcgtgggcaggagggctgataactagatggagatcctcatcatctcagtctccccgccccgccccgccccttgcagatgctggatgaggccatccaagtggccaaggctctggaaaagaacaacccgggttgggtgtacatttccccctttgatgaccctctcatctggtaagttggagccaccccactccgatctgccatgtgtggcttctgctcctgccctctccggcacacccaatgtgtgcaggagagaacacacaaacgcacagatgtacacatcagcgatgaggggtccccagatgcagagacttggggagatcagctcatctgggggtgactaggaacaaacgggctagggaagcatccgtggaggtggctctcgagtgccagcatctgtctgccatgagcaactgaagcagggtccctgggaaccctgggagttggtggggagtaagcatccaaggatgtatccactggtgggcaggaaatggagatttatccaccaacccccacagccatctcctctctccatgagtgggggaggggggcttgtctcctacacaatcagtgtcctgtgaggcaggctggacagcaggaaggtcagaagccaggttcatcattgcctgggatagaaggaaataaagaccttagtcagcatctttggccagccattgtgtcttcgtgaagccctggcctgaccctgctacccttctcctgcccagggaaggccacacttcccttgtgaaagagctgaaggagacgctgagcgccaagcccggggctatcgtgctgtctgtgggcggtggaggcctgctgtgcggagtggtccaggggctgcgggaggtgggctgggaggatgtgcccatcatcgccatggagacctttggcgcccacagcttccatgctgccatcaaggaaggaaagctggtcaccctgcccaagatcaccaggtaaacccggggcatctctccatcacatgagcagtgggattgaggcttcgggagctgcgtgcctatgtgtagcccgtctaacacagcgtggggggggggggaaacgccctctttgcctgcaaagaatttataaccaacccccacaccagtagcttaaaccaaattgaactctagttctattagtggcaactccttagagcaggaagctgagttcctggagtatcttctcctatcactctgagtatgctgacctagggtccaaaatggctgcttttgtgccagccaccacacccacactccatctgagaagagggagagcagaaggatgctcctaaactttaaagactaagtggagctgctccccagctgacagcggggtcacacccagcaaaccaatcattagagctgagagtgttgtgaacgagttgaacctggtctgctgggcaccttacctaagctgtttggcacacagtatggaactggccgtttcttgctgacaggctgatggagagctgctacccagcatcccgagagtgaactgtaccacaaacctatagttggggtgtgtgtgggggggtcacagttcaactctcaaagtctatcttgtaccaagtgtagatggtttgttgtgccaccacaatgttgaactctcatagacgatccatcagaattcagagctatctatacttgctcctatcatgcattgggcagagctgggtcgcatggtcacacctagctgcaagggaggctaggtaaggtttgccgtagacatccacaagtacggctagaagggatttcaagggagaagacagcttctgtcataggtagggaacactgagattctttctggatttctgggcaacatgtccctattagactgatgctggtctgctaccactattctttcctgccagatggaatacgggtgcaatagttggcactccagcagccattttggaccctaagttaatataaccctggagcagctattgcctaatgtttcccattctgtacaactgagcttggaaagagcatgacacctggcattttaggaaggactagctctccctgttcattagggtgtgggagggatgtctgagaacctcaactaatccatgggaccaaggagtccctctggggggcttcccggaagaggaggcaagtgagcaaagcaggcagaggttacaagctggggcggggggcagcagggtcttcaggcatttgagtcaggccagtttgatgccctagaccactgtgcaggatcccagggctgggcaggggttgccactcccaccactcaccttaagtcctcacctctcacatgcacccttctaccttgtctctccccctgtgcccacccccctcctcatccccacctcccagtgtcgccaaggccttgggcgtgaacactgtcggggcgcagaccctgaagctgttttacgaacaccccattttctccgaggtcatctcagaccaggaggctgtgtctgctcttgagaagttcgtgggtacgttctgtggaatctttttgccccttgactttctccctcctcagcctagactctcatgccagtccctggtaaatgcaaaagccactcacaaaaatgtgagtctggtaatcatgatagcagtagctgggatcaaaggggagaggaccaagatgagctggacttggagatgatctttagcagaactctctggggccagccttgaagatagccaaagagaacaagagtaagaacacgcactgctgttaatcatctcagcttgctgccacacaggagtgaccttctcaggtgcctgagtgcccttagcacaggctgtaggaacaagcagttccagccatagggagatgttgagagcttaccagccgcctccccctccccccgtagacgatgagaagattctggtggagcccgcgtgcggcgcggcgctggctgctgtgtacagccgcgtggtgtgcaggctgcaggatgaaggccggctgcaaaccccactggcctcgctggttgtcatcgtgtgtggcggcagcaacataagcctggcgcagctgcaggcactcaaggtgcagctgggcctgaacgggctgcccgagtga

SEQ ID NO: 692 - DNA, 7656 bp, Rattus norvegicus
  source 1..7656: mol_type = genomic DNA
gggttcatgaagctgagccccatactccccgccctttttctttctattttgagacaggctctcactccttagtccaggctgtccttctgcctcagcttctattgttctggcttgaaggtgtgagttgccatatggctggcctgtggctgtttcttggccataaactacgtgtccagcaactgtccggcttttctgttattctgggattgacccctgggactctggcaggagctcctcagtgctgcgctgcccgcccacgagtcctataggcctttcggagatgaatgtcttcatttgcaacggaaaaaaaaaaaaaaaaaaaaaaaaagaacggggagagaaggtgaggcggaccttggagggcagaacaagtggtgcagcatggaccagggggcggggatttctgggggcgtggcctgcctgggccctcattggctgcccggaggctcagcccggagaataaagccctggggtgtcctgccaactttccactctcccaggacacagacaaaatctacccttctcgctcgctctgtccagctcctcctgtgccgggtatgtggccctcagcctcagtctcaccccaagattgacagccagagcagggtaccggggaagggaagagggactgggactcatccagttcaagtctccatgtactgatctgtaaaatgggacgatgataggaccctctagatcaggacgtcgccggggtggcgtgtgacttggccaagtgctcgcatgagtcaaatgacaaggaagagacttctgccgtggaacccatgccgcaccggccacctttgccaagaccgcctgtgcctttttctctcgcaggtcatatcacctatagaggcggatgcgactggcctctggggtgggaatcagtatggagtacactgcccattttcccaaaggtttccaatgctacagcctgggacaggaaccaggcagggttcgccaaagccacctggaaggggtctgcgtggcggaaactatccactagccaaaggctgcatcggctgggccttttgggtccagagaagacctagtatgtcaggatggggctagctaggacaaagcaaagaaatttggagttactcttgtgtgccattgtttccgactgcctttcaagaggtcttctagaaagaggagtctggtgggcttgcctataatctcagcacttgagagactgaggcaggaggatcacaagtctgtctctgtctgtctgtctcttgactgaggcaggagaatcacaaggagtttgaatctctcctacactcacacacatgcactcgaattcgtgtagatgctgggaagagacaccacctgcctaggtttggtcccacttgactctgtatctctcttggggcttctcacagggctccaaggtgaaagtggctgagccggtgttaacccaaaggcttcctcactgtcctggctcccgaggggacctcagggtgccattagccaaacttgccgtgcagcctctatctgcatcgaggttccctctcattgtggacgctgtatgacgagggcaagccagaggtctctgttgcctcttccccatgccagaaaaagcaattccataattctatctttttaaaagaatgagagcctttcagagctcgatgctggatttccaggtcttttgggggttttgttttgttttgaaacagactcctatacagcccagggtggcctcaaactccctacatagccaaggttgaccttgaatggctagtcctcttgcccttgcccccaagcaatgggattacaggcctcctgcaccaagcccatttttttcttcaaaatatgtatttgtcgaacaccaggtgcggcggggcatacctgtgatcccagcaattgggagactgagacaggaggatccaaccttcaaagctacatggtaagaacatactgagtttgttttaaagcatgtgtggggttgggatatggctcagtctatgaagggcttcctgggaaagcatgagggcctgtgttcatattcccagaatgcacctcaaaaatgctgagctcaacaataaatggctatcatccctgcactagggagcaagagacagaaggacctcaagggacatccaggggcctctggaacctcaggggacttctgggaacctctggggacctcagagcctccggggttctctggccagccagttggactgaatcagggtgctcctggttcagtgcaagaccctgcctcaaaaatagtgggcagctgaggaggctactgactgatgttgacctctggtttctgcacacacacacacacacacacacacacacacgcacgcacgcacgcgcgcgcgcgcgcgcgcacgcacacacacacacatgtttaagtaatatataatagggcaaaatgctgagcacggaggggtgctgtggcccggagggtcactcttcacaggtaccccatttggcccattttgtgtgtgtgtgtgtgtgtgtgtgtgtgtgtgtgtgtgtgtgtgtgtgtgtgtgtgctgacatactctgagctcatgggaacaggtcagcccgggttggttcctgaggactttgcagaaacagaccctggctgaccctggctgtgctttgttcccatcttgtggctcattctgagccagtttgaacaagcccctccctctgaacttgcccaacgctgcagcctaaagatgtttctgagcttgtgaccttctaactttgggccactgggtagttggagctgagctttgatttggtgtcctttccttatcattatgggtatgaaagagtggtcctagagcccaggagggcagcctctcttgaccctctgtatctcctctctgcccccagccatggctgcccaggagtccctgcacgtgaagaccccactacgtgacagcatggcattgtccaaagtggccggcactagtgtgttccttaagatggacagctctcagccctctggctccttcaagatccgaggcattgggcatctctgcaagatggtacaaggcagggtggcatactggcaggagaggcagtccctcctacctgacagctgtagaggcaggtgtgcagatacacacccaggattccaaacggaggcaggaaggtggaaagtgaatggtatcccagagtgcatagcaagaacctgtctctaaggaagtaataataattttagggactgcagttgagacagctcggcggctgagagcactggctgcttttgcaggggacttgagctcagttcccgcaaccacatggtggctcacgatcaactataactccagctccaggagatccaacactctcttctgatttcccggagcaactgcactcacatgcatataattcaaaataagtaaatattgggctggagcgataattcagtggttaggaatacttggggctctttcagaacagccatgctcaccctcagcatccaagtgagcgaggggctgtaactccagctgcagggcatcaaatgctctcctctgccctctacaggcaatacgtacacacagacacaagtgcacataaagaaaacacatcttttttttttctttttaagaatttttatttattttacttatatgagcacactgttgctgtcttcagaaacaccagaagagggcaccagatcccatcgcagatggttgtgagccaccatgtggttgctggggattgaactcaggacctctggaagagcagtcagtgctcttaacccctaagccatctctccagcccaagaaagcacatcctaaataaataaataaaatatgttttaaagaaaataataattttagaataggtttcacacatcagggctctctctggctaaggggtgcccaccctacagactgacacctgcctccttaccccagttaatcaccttgggggagatgagcctgagagagtggttcagataccaagtgggcaagctcagatgttccagtggtgaggacgtgcctaggcagggacttgggggtggcctgatccttcctcttgtccccacagaaggcaaaacaaggctgtaaacatttcgtctgctcttcaggtgagtgcccagttcgttttctttcccttcctaccctgttctgtgtgtcctgtggcccagcaccaggcattctccctggactctgggctctgcaacaacacatccattcgtagtcgtccagatttggggttccagaatgaggcagaagtcactctggagatgagcagccccacgtgaggtcccaggccctccttcctgatacaccctctccactgacagcgggcaacgcgggcatggcgactgcctatgctgccaggaggctgggcctcccagccactattgttgtgccaagcaccacacctgccctcaccattgagcggctgaagaacgaaggggccacagttgaagtggtgggagaggtgagtgcagacctattgtgggcagcagagctggcgggtggaggtggggggctgataactggatagagccctcgttcatctcaatctcttcctgcccccacccctgcagatgctggatgaggccatccaactggccaaggctctggaaaagaacaacccaggttgggtgtacatctcccccttcgatgaccctctcatctggtaagttggagccaccccactccctactgcatgtgtggtctctgctcatgttctcgccggcacaccagccatgaggggtccccagatgcagagattcggagagatcaactcatcttggggtggctaggagcaaactaactagggaacgcatcggtggaggtggctttcatgtgccagcatctgccctgaacaactgaagcttggcccctgggaaccctgggagtcggtggggaatgagcatctaaggatgtgtccactggtgggcaggaaatgggggtttatctaccacacccacagccatctcctctctgggagtgcgtggggtgagggggtggggaggtggggtcttgtctcctaccctgtgccctgagcagcagggaggtcagaggtcaggttcatcatggcctggcatagagggaaaaacagaccttagctggtatctttgaccagcccttgtgtcttcaggaagccctgacctgacctgccacccctctcttgcccagggaaggccacacttcccttgtgaaggagctgaaggagacactgagcgccaagcccggggccattgtgctgtctgtgggcggtggaggcctgctgtgcggagtggtccaggggctgcgggaggtgggctgggaggatgtgcccatcatcgccatggagaccttcggcgcccacagcttccacgctgccgtcaaggaaggaaagctggtcaccctgcccaagatcaccaggtgagcccggggcacctctccagcacacaggcggtggagctgagcctttggtcctttgcccgaagccacactctcgcgcggcccgtctgggccttgaacgtgagtccgttctttatgtgacagtgcttaagtgacgtccttcgcttcttgaggtcctcacgcctcatttcttttgctgctgtttgatctggcctccgaaatgtgccagaccctggctggtcctggactcagcctgggagaaaacaatctctggcctgcaaagaatttataaccaaaccccaccgcggcggctcaaaccaaactgagctctagttctatttaagactattaggggcaactcttcgaaccaggggctcagttcctttagtctcttgtcccgccactgagagtgttgacctagggtccaaagtggctgctttggtgccagccaccgcacacacactccctctgaaaagaggaagcaagagcagaagttgtttctaaactttaaagaccaagtagagctgctccccagctgacaatggggtcacacccaatctttaaccattcgttaaagattgctatgaatgagttgaacctggtctgctggacgccttacctaagctttttggcacacggtacagaactggctgcttcttgctgataggctgatgggagaggtctggctcaaggctgccactgggcaacccaagagcggattgtactgcaaacccatagtctgagaaaaggtctgagttcaactctcaaagtttggtttgtaccaaatgtggattttttttttttttgacaccacaacgttgaactctcatagaccaaccctcagaaatcagagctgtctgtacttccctcttatcatgcactgggcagtgcttggtcacatgaccatacctagctgcaagggaggcagggcaaggttcacactaggcatccacaagaacagctagaagggatttctagagagaggatggcttctgtcataggtagggaacaccgaatcagttaagagactcttccatatttttgggcaccatggtgagactgatgccatcctgctaccactattctttcctgccagatggaatacagatgcagtagctggcactcaagcagccattttggaccctaagttaataggaccctggggcagatattgcctcatctgtttcccactctatacaactgagcttggaaagtgtgtgacagacacctgatactttaggaaggactagttctccctgtttgttagagtgtgggagggctgtctgagagcctcaactaatccatgagaccaaggagtccctctgggagtctccctggaagaggtagcaagtgagcaaagcaggcggaggttacaagctggcaggctgggggtgggggtggggcagcagagtcttctggtatctgaatcaggccagtttgatatcctagaccactgtacaggttcctggggctgggcagggttgcccactcccactgttcacctgtagtccccgcatctcacgtgcacccttctaccttgtctctccccatgtccacccccctctcattcccacctcccagtgttgccaaggccttgggtgtgaacactgtgggggcacagaccctgaagctgttttacgaacaccccattttctctgaggtcatctcagaccaggaggctgtgactgctatcgagaagttcgtaggtatgttctgtgggatctttttgtccttgattttctccctcctcagcctagaccatctcatgaaaggaaactaaagccagtcagtggtaaacgcaaaagctgctcgtgaaaacatgaacctggttaaccatgatagcagggaggacaaactagttttataagtgccagttttgagctttctggtagccagagtcaaaagggaaagggccaagatgaggtgatctctctgggcttagcctgggagatggtcaaaggaacgagagtagtgcacactgccattaataacttcagcttgctacaccactagagagcgaccttctcaggggcccaggaaccccaagcacagactgtagaaaccgagactgggttcagaacaaacagtcctggccacagtgactgacgagagctgacgagccccctcccctacagacgatgagaagatcctggtggagcccgcgtgtggcgctgccctggctgcagtgtacagcggtgtggtgtgcaggctgcaggctgagggccgactgcaaaccccactggcctcgctggttgtcattgtgtgtggtggcagcaacatcagcctggcacagctgcaggcactcaaggcacagctgggcctgaatgagctactcaagtga

SEQ ID NO: 693 - DNA, 8827 bp, Equus caballus
  source 1..8827: mol_type = genomic DNA
aaagagtcctaaaagatgacaggggaaggcaataaggaactgcgttcggattgcaggtttgggagtccaactgctggattcaagtgtcggctcatccctctcactagctgtgtgcctcagtttccccttctgtaagatggggataatgatagtacccacttcctcctggggtttgttccttgggtgcctactatgtgccatgctgacgactttaagtgccttctcctggccctcctcctgtaagctcctcacagcccttgggaagtaggtatgggccccattcgcaagtaaggaagctgaggaccgggaagaggaagtggccatgcggagaggagagggagggagagatgggaggaagctgccggagggaaatgcacagccagcctccctgccccctcctggaagcccccagcggcttcaggccagggggcggggcctggcgggggcggggccagccctggggccccattggctgcgcggaggcacccgctgaataaagcccggatgggcagcccctgggacagttcagaagacagtccacctgtccacctaccctccatctgtccatgggccatctgcccagtctatccatctggaccatctccagtcatccctccttggcttactcgctctacccagcgccccgctccatcgctttgccaggtgagcggccccatcctcagccccaccccaggaccggagtgcctgcgccagccggggcttcccaggggaagaagggcaggtagaaagagtctgttgggcataaaaacccatgctttggagccagactgcctgggttcacatccagctctgtgcctcagtttcctcatctataaaatggggacggtcataggaccttcctcataaggtttctgtgagggtgaaatgagatagtgcatttaaagtgtccggtccagaagtcatcatatttcgtttttactgagctctgactgtgagccgagtgagaggggagtgacttctgacggtggggcgcctggctagcgtccctttctgaagtctccccttcaggggcgagagtagcagggtgggaatggggtgggggcagtttacaggtgctggcttcacacagggcgaggggtgatgcccgcgaggggtccctgttgcctcactgaaggcatctgatgctgcaacagaggcaggtggtgggaggggcttcccaaaccagcggtaaggggtcttcgtggttgacacttcccacctccaaccgggagagatccctgaggccagggcttggtgacttcacaagacccagggctttaaggtccagtccgccagaacagggggtgggggggtggctgggggggaggccatggcttggggaaagcccccaggagctgagagctattcagaccgatgggggaagccccttgtggagtgcctccagtctctagctttcattcacaagatctagaggggaggcggtgtctgtctgggccccctcccaggccctgaatgtcggttaatttggacagacatgggataccaacatttggggggttctaaggccggggggtgtcaggaatcccaccgcctactttccttctgacccaggcatatctggcccaatctgggagaggcgatgcctgagccaggagggccagggcagggggctggaccagacagtgtcactggaaacgcggtgggggcttggccttccgggatggtacagggcgggggggggaccagagaggccgctcacccaactacccgctgatcaattctggctcagcccaagtcggggggcatcagcctcccccatgattctcagcaggagagaggacaagatggaaaggctgggccacctcaacttctccgtctcccagggggcctgagccacccccacgtgtcctagagggagcctcggccctggaaccggactgctgggttcagatcctgcccccccccaaccccccgctgtgtgatcttgggcaaggcacattaatgttccagtccccatcgctgggtgacaaactactgcaagatgtagcggcttcaaatgacagccatcatcacgctgttacctgtcgtggttctgggctcagccaggcagcccccactgggctctcatatggttgcagtgtgacgggggctgggctggagccatctcaaagtggtgtcccctatctggcagttgacattggccatcagtggtcactgggacgcccatatgtggcctccgcacgtggcctgggcttactcccatctcggtggctgcgccttgagagggagcttcccggagagatggccaggctgtgtggcctctgtgaccccgcctgggggcggtggggccctggagctggcttacactggctctgggagtggattgtgcctgtccgtgttcattgcatcatgttggcctcggcgagggacgtgcgtcacggaagggcacgccctgcagtcggggcctcgctttccttcagagatccccttgttaaccattcaccagcacaccactgctcggacgtcacaccgtcacctccattgcagccacaggccccccgggggccaggggaggacacatagacccctcctcccgcagagagctcgtcagtggcgcctcgaaagcggtgcctgtgggatgggaggaggttatgtagccatcctggaaaacatgatgggagcctgtctcctgatcagaagaatggggcggtagcggcatctctagggttgtccaaaggatgcaatgaattaatatctgccaaactgaaaacagtgcctggcacatggtgatcactttataagtgatcattaagtactatagtgattattattaaaacatgataagaagaagaagctggataataataaataataataaaataataaatataaataaaataatttaaaacagggaacagggctttccaaaagtttgttttaaaatacacacatatttaaataaggaatatgtgcccgcggccaatcgaacagcacaaaagggaactcggagcgcccgctctccccagaggccccgcccggactagttcctcgcgtgtgtgtcctgggatgctccatgcacgtggaagcaagtgaactgtgtgtgtccacatttttcgcacacatagcccagacctgatgtcccgcccctgctttacccactgagccatcctgccattccttctgagtcagcacctgcaccgccctcccccccacgcccctccaaagctgtggggtctgagtgttcttgctgagggtcgagcctcccagcagggtgggtgttgggggggtccttatccccattttacagatgaggcagctgaggccctggggagatgacttgtctgatgtcacccggtgaggaggaggccgagttgagatttgaccccgtctccaggacattgtgatccgggccctgtcccagcctcctgggaagggaaggaacagggtcactcacagccacaccgagaggctgcgctggcaccagggccaccctccaaccctccgggtttccttccttcctccagcaatgacgtccacagagcccctgcacgtgcagaccccggtccgggacagcccgtccctgtccaaagtggccggcaccagggtctacctcaagatggacagcgcccagccgtccggctccttcaagatccggggcatcgggcacctctgcaggacggtacaggacgggctccgtcagcagggcgggggcgccaggtcctctctccttgacagggcagggcgagtgctgcgtgaagtctcgggcttccaggctgggcctctaactccccacaatgcctgagcatccaccattagcacctcctcgggttgaggggtgcccgtccgtgaggtcccctcctgggctgaccccagagccaccaggtgccggcctctccctcacccgtccctgaaccagcctcttcggggagggaggatgtcagaggccagggcagtacaattctgtgtgcaggaaggagaggtcagcataacttggaaacagtaggaggtattccaggtgggaggggaaagccctgggacagactttgaggtgggcctgacccctccccacttacctccacagtgggctgagcgaggctgtaaacatttcatctgctgctcgggtaggtggcctgcatgcctcttcctgtcctctgctgtgtgtcccggggccaagtgctccacctctctgggccctggtttccccagcaatgagctgtggcaaacatctatagcagtcccaacttggattccagggggaggccggcggtcacccaggggagttcagagccctgaggttgggttgaatggtcccagccaggctcccggttcctcccacactgacgctctctccaccccctacccctggcagcgggcaacgcaggcatggcagctgcctacgccgccaggaagctgggcgttcccgccaccatcattgtgcccagcaccacccctgccgtcaccatcgagcggctcaagaatgagggtgccatggtcaaggtggcgggtgaggtgagtgctgaccggggcgggggccacagagggcacctggtggcagggcagggtcctccggcgcctcaccccatgccacctcccctgtggtcttgcagatgctggatgacaccttcgagctggccaaggccctggcgaggaacaaccccagctgggtctacatctctcccttcgacgaccccctcatctggtacgcagagcccggggccacccctccacccctgggcacctaccacaaagcacaccatcatgtgcggcagttgtacacgcatgtgcacggcatcgtgtagaagcacacgcacaagcagatagaaaacgcacacgcacgtgccccaacgcccattcgccgtggccacacccaacactcccatgagccaacacacaagctcagagacacaggaatctgccactgggtcccccgggaagcagacccgagacaaagatttcacagaaagcagtttgcgtgggaggtgattcgttagcggcgtggggaaggaaggaagccactaaggctcaactgggcaggcggctgctgggggcagctggagccgaaaccctcggggggcccctgggagccagtgtggaacatgaaccagagttatcccacccgtgcgagaggatgctggagtttagacagcaacacccgcaactcttcattctcatcatcactggctgggggctattctcagggggtgtccttgggggctgtcgtctgctggcaccgccggttgtcacattcataggcagctgcggtggcagagagagccctcgggggagaggcagctgctggcagctggagatctgggcggagtcggggaggaggggactagccccgaggcccgtcagctgcctgggataagggcaagtggcaccgccaggagccccgcctgctgtccccaagaagcccaaccctgacctgccaccctcccaccccctgcagggaaggccaccgttccatcgtgagagagctgaaggagaccctgagcacaaagccgggggccatcgcgctgtcggtgggcggtgggggcctgctgtgcggagtggtccaggggctgcaggaggtgggctggggggatgtgcccgtcatcgccatggagaccctcggagcccacagcttccacgccgccaccactgccggcaagctcgtctccctgccccagattaccaggtgagcagccgagggagggccccgtcgggtggtcagccagcctgggggctgccgggcaggtcccgacaccagtaccatttcacaggtgatgacaccaaggcttcggtcacctgcccagagtcacagtccatgggtggtactgctgggacccaacccaagtctatgtggctcagcaccgtgaacgtcacacttctgcacatgaaaagtctaacactagtcattcattctgatccttgggggagggctccacacctcattaatttgccagccatttaccgagcccctaatctgtgcaagacgctggctgatcctgggggacagtgaggaataaacacagtgcccgggctcatgtcccagccccacacctttgatggtgtttcattaggatcaggtttggccgcattatacaggagcccaaaataacagtggctcaaacaagattgaacttcatttcctcttttatattaaaaaaaaaaagtctggggggcgggcgatgggcgaaatgagtcaagggggtcaaaaggtacaaactcccagttaaaaaataaataagccctggggctgcaatgtacagactggtgactatcgtcaatgataccgtattgcatattcgaaagttgctaacagaatagatcttaaaagttctcatcgcaagaaaaaaattgtaactatgtgaggtgacagaagttacctagacgtattgtggtgatcatttcacaatatatacaaatagtgaatcattatgttgcacacctgaaactccatataatgttatatgtcaattatacctcaatttaaaaaaagtctagaggcaggcaggcagtcccagggcagcaccctgaaggcagcggcccagattccttgagttttttgccccatcatcctcatcatgtagcctatggcccaaaatggctgcttaagtgccaactatcacacccatactccatgtggcaggaaggtggaagcaagggcagaagctatgcccctcctttaaagacagacacaatacttccacttacctctcattggccagaacttggtcacatggccacacctactgcaggggtggctgcgaaatgtcttcccaccaggcctccatgtgtgcagctggaatagatatggggccgacatgtctgccacagataggggccactgagtctgagtaagaacaacttctggattgccatgcccggtgccccggagccaccgtgctggcctctgatcccacactgttgggactgaacaaataattgttgagcgccaagtctgagccacacgctgggctaagggctaaggtggtccagaccccgggatgcacatgggcgagtcggggagatgggagcagccagggatgactcggggaggtgggggatggaacaggctggttacacacgcaggtcctagtcacaggaccctgccggcagccacagcaccactgctccctccacgcgggacctcgggcaagtcactgcgtgctctctgcctcagtttctccttctataaaatcgaaccaactaacagcaggaactgttgcgagagttaaatgacacctgtgaagtgctcagcacccagtacttctcaagaagtcagcgctatgcttgtgttcagggggaggaccgttggagaattccacgtggggccgcgggactgagtgctccccctggaggactcctgggaggaggtggccttaatcctgagccttgaagggcgggcaggccttgggccagcagagcggtgctaagctcagtgtgggtgaagcaggtggcggtgacagcccggagcaaaggctgggagtccctctgggggaccgcaagggggccttccaggcacttgctcctgagctgatgttgaacagaaaatgacaccgtttgccccactgaagcaacctgtgcctcattagaggaccccactcgctccggggagcccccgggacctctcagtgcttcacggctgcggagggtacgccccgtgcccccgcctgcggagccccagttgcctcctccctctcacacccatccccgtctccgtctctcccccccatcccgacctctgcctccctcctctctctgtttcccccattcctgtcccctcctccatcactcccctgccagtgtcgccaaggccctgggtgtgaagactgtggcggctgaggccctgaagctgttttgggaacaccccgttttctctgaagttgtctcggaccaggaggcagtggccgccgtggagaagttcctgggtacgtgccaagtcctttcaagacctgacccactggtccaggaactaatttgctgtccacttgaggccacaggattgcattcaccacaaacttccctcacagttctgtctcctgtcccttcctagaacttcccgttaatgaagagaatacagtgcagtggtaaaaatgggagggccaaagccacaaatgaagatgtttgataaccatgacagtaggggacagtaactgagtattttaaaaaggttgcttctgagcttcctggtagccagggtgaaaaaggaaagcatgggcctcagctgtgggctcaggggtgggtctggaagtacgtgggactctttctaggttcagcctaggacagggatggcaaacagacacacacacgcacacacacacctgcgtgcacacacatgcatacagggtcgcagatatgctgccacctaattctcctgcactggcagtcagtttggctaatcaaccacggctctcctactcaggcgatgcctcagaatccttctcaacacagtgctcaggcgactatcaccattttatcaatgttagcatcaggatgaaacctagccctggctcaggttgggaggtccgtctagggtaacgatggggctggtccaggacctgagtctgtggccaggccaagagtggactggctcataaaccccacgctttacccagggcccgacagtccccttgcaccctgtggaactcgggccccgccctcacctccccctcctcgggcagatgacgaaaagatcctggtggaggccgcctgcggggcagccctggccgccgtctacagccacgtggtgcagaagctgcagggcgaggggaagctccgtgccccgctgtcctcgctcgtggtcatcgtctgtgggggcagcaacatcagcctggcccagctgagggagcagctgggcatgaaggacgggctgccccagtga

SEQ ID NO: 694 - DNA, 9099 bp, Felis catus
  source 1..9099: mol_type = genomic DNA
ggaggcaaacggacctgcccagggcaacacagctagtaaccggcagagccaggacttgaaccccgacccgccagggtggtccccagaaggccatctgccccttcgagacgaaagcgtcaccgagagcgccagaggaaggcaacggagacctgtgctcacactgcagcccactggggtccggggccggctcatctctgtcaccacgtgggtctccgcttcccttgtcgtcaaatgagggtagtgacagtacccactttcccctgggggcccactaggtgccaagcacctgctagtgactaagtgcaccgtctcacgccatcctcctgcaggccctggggggggggggggtggggaagggaggtggccaccgaggacagagacccgagggagccgcaaggcaggacaagcccggccagcttccccctcccctcccggaagcccccgggccagcggccaggggcggggcctggcggggggcgtggccagccccggggctccattggctgccgggaggcgcccggggaataaagcctgggaccgaaggcccagaccggtagtccccgggacaaatcgggagaccctctacctgccccgctagcctccatctgtccgaggcccatctacccagtccatccatctagatcacctcctggccgtcccttcccggcttgctcgctctgtccagccgccttggccaggtaagtacccctcctcagccccaccccagactggagtgcctgcagcccagcgagggggcaggttccaggccaaggcgaggaggtagagtccgtggagcgtaaaaacccatgctctgaagccagaccggctgcgttggcatctagctctgggcctcagtggtctcttctctaaaatgggggtcatgacggacccttcctcatcaggcagtgggaagggtgagatagtacactcaaaatgagagttcgtttttattgagctcttgctatgagagggggagtgacttcttcttctccttcttcttaatttatttttgagagacggaatgcaagtgggttacgggcagagagagagggagacacagaatccgaagcgaggtccaggctctgagctgtcagcacagacgcagggcttgaacccacgaactgggagatcatgacctgagccgaagtcatgatgctcagccgactgagccgcccaggcgccccgagggggagtgacttctgagcgagaggggacacatgacagcagcttccagccccctccctcctccgtgtttggcaccccccacccaggatgttgtgctggagggagcagtgcccatagggggtccctgcccctcatctgaaggcatccgtggtgcaaccagacccaggtgggggcagaggcttcccaaatccgcccgaaagcagtcttgggggtatgccccccactgccaacccggagagctccctgaggccagagattggccacacacacacggggtcccgtcagccagaagcgggggtgccgttgtttcaggaataacctccaggagggtaataatcaaatagatatggggacgcttctgtaatggggtgcatctagtctccagcttttgttcaggagacctaggggagaagtggtatccgttcagggcctcccgctcccaggccctggatgttagttaattcacacagacatcagttgtccgtatgtgggggagctaaggctggggaggtggtcaggaaccccatccctcgcttttcttctgagccaggcatatatggccctctctgggagccagaggcctgagctggaggccgtggccagggcaggctgcaccgggtgggtcactggaaatggggcaggggtgggcggggaggggcagccagccttgtgtgggacggagaaggacatggggagggccagacaggctccccacgcaaccacccgctgatcagttctggctcagcctgagttggggagactccaggaggagaagggacatgtgtggtggccagactctgggccacttcagcttctcaatcacccagagcccgtgcagaaggggagccctgagccgcgcccacaggtgctagactgtctcatccagtcctgcctcccccactaactagctgtgtggccttgggcaagtcacattaatattccggtccctgttgctgtgtgaccaaccgccccaaggtcaacagcgttcatcgtattgttgtctgtcacggccctggggcttatcggggcagttcgcactggggtctctcagatggctgcagggtgacatctccaggtcaccccgccatgtctggccatcagctgtcaggtagggcacctacatgtggcctctgcatgcggcctgggcttactcccatcctggcagccgggtttcgagaggggagctgccccagagagacggccaggcggaggctgtgtccccttctgtgacttggcctggggagcagggctgcactggagccagcagattgtgccgtctcttcccagctccacaggcaatgacgtcatgttggtaactcgagagtggccttggagaaggttacgtacagaacagaagtgcgcccgctacaaatcggggctttgtcttgggagagtccattgctgaacgtttacaagcacaccaccgcttggaaatcgcatcgcgtcttttccagctgcggccaccgaccacccccctcctccagggttcaaggggaggaaacacagagcccacctctcactgagaaaagcatctgtgtcccatcatgaagggatgggaagggcttgcagggaccatccatcctgggaaacacaatccgagcctgtgttctcatcgggagaatgggataatgatggcacctcccaggattggtcaaaggaataagtgaattgatattggtaaagcttaggggcgcccggatggctcagtcggttaagcgtctgactcttgattttggctcaggtcatgatctcaccgctcatgagactgagccccgcattgggctctgggctgacagcccagagcctgcttgggattctctctctccttctctctctctgcccctctcctgctcatgttctctctctctctcaaaataaataggtaaacttaaactatatatatctgtcaagtttaaaatagtgcctggcacatggtaatcgcttcagaagcaattaataaacaatatagtaattattataaaataataataattggggcgcctgggtggcgcagtcggttaagcgtccgacttcagccaggtcacgatctcgcggtccgtgagttcgagccccgcgtcaggctctgggctgatggcttggagcctggagcctgtttccgattctgtgtctccctctctttctgcccctcccccgttcatgctctgtctctctgtgtcccaaaaataaataaacgttaaaaaaaaaatttaaaataataataataataataagttttccctggttgggtacagggctttccaaaggttagtataaaaaatacacgtgtttaaataaagaggcaatacctcttcgtagcaaaatagttaactagcacaaaaggggaggccgagtgccggctctccccagagagcgtttctcaccctagcgagtttcttgtgtgtttcctgcgatgttctatgcccgcagaagcctatggattgtgtgtgtgtgtgtgtgtgtgtgtgtgtgtgtgcgcgcgcgtgtccctgtgtcctcgcacagactatacacacccgccgtcctgccccttctttagcacgtaaccgtctcgcagctcgctctgcatccgcccctgcgaaaccatcggagtcacgccccttgacaggtgccaggtcccaatgctatttctgagttcacagcttcccagcacagtgtgccctgggggggggggggggcttcccccccgttttacagacgaggcagctggagctgagggggcatcgctcgtctgatgtcacctggtgagtaggtggctcagttgagatgggaatccaggcgtccgggacgcttctcatgtgggcgtttccctcggcctcgtgggacgggagggcagggctcactggcaggcgcgccgagggccccggggccggctctcccgaccatctgggtttccttcccttctcccagcaatgatgtctggagagcccctgcacgtgcggacccccgtccgtgacagcatgtccctgtccaaagtggcgggcaccagcgtctatctcaagatggacagcgtccaaccttcaggctccttcaagatccggggcatcggacatctctgcaggatggtacgggcgggatgggctccacgtcgtctgagggagggggccagtcccacgacgcatccgcggttagcactccccccaaagaggcggtgcccgcccgggagggtctgcccgtggactcatcccagagccgtcatttgcttgcctttcccccgcacccttccctgagccaggccctttggggaggggcgatgtccgcggagaggcaagggcaatacaaagtcctactccctgggggaggggagagcccggggacagcctgggacgtgggcctgaccctccccccttgtctccacagtgggctgagcaaggctgtgaacatttcgtctgctcctcgggtcagtggcggtgtgcctcctcctgtgtggcccagggccgggcgctctacctctctgggccctggtttccccagcgacgagccattacaaacgcctgcggcagccgccgttcagaattccagggggggaggcgggaaggcactcgtaggggggggtgttcggagccccgaggctggggacgacagtcccagctgggctcccaggccatccttcactgacacgctctccaccccatccccggcagcgggcaacgcaggcatggcagccgcctatgccgccaggaagctgggcatccctgccaccatcgtcgtgcccagcaccacacctgccctcaccatcgagcggctcaagaatgagggtgccgtggtcaaggtggtgggtgaggtgagcaccaacccgggggcggggaccacggggggggggcacctggtggcagggcagggtccccccagcccctcaccctctccaacctcccctgagcaccaacccgggggcggggaccacggaggggggggcaacctggtggcaggcagggtccccccagcccctcaccctctccaacctcccctgagcaccaacccgggggcggggaccacggaggggggggcacctggtggcagggcagggtccccccagcccctcaccctctccaacctcccctgagcaccaacccgggggcggggaccacggaggggggggcacctggtggcagggcagggtccccccagcccctcaccctctccaacctcccctgagcaccaacccggggcagggaccacggggggcggcacctggtggcagggcagggtccccccagcccctcaccctctccaacctcccctgagcaccaaccgggggcggggaccacggaggggggggcacctggtggcagggcagggtccccccagcccctcaccctctccaacctcccctgagtaccaacccggggcagggaccacggggaggggggggcacctggtggcagggcagggtccccccagcccctcaccctctccaacctcccctgagcaccaacccggggcagggaccacggggggcggcacctggtggcagggcagggtccccccagcccctcaccctctccaacctccccagagcaccaacccggggcagggaccacgggggggggcacctggtggcagggcagggtccccccagcccctcaccctctccaacttcccctgtggtcttgcagatgttggatgaggcctttgagctggccaaggccctggcaaagaacaacccaggctgggtctacattcctccctttgacgaccccctcatctggtatgtggagccccaaggcacccggtgcagggggcgacagagggctctcgtgggcaggtgactgatgcgggcagccggaaacccgggtggaggtggggaggaggcgactggcccaaagcccgtcagccgcctgggttgaggacgagcgtctccaggatcgcccccagcccccgtggggtctccagggagcccagccctgacccgccccccacccccacccccgtccctaactctccagggaaggccacacttccatcgtggaagaactgaaggagacgctgagtgcaaagccgggggccatcgcgctgtcggtgggtggcgggggcctgctgtgcggagtggtccaggggctgcagaaggtgggctggggggacgtgcccgtcatcgccatggagaccgccggagcccacagcttccacgccgccaccaccgcgggcaagctcgtctccctgccccaggttaccaggtgagcagcggggcccctctggtggatgttcagcaggccttgggaagcccacctcggtgcccgctgcgaggcagagccccgccacctgtcccattttacagacgaggaaactgaggctttccacttgcccagagtcacacggccagagagtgggtctgctgggaccgagcccaggtctgtgtgactctgtaggaggaacgtcacatttcctcacgtgaagagttcactgttaatcactgaccgaggcccctgccacagggttttacacccctcgttaatctgccaaatggttggcccctaacaggggccagtctgagcccctaacaggtgcaagcattgctgatcccggggacagggtggtgaacaaacacagtccctgctcctaaggagttcatattccagccccgaacctttgacagatggtgtgtaattaggattaggtttggcacattacacaggaggcccaagtaacagtgacttaaacaagatggaactttacttcctctctgtaaagtgaaaaaaatgtctagaggtacacagtctcggggcagggagctccctgaagccaggggtccaggttcctcgagtttgttctccctcatcctcatcatgtagccttacggcccaaaagggctgcctaagtgccaactgtcacacccacagtccatctggccaggaagagccatgccccctgcctttaaagatacagtacttccactgacctcttactggccagcatttagtcacgtggccacaactactgcaggggaggctgggaaatgtcttcctatcgggtctccacgtgtgcagctaggatagatattgggtagacaggtccctacctcagatagggaagactgagtccaggtgagacctccttgtggattcctgagcctggtacccagcagccaccatgttggcctctgatcctgcgccgtaggcattgaaataatggcggagtgcctaccacgagtcacgtgccagggctcagggctcaggtggttcaggctgcaggtgctcatgggagatgtgagctgggaataaggccggcgatgactccagggagggcggggagaaagacaaggcaccgattacgcgtgcaggtcccagggttacgtggatcctgctccgggtgcccatgccaccactccctgaacacataaccgtgggccagtgacccgcgtgctctctgcctgggtttcctcttccacggaatcgaaccagtggaacggcaaacccagccgtgaggatttaaaaatgcgcacaaaacgggtggtacctcagactccctcgagaaggggtcgctatgtctgtttatgggggaggaccgaccgttcgagagttccgtccggagacacaggcactggagtccctgggggactccttggaggaggtggccttaatcctgagccctgaaggacagggcgggacttggacgggcagagtggtgcccagctgaggggagggtggagcaggtggcgtcctttcagggaaccgcaagggggcttctccgtccgcgtggatcaggataggctatttaccccactcacgcctccacgtcttatgagacctcactctggcggggagcctctggggcctttcagtgctttacagccacagagggcatgcccgcgcccctgcccgtggaccccacttctctcctccctctcaaatccatttttctctttctatccctcctcgtcccgatctctgccgccttcctccctcctgttttcccccgttccggccttcttctccatcgccacctctcccagcgtggccaaggccctgtgcgtgaagaccgtgggggctcaggccctgaagctgtttcaggaacacccccattttctctgaggtgatctcggaccaggaggctgtggccgccattgagaagtttgtgggtatgtgccaagtccttccaagccctgctggctcagggactaactggtgtcctaacagatcccattcacgacaaattttcctcacagttctgtctcccaccccttgctagaacttcccgttaatgaaagataatacaaggcagcggtataactagaaaggccagagccacaagtgaagaggactgataaccatgatagcagggagcttcctggtagccagggtgaaaaaggaaagccaaccccagggttggcttcagaagtgggtctgtgatgccgttggactcttctgggttcagcctaggacagacaccgcacacacacacacacacacacacgaaagggagcacttaatcctcctgtaccagccgtcagtttggctaatcacccatggcggtcttgctcaagcgacggcccaagccttctcaacacagtgctcgggcagttaacatgcatcggtgggagcgcgctccgggtgaaacctagccctgcctcagggctccgaaggtcaactaggatagcgatgggaggtttggtgcccagactcctagccccgagtctttcccagggacccaacagtccccttgcccccccgcggaagccaggccccaccctcatcaccccctcctctggcagatgacgagaagatcctggtggagcccgcctgcggggcggccctggccgccgtgtacagccgcgtggttcagaagcttcaagcggaggggaagctccgtgccccgctgtcctccctcgtggtcatcgtctgtggcggcagcaacatcagtctgggccagctgcgggccctcaaggaacagctgggcatgaagaacgggctgcccgagtga

SEQ ID NO: 695 - DNA, 7920 bp, Canis lupus
  source 1..7920: mol_type = genomic DNA
attaagccatctgccccttcaagacaaaagaatcataaaggatgacaggagaaggtaattcacattgaggcaggcccactgggttcaaatacctgctcacgtctcttactctctgggtctcagcctccctttctgtaaaacaaggatgatcataggacccacttcctcccatggtttgtttcctgggtgcctactatgtgccaagcacatgctagtgacatgaagtgcaccgtctcccaccctgcaaactcttgggaacaatcctggggaagtcagtattattcccacttgccagtgaagacgctgagaatggggaagggaaagggtgaatgaggggagagaggagaggaagctgccagcaggggaagcacagccagcttccctctctccttctggaagccccagggccacaggccagggggcgggcctggcgggggtgtggccagccctgggtccccattggctgctggacggcgccctgggaataaagctgggaaccaaagacccagacccagacccataggccctgggacgatccagcagacattctaactgtccatctacctccatccgtgagaggaccatctatccagtctatacatctagaccacctcccgtctatccctcctcggcttgctccctctgcctgggcggcccaccttcactgccttggtcaggtaagtacccccacctcagccccaccccagactggagtgcctgtggaatgccaggagattccaggagaagaggaggggatagagtcagttgagcataaaaacccatgctttgaagccagactggctgggtcggcatctagctctgggcctcagtttcccatttccctaaaatgggaacaataactgacccttcctcatcgagctgttgtaaagatgaaatgagatcatgcatttaaaatgtccagtccatggacagcccgggtggctcaggagttaagcgcctgcctacagcccagggagtgatcctggagtcccaggatcgagtcccacatcaggctctctgcatggagcctgcttctccctctgcttgtgtctctgcctctctctcatgcataaatttttaaaatcttaaaaaaaaaataaaatgtccagcccagatataatgatagttcatttttattgagctcttgctatataagccaagtgagagggggagtgacttctgaccaggagggaacattaggtagatagcagcttctagtaccctctgtcctgtgtttggcaaaacctcaggatgttatgctggagggagtggtgaccacagggggtccctacccctcccctgaaggcattgcatggtgcaaccagaggtggaggaggcttcccaaatccacccaaaaggggtcttgtgggggttgcctcccacatccagccaggacagctctttgaggctggggctgggcaacgtgcacaaggtccactcagccagaatgagggtgcccttgtttcagggttaacctccagaaggttcattaaacttaattcagacagatatgggatgctcccgtaatagagtgcctctagtccctggcttttgctcaagagatcctaggggagagatggtatctgttgagcacctctcttaggccccgaatgtcagttaattcagacagacgagcgatccatatttgggggtgctaagggtggtggagtggtcaggaagcctacccctcactttgcttctgaccaaggcatatctagcccagtctgggagtggcaatggcccagggctagggcggcaccaggtagtgtcattggaaatagggtgggaggtagaaagagggagccagtcttgcttggccttgtgggatgggggaggtggtagtggtgggggaccagagaggtgacttacccaaccatctgtggatcagttctggttcagcccaagttggggagaatcagaaaaagaagagaaaaaaaggctgcatgtatgtggccagactccggaccatggcagcttcttcatcactcagagtcagtccagaccgggaggcctgagccatgcccatgtgtcctggagacagcttagttcaggtgctagactgtctgaattcaaatcctgcctcccccactcactagctgtgtgaccttgggcaagtcacattaatattccgatccctattgctatgtgacaaactgcccgaaggtatagtgaccaaaaacaacagcaatcattgtattgttgtctcatggctctggggcttaattgggctctgcaggcagttctcgctggactctctcaggcggctgcagtgtgacagtggctggtgatgtcccaaggtcacacacacacacagacacctacccgtctcacaagtgacgctggccatcagctgtcaggtaggggcacctccatatggcctctgcatgaggcctacgcttacccgcctcacggcagctgggttttggaagagagttgctcaagagaaatagccaggtggaggctgtgtccccttctgtgacccagccctgggtgaggggctgccctgaagccgactcacacagactctggaaagcggactgtgccatctcttcccagctccacactcatgacatcgtgttggtagtgtgaacgcggccttggtgaagcttatgtacagcacagaggtgcacacactacaaatcagggctttatcttggagagtaaacatttttaccagcactttgctgcttggaagccacatagtgtcactgcagccacagacaccctctgcccctccagggctcatgtggaggaagcacagaccccacctctcactgagatgatcactcataagagatgcgtgtgggatggggagagcttgcagcggccatcccgggaaacacaatccgagcctttttcctcatcaggagaatgggatgataaggcacctcctaggaaggctaaaaggattaaatgaattaatatctgtaaggcttaaaacattgtgtggcacgtggtgatcactttagaagtgattatcacataatataatgactataaaaagtctctctaactgggtgcggggctttccaaaggttagtataaaaaaaatgcatgtatctaaacagataatccctaaacacgacaaaacattcaaacagtacaaaagggatgctgagtgccagctctccccagaagccccttgccctaacaagtttcttgtgtgtatcctgagacggcctatgcacacagaagcttatggattatgtacgtggtgggggtgtccccatattttttcacaaatagactctctctgctatcctgccccttctttaccactaccacttaagcatcttgaagctcactctgaatcagcacctgcaaatccatctccacctcctgcccctggaaaggtgcaaggtctgaacgttatttctaatgttcacagcttcccagcatgctgtgtgcatggggtgggtggcggtgctggtgatggtaagaatccttgtccccattttacagatgaggcagctgaggcagagggggggattggttgtgtgatgtcacctggtacgtaggtggctcagttgagatttgaattcaggtctccaggatgctcctcatctgggctctttcctcggcttcgtgggcagtgaggggaggagtcactcagaggcacactgagggctgtatggcagcggctggctctcctgaccatctgggtgtccttccttcctcccagcgatgacgcctggagagcccctgcacgtgaagacccccatccgtgacagcatgtcgctgtccaaagtggcaggcaccagcgtctacctcaagatggacagtgcccaaccttcaggctccttcaagatccggggcattggacacctctgcaagatggtacaggagggctccgcagcatctgagtgagggtggcatgtcctttatcttcaaaggacaaggccagtgttgtgtgctcactctcacaaagcccttgtatctgccattagcactccctctaggaaggggggccacccaagggggtctccttatggactgagcccagagccatcatttgcctgcttcccttcaccccaacccttccctgagccagtctccggggcggggcgatgtcatctgagaggctagggcaatacagagtctttgtgaaggaaagagggcctggtgcatattggaatcactgggaagcactccggacaggagagctgtctgggcggtggcctgacctcccctcttgtctccacagtgggctgagcaaggctgtgaacatttcgtctgctcctcgggtgagtggcctgcatgcttcctcctgtgccctgctgtgtgtcccaggccaggcacccccccggcgcaccccccctcccggggccttggtttccgtagcaatgagccattgcaaacatctgcagcggccccaatttggaactccggggcgggggggggggggggggggggaggcgagctgtcactcagggaggtgtgcagagccccagggttgggggcgagcaggcccagctgggctcccaggcctttctgcactgacatactctccacccccatccctggcagcgggcaatgcaggcatggcagccgcctatgctgccaggaagctgggcatccctgccaccatcgttgtgcccagcaccacccctgccctcaccattgagcggctcaagaatgagggtgccatagtcaaggtggtgggtgaggtgagtaccaacaacccagggcaggggccgcggagggcacccggtggctgggcagggtctccagcccctcaccccctccaacctccctgtggtcttgcagatgttggatgaggccttcgagctggccaaggccctggccaagaacaaccctggctgggtgtacattcctccctttgacgaccccctcatctggtatgtggagcccagtcccctggtacaggtggcgttagagaaaggggaggtgagccgaagccggaagggagccagtaaagggctctcatgggcaggtgaccgctatgggcccactcggggcccctgggagccagtgtggatcccaaaccagaattattccacctgcgggagagcgtgctggggtttatacaccaactgccaccgttctggactcccgcagacattggttgagctgctttccgggggtgtcctctcctgccacagttgtaagcagctccagtggccagagaaagcccttgggtagtcacgcagatgttggcagctggaagtcggggtggagttggggatcaggcaactagcccaaagcccatcagctgcctgggaggacaggtggcacctccaggacccctgcgtggtgtctccagggagcccagccctgacctgtgccccctccccccctccccagggaaggccacacttccatcgtgaaggagctgaaggagaccctgagtgcaaagccaggggccatcgcgctgtcagtgggcggcgggggcctgctgtgcggagtggtccaggggctgcaagaggtgggctggggggacgtgcctgtcatcgccatggagaccgccggagcccacagcttccacgctgccaccactgccggcaagctggtctccctgtcccaggttaccaggtgagcagagggcctgcctctgtcggtgttcggtaagccttggggagcccgcctcggtgttggctgcaaggtagacagaccacctccatgtcagtcccattttacagatgaggaagctgaggctttggtcacttgcccagagtcacaagccagcaagtggtactgctgagactgaatgcgaccgagtctacgtgactcagtaccatgaacattacactcctcacatgaagaggccaatattacgcatttattgagtttcctgcagcagggttttacacaccttgttgatttgccaaccatttactgggcccctaaaatgtgcaagcggtgctgatcctggggacagagtggtgaataaacacagcccccggtccccaggagctcatatcccagccccaaacctttgagagatgatacttaagattaggtttggcacattatacaagtaccccaaatagcagtgacttaaaccagattcaactttactttctcttatataagtggaaaaaaaaaaaaaaaaaaagcctagaggtaggcggtccagggcagctccatgaagccagaggcccaggttcgttgcgtttgttctccaccgtccttatcatgtagccctatggcccaaaatagctgctcatgtgccgactatcacacccatgctcgttctggcaggaggagccccctccctttaaagacaggcacaggggatgcctgggtggctcagcggttgagcgtctgccttccactgagggcgtgatcctggtcctgggatcgagtcccgcctcagggtccttgcggggagcctgcttcttcctctgcctgtgtctctgcctctctctgtgtctctcatgaataaataaatctttaaaaaaaaataaagacaggcacagtacttccacttacctctcattggccagaacttagtcatgtggccacgactactgcaagggaggctgggaaatatcttcctaccacgtctccacatgcagctgggttggatactgggtagacaagtccttaccgcagatagggaagactgagcccaggtaagaactcctgggctcctgaacctggcacctgggagctaccatgttgggctctgatcctgcagggttgggattgaggataattgtcgcgtgcctacatgtgccacatgctgggcttagggctaaggtggttcaggccccaggggctccccagtgagctgggaatagaaccggtgatgactccagggagaggggggagaaagtgaatgcactgaataggagtgcaggtcctggagttacatggatcctgcttaaggatctttctcctgcctcctgtttgccccattcccgtcctcttctctatccccacctctcccagcgtggccaaggccctgtgcgtgaagactgtgggggctcaagccctgaagctgtttcaggaacaccccatcttctctgaagtcatctcggaccaggaggctgtggctgccattgagaagttcgtgggtatgtttcaagacctgaccccctagcttggggattaactggctgtctgctcagtggcctaagagatcccattcgctgtaaattttcttcatcgtcctatctccccatcccttcctagaacctcccactatgaaagataatacaatgcagcagtaaaaatagaaaggccaaagccacaagtgaagaaacctgataaccatgacagcaaggaataataactgtttaaaatgctgtgagcttcctggtagccagggtgaaaaaggaaaatacaggcctcagggttggcttcagaaataggtctataactaggctggactcctaggttcagtctaggacaggggtggggtgcatgcatatgtgcgcatgcacacacgtgcacacacacacatacacacacgcaaaggtagcactaacactgccagttaactcctgtgccagcagtcagttgggctaaccgaccacagcagtcttgctcaaatgatgactcaagccttctcaacccagtgcttgggcagttacaatcgtttcatcagtgcgagcactcgagatgagacctcgcctcggaaggtcaatctagcatagtgacgggaggtgtggcccggacccagtctgcggccctgccgttcccttgcagcctgcacccaggcctcaccctcaccacccttccccggcagatgacgagaagatcctggtggagcccgcctgcggggcagccctggccgccgtgtacagccgcgtggtgcagaagctgcaaggggaggggaagctccaggccccgctgtcctccctcgtggtcattgtctgcgggggcagcaacatcagtctgagccagctgcgggccctcaaggaacagctgggcatgaatgggctgcccaagtga

(19) United States
(12) Patent Application Publication
(10) Pub. No.: US 2026/0035701 A1
(43) Pub. Date: Feb. 5, 2026

(54) METHODS AND COMPOSITIONS FOR TARGETING SERINE DEHYDRATASE

(71) Applicants: Salk Institute for Biological Studies, La Jolla, CA (US); The Regents of the University of California, Oakland, CA (US)

(72) Inventors: Christian Metallo, San Diego, CA (US); Prashant Mali, San Diego, CA (US); Emeline Joulia, San Diego, CA (US); Joseph Rainaldi, San Diego, CA (US); Patrick Tseng, San Diego, CA (US); Ethan Ashley, San Diego, CA (US)

(21) Appl. No.: 19/243,500

(22) Filed: Jun. 19, 2025

Related U.S. Application Data
(60) Provisional application No. 63/662,174, filed on Jun. 20, 2024.

(51) Int. Cl. C12N 15/113; A61K 38/26; A61K 48/00; A61P 3/04; A61P 17/02; A61P 25/02; C12N 9/22; C12N 15/11; C12N 15/86; C12N 15/88; G01N 33/68

(52) U.S. Cl. CPC C12N 15/1137; A61K 38/26; A61K 48/005; A61K 48/0083; A61P 3/04; A61P 17/02; A61P 25/02; C12N 9/222; C12N 15/111; C12N 15/86; C12N 15/88; G01N 33/6812; C12N 2310/11; C12N 2310/14; C12N 2310/20; C12N 2310/351; C12N 2310/531; C12N 2320/31; C12N 2750/14143; C12Y 403/01017; G01N 2800/52

(57) ABSTRACT

Disclosed herein are methods and compositions for increasing serine levels, increasing ceramide levels, and/or decreasing deoxyceramide, such as deoxydihydroceramide, levels in one or more tissues in a subject in need thereof. In particular, the technology of the present disclosure relates to biological approaches for disrupting serine hydratase (SDS) activity in a subject.

### CROSS-REFERENCE TO RELATED APPLICATIONS

This application claims the benefit of and priority to U.S. Provisional Patent Application No. 63/662,174, filed Jun. 20, 2024, the content of which is incorporated herein by reference in its entirety for any and all purposes.

### STATEMENT REGARDING FEDERALLY SPONSORED RESEARCH OR DEVELOPMENT

This invention was made with government support under 1R01CA234245 and R01HG012351 awarded by the National Institutes of Health, and under W81XWH-22-1-0401 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created on Sep. 3, 2025, is named 138239-0108_SL.xml and is 660,572 bytes in size.

### TECHNICAL FIELD

The present technology relates generally to methods and compositions for increasing serine levels, increasing ceramide levels, and/or decreasing deoxyceramide levels in one or more tissues in a subject in need thereof. In particular, the present technology relates to biological approaches for disrupting serine hydratase (SDS) activity in a subject.

### BACKGROUND

The following description is provided to assist the understanding of the reader. None of the information provided or references cited is admitted to be prior art.

Amino acid dysregulation has emerged as an important driver of disease progression in various contexts. l-Serine lies at a central node of metabolism, linking carbohydrate metabolism, transamination, glycine, and folate-mediated one-carbon metabolism to protein synthesis and various downstream bioenergetic and biosynthetic pathways. l-Serine is produced locally in the brain but is sourced predominantly from glycine and one carbon metabolism in peripheral tissues via liver and kidney metabolism. Compromised regulation or activity of l-serine synthesis and disposal occurs in the context of genetic diseases as well as chronic disease states, leading to low circulating l-serine levels and pathogenesis in the nervous system, retina, heart, and aging muscle. Dietary interventions in preclinical models modulate sensory neuropathy, retinopathy, tumor growth, and muscle regeneration. Serine tolerance tests may provide a quantitative readout of l-serine homeostasis that identifies patients who may be susceptible to neuropathy or responsive to therapy.

However, while dietary interventions have shown some benefits they require substantial lifestyle changes and commitment from subjects and they only address a single source of serine. There is a diverse array of sources and sinks of l-serine present in mammals, including diet, synthesis from glucose, regeneration from glycine, biosynthesis, and catabolism to glucose, with several organs involved in the maintenance of l-serine homeostasis. There are additionally numerous biological fates of l-serine, which contribute to many downstream pathways important for cell function, including the synthesis of sphingolipids, ceramides, and other important biological signaling molecules. Accordingly, there is a need for better compositions and methods for regulating serine homeostasis.

### SUMMARY

In one aspect, the present disclosure provides a method for increasing serine levels in one or more tissues in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In some embodiments, the agent that disrupts SDS activity is delivered via a vector selected from the group consisting of: an adeno-associated virus, a virus-like particle, and a lipid nanoparticle. In some embodiments, the vector is targeted to the liver and/or the kidney. In some embodiments, administration of the agent to the subject results in one or more of inhibiting the transcription of the SDS gene or translation of mRNA transcribed from the SDS gene. In some embodiments, the one or more tissues is selected from blood tissue, liver tissue, kidney tissue, ocular tissue, muscle tissue, and skin tissue, or any combination thereof. In some embodiments, the one or more tissues is blood tissue, and administration of the agent to the subject increases plasma serine levels in the subject in both fed and fasting states. In some embodiments, the one or more tissues is blood tissue, and administration of the agent to the subject increases plasma serine levels in the subject by at least about 10% to at least about 300%. In some embodiments, administration of the agent to the subject increases glycine and/or threonine levels in the one or more tissues. In some embodiments, the one or more tissues is blood tissue, and administration of the agent to the subject increases plasma glycine and/or threonine levels in the subject in either fed or fasting states. In some embodiments, the one or more tissues is blood tissue, and the administration of the agent to the subject increases plasma glycine levels by at least about 10% to at least about 300% and/or plasma threonine levels by at least about 10% to at least about 600%. In some embodiments, administration of the agent to the subject decreases 1-deoxydihydroceramide (doxDHCer) levels in the one or more tissues. In some embodiments, the one or more tissues is blood, and the 1-deoxydihydroceramide is selected from the group consisting of: doxDHCer m18:0/22:0, doxDHCer m18:0/24:0, doxDHCer m18:0/24:1, and doxDHCer m18:0/26:0. In some embodiments, the subject is diagnosed with, suspected of having, or at an increased risk of, diabetic peripheral neuropathy, macular telangiectasia (MacTel), one or more wounds, liver damage, nerve damage, and muscle damage. In some embodiments, the agent is administered orally, topically, systemically, intravenously, subcutaneously, transdermally, iontophoretically, intranasally, intraperitoneally, or intramuscularly. In some embodiments, the subject is human, canine, feline, murine, or equine. In some embodiments, the method further comprises administering an additional therapeutic agent selected from the group consisting of serine, glycine, threonine, glucose, a Glucagon-like peptide-1 (GLP-1) agonist, or glutamine to the subject. In some embodiments, the method does not comprise administering serine to the subject. In some embodiments, the subject is at risk for or has been diagnosed with Type 1 diabetes or Type 2 diabetes. In some embodiments, the method further comprises administering a Glucagon-like peptide-1 (GLP-1) agonist to support maintenance of muscle mass. In some embodiments, the agent is a shRNA, RNAi, or anti-sense oligonucleotide selected from any one or more of the oligonucleotides as set forth in SEQ ID NOs: 2-6, Table 12, or Table 13. In some embodiments, the agent is a zinc finger nuclease comprising a zinc finger having the amino acid sequence as set forth in Table 1, Table 2, Table 3, Table 4, or Table 5. In some embodiments, the agent is a Clustered Regularly Interspaced Short Palindromic Repeats (CRISPR)-associated (CRISPR-Cas) system comprising a guide RNA comprising an oligonucleotide selected from any one or more of the oligonucleotides as set forth in Table 6, Table 7, Table 8, Table 9, Table 10, or Table 11. In some embodiments, the agent is conjugated to at least one sugar moiety, optionally N-acetyl galactosamine (GalNAc), in particular triantennary GalNAc.

In one aspect, the present disclosure provides a composition comprising an agent the disrupts serine dehydratase (SDS) activity, wherein the agent is selected from the group consisting of: (a) an antisense oligonucleotide, or a nucleic acid encoding the same, comprising a nucleotide sequence selected from any one of the oligonucleotides as set forth in Table 12 or Table 13; (b) a siRNA, or a nucleic acid encoding the same, comprising a nucleotide sequence selected from any one of the oligonucleotides as set forth in Table 12 or Table 13; (c) a shRNA, or a nucleic acid encoding the same, comprising a nucleotide sequence selected from any one of the oligonucleotides as set forth in SEQ ID NOs: 2-6, Table 12, or Table 13; (d) a zinc finger peptide, or a nucleic acid encoding the same, as set forth in Table 1, Table, 2, Table 3, Table 4, or Table 5; and (e) a nucleic acid encoding a CRISPR-Cas system, wherein the nucleic acid encodes a guide RNA comprising the oligonucleotide selected from any one of the oligonucleotides as set forth in Table 6, Table 7, Table 8, Table 9, Table 10, or Table 11. In some embodiments, the agent comprises an antisense oligonucleotide, or a nucleic acid encoding the same, comprising a nucleotide sequence selected from any one of the oligonucleotides as set forth in Table 12 or Table 13. In some embodiments, the agent comprises a siRNA, or a nucleic acid encoding the same, comprising a nucleotide sequence selected from any one of the oligonucleotides as set forth in Table 12 or Table 13. In some embodiments, the agent comprises a shRNA, or a nucleic acid encoding the same, comprising a nucleotide sequence selected from any one of the oligonucleotides as set forth in SEQ ID NOs: 2-6, Table 12, or Table 13. In some embodiments, the agent comprises a zinc finger peptide having the amino acid sequence as set forth in Table 1, Table 2, Table 3, Table 4, or Table 5, or a nucleic acid encoding the same. In some embodiments, the agent comprises a nucleic acid encoding a CRISPR-Cas system, wherein the nucleic acid encodes a guide RNA selected from any one of the oligonucleotides as set forth in Table 6, Table 7, Table 8, Table 9, Table 10, or Table 11. In some embodiments, the agent is conjugated to at least one sugar moiety, optionally N-acetyl galactosamine (GalNAc), in particular triantennary GalNAc. In some embodiments, the composition further comprises a pharmaceutically acceptable carrier.

In a different aspect, the present disclosure provides a method for increasing ceramide levels and/or decreasing deoxyceramide levels in one or more tissues in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger, and a nucleic acid encoding a CRISPR-Cas system. In some embodiments, the agent that disrupts SDS activity is delivered via a vector selected from the group consisting of: an adeno-associated virus, a virus-like particle, and a lipid nanoparticle. In some embodiments, the vector is targeted to the liver and/or the kidney. In some embodiments, disrupting the activity of SDS comprises one or more of inhibiting the transcription of the SDS gene or translation of mRNA transcribed from the SDS gene. In some embodiments, the one or more tissues is selected from liver tissue, kidney tissue, and skin tissue, or any combination thereof. In some embodiments, the tissue is liver tissue, and: (i) the increased ceramide levels comprise increased levels of one or more of dihydroceramide d18:0/24:0, ceramide d18:1:22:0, ceramide d18:1/24:0, and ceramide d18:1/24:1; and (ii) the decreased deoxyceramide levels comprise increased levels of one or more of deoxydihydroceramide m18:0/22:0, deoxydihydroceramide m18:0/24:0, and deoxydihydroceramide m18:0/24:1. In some embodiments, the tissue is kidney tissue, and: the decreased deoxyceramide levels comprise increased levels of one or more of deoxydihydroceramide m18:0/20:0, deoxydihydroceramide m18:0/22:0, deoxydihydroceramide m18:0/23:0, deoxydihydroceramide m18:0/24:0, and deoxydihydroceramide m18:0/24:1. In some embodiments, the tissue is skin tissue, and: (i) the increased ceramide levels comprise increased levels of one or more of ceramide d18:1/16:0 and ceramide d18:1/16:1; and (ii) the decreased deoxyceramide levels comprise increased levels of one or more of deoxydihydroceramide m18:0/22:0, deoxydihydroceramide m18:0/24:0, and deoxydihydroceramide m18:0/24:1. In some embodiments, the subject is diagnosed with, or at an increased risk of, neuropathy, muscle damage, MacTel, or one or more wounds. In some embodiments, the agent is a shRNA, RNAi, or anti-sense oligonucleotide selected from any one or more of the oligonucleotides as set forth in SEQ ID NOs: 2-6, Table 12, or Table 13. In some embodiments, the agent is a zinc finger nuclease comprising a zinc finger having the amino acid sequence as set forth in Table 1, Table 2, Table 3, Table 4, or Table 5. In some embodiments, the agent is a Clustered Regularly Interspaced Short Palindromic Repeats (CRISPR)-associated (CRISPR-Cas) system comprising a guide RNA comprising an oligonucleotide selected from any one or more of the oligonucleotides as set forth in Table 6, Table 7, Table 8, Table 9, Table 10, or Table 11. In some embodiments, the agent is conjugated to at least one sugar moiety, optionally N-acetyl galactosamine (GalNAc), in particular triantennary GalNAc.

In another aspect, the present disclosure provides a method for selecting a subject for treatment with a therapeutically effective amount of an agent that disrupts serine dehydratase (SDS) activity, the method comprising: (a) detecting levels of serine in a blood sample from the subject; (b) selecting the subject for treatment with the agent that disrupts SDS activity where the level of serine and/or glycine in the blood sample from the subject is reduced by about 20% compared to the normal control sample; and (c) administering to the subject a therapeutically effective amount of the agent that disrupts SDS activity, wherein the agent is selected from the group consisting of: an antisense oligonucleotide, a shRNA, a siRNA, a zinc finger nuclease, and CRISPR-Cas system. In some embodiments, the agent is a shRNA, RNAi, or anti-sense oligonucleotide selected from any one or more of the oligonucleotides as set forth in SEQ ID NOs: 2-6, Table 12, or Table 13. In some embodiments, the agent is a zinc finger nuclease comprising a zinc finger having the amino acid sequence as set forth in Table 1, Table 2, Table 3, Table 4, or Table 5. In some embodiments, the agent is a Clustered Regularly Interspaced Short Palindromic Repeats (CRISPR)-associated (CRISPR-Cas) system comprising a guide RNA comprising an oligonucleotide selected from any one or more of the oligonucleotides as set forth in Table 6, Table 7, Table 8, Table 9, Table 10, or Table 11. In some embodiments, the agent is conjugated to at least one sugar moiety, optionally N-acetyl galactosamine (GalNAc), in particular triantennary GalNAc.

In another aspect the present disclosure provides a method for treating diabetes in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In one aspect the present disclosure provides a method for treating diabetic peripheral neuropathy in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In another aspect the present disclosure provides a method for treating obesity in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In one aspect the present disclosure provides a method for treating one or more wounds in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In some embodiments, the agent that disrupts SDS activity is delivered via a vector selected from the group consisting of: an adeno-associated virus, a virus-like particle, and a lipid nanoparticle. In some embodiments, the vector is targeted to the liver and/or the kidney. In some embodiments, administration of the agent to the subject results in one or more of inhibiting the transcription of the SDS gene or translation of mRNA transcribed from the SDS gene. In some embodiments, the subject as compared to an untreated control subject, exhibits one or more of (i) increased serum serine abundance; (ii) increased serum threonine abundance; (iii) increased serum serine:alanine ratio; (iv) increased rate of wound healing; (v) reduced weight gain and/or blood glucose levels; (vi) reduced iWAT and/or eWAT weight; (vii) reduced serum palmitate and/or cholesterol levels; and (viii) reduced peripheral neuropathy. In some embodiments, the subject is diagnosed with, suspected of having, or at an increased risk of, diabetes, diabetic peripheral neuropathy, obesity, or one or more wounds. In some embodiments, the agent is administered orally, topically, systemically, intravenously, subcutaneously, transdermally, iontophoretically, intranasally, intraperitoneally, or intramuscularly. In some embodiments, the subject is human, canine, feline, murine, or equine. In some embodiments, the method further comprises administering an additional therapeutic agent selected from the group consisting of serine, glycine, threonine, glucose, a Glucagon-like peptide-1 (GLP-1) agonist, or glutamine to the subject. In some embodiments, the method does not comprise administering serine to the subject. In some embodiments, the subject is at risk for or has been diagnosed with Type 1 diabetes or Type 2 diabetes. In some embodiments, the method further comprises administering a Glucagon-like peptide-1 (GLP-1) agonist to support maintenance of muscle mass. In some embodiments, the agent is a shRNA, RNAi, or anti-sense oligonucleotide selected from any one or more of the oligonucleotides as set forth in SEQ ID NOs: 2-6, Table 12, or Table 13. In some embodiments, the agent is a zinc finger nuclease comprising a zinc finger having the amino acid sequence as set forth in Table 1, Table 2, Table 3, Table 4, or Table 5. In some embodiments, the agent is a Clustered Regularly Interspaced Short Palindromic Repeats (CRISPR)-associated (CRISPR-Cas) system comprising a guide RNA comprising an oligonucleotide selected from any one or more of the oligonucleotides as set forth in Table 6, Table 7, Table 8, Table 9, Table 10, or Table 11. In some embodiments, the agent is conjugated to at least one sugar moiety, optionally N-acetyl galactosamine (GalNAc), in particular triantennary GalNAc.

### BRIEF DESCRIPTION OF THE DRAWINGS

FIG. 1 shows a non-exhaustive diagram of several sources of serine.

FIG. 2 shows a diagram of several serine metabolic pathways.

FIG. 3A is a series of graphs showing the following for wild type male mice (WT) and diabetic model male mice (db/db) after a serine tolerance test: (left) plasma serine levels over time; (center-left) the area under the curve for the plasma serine levels; (center) plasma pyruvate levels over time; (center-right) the area under the curve for the plasma pyruvate levels; and (right) relative SDS activity in liver and kidney samples. FIG. 3B is a series of graphs showing the following for wild type female mice (WT) and diabetic model female mice (db/db) after a serine tolerance test: (left) plasma serine levels over time; (center-left) the area under the curve for the plasma serine levels; (center-right) plasma pyruvate levels over time; and (right) the area under the curve for the plasma pyruvate levels. FIG. 3C is a series of graphs showing the following for type 1 diabetes model mice treated with streptozotocin (STZ) or vehicle (Veh): (left) plasma serine levels over time; (center) the area under the curve for the plasma serine levels; and (right) relative SDS activity in liver and kidney samples.

FIG. 4A is a series of graphs showing plasma serine levels over time after a serine tolerance test (STT) in mice treated with an adeno-associated virus SDS overexpression construct (AAV-SDS) or an adeno-associated virus control (AAV-CTRL) at 10 days, 4 weeks, and 8 weeks post treatment. FIG. 4B is a series of graphs showing mouse plasma glycine (left), methionine (center-left), and pyruvate (center-right) levels and blood glucose (right) levels over time in mice treated with AAV-SDS or AAV-CTRL at 10 days post treatment. FIG. 4C is a series of graphs showing the following for mice treated with AAV-SDS or AAV-CTRL: (left) the baseline (levels after an overnight fast) concentration of serine (Ser) glycine (Gly), methionine (Met), alanine (Ala), and threonine (Thr); (center) relative plasma pyruvate abundance; and (right) baseline glucose concentration.

FIG. 5A shows graphs of (left) plasma serine levels over time and (right) the area under the curve of plasma serine levels over time in wild type mice (WT), heterologous knockout of SDS mice (SDS-Het), and full knockout of SDS mice (SDS-KO) after a serine tolerance test. FIG. 5B shows graphs of (left) plasma glycine levels over time, (center) plasma methionine levels over time, and (right) plasma threonine levels over time in WT, SDS-Het, and SDS-KO mice after a serine tolerance test. FIG. 5C shows graphs of (left) fasted blood glucose levels over time in WT and SDS-KO mice after a serine tolerance test, (center) fasted baseline glucose concentrations in WT and SDS-KO mice, and (right) fed glucose concentration in WT, SDS-Het, and SDS-KO mice. FIGS. 5D-5I show graphs of the following for WT, SDS-Het, and SDS-KO mice: fasted plasma concentration of serine (Ser), glycine (Gly), methionine (Met), threonine (Thr), and alanine (Ala); fed plasma concentration of Ser, Gly, Met, Thr, and Ala; fasted plasma concentration of glutamine (Gln), glutamic acid (Glu), asparagine (Asn), aspartate (Asp), and lysine (Lys); fed plasma concentration of Gln, Glu, Asn, Asp, and Lys; fasted plasma concentration of valine (Val), leucine (Leu), isoleucine (Ile), phenylalanine (Phe), and tyrosine (Tyr); and fed plasma concentration of Val, Leu, Ile, Phe, and Tyr.

FIGS. 6A-6H show graphs of the following for WT, SDS-HET, and SDS-KO mice: plasma serine levels over time after a serine tolerance test (STT); area under curve for plasma serine levels; plasma glycine levels over time after STT; area under curve for plasma glycine levels; plasma threonine levels over time after STT; area under curve for plasma threonine levels over time; plasma methionine levels over time after STT; area under curve for plasma methionine levels.

FIGS. 7A-7F show graphs of the following for blood samples taken from WT, SDS-HET, and SDS-KO mice, with blue highlighted points indicating male mice and unhighlighted points indicating female mice: individual dihydroceramide (DHCer) abundance; individual ceramide (Cer) abundance; individual deoxydihydroceramide (doxDHCer) abundance; individual deoxyceramides (doxCer) abundance; individual glucosyl/galactosylceramine (Gluc/Gal-Cer) abundance; and individual sphingomyelin (SM) abundance. FIGS. 7G-7M show graphs of the total relative abundance of the following compounds for blood samples taken from WT, SDS-HET, and SDS-KO mice, with blue highlighted points indicating male mice and unhighlighted points indicating female mice: SO-C18:1; DHCer, Cer, doxDHCer, doxCer, Gluc/Gal-Cer, and SM.

FIGS. 8A-8C show graphs of the abundance of serine (Ser), glycine (Gly), methionine (Met), threonine (Thr), and alanine (Ala) in SDS-WT and SDS-KO fed mice in the liver, kidney and skin. FIGS. 8D-8I show graphs of the abundance of the following for SDS-WT and SDS-KO fed mice: liver glutamine (Gln), glutamic acid (Glu), asparagine (Asn), aspartate (Asp), and lysine (Lys); kidney Gln, Glu, Asn, Asp, and Lys; and skin Gln, Glu, Asn, Asp, and Lys; liver valine (Val), leucine (Leu), isoleucine (Ile), phenylalanine (Phe), and tyrosine (Tyr); kidney Val, Leu, Ile, Phe, and Tyr; and skin Val, Leu, Ile, Phe, and Tyr.

FIGS. 9A-9F show graphs of the following in the liver of fed SDS-WT and SDS-KO mice, with blue highlighted data points indicating male mice and unhighlighted points indicating female mice: sphinganine d18:0 (CAS number 764-22-7) abundance (SA d18:0); individual dihydroceramide abundance (DHCer); individual ceramide abundance (Cer); individual sphingosine (SO) abundance; composite dihydroceramide (DHCer) abundance; composite ceramide (CER) abundance. FIGS. 9G-9I show graphs of the following in the liver of fed SDS-WT and SDS-KO mice, with blue highlighted data points indicating male mice and unhighlighted points indicating female mice: deoxysphinganine m18:0 (CAS number: 196497-48-0) abundance (doxSA m18:0); individual deoxydihydroceramides abundance (doxDHCer); and composite deoxydihydroceramides (doxDHCer) abundance. FIGS. 9J-9O show graphs of the following in the kidney of fed SDS-WT and SDS-KO mice, with blue highlighted data points indicating male mice and unhighlighted points indicating female mice: SA d18:0 abundance; individual DHCer abundance; individual Cer abundance; individual SO abundance; composite DHCer abundance; composite Cer abundance. FIGS. 9P-9R show graphs of the following in the kidney of fed SDS-WT and SDS-KO mice, with blue highlighted data points indicating male mice and unhighlighted points indicating female mice: doxSA d18:0 abundance; individual doxDHCer abundance; and composite doxDHCer abundance. FIGS. 9S-9X show graphs of the following in the skin of fed SDS-WT and SDS-KO mice, with blue highlighted data points indicating male mice and unhighlighted points indicating female mice: SA d18:0 abundance; individual DHCer abundance; individual Cer abundance; individual SO abundance; composite DHCer abundance; and composite Cer abundance. FIGS. 9Y-9AA show graphs of the following in the skin of fed SDS-WT and SDS-KO mice, with blue highlighted data points indicating male mice and unhighlighted points indicating female mice: doxSA d18:0 abundance; individual doxDHCer abundance; and composite doxDHCer abundance.

FIGS. 10A-10C show Western blots depicting expression of SDS or Vinculin loading control, normalized densitometry measurements for SDS showing shRNA72 and shRNA74 have the greatest knockdown, and relative abundances of serine, threonine, and glycine in cells expressing human SDS and SDS-targeting shRNA. Data indicate that shRNAs 72 and 74 induce the strongest reduction in serine, glycine, and threonine catabolism.

FIGS. 11A-11D show that Serine dehydratase knock-down in BKS-db/db mice decreases serine catabolism and mitigates peripheral neuropathy. FIG. 11A shows the results of a serine tolerance test on BKS-db/db mice 4 weeks after shRNA injection. FIG. 11B shows the plasmatic amino acid concentration and FIG. 11C shows the serine/alanine ratio 4 weeks after shRNA injection. FIG. 11D is a graph showing the thermal latency assessed 8 weeks post shRNA injection.

FIGS. 12A-12F shows that serine dehydratase deletion in db/db mice decreases serine catabolism and improves wound healing. FIGS. 12A-12B are graphs showing the plasmatic (FIG. 12A) and hepatic (FIG. 12B) amino acid concentration in db/db-Sds-WT and db/db-Sds-KO mice. FIG. 12C is a graph showing the results of a serine tolerance test perform on 12-14 week old female and male mice. FIGS. 12D-12E are graphs showing the ratio between serine and alanine in the liver (FIG. 12D) and skin deoxydihydroceramides (FIG. 12E). FIG. 12F is a graph showing the percent of initial wound area measured in 15-16 week old db/db-Sds-CTRL and db/db-Sds-KO female mice.

FIGS. 13A-13E show that serine dehydratase deletion protects from High Fat Diet-induced obesity. FIG. 13A is a graph showing the percent of initial body weight of females Sds-WT and Sds-KO fed with HFD for 16 weeks. FIG. 13B is a graph showing the inguinal and epididymal white adipose tissue (iWAT and eWAT) weights and FIG. 13C shows H&E staining on liver and iWAT slices from Sds-WT and KO mice fed with HFD for 16 weeks. FIG. 13D is a graph showing liver palmitate and FIG. 13E is a graph showing cholesterol from Sds-WT and KO mice fed with HFD for 16 weeks.

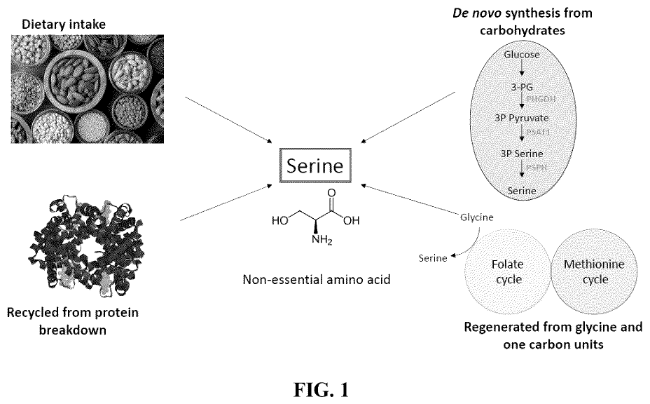

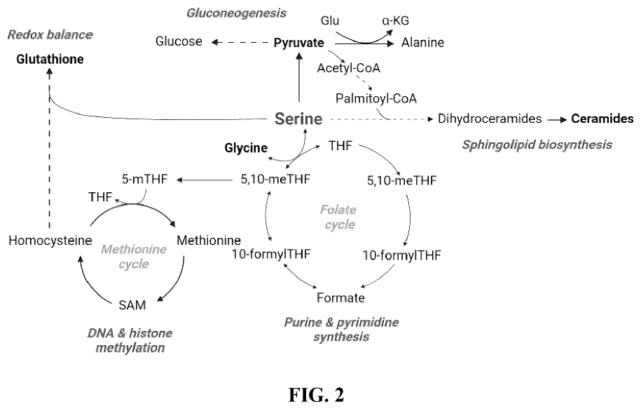

### DETAILED DESCRIPTION

It is to be appreciated that certain aspects, modes, embodiments, variations and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, “administering” or “administration” of an agent to a subject includes any route of introducing or delivering to a subject the agent to perform its intended function. Administration can be carried out by any suitable route, such as oral administration. Administration can be carried out orally. Administration can be carried out subcutaneously. Administration can be carried out intraperitoneally. Administration can be carried out topically. Administration can be carried out intraocularly. Administration can be carried out ophthalmically. Administration can be carried out systemically. Alternatively, administration may be carried out intravenously, intranasally, intradermally, transdermally, intrathecally, intracerebroventricularly, iontophoretically, transmucosally or intramuscularly. Administration includes self-administration and the administration by another.

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

The terms “disruption” as used herein to refers to a disrupted gene with decreased levels of expression of a gene product (e.g., protein or RNA) as compared to a wild-type levels. Disruption may include decreased expression of a gene product via a therapeutic nucleic acid or oligonucleotide agent, such as an antisense oligonucleotide (ASO), RNA interference (RNAi), including anti-sense, sense co-suppression, microRNA (miRNA), short interfering RNA (siRNA), short hairpin RNA (shRNA), a gRNA, or an sgRNA. In some embodiments, disruption of a gene results in decreased translation of an RNA gene product. Disruptions may include mutations, including insertions, deletions, substitutions (e.g., transitions, transversion), transpositions, inversions, knockouts, and combinations thereof. Mutations may involve only a single nucleotide (e.g., a point mutation or a single nucleotide polymorphism) or multiple nucleotides. In some embodiments, the mutation causes a phenotypic change, for example, the expression level of the encoded product is altered, or the encoded product itself is altered. In other embodiments, a disruption or mutation may result in an expressed protein with activity that is lower as compared to the activity of the expressed protein from the wild-type. In some embodiments, disruption is achieved with a CRISPR/Cas system, a meganuclease, a TALEN, a ZFN, or any combination thereof. As used herein, in some embodiments, a mutant gene may comprise, but is not limited to, a deletion of all or a portion of the gene; deletion of a regulatory element that controls gene expression, a frameshift mutation of the gene, or replacement of all or a portion of a gene.

“Endogenous nucleic acid” or “endogenous sequence” is “native” to, i.e., indigenous to, the organism that is to be genetically engineered. It refers to a nucleic acid, gene, polynucleotide, DNA, RNA, mRNA, or cDNA molecule that is present in the genome of an organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof.

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell, has been increased by genetic engineering, relative to a control cell.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Loss of function” refers to the loss of function of an SDS gene in a host tissue or organism, and encompasses the function at the molecular level and also at the phenotypic level.

The terms “modification,” “genomic modification,” “modified nucleotide,” or “edited nucleotide” as used herein refer to a nucleotide sequence of interest that comprises at least one alteration when compared to its non-modified nucleotide sequence. Such “alterations” include, for example: (i) replacement or substitution of at least one nucleotide, (ii) a deletion of at least one nucleotide, (iii) an insertion of at least one nucleotide, or (iv) any combination of (i)-(iii). In some embodiments, such modifications to a gene reduce or eliminate the expression of the gene product and/or its activity.

“Promoter” connotes a region of DNA upstream from the start of transcription that is involved in recognition and binding of RNA polymerase and other proteins to initiate transcription. A “constitutive promoter” is one that is active throughout the life of the cell and under most conditions. Tissue-specific, tissue-preferred, cell type-specific, and inducible promoters constitute the class of “non-constitutive promoters.” Tissue-specific regulatory elements are known in the art. In some embodiments, the tissue-specific promoter is liver-specific (e.g., TBG), kidney-specific, macrophage-specific, or dendritic cell-specific. “Operably linked” or “operatively linked” refers to a functional linkage between a promoter and a second sequence, where the promoter sequence initiates and mediates transcription of the DNA sequence corresponding to the second sequence. In general, “operably linked” or “operatively linked” means that the nucleic acid sequences being linked are contiguous. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame).

The term “regulatory element” is intended to include promoters, enhancers, internal ribosomal entry sites (IRES), and other expression control elements (e.g., transcription termination signals, such as polyadenylation signals and poly-U sequences). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, kidney), or particular cell types (e.g., macrophage, dendritic cell). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I; SV40 enhancer; and the intron sequence between exons 2 and 3 of rabbit β-globin. It will be appreciated by those skilled in the art that the design of the expression vector can depend on such factors as the choice of the host cell to be transformed, the level of expression desired, etc. A vector can be introduced into cells to thereby produce transcripts, proteins, or peptides, including fusion proteins or peptides, encoded by nucleic acids as described herein (e.g., antisense oligonucleotides, siRNA, shRNA, zinc finger peptides, clustered regularly interspersed short palindromic repeats (CRISPR) transcripts, proteins, enzymes, mutant forms thereof, fusion proteins thereof, etc.).

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, California, USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell, has been reduced by genetic engineering, relative to a control cell.

As used herein, “transformation” refers to the introduction of exogenous nucleic acid into cells, so as to produce transgenic cells stably transformed with the exogenous nucleic acid.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence, may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

As used herein, the terms “vector,” “vehicle,” “construct,” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites. In some embodiments, mammalian expression vectors are capable of directing expression of the nucleic acid preferentially in a particular cell type (e.g., tissue-specific regulatory elements are used to express the nucleic acid). Tissue-specific regulatory elements are known in the art.

### II. Serine Metabolism

l-Serine or serine (symbol Ser or S; molecular mass=105.093 g/mol) contains an α-amino group, a carboxyl group, and a side chain consisting of a hydroxymethyl group. Under physiological conditions of pH˜7.4, the α-amino group and carboxyl group are protonated and deprotonated, respectively, classifying l-serine as a polar amino acid. Serine can be ingested dietarily, or synthesized. De novo l-serine synthesis from glucose is initiated by conversion of the glycolytic intermediate 3-phosphoglycerate into 3-phosphohydroxypyruvate in an NAD-dependent reaction catalyzed by 3-phosphoglycerate dehydrogenase (PHGDH). Phosphoserine aminotransferase 1 (PSAT1) and phosphoserine phosphatase (PSPH) catalyze subsequent reactions that yield 3-phosphoserine and l-serine, respectively. In vitro tracing experiments with stable isotope tracers in rapidly proliferating cells have shown extensive labeling of l-serine from [U-13C]glucose. De novo l-serine biosynthesis is highly active in astrocytes, macrophages, and epidermal stem cells, suggesting that, at least under in vitro conditions, a wide variety of cell types are capable of synthesizing l-serine from glucose. Intracellular l-serine biosynthesis is coupled with redox state, l-glutamate availability as a source of α-amino group, and folate-mediated one-carbon metabolism (FOCM), highlighting the complex relationships between these metabolic pathways in the maintenance of l-serine homeostasis. For instance, inhibiting Complex I of the mitochondrial electron transport chain reduces de novo l-serine biosynthesis from glucose in cancer cell lines, providing experimental evidence that links l-serine synthesis to mitochondrial function and NAD+/NADH balance. In addition, de novo l-serine synthesis from glucose is coupled to l-glutamate availability and α-ketoglutarate. Expression of PHGDH is highly sensitive to exogenous l-serine availability in cultured cells, but the mechanisms underlying this transcriptional response have yet to be elucidated.

Beyond serving as a precursor for protein synthesis, l-serine directly supports the generation of pyruvate for gluconeogenesis, biosynthesis of lipid headgroups (sphingolipids, phosphatidylserine, phosphatidylethanolamine), and production of the neurotransmitter d-serine. Via a methylcobalamin-dependent 5-methyltetrahydrofolate-homocysteine methyltransferase reaction, l-serine-derived one-carbon units can also support l-methionine regeneration and methylation, further demonstrating the diverse metabolic pathways that are dependent on l-serine availability in the body. Furthermore, l-serine contributes to the production of l-cysteine and glutathione (GSH) in the transsulfuration pathway. The activity of methylene tetrahydrofolate reductases (MTHFRs) can also serve to regenerate NAD(P)H cofactors within the mitochondria and cytosol, although the directionality of these reactions is likely to be cell and context dependent. As noted above, l-serine is readily converted to one-carbon units and glycine to support synthesis of thymidylate and purines, heme, and creatine, all of which are critical for cell growth, redox homeostasis, and mitochondrial function.

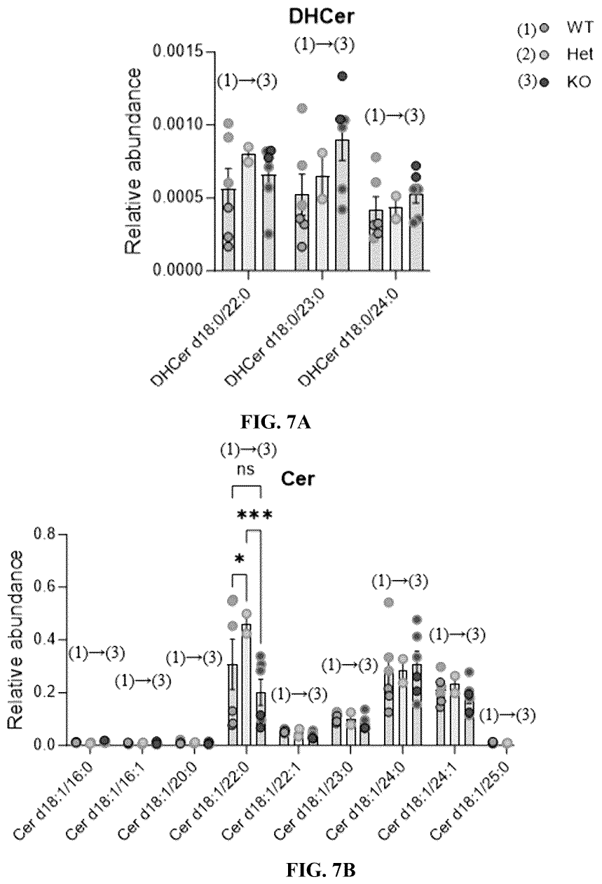

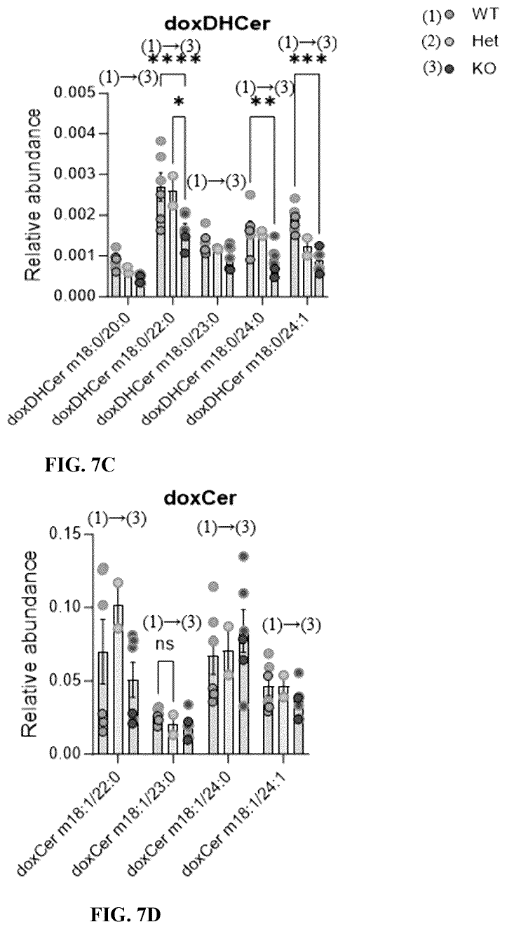

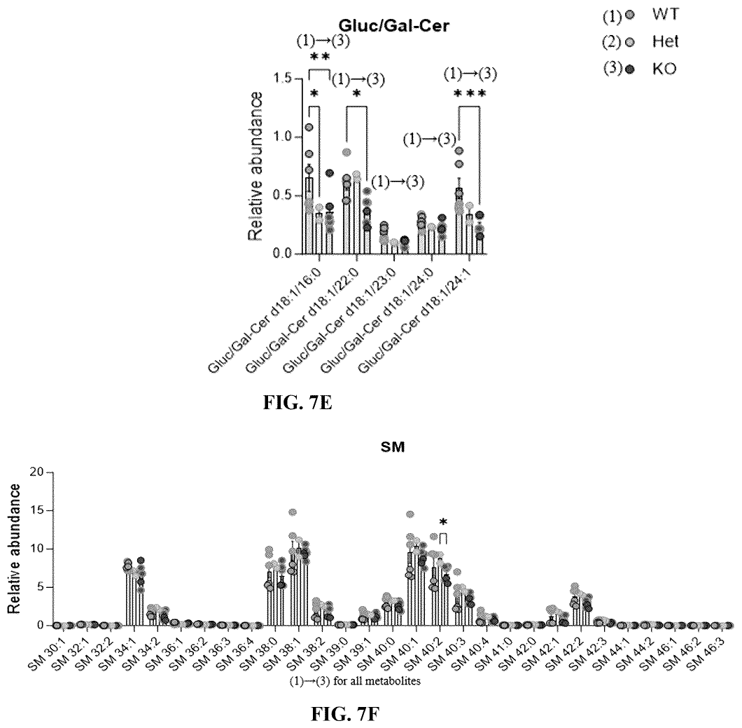

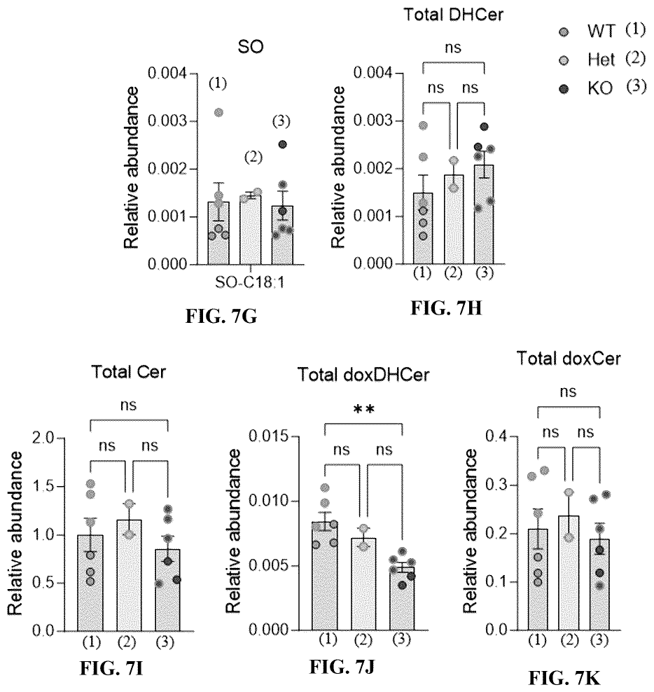

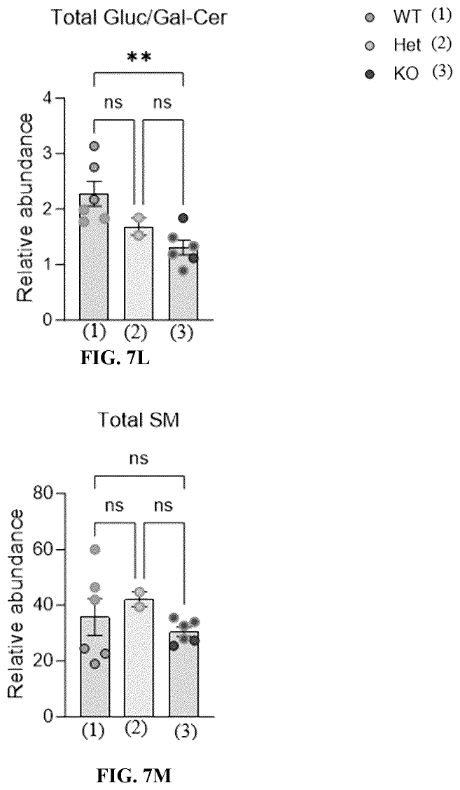

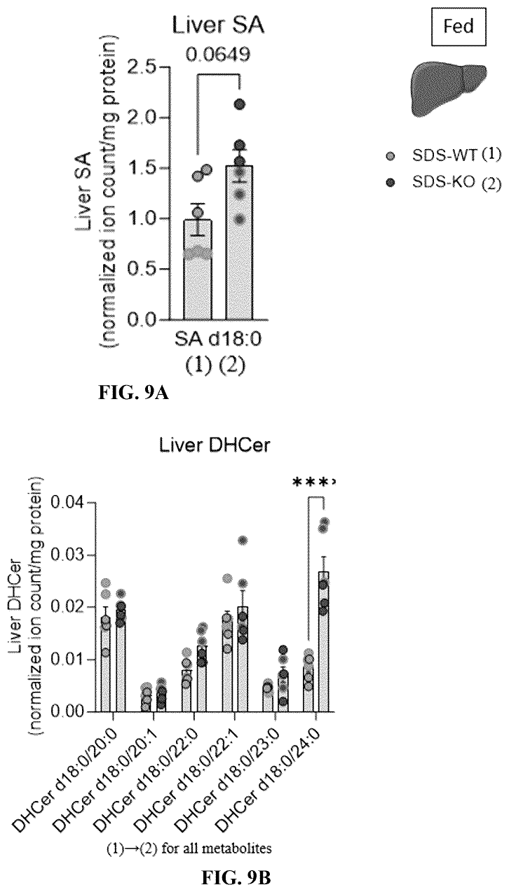

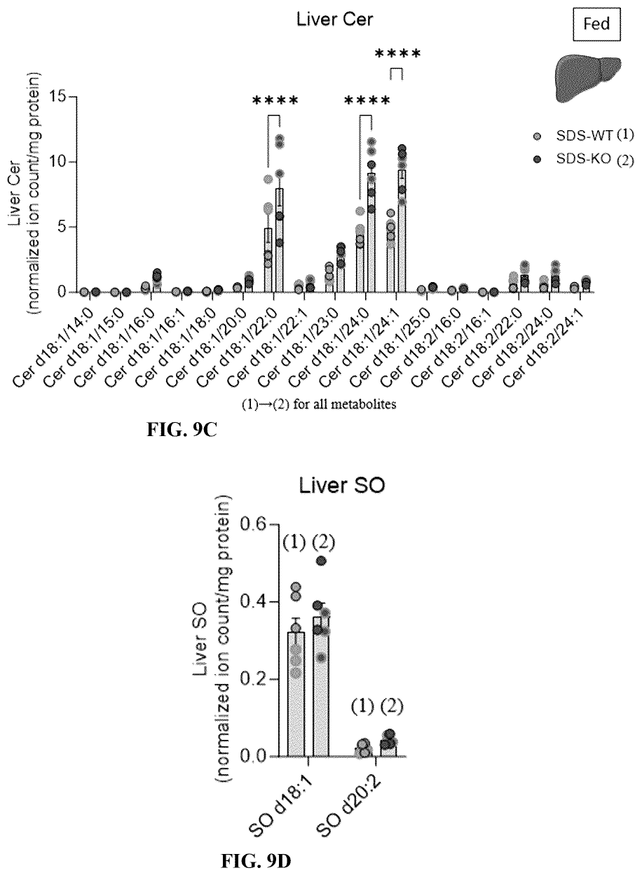

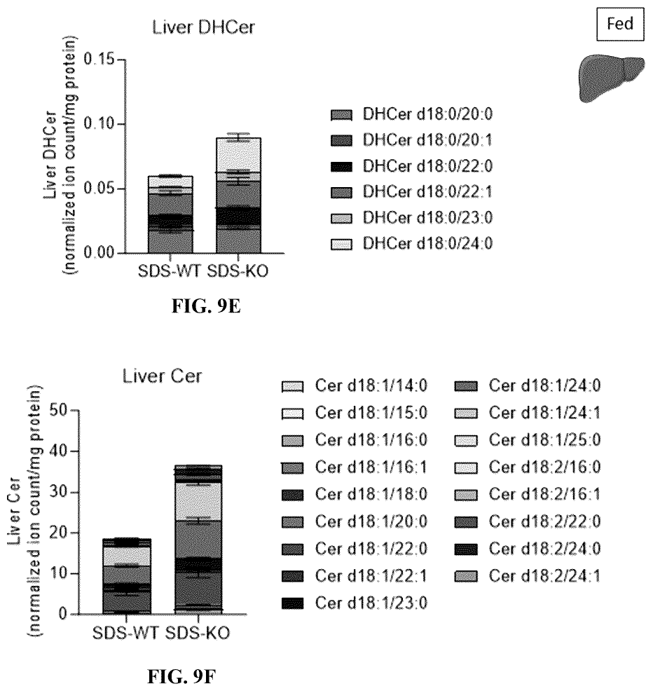

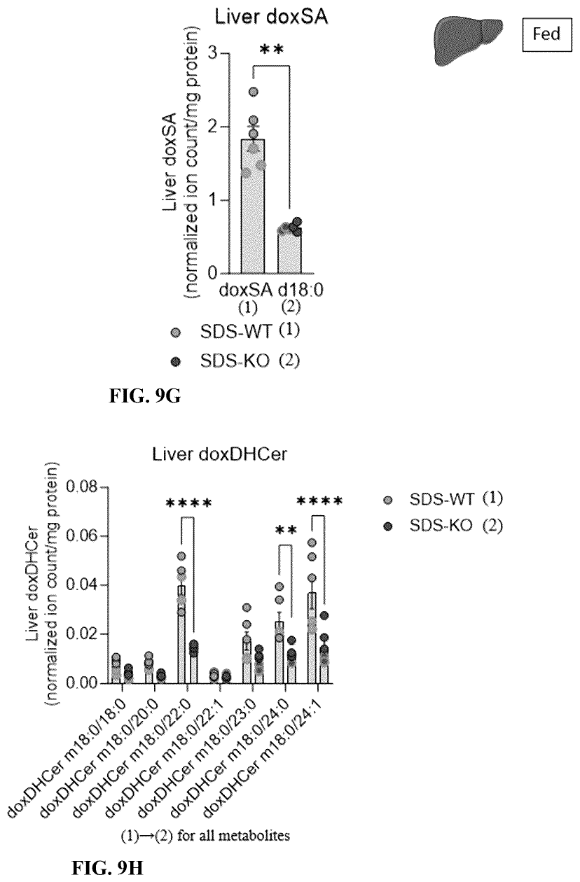

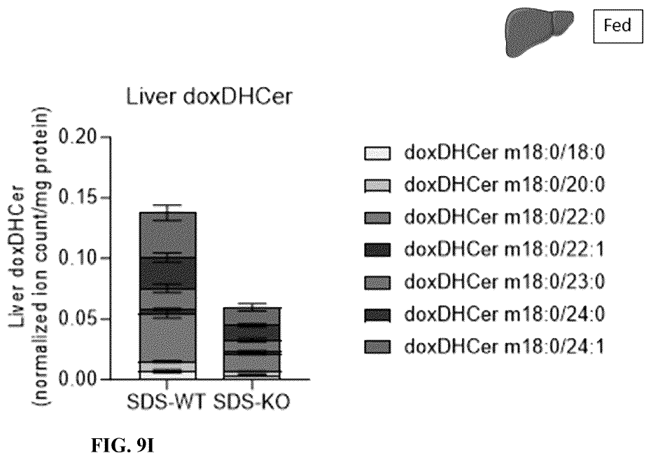

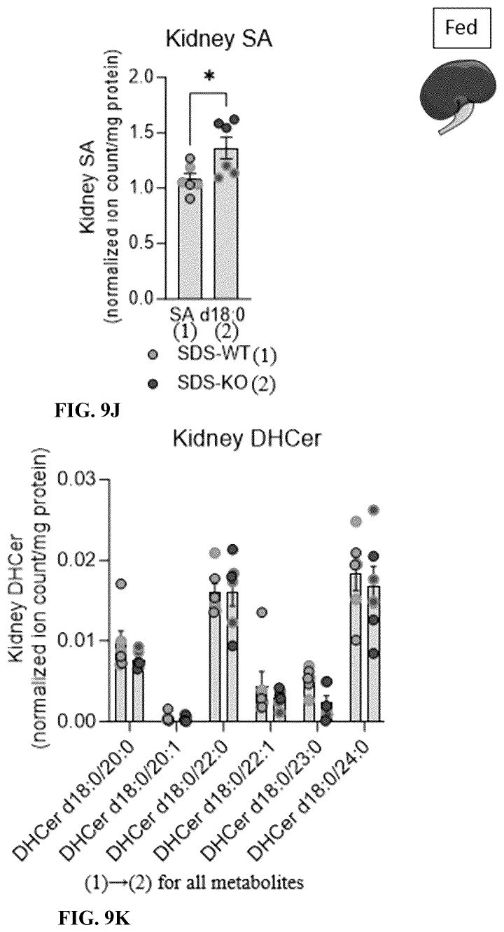

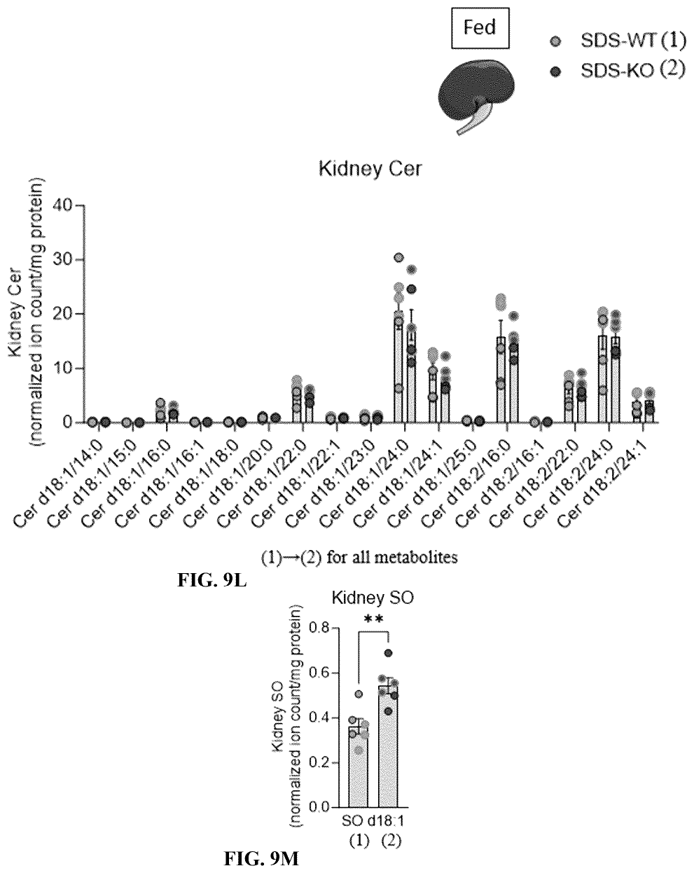

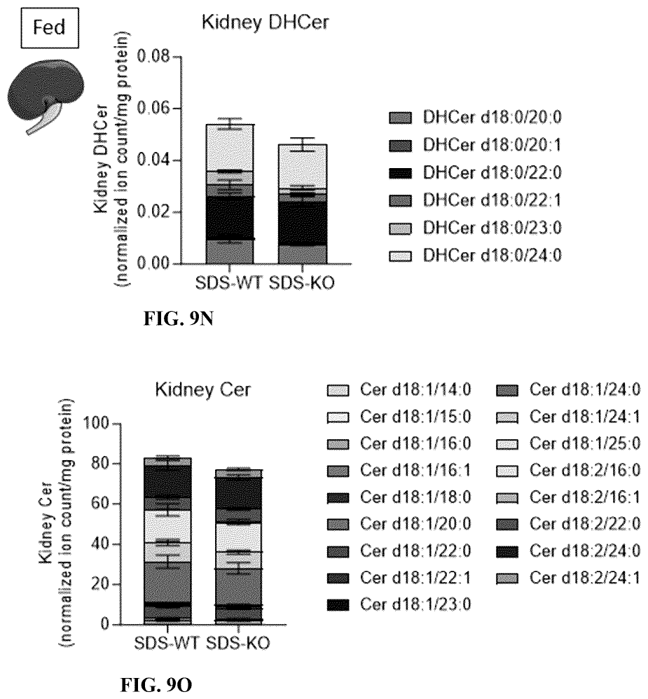

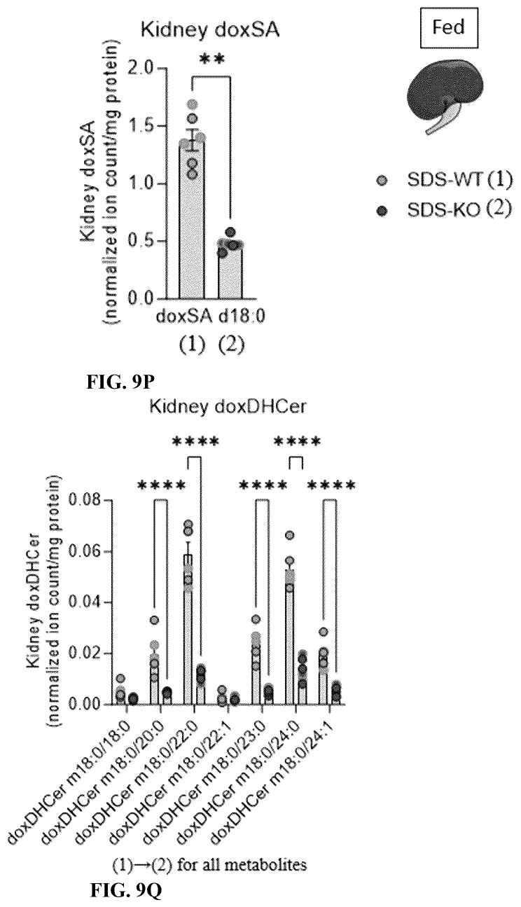

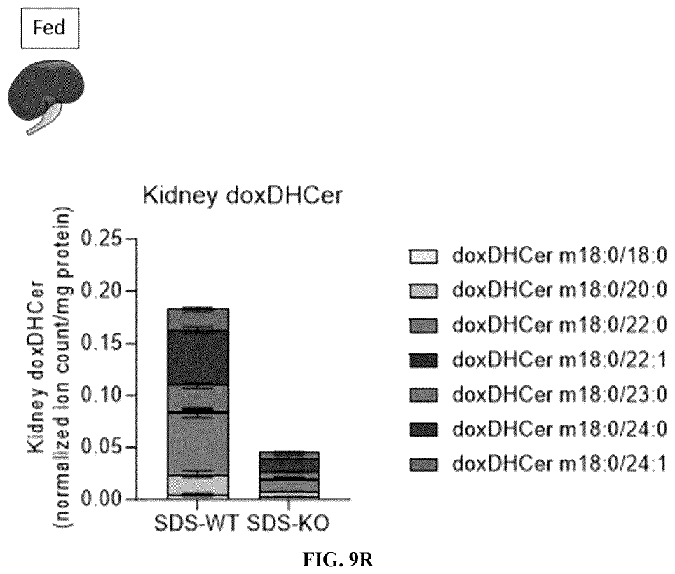

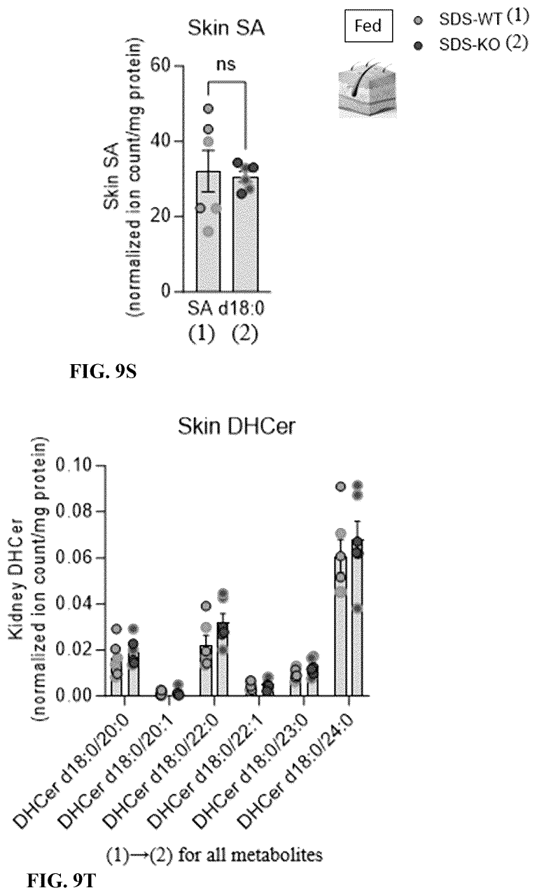

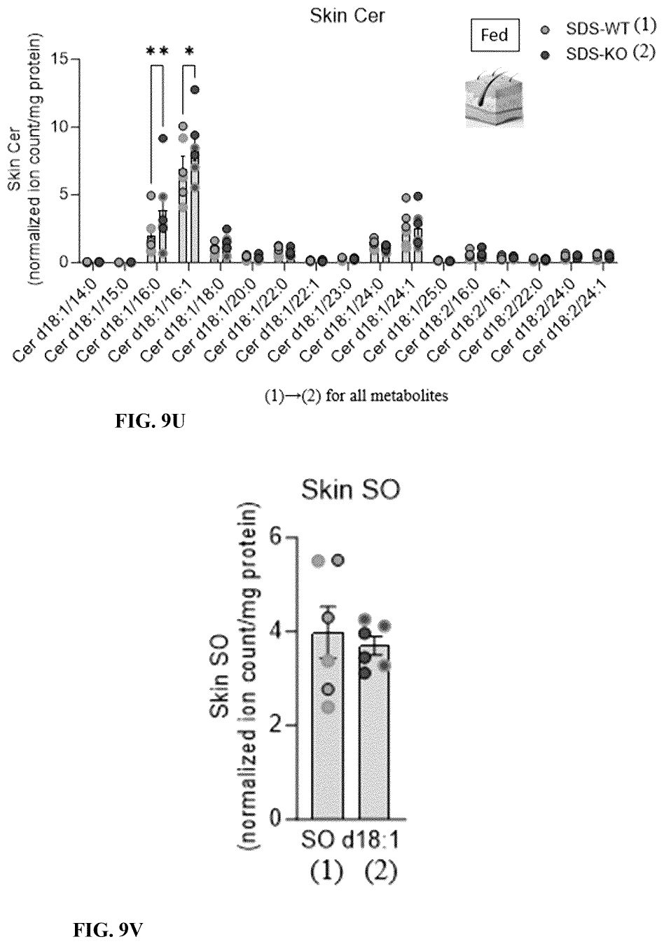

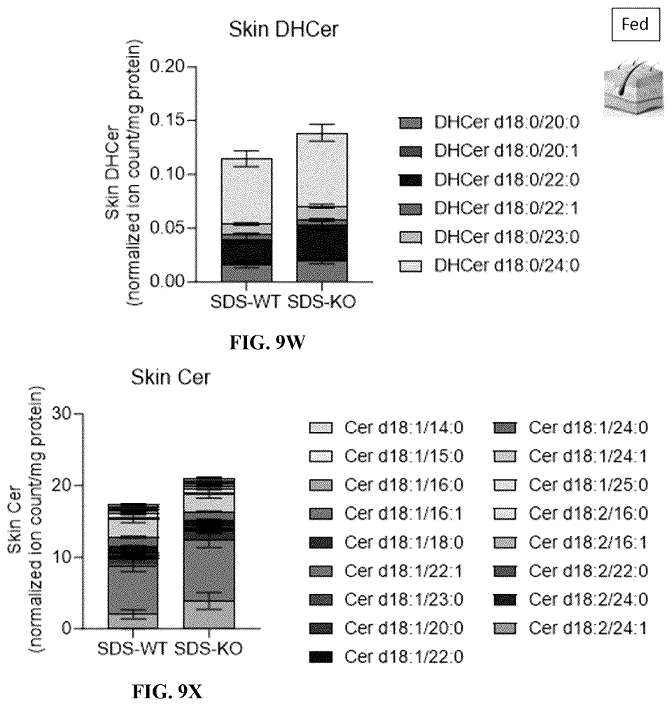

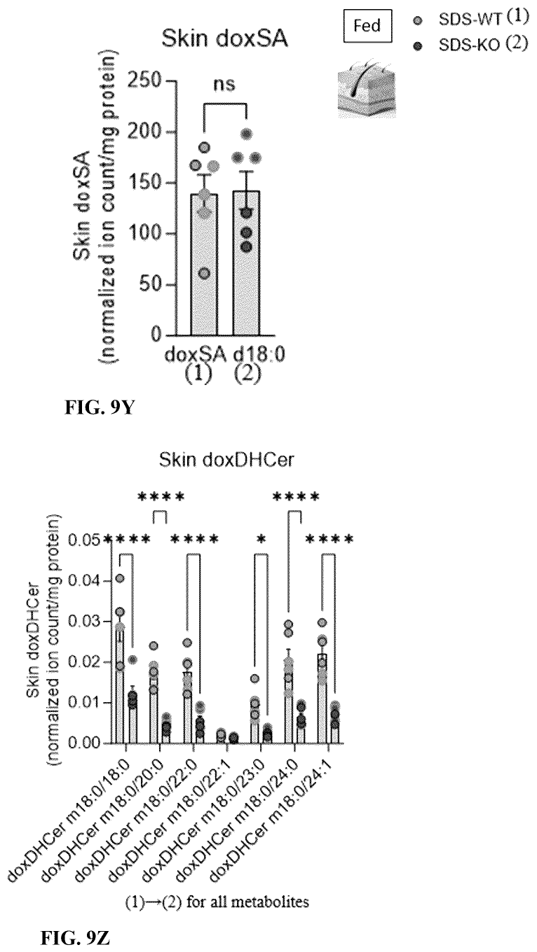

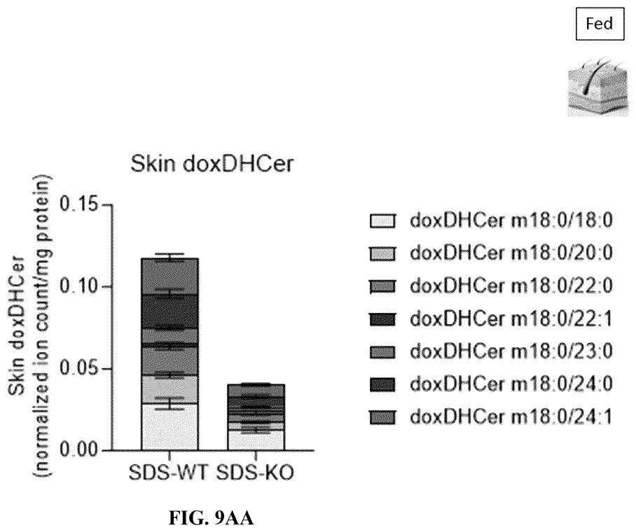

Some essential and nonessential amino acids, including glycine, l-cysteine, l-alanine, and l-threonine, share structural similarities with l-serine. Prolonged aberrant amino acid homeostasis may therefore lead to differential substrate usage owing to minor differences in their chemical potential. For example, serine palmitoyltransferase (SPT) condenses l-serine with palmitoyl-CoA to synthesize sphingolipids including ceramides. When l-serine availability is limited, or in sensory neuropathy patients harboring gain-of-function variants in SPTLC1 or SPTLC2, SPT incorporates other amino acids, including l-alanine, glycine, and l-threonine, to produce noncanonical sphingolipids that cannot be further metabolized to complex sphingolipids or degraded via phosphorylation. Promiscuous activation of tRNA synthetases caused by editing defects or substrate availability between l-serine, l-alanine, and noncanonical amino acids may also contribute to neurodegeneration in some contexts. Accordingly, doxSA and doxDHCer species will be elevated in these contexts and therefore these metabolites correlate with peripheral sensory neuropathy and macular disease. On the other hand, given the high serine concentration in SDS-KO mice, canonical sphingolipids such as SA, DHCer and Cer are increased in SDS KO mice (FIG. 9A) and non-canonical sphingolipids such as doxSA and doxDHCer are reduced in SDS KO mice (FIGS. 7A, 7B and FIG. 9B).

### III. Tools for Genetic Manipulation of Serine Dehydratase (SDS)

The present technology contemplates methods and compositions for disrupting serine dehydratase (SDS) activity. In particular, the present technology relates to targeted genome engineering (also known as genome editing) and RNA interference (RNAi) methods and compositions for altering the expression of an SDS gene. Provided herein are methods and compositions for using RNAi or modifying a target genomic locus in a cell to modulate the expression of an SDS gene. Targeted genome engineering techniques described herein include the CRISPR (clustered regularly interspaced short palindromic repeats)/Cas (CRISPR-associated) system, meganucleases, zinc finger nucleases (ZFNs), and TAL effector nucleases (TALENs). Such techniques may be employed to bind to and/or cleave a genomic region of interest of or adjacent to an SDS gene. In some embodiments, the agent that disrupts SDS activity comprises a therapeutic nucleic acid or oligonucleotide selected from an antisense oligonucleotide (ASO), an aptamer, an siRNA, a shRNA, a miRNA, a gRNA, or an sgRNA. In some embodiments, therapeutic nucleic acids or oligonucleotides are codon optimized for enhanced expression and efficacy in a chosen subject species. In some embodiments, the genome editing techniques described herein generate a specific sequence change or gene edit (e.g., insertion, deletion, or substitution) in the 5′-UTR of an SDS gene, such as generating a single nucleotide gene edit to form an out-of-frame start codon upstream of the gene's ORF, thereby suppressing expression of the SDS gene. In some embodiments, the gene edit (e.g., deletion, insertion, or substitution) results in production of an upstream, out-of-frame start codon that may result in the elimination of protein production or a nonfunctional protein. In some embodiments, the genome editing techniques described herein generate a specific sequence change or gene edit (e.g., insertion, deletion, or substitution) in the coding region or a non-coding region of an SDS gene, such as generating a large deletion to form (1) an out-of-frame start codon upstream of the gene's ORF, thereby suppressing expression of the SDS gene, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases. In some embodiments, the large deletion is generated in a SDS gene. RNAi techniques include anti-sense oligonucleotides (ASOs), sense co-suppression, microRNA (miRNA), short interfering RNA (siRNA), and short hairpin RNA (shRNA).

In some embodiments, provided herein are methods and compositions for facilitating or enhancing the delivery of therapeutic agents, such as oligonucleotides into cells or tissues. In some embodiments, the oligonucleotides optionally comprise a galactosamine. In some embodiments, the galactosamine is N-acetylgalactosamine (GalNAc) attached to the 3′ end or 5′ end of the oligonucleotide. In some embodiments, GalNac conjugated to the oligonucleotide enhances the efficiency and/or specificity of the oligonucleotide to a particular target tissue. In some embodiments, for example, GalNac-modified-oligonucleotides may achieve enhanced targeting and may facilitate the precise suppression of SDS mRNA (and subsequent SDS protein expression) within liver and kidney tissues.

Delivery systems or carriers/vehicles for oligonucleotide agents of the present technology are well known in the art. In some embodiments, oligonucleotides may be delivered to a cell by means of a viral vector (such as an adeno-associated virus (AAV) or lentivirus) and/or particle and/or nanoparticle delivery (such as a virus-like particle (VLP) or lipid nanoparticle (LNP)). Thus, in some embodiments, oligonucleotides used in any one or more of the methods for disrupting serine dehydratase (SDS) activity described herein may be formulated in a carrier, such as, but not limited to, an AAV, a VLP, or an LNP. The effective amount of the agents of the present technology may be determined during pre-clinical trials and clinical trials by methods familiar to physicians and clinicians. Doses may be based on or extrapolated to an average 70 kg individual (e.g., an adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. Compositions comprising the agents of the present technology (e.g., a formulation or medicament) can be formulated to be compatible with their intended route of administration.

### CRISPR Cas Systems

In some embodiments, the methods of the present technology relate to the use of a CRISPR/Cas system that binds to a target site in a region of interest in a genome (e.g., an SDS gene), wherein the CRISPR/Cas system comprises a CRISPR/Cas nuclease and an engineered crRNA/tracrRNA (or single guide RNA (sgRNA) or guide RNA (gRNA)). In some embodiments, the CRISPR system generally comprises (i) a polynucleotide encoding a Cas protein, and (ii) at least one sgRNA for RNA-guided genome engineering.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Cys3, Cse1, Cse2, Csc1, Csc2, Csa5, Csn2, Csm1, Csm2, Csm3, Csm4, Csm5, Csm6, Smr1, Cmr3, Cmr4, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Streptococcus pyogenes Cas9 protein. In some embodiments, the Cas protein is a Cas12a (Cpf1) protein. In some embodiments, the Cas protein is a Csm1 protein. These enzymes are known. For example, the amino acid sequence of S. pyogenes Cas9 protein may be found in the SwissProt database under accession number Q99ZW2. The amino acid sequence of Francisella tularensis subsp. novicida Cpf1 protein may be found in the UniProt database under accession number A0Q7Q2. The amino acid sequence of Thermococcus onnurineus Csm1 protein may be found in the UniProt database under accession number B6YWB8.

The sgRNA molecules comprise a crRNA-tacrRNA scaffold polynucleotide and a targeting sequence corresponding to a genomic target of interest.

In some embodiments, the CRISPR/Cas system recognizes a target site in an SDS gene. In some embodiments, the CRISPR/Cas system recognizes a target in one or more regulatory elements that impact expression of an SDS gene. The CRISPR/Cas system as described herein may bind to and/or cleave the region of interest in a region upstream of the coding region of an SDS gene. In some embodiments, the CRISPR/Cas system generates a specific sequence change in the 5′-UTR of an SDS gene, such as generating a single nucleotide gene edit to form an out-of-frame start codon upstream of the gene's ORF. In some embodiments, the gene edit (e.g., deletion, insertion, or substitution) results in production of an upstream, out-of-frame start codon that may result in the elimination of protein production or a nonfunctional protein. In some embodiments, the CRISPR/Cas system generates a specific sequence change or gene edit (e.g., insertion, deletion, or substitution) in the coding region or a non-coding region of an SDS gene, such as generating a large deletion to form (1) an out-of-frame start codon upstream of the gene's ORF, thereby suppressing expression of the SDS gene, or (2) a non-functional protein product resulting from a frame shift downstream of the gene edit. In some embodiments, the large deletion is greater than 50 bases, greater than 100 bases, greater than 200 bases, greater than 500 bases, greater than 1000 bases, greater than 2000 bases, greater than 5000 bases, or greater than 10000 bases.

The CRISPR/Cas system can be based on the Cas9 nuclease and an engineered single guide RNA (sgRNA) that specifies the targeted nucleic acid sequence. Cas9 is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tacrRNA). The precise sequence and length requirements for the PAM differ depending on the CRISPR enzyme used, but PAMs are typically 2-5 base pair sequences adjacent the protospacer (that is, the target sequence). Typically, in currently characterized CRISPR-Cas systems, there are two requirements for DNA interference: (i) the target sequence has to match one of the spacers present in the respective CRISPR array, and (ii) the target sequence complementary to the spacer (protospacer) has to be flanked by the appropriate PAM.

The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can induce site-specific double strand breaks (DSBs) into genomic DNA of live cells. See, e.g., Mussolino, Nat. Biothechnol., 31:208-209 (2013). In some embodiments, the Cas9 protein is expressed in a cell as a fusion to a nuclear localization signal (NLS) to ensure delivery into nuclei. In some embodiments, the Cas9 protein is tagged (e.g., FLAG- or GFP-tagged). In some embodiments, promoters may be used to drive Cas9 expression in a cell. In some embodiments, the Cas9 enzyme is S. pneumoniae, S. pyogenes, or S. thermophiles Cas9, and may include mutated Cas9 derived from these organisms. The enzyme may be a Cas9 homolog or ortholog. In some embodiments, the CRISPR enzyme (e.g., Cas9 enzyme) is codon-optimized for expression in a mammalian cell, such as a human cell.

The CRISPR/Cas system can be based on the Cpf1 nuclease and an engineered single guide RNA (sgRNA) that specifies the targeted nucleic acid sequence.

Cpf1 is distinguished from Cas9 by a its single RuvC endonuclease active site, its 5′ protospacer adjacent motif preference, and for creating sticky rather than blunt ends at the cut site. The Cpf1 protein has a RuvC-like endonuclease domain that is similar to the RuvC domain of Cas9. Cpf1 does not have a HNH endonuclease domain, and the N-terminal of Cpf1 does not have an alpha-helical recognition lobe, unlike Cas9. In some embodiments, the Cpf1 protein is tagged (e.g., FLAG- or GFP-tagged). In some embodiments, promoters may be used to drive Cpf1 expression in a cell. In some embodiments, the Cpf1 enzyme is Francisella tularensis subsp. novicida Cpf1, and may include mutated Cpf1 derived from these organisms. The enzyme may be a Cpf1 homolog or ortholog. In some embodiments, the CRISPR enzyme (e.g., Cpf1 enzyme) is codon-optimized for expression in a mammalian cell, such as a human cell.

The CRISPR/Cas system can be based on the Csm1 nuclease and an engineered single guide RNA (sgRNA) that specifies the targeted nucleic acid sequence.

Csm1 belongs to the Cas10 family of endonucleases. Csm1 is the largest subunit of the Csm interference complex in the type III-A CRISPR system. Csm1 exhibits ssDNA-specific endo- and exonuclease activity. In some embodiments, promoters may be used to drive Csm1 expression in a cell. In some embodiments, the Csm1 enzyme is Thermococcus onnurineus Csm1, and may include mutated Csm1 derived from these organisms. The enzyme may be a Csm1 homolog or ortholog. In some embodiments, the CRISPR enzyme (e.g., Csm1 enzyme) is codon-optimized for expression in a mammalian cell, such as a human cell.

The single guide RNA (sgRNA) is the second component of the CRISPR/Cas system that forms a complex with a Cas nuclease. The sgRNA is created by fusing crRNA with tacrRNA. The sgRNA guide sequence located at the 5′ end confers DNA target specificity. By modifying the guide sequence, sgRNAs with different target specificities can be designed to target any desired endogenous gene. In some embodiments, the target sequence is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence is about 1 to about 10 base pairs upstream of the transcription start site (e.g., positions −10, −9, −8, −7, −6, −5, −4, −3, −2, or −1). In some embodiments, the target sequence is located within the open reading frame of the gene of interest. In some embodiments, the target sequence is located within a coding region of the gene of interest.

In some embodiments, the CRISPR/Cas system comprises at least two sgRNAs. In some embodiments, a target sequence of at least one of the at least two sgRNAs is about 1,000, about 975, about 950, about 925, about 900, about 875, about 850, about 825, about 800, about 775, about 750, about 725, about 700, about 675, about 650, about 625, about 600, about 575, about 550, about 525, about 500, about 475, about 450, about 425, about 400, about 375, about 350, about 325, about 300, about 275, about 250, about 225, about 200, about 175, about 150, about 125, about 100, about 90, about 80, about 70, about 60, about 50, about 40, about 30, about 20, or about 15 base pairs upstream of the transcription start site, or the target sequence may be any number of base pairs in-between these values upstream of the transcription start site. In some embodiments, the target sequence of at least one of the at least two sgRNAs is about 1 to about 10 base pairs upstream of the transcription start site (e.g., positions −10, −9, −8, −7, −6, −5, −4, −3, −2, or −1). In some embodiments, the target sequence of at least one of the at least two sgRNAs is located within the open reading frame of the gene of interest. In some embodiments, the target sequence of at least one of the at least two sgRNAs is located within a coding region of the gene of interest. In some embodiments, the target sequences of at least two of the at least two sgRNAs are located within the open reading frame of the gene of interest. In some embodiments, the target sequences of at least two of the at least two sgRNAs are located within a coding region of the gene of interest. In some embodiments, the CRISPR/Cas system comprises two sgRNAs, wherein the two sgRNAs have non-overlapping target sequences. In some embodiments, the target sequences of the two sgRNAs are separated by at least 50 bases, at least 100 bases, at least 200 bases, at least 500 bases, at least 1000 bases, at least 2000 bases, at least 5000 bases, or at least 10000 bases.

It is not intended that the present technology be limited to any particular distance restraint with regard to the location of the guide RNA target sequence from the gene transcription start site. In some embodiments, the target sequence lies “in proximity to” a gene of interest, where “in proximity to” refers to any distance from the gene of interest, wherein the Cas-regulatory domain fusion is able to exert an effect on gene expression. In some embodiments, the target sequence lies upstream of the ORF of the gene of interest.

The canonical length of the guide sequence is about 20 bp and the DNA target sequence is about 20 bp followed by a PAM sequence having the consensus NGG sequence. In some embodiments, sgRNAs are expressed in a cell using RNA polymerase promoters.

When the DSBs are repaired by either NHEJ or HDR, the sequence at the repair site can be modified or new genetic information can be inserted (e.g., donor DNA comprising a desired gene edit can be inserted into the target gene at the break site). Although HDR typically occurs at lower and more variable frequencies than NHEJ, it can be leveraged to generate precise, defined modifications at a target locus in the presence of an exogenously introduced repair template. Accordingly, exogenous repair templates, designed by methods known in the art, can also be delivered into a cell, most often in the form of a synthetic, single-stranded DNA donor oligo or DNA donor plasmid, to generate a precise change in the genome. Single-stranded DNA donor oligos are delivered into a cell to insert or change short sequences (SNPs, amino acid substitutions, epitope tags, etc.) of DNA in the endogenous genomic target region. The benefits of using a synthetic DNA donor oligo is that no cloning is required to generate the donor template and DNA modifications can be added during synthesis for different applications, such as increased resistance to nucleases. Traditionally, the maximum insert length recommended for use with a DNA donor oligo is about 50 nucleotides.

In some embodiments, the present technology provides an engineered, programmable, non-naturally occurring CRISPR/Cas system comprising a Cas9 protein and one or more single guide RNAs (sgRNAs) that target the genomic loci of DNA molecules encoding an SDS gene, and the Cas9 protein cleaves the genomic loci of the DNA molecules encoding the one or more gene products, whereby expression of the one or more gene products is altered. In some embodiments, Cas9 introduces multiple DSBs in the same cell (i.e., multiplexes) via expression of one or more distinct guide RNAs.

In some embodiments, the present technology provides a method for targeted genomic modification of cells to alter the expression of an SDS gene, the method comprising introducing into a cell, comprising and expressing a DNA molecule having a target sequence and encoding the SDS gene involved, an engineered CRISPR/Cas system comprising (a) an expression construct comprising a first polynucleotide encoding a Cas9 protein, or a variant thereof or a fusion protein therewith, and a second polynucleotide encoding a guide RNA comprising: (i) a crRNA-tracrRNA scaffold polynucleotide, and (ii) a targeting sequence operably linked to the crRNA-tracrRNA scaffold polynucleotide, where the targeting sequence corresponds to a genomic locus of interest, and (b) delivering the expression construct into the cell, where the first and second polynucleotides are expressed (transcribed) within the cell. This method can optionally further include visualizing, identifying, or selecting for cells having a genomic modification at the genomic locus of interest that is induced by the delivering the expression construct into the cell.

In some embodiments of the methods of the present technology, the Cas9 polypeptide and one or more guide RNA are encoded on a single vector. In some embodiments, the single vector is a plasmid. In some embodiments of the methods of the present technology, the Cas9 polypeptide and the one or more guide RNA are encoded on two separate vectors. In these methods, the steps generally follow the sequence of introducing into a cell containing and expressing a DNA molecule having a target sequence and encoding the SDS gene an engineered CRISPR/Cas system comprising (a) a Cas9 polynucleotide or a conservative variant thereof, and a guide RNA comprising (i) a crRNA-tracrRNA scaffold polynucleotide, and (ii) a targeting sequence operably linked to the crRNA-tracrRNA scaffold polynucleotide, with the targeting sequence corresponding to a genomic locus of interest, and (b) delivering the two polynucleotides into the cell. In variations of this method, a donor polynucleotide having homology to the genomic target of interest is included in a co-transfection. In some variations of these methods, the transfected material can be either plasmid DNA or RNA generated by in vitro transcription. In still other variations, the methods for targeted genomic modification are multiplexed, meaning that more than one genomic locus is targeted for modification. In still other variations of these methods, the transformation of the cells can be followed by visualizing, identifying, or selecting for cells having a genomic modification at the genomic locus of interest.

### Meganucleases

In some embodiments, the compositions and methods described herein employ a meganuclease DNA binding domain for binding to a region of interest in the genome of a cell. Meganucleases are engineered versions of naturally occurring restriction enzymes that typically have extended DNA recognition sequences (e.g., about 14 to about 40 base pairs in length). Meganucleases (also known as homing endonucleases) are commonly grouped into five families based on sequence and structure motifs: the LAGLIDADG family (SEQ ID NO: 7), the GIY-YIG family, the His-Cyst box family, the PD-(D/E)XK family, and the HNH family. In some embodiments, the meganuclease comprises an engineered homing endonuclease. The recognition sequences of homing endonucleases and meganucleases such as I-Sce, I-CeuI, PI-PspI, PI-Sce, I-SceIV, I-CsmI, I-PanI, I-SceII, I-PpoI, I-SceIII, I-CreI, I-TevI, I-TevII, and I-TevIII are known.

In some embodiments, the meganuclease is tailored to recognize a target in an SDS gene. The meganucleases as described herein may bind to and/or cleave the region of interest in a region upstream of the coding region of an SDS gene. Gene insertion or correction can be achieved by the introduction of a DNA repair matrix containing sequences homologous to the endogenous sequence surrounding the DNA break. Gene edits can be created either at or distal to the break. In some embodiments, the meganuclease generates a specific sequence change in the 5′-UTR of an SDS gene, such as generating a single nucleotide gene edit to form an out-of-frame start codon upstream of the gene's ORF.

### TALENs

In some embodiments, the compositions and methods described herein employ transcription activator-like effector nucleases (TALENs) to edit genomes by inducing double-strand breaks (DSBs). TALENs are restriction enzymes that can be engineered to cleave specific sequences of DNA. TALENs are constructed by fusing a TAL effector DNA-binding domain to a DNA cleavage domain (e.g., a nuclease domain such as that derived from the FokI endonuclease). Transcription activator-like effectors (TALEs) can be engineered according to methods known in the art to bind to a desired DNA sequence, and when combined with a nuclease, provide a technique for cutting DNA at specific locations. For example, after a target sequence in an SDS gene is identified, a corresponding TALEN sequence is engineered and inserted into a plasmid. The plasmid is inserted into a target cell where it is translated to produce a functional TALEN, which then enters the nucleus where it binds to and cleaves its target sequence. Such an approach can be employed to introduce an exogenous DNA sequence into the target gene as the DSB is being repaired through either homology-directed repair or non-homologous end-joining. For example, in some embodiments, the use of TALEN technology generates a specific sequence change (e.g., insertion, deletion, or substitution) in the 5′-UTR of an SDS gene, resulting in the production of an out-of-frame start codon upstream of the gene's ORF.

### ZFNs

In some embodiments, the compositions and methods described herein employ zinc finger nucleases (ZFNs) to edit genomes by inducing double-strand breaks (DSBs). ZFNs are artificial restriction enzymes generated by fusing a zinc finder DNA-binding domain to a DNA cleavage domain (e.g., a nuclease domain such as that derived from the FokI endonuclease). ZFNs can be engineered to bind and cleave DNA at specific locations. ZFNs contain two protein domains. The first domain is the DNA-binding domain, which contains eukaryotic transcription factors and the zinc finger. The second domain is a nuclease domain that contains the FokI restriction enzyme responsible for cleaving DNA. ZFNs can be engineered according to methods known in the art to bind to a desired DNA sequence and cleave DNA at specific locations. For example, after a target sequence in an SDS gene is identified, a corresponding ZFN sequence is engineered and inserted into a plasmid. The plasmid is inserted into a target cell where it is translated to produce a functional ZFN, which then enters the nucleus where it binds to and cleaves its target sequence introducing a double strand break (DSB). Such an approach can be employed to introduce an exogenous DNA sequence into the target gene as the DSB is being repaired through either homology-directed repair or non-homologous end-joining. For example, in some embodiments, the use of ZFN technology generates a specific sequence change in the 5′-UTR of an SDS gene, such as the insertion of an out-of-frame start codon upstream of the gene's ORF.

### RNA Interference

In one aspect of the invention, methods and constructs are provided for suppressing an SDS gene. While any method may be used for suppressing an SDS gene, the present invention contemplates anti-sense, sense co-suppression, microRNA (miRNA), short interfering RNA (siRNA), and short hairpin RNA (shRNA).

shRNA techniques involve stable transformation using shRNA plasmid constructs (Helliwell and Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter is integrated into the genome and subsequent transcription of the transgene leads to an RNA molecule that folds back on itself to form a double-stranded hairpin RNA. Non-limiting examples of suitable promoters include RNA Pol III promoter (such as U6 or H1) or an RNA Pol II promoter (such as CMV). This double-stranded RNA structure is recognized by the cell and cut into small RNAs (about 21 nucleotides long) called small interfering RNAs (siRNAs). siRNAs associate with a protein complex (RISC) which goes on to direct degradation of the mRNA for the target gene.

microRNA (miRNA) techniques exploit the miRNA pathway that functions to silence endogenous genes. In this method, 21 nucleotide long fragments of the gene to be silenced are introduced into a pre-miRNA gene to form a pre-miRNA construct. The pre-miRNA construct is transferred into the genome using transformation methods apparent to one skilled in the art. After transcription of the pre-miRNA, processing yields miRNAs that target genes, which share nucleotide identity with the 21 nucleotide miRNA sequence.

In RNAi silencing techniques, several factors can influence the choice of length of the fragment. The shorter the fragment the less frequently effective silencing will be achieved, but very long hairpins increase the chance of recombination. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences.

Antisense techniques involve introducing into a cell an antisense oligonucleotide (ASO) that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Sense co-suppression techniques involve introducing a highly expressed sense transgene into a cell resulting in reduced expression of both the transgene and the endogenous gene. The effect depends on sequence identity between transgene and endogenous gene.

### IV. Methods and Compositions for Increasing Serine Levels

The present technology relates to methods and compositions for disrupting serine dehydratase (SDS) activity. In some embodiments, the methods and compositions relate to the use of one or more gene editing techniques, such as those described in Section III, to disrupt SDS activity and increase serine levels in one or more tissues in a subject in need thereof, and/or any one or more of the targeting moieties for influencing SDS expression provided in Tables 1-13. In some embodiments, the targeting moieties for influencing SDS expression are any one or more of the SDS zinc finger variable binding domain amino acid sequences as set forth in Tables 1-5 (“r” in connection with the positions listed in Tables 1-5 means “reverse,” which is in reference to the strand of DNA (Watson or Crick) the zinc finger targets with the base pair position away from the transcriptional start site as the adjoining number). Accordingly, the agents of the present technology may include zinc finger nucleases comprising any one of the zinc finger regions as set forth in Tables 1-5, and nucleotides encoding the same. In some embodiments, the zinc finger nucleases are capable of disrupting SDS activity. In some embodiments, the targeting moieties for influencing SDS expression are any one or more of the CRISPR sgRNA spacer oligonucleotides as set forth in Tables 6-11. Accordingly, the methods of the present technology may include methods for producing an edited genome in a mammalian cell comprising introducing a CRISPR system (e.g., a CRISPR-Cas system) into the cell, wherein the system comprises one or more of the sgRNA oligonucleotides or at least one polynucleotide encoding the one or more sgRNA oligonucleotides. In some embodiments, the edited genome results in a disruption of SDS activity. In some embodiments, the targeting moieties for influencing SDS expression are any one or more of the RNAi, shRNA, and anti-sense oligonucleotides as set forth in Tables 12-13. Accordingly, the agents of the present technology may include RNAi, shRNA, and anti-sense oligonucleotides comprising the nucleotide sequences as set forth in Tables 12-13. In some embodiments, the RNAi, shRNA, and anti-sense oligonucleotides are capable of disrupting SDS activity.

TABLE 1
hSDS Zinc Finger Regions for Targeted Repression

| Position | ZF1 | ZF2 | ZF3 | ZF4 | ZF5 | ZF6 |
|---|---|---|---|---|---|---|
| r110 | DRGDLTR | TSGALVR | RSDNLAR | RSDALAR | RSDNLAR | RSDNRTN |
| | (SEQ ID | (SEQ ID | (SEQ ID | (SEQ ID | (SEQ ID | (SEQ ID |
| | NO: 8) | NO: 9) | NO: 10) | NO: 11) | NO: 10) | NO: 12) |
| r508 | DRSNLSR | RSDNLTR | QSSDLSR | RSDHLSR | RSDNLAR | QSGHRKT |
| | (SEQ ID | (SEQ ID | (SEQ ID | (SEQ ID | (SEQ ID | (SEQ ID |
| | NO: 13) | NO: 14) | NO: 15) | NO: 16) | NO: 10) | NO: 17) |
| r113 | RSDSLTR | QSGDLTR | TSGDLSR | RSDNLAR | RSDSLAR | RSDNLAR |
| | (SEQ ID | (SEQ ID | (SEQ ID | (SEQ ID | (SEQ ID | (SEQ ID |
| | NO: 18) | NO: 19) | NO: 20) | NO: 10) | NO: 21) | NO: 10) |
| r381 | DRSALTR | QSSTLAI | RSSALST | DRSNLTR | RSDHLSE | DSRDRTK |
| | (SEQ ID | (SEQ ID | (SEQ ID | (SEQ ID | (SEQ ID | (SEQ ID |
| | NO: 22) | NO: 23) | NO: 24) | NO: 25) | NO: 26) | NO: 27) |
| r511 | RSDHLAR | DRSNLTR | RSDNLTR | QSSDLRR | RSDHLSE | RNDNRKT |
| | (SEQ ID | (SEQ ID | (SEQ ID | (SEQ ID | (SEQ ID | (SEQ ID |
| | NO: 28) | NO: 25) | NO: 14) | NO: 29) | NO: 26) | NO: 30) |
| r440 | QSAHLAR | QSGDLTR | QSGDLRT | DRSSRTR | RSDHLSQ | TSATRTT |
| | (SEQ ID | (SEQ ID | (SEQ ID | (SEQ ID | (SEQ ID | (SEQ ID |
| | NO: 31) | NO: 19) | NO: 32) | NO: 33) | NO: 34) | NO: 35) |
| r514 | RSDHLSA | RNAHRKR | RSAHLSR | RSDNLTR | QSSDLSR | RSDHLSR |
| | (SEQ ID | (SEQ ID | (SEQ ID | (SEQ ID | (SEQ ID | (SEQ ID |
| | NO: 36) | NO: 37) | NO: 38) | NO: 14) | NO: 15) | NO: 16) |
| r432 | QSGDLTT | RSDHLTT | TSSNLTR | RSDHLTR | RSDNLSA | QSTHRIK |
| | (SEQ ID | (SEQ ID | (SEQ ID | (SEQ ID | (SEQ ID | (SEQ ID |
| | NO: 39) | NO: 40) | NO: 41) | NO: 42) | NO: 43) | NO: 44) |
| r157 | QSGHRKA | RSHHLAR | QSGSLTR | RNASRTR | RSDHLSQ | DSSTRKK |
| | (SEQ ID | (SEQ ID | (SEQ ID | (SEQ ID | (SEQ ID | (SEQ ID |
| | NO: 45) | NO: 46) | NO: 47) | NO: 48) | NO: 34) | NO: 49) |
| r78 | QSAHLSR | QSGDLTR | RSDNLAR | QSSDLSR | RSDNLSR | DSSTRKT |
| | (SEQ ID | (SEQ ID | (SEQ ID | (SEQ ID | (SEQ ID | (SEQ ID |
| | NO: 50) | NO: 19) | NO: 10) | NO: 15) | NO: 51) | NO: 52) |
| r402 | RSDHLST | TSGHLSR | QSGDRKT | DPSSLVR | RSDHLST | RSDHRTK |
| | (SEQ ID | (SEQ ID | (SEQ ID | (SEQ ID | (SEQ ID | (SEQ ID |
| | NO: 53) | NO: 54) | NO: 55) | NO: 56) | NO: 53) | NO: 57) |
| r116 | QSGHLIR | RSDNLAR | QSGDLTR | RSSALSR | RSGHLTR | RSDALAR |
| | (SEQ ID | (SEQ ID | (SEQ ID | (SEQ ID | (SEQ ID | (SEQ ID |
| | NO: 58) | NO: 10) | NO: 19) | NO: 59) | NO: 60) | NO: 11) |
| r107 | DRSALSR | RSDNLAR | RSDSLSR | RSDNLAR | RSDNLST | DRSNRTK |
| | (SEQ ID | (SEQ ID | (SEQ ID | (SEQ ID | (SEQ ID | (SEQ ID |
| | NO: 61) | NO: 10) | NO: 62) | NO: 10) | NO: 63) | NO: 64) |
| r84 | RSDVLSE | QSANLAR | QSGNLAR | QSSDLRR | QSSDLSR | QSSDLSR |
| | (SEQ ID | SEQ ID | (SEQ ID | (SEQ ID | (SEQ ID | (SEQ ID |
| | NO: 65) | NO: 66) | NO: 67) | NO: 29) | NO: 15) | NO: 15) |
| r456 | RSDSLTM | QNATRKK | QSGDLRK | RSANLSR | QLGDLST | RSANLTR |
| | (SEQ ID | (SEQ ID | (SEQ ID | (SEQ ID | (SEQ ID | (SEQ ID |
| | NO: 68) | NO: 69) | NO: 70) | NO: 71) | NO: 72) | NO: 73) |
| 183 | DRSNLSR | QSGHLTR | RSDNLAR | QSGNLSR | RSDHLSR | RKGDRKN |
| | (SEQ ID | (SEQ ID | (SEQ ID | (SEQ ID | (SEQ ID | (SEQ ID |
| | NO: 13) | NO: 74) | NO: 10) | NO: 75) | NO: 16) | NO: 76) |
| 186 | RSDHLSE | QSANRNR | QSGHLAR | RSDNLAR | QSGHLSR | RSDHLSR |
| | (SEQ ID | (SEQ ID | (SEQ ID | (SEQ ID | (SEQ ID | (SEQ ID |
| | NO: 26) | NO: 77) | NO: 78) | NO: 10) | NO: 79) | NO: 16) |
| 261 | DRGHLVR | QSGHLTR | RSDHLSR | RSGNLSR | QSSDLSR | RSDNRIT |
| | (SEQ ID | (SEQ ID | (SEQ ID | (SEQ ID | (SEQ ID | (SEQ ID |
| | NO: 80) | NO: 74) | NO: 16) | NO: 81) | NO: 15) | NO: 82) |
| 221 | TSSNLAR | QSSDLRR | RSDALSR | RLDARKS | TSGHLSR | RSDARTK |
| | (SEQ ID | (SEQ ID | (SEQ ID | (SEQ ID | (SEQ ID | (SEQ ID |
| | NO: 83) | NO: 29) | NO: 84) | NO: 85) | NO: 54) | NO: 86) |
| 182 | QSAHLAR | QSAHLAR | QSAHLAR | QSGNLAR | RSDHLSE | DSSNRKK |
| | (SEQ ID | (SEQ ID | (SEQ ID | (SEQ ID | (SEQ ID | (SEQ ID |
| | NO: 31) | NO: 31) | NO: 31) | NO: 67) | NO: 26) | NO: 87) |

TABLE 2
mSds Zinc Finger Regions for Targeted Repression

| Position | ZF1 | ZF2 | ZF3 | ZF4 | ZF5 | ZF6 |
|---|---|---|---|---|---|---|
| 199 | QSGHLAR | QSGALAR | RSDALTR | QSGNRKR | DRSDLSR | RSDHLTT |
| | (SEQ ID | (SEQ ID | (SEQ ID | (SEQ ID | (SEQ ID | (SEQ ID |
| | NO: 78) | NO: 88) | NO: 89) | NO: 90) | NO: 91) | NO: 40) |
| 196 | QSGALSR | RSDALAR | QSGNLRR | DRSNLTR | RSDHLSQ | QSATRKT |
| | (SEQ ID | (SEQ ID | (SEQ ID | (SEQ ID | (SEQ ID | (SEQ ID |
| | NO: 92) | NO: 11) | NO: 93) | NO: 25) | NO: 34) | NO: 94) |
| 230 | QSGSLTR | RSDALAR | RSDHLSE | ASSTRKK | DRSDLSR | HRANLTR |
| | (SEQ ID | (SEQ ID | (SEQ ID | (SEQ ID | (SEQ ID | (SEQ ID |
| | NO: 47) | NO: 11) | NO: 26) | NO: 95) | NO: 91) | NO: 96) |
| 224 | RSDHLSR | TSSTRTS | QSSALSR | LKSNLAR | RSDNLTE | TSSNRKK |
| | (SEQ ID | (SEQ ID | (SEQ ID | (SEQ ID | (SEQ ID | (SEQ ID |
| | NO: 16) | NO: 97) | NO: 98) | NO: 99) | NO: 100) | NO: 101) |
| 146 | ERSDLRE | QSDDLAR | RSDALSV | QSGNLAR | RSDNLAR | QSGHLAR |
| | (SEQ ID | (SEQ ID | (SEQ ID | (SEQ ID | (SEQ ID | (SEQ ID |
| | NO: 102) | NO: 103) | NO: 104) | NO: 67) | NO: 10) | NO: 78) |
| 240 | DRSHLTR | QSGDLTR | RSDSLSR | LRSNRKS | TSGHLTR | TSHHLTR |
| | (SEQ ID | (SEQ ID | (SEQ ID | (SEQ ID | (SEQ ID | (SEQ ID |
| | NO: 105) | NO: 19) | NO: 62) | NO: 106) | NO: 107) | NO: 108) |
| 202 | DRSNLRR | QSGNLAR | QSGALAR | RSDALTR | QSGNLTR | DRSNLRR |
| | (SEQ ID | (SEQ ID | (SEQ ID | (SEQ ID | (SEQ ID | (SEQ ID |
| | NO: 109) | NO: 67) | NO: 88) | NO: 89) | NO: 110) | NO: 109) |
| 135 | RSDHLSA | QSAHLTR | RSDALSR | QSGNLAR | RSDHLSK | DSSNRTT |
| | (SEQ ID | (SEQ ID | (SEQ ID | (SEQ ID | (SEQ ID | (SEQ ID |
| | NO: 36) | NO: 111) | NO: 84) | NO: 67) | NO: 112) | NO: 113) |
| 469 | RSDALSV | DRSNLSR | RSDNLSV | DRSDRTR | RSDHLSQ | DSSTRKK |
| | (SEQ ID | (SEQ ID | (SEQ ID | (SEQ ID | (SEQ ID | (SEQ ID |
| | NO: 104) | NO: 13) | NO: 114) | NO: 115) | NO: 34) | NO: 49) |
| 466 | QSSNLSR | RSDHLST | DRSDLTR | RSDHLSR | QSSTLLR | QSGTLTR |
| | (SEQ ID | (SEQ ID | (SEQ ID | (SEQ ID | (SEQ ID | (SEQ ID |
| | NO: 116) | NO: 53) | NO: 117) | NO: 16) | NO: 118) | NO: 119) |
| r48 | RSDHLSR | QSGDLTR | RSDHLNQ | QSGNLTR | RSGHLSR | RSHHRKT |
| | (SEQ ID | (SEQ ID | (SEQ ID | (SEQ ID | (SEQ ID | (SEQ ID |
| | NO: 16) | NO: 19) | NO: 120) | NO: 110) | NO: 121) | NO: 122) |
| r47 | RSDTLTV | DRSHLAR | RSDARKV | QSGNLTR | RSDHLSR | RNDHRKT |
| | (SEQ ID | (SEQ ID | (SEQ ID | (SEQ ID | (SEQ ID | (SEQ ID |
| | NO: 123) | NO: 124) | NO: 125) | NO: 110) | NO: 16) | NO: 126) |
| r162 | RSSDLSE | RNASLSR | QSAHLSE | TSQVLRR | RSDSLLR | RLDNRTA |
| | (SEQ ID | (SEQ ID | (SEQ ID | (SEQ ID | (SEQ ID | (SEQ ID |
| | NO: 127) | NO: 128) | NO: 129) | NO: 130) | NO: 131) | NO: 132) |
| r425 | RSDNLSR | RSDNLTR | RSDNLSE | QNSTRTK | RSDDLSK | DSATRTK |
| | (SEQ ID | (SEQ ID | (SEQ ID | (SEQ ID | (SEQ ID | (SEQ ID |
| | NO: 51) | NO: 14) | NO: 133) | NO: 134) | NO: 135) | NO: 136) |
| r460 | QSGDLTA | RSGSLTR | TSGNLRT | RSANLRT | RSDNLAR | RSDNLTR |
| | (SEQ ID | (SEQ ID | (SEQ ID | (SEQ ID | (SEQ ID | (SEQ ID |
| | NO: 137) | NO: 138) | NO: 139) | NO: 140) | NO: 10) | NO: 14) |
| r49 | TSGHLSR | RSDNRTR | RSDHLSE | RNDNRKK | QSGHLTR | RSDHLTT |
| | (SEQ ID | (SEQ ID | (SEQ ID | (SEQ ID | (SEQ ID | (SEQ ID |
| | NO: 54) | NO: 141) | NO: 26) | NO: 142) | NO: 74) | NO: 40) |
| r150 | RSDALSR | RNDNRKT | QSGDLSV | RSDVRKV | DRSHLSR | RSSHLSR |
| | (SEQ ID | (SEQ ID | (SEQ ID | (SEQ ID | (SEQ ID | (SEQ ID |
| | NO: 84) | NO: 30) | NO: 143) | NO: 144) | NO: 145) | NO: 146) |
| r45 | QSGSLTR | RSDHLTT | QSSNLTR | RSGHL VR | RSDHLSQ | TSGDL VR |
| | (SEQ ID | (SEQ ID | (SEQ ID | (SEQ ID | (SEQ ID | (SEQ ID |
| | NO: 47) | NO: 40) | NO: 147) | NO: 148) | NO: 34) | NO: 149) |
| r87 | RSDHLSE | QSSTRTK | RSDHLSQ | DRSSLAR | RSDNLAR | QKGTLGE |
| | (SEQ ID | (SEQ ID | (SEQ ID | (SEQ ID | (SEQ ID | (SEQ ID |
| | NO: 26) | NO: 150) | NO: 34) | NO: 151) | NO: 10) | NO: 152) |
| r400 | RSDHLTR | QSSDRKR | QSAHLSR | QSSDLRR | QSGHLAR | QSGHLSR |
| | (SEQ ID | (SEQ ID | (SEQ ID | (SEQ ID | (SEQ ID | (SEQ ID |
| | NO: 42) | NO: 153) | NO: 50) | NO: 29) | NO: 78) | NO: 79) |

TABLE 3
canine-Sds Zinc Finger Regions for Targeted Repression

| Position | ZF1 | ZF2 | ZF3 | ZF4 | ZF5 | ZF6 |
|---|---|---|---|---|---|---|
| r358 | QSGHLAR | RSDNLAR | QSGDLTR | RLDNLPM | DRSDLSR | DRGTLTR |
| | (SEQ ID | (SEQ ID | (SEQ ID | (SEQ ID | (SEQ ID | (SEQ ID |
| | NO: 78) | NO: 10) | NO: 19) | NO: 154) | NO: 91) | NO: 155) |
| r67 | TSGHLAR | TSGNLAR | RSDNLSE | DRSDRIT | RSDHLST | TSSTRKK |
| | (SEQ ID | (SEQ ID | (SEQ ID | (SEQ ID | (SEQ ID | (SEQ ID |
| | NO: 156) | NO: 157) | NO: 133) | NO: 158) | NO: 53) | NO: 159) |
| r345 | DRDHLSE | DRSHRIR | RSDSLLR | RLDNRKA | QSGHLAR | QSGDLTR |
| | (SEQ ID | (SEQ ID | (SEQ ID | (SEQ ID | (SEQ ID | (SEQ ID |
| | NO: 160) | NO: 161) | NO: 131) | NO: 162) | NO: 78) | NO: 19) |
| r371 | RSDNLSR | RSDNLAR | DRDHLAQ | QSGNRKT | RSDNLAR | RSDHRKT |
| | (SEQ ID | (SEQ ID | (SEQ ID | (SEQ ID | (SEQ ID | (SEQ ID |
| | NO: 51) | NO: 10) | NO: 163) | NO: 164) | NO: 10) | NO: 165) |
| r247 | DRGDLTR | RNHHLSR | QSGDLTR | RSSNLAR | QSSALSR | RSANLAR |
| | (SEQ ID | (SEQ ID | (SEQ ID | (SEQ ID | (SEQ ID | (SEQ ID |
| | NO: 8) | NO: 166) | NO: 19) | NO: 167) | NO: 98) | NO: 168) |
| r352 | QSGDLTR | RSDNRKK | DRSDLSR | DRSDLSR | RSDHLSQ | RSDHLSR |
| | (SEQ ID | (SEQ ID | (SEQ ID | (SEQ ID | (SEQ ID | (SEQ ID |
| | NO: 19) | NO: 169) | NO: 91) | NO: 91) | NO: 34) | NO: 16) |
| r142 | RSDSLAR | QNGNLTR | DRSNLSR | QSGDLRR | QSGHLAR | QSGDLRT |
| | (SEQ ID | (SEQ ID | (SEQ ID | (SEQ ID | (SEQ ID | (SEQ ID |
| | NO: 21) | NO: 170) | NO: 13) | NO: 171) | NO: 78) | NO: 32) |
| r361 | QSGSLTR | QSGHLTR | RSDNLAR | QSGDLTR | RSGDLTR | DSSNLSR |
| | (SEQ ID | (SEQ ID | (SEQ ID | (SEQ ID | (SEQ ID | (SEQ ID |
| | NO: 47) | NO: 74) | NO: 10) | NO: 19) | NO: 172) | NO: 173) |
| r187 | DRGDLTE | QSDNLAA | QNGDLTT | RSASLTR | RSDNLAR | QSGTLTR |
| | (SEQ ID | (SEQ ID | (SEQ ID | (SEQ ID | (SEQ ID | (SEQ ID |
| | NO: 174) | NO: 175) | NO: 176) | NO: 177) | NO: 10) | NO: 119) |
| r437 | TSSNLSR | QSATLAR | RSDNLAR | RSDALRS | TSSHLSR | LKSYRIT |
| | (SEQ ID | (SEQ ID | (SEQ ID | (SEQ ID | (SEQ ID | (SEQ ID |
| | NO: 178) | NO: 179) | NO: 10) | NO: 180) | NO: 181) | NO: 182) |
| 175 | RSDALTR | DSATRKK | RSDSLST | DRATRKT | RSDNLAR | QSGHLSR |
| | (SEQ ID | (SEQ ID | (SEQ ID | (SEQ ID | (SEQ ID | (SEQ ID |
| | NO: 89) | NO: 183) | NO: 184) | NO: 185) | NO: 10) | NO: 79) |
| 165 | DRGDLTR | QSGHLAR | QSAHLAR | QKSTRKK | DRGNLSR | TSSNLTR |
| | (SEQ ID | SEQ ID | (SEQ ID | (SEQ ID | (SEQ ID | (SEQ ID |
| | NO: 8) | NO: 78) | NO: 31) | NO: 186) | NO: 187) | NO: 41) |
| 14 | DRSNLSR | QSGDLTR | QSGSLSR | LKQHLRT | DRSNLSR | RSDHLTT |
| | (SEQ ID | (SEQ ID | (SEQ ID | (SEQ ID | (SEQ ID | (SEQ ID |
| | NO: 13) | NO: 19) | NO: 188) | NO: 189) | NO: 13) | NO: 40) |
| 367 | ERGTLAR | DRSDLAR | QSGDLTR | LKQHLRS | DRGTLTR | QSGTLTR |
| | (SEQ ID | (SEQ ID | (SEQ ID | (SEQ ID | (SEQ ID | (SEQ ID |
| | NO: 190) | NO: 191) | NO: 19) | NO: 192) | NO: 155) | NO: 119) |
| 478 | TSGHLSR | RSDALAR | RSDALTR | RSDNRKA | RSDTLSE | QSATRTT |
| | (SEQ ID | (SEQ ID | (SEQ ID | (SEQ ID | (SEQ ID | (SEQ ID |
| | NO: 54) | NO: 11) | NO: 89) | NO: 193) | NO: 194) | NO: 195) |
| 481 | RSDNLSR | HSGHLSR | RSDSLSR | RSDALAR | RSDVLSQ | DNHHRKT |
| | (SEQ ID | (SEQ ID | (SEQ ID | (SEQ ID | (SEQ ID | (SEQ ID |
| | NO: 51) | NO: 196) | NO: 62) | NO: 11) | NO: 197) | NO: 198) |
| 153 | DSSNLTR | TSSNLSR | TSGHLSR | TSSSLAK | RSDALSQ | DRSSLTR |
| | (SEQ ID | (SEQ ID | (SEQ ID | (SEQ ID | (SEQ ID | (SEQ ID |
| | NO: 199) | NO: 178) | NO: 54) | NO: 200) | NO: 201) | NO: 202) |
| 11 | QSGDLTR | QSTSLRK | TPSALSR | DRSNLTR | RSDHLSA | DRATRTK |
| | (SEQ ID | (SEQ ID | (SEQ ID | (SEQ ID | (SEQ ID | (SEQ ID |
| | NO: 19) | NO: 203) | NO: 204) | NO: 25) | NO: 36) | NO: 205) |
| 393 | RSDVLSE | QNGDLTR | RSDHLST | TSSTRIK | QSGNLAR | RSDNLKD |
| | (SEQ ID | (SEQ ID | (SEQ ID | (SEQ ID | (SEQ ID | (SEQ ID |
| | NO: 65) | NO: 206) | NO: 53) | NO: 207) | NO: 67) | NO: 208) |
| 364 | ERGTLAR | QSGDLTR | RSDSLSV | DRSTRKR | RSGTLTR | QSQDRKT |
| | (SEQ ID | (SEQ ID | (SEQ ID | (SEQ ID | (SEQ ID | (SEQ ID |
| | NO: 190) | NO: 19) | NO: 209) | NO: 210) | NO: 211) | NO: 212) |

TABLE 4
equine-Sds Zinc Finger Regions for Targeted Repression

| Position | ZF1 | ZF2 | ZF3 | ZF4 | ZF5 | ZF6 |
|---|---|---|---|---|---|---|
| r429 | QSGSLTR | TSGNL VR | RSDALRV | DRGNLTR | QSGHLAR | TSSHLAR |
| | (SEQ ID | (SEQ ID | (SEQ ID | (SEQ ID | SEQ ID | (SEQ ID |
| | NO: 47) | NO: 213) | NO: 214) | NO: 215) | NO: 78) | NO: 216) |
| r66 | TSGHLVR | RRDALTT | RLGNLSE | QNANRIR | RSDNLSA | RSHHRIN |
| | (SEQ ID | (SEQ ID | (SEQ ID | (SEQ ID | (SEQ ID | (SEQ ID |
| | NO: 217) | NO: 218) | NO: 219) | NO: 220) | NO: 43) | NO: 221) |
| r421 | RSDDRKE | RNSNRIK | RSDSLSR | RSDHLSR | RSDNLAR | QSGHLSR |
| | (SEQ ID | (SEQ ID | (SEQ ID | (SEQ ID | (SEQ ID | (SEQ ID |
| | NO: 222) | NO: 223) | NO: 62) | NO: 16) | NO: 10) | NO: 79) |
| r420 | DRSNLSR | QSGHLSR | RSDHLSR | RSDALAR | RSGHLAR | RSAHLSR |
| | (SEQ ID | (SEQ ID | (SEQ ID | (SEQ ID | (SEQ ID | (SEQ ID |
| | NO: 13) | NO: 79) | NO: 16) | NO: 11) | NO: 224) | NO: 38) |
| r435 | RSDHLSE | QNANRTK | QSSNLSR | LKSNRTR | RSDSLSQ | DRSNRTK |
| | (SEQ ID | (SEQ ID | (SEQ ID | (SEQ ID | (SEQ ID | (SEQ ID |
| | NO: 26) | NO: 225) | NO: 116) | NO: 226) | NO: 227) | NO: 64) |
| r354 | QSGNLSR | TSSNLSR | QSSDLSR | TSQHLSK | QSSALSR | QSANLTR |
| | (SEQ ID | (SEQ ID | (SEQ ID | (SEQ ID | (SEQ ID | (SEQ ID |
| | NO: 75) | NO: 178) | NO: 15) | NO: 228) | NO: 98) | NO: 229) |
| r56 | QSGHLSR | QSGNLAR | RSDALSR | RSDHLSR | RSDHLSK | QSATRTT |
| | (SEQ ID | (SEQ ID | (SEQ ID | (SEQ ID | (SEQ ID | (SEQ ID |
| | NO: 79) | NO: 67) | NO: 84) | NO: 16) | NO: 112) | NO: 195) |
| r57 | QSGNLSR | RSDNRKA | RSHHLAN | RSDALAR | RSDHLSQ | QSSNRTK |
| | (SEQ ID | (SEQ ID | (SEQ ID | (SEQ ID | (SEQ ID | (SEQ ID |
| | NO: 75) | NO: 193) | NO: 230) | NO: 11) | NO: 34) | NO: 231) |
| r405 | RSDHLSR | QSGNLAR | RSDNLAR | QKGDRKS | DRSDLSR | QAGNLSK |
| | (SEQ ID | (SEQ ID | (SEQ ID | (SEQ ID | (SEQ ID | (SEQ ID |
| | NO: 16) | NO: 67) | NO: 10) | NO: 232) | NO: 91) | NO: 233) |
| r432 | RSDHLAE | QSSTRNK | TSGSLAR | RSDALRA | TSGTLTR | QSGHLTR |
| | (SEQ ID | (SEQ ID | (SEQ ID | (SEQ ID | (SEQ ID | (SEQ ID |
| | NO: 234) | NO: 235) | NO: 236) | NO: 237) | NO: 238) | NO: 74) |
| r413 | RSDHLAA | QNDNLAR | RSAHLAR | RSDHRKK | QSSHLAR | QSAHRKT |
| | (SEQ ID | (SEQ ID | (SEQ ID | (SEQ ID | (SEQ ID | (SEQ ID |
| | NO: 239) | NO: 240) | NO: 241) | NO: 242) | NO: 243) | NO: 244) |
| r60 | RSDTLST | QNANRTR | RSDNLSA | RNQNRTN | RSDHLSR | TSDHRKT |
| | (SEQ ID | (SEQ ID | (SEQ ID | (SEQ ID | (SEQ ID | (SEQ ID |
| | NO: 245) | NO: 246) | NO: 43) | NO: 247) | NO: 16) | NO: 248) |
| r65 | RSDHLSE | RNASRTR | QSSNLRT | RSDNLRA | QSGNLAR | RSDALAR |
| | (SEQ ID | (SEQ ID | (SEQ ID | (SEQ ID | (SEQ ID | (SEQ ID |
| | NO: 26) | NO: 48) | NO: 249) | NO: 250) | NO: 67) | NO: 11) |
| r90 | RSDHLSE | TSSNRTK | RSDTLSV | TSSNRTK | QSDVLSQ | TSSNRTT |
| | (SEQ ID | (SEQ ID | (SEQ ID | (SEQ ID | (SEQ ID | (SEQ ID |
| | NO: 26) | NO: 251) | NO: 252) | NO: 251) | NO: 253) | NO: 254) |
| 308 | RSDVLTA | QNATRTK | RSDTLSK | TSSNRKQ | QSGNLAR | RSDNLST |
| | (SEQ ID | (SEQ ID | (SEQ ID | (SEQ ID | (SEQ ID | (SEQ ID |
| | NO: 255) | NO: 256) | NO: 257) | NO: 258) | NO: 67) | NO: 63) |
| 273 | RSDHLAA | QSATRTK | QSGSLSR | TSQNLTK | TSSALSR | HRSYLTD |
| | (SEQ ID | (SEQ ID | (SEQ ID | (SEQ ID | (SEQ ID | (SEQ ID |
| | NO: 239) | NO: 259) | NO: 188) | NO: 260) | NO: 261) | NO: 262) |
| 454 | QSGDLSQ | QSSNLSR | TSSALSR | QSGNLTR | RSGHLSE | DSGHLTR |
| | (SEQ ID | (SEQ ID | (SEQ ID | (SEQ ID | (SEQ ID | (SEQ ID |
| | NO: 263) | NO: 116) | NO: 261) | NO: 110) | NO: 264) | NO: 265) |
| 337 | QSGHLSE | RNDARTK | DRDTLSA | TSGNLTR | DRDTLSQ | RSANLTR |
| | (SEQ ID | (SEQ ID | (SEQ ID | (SEQ ID | (SEQ ID | (SEQ ID |
| | NO: 266) | NO: 267) | NO: 268) | NO: 269) | NO: 270) | NO: 73) |
| 353 | QSSNLSR | QSANRTK | QSGALAR | TSHNRTH | RSSNLSR | TSANRIN |
| | (SEQ ID | (SEQ ID | (SEQ ID | (SEQ ID | (SEQ ID | (SEQ ID |
| | NO: 116) | NO: 271) | NO: 88) | NO: 272) | NO: 273) | NO: 274) |
| 388 | RSDNLST | QSSNRTT | QSSNLAR | LKANLRK | DRSNLSR | RSDALRQ |
| | (SEQ ID | (SEQ ID | (SEQ ID | (SEQ ID | (SEQ ID | (SEQ ID |
| | NO: 63) | NO: 275) | NO: 276) | NO: 277) | NO: 13) | NO: 278) |

TABLE 5
feline-Sds Zinc Finger Regions for Targeted Repression

| Position | ZF1 | ZF2 | ZF3 | ZF4 | ZF5 | ZF6 |
|---|---|---|---|---|---|---|
| r231 | TSGHLAR | TSGNLAR | DRSNLTR | DRSNLRK | RSDHLST | TSSTRKK |
| | (SEQ ID | (SEQ ID | (SEQ ID | (SEQ ID | (SEQ ID | (SEQ ID |
| | NO: 156) | NO: 157) | NO: 25) | NO: 279) | NO: 53) | NO: 159) |
| r237 | RSDALSV | DRSHLAR | TSGHLSR | TSGNLAA | DRSNLSR | DRSNRTR |
| | (SEQ ID | (SEQ ID | (SEQ ID | (SEQ ID | (SEQ ID | (SEQ ID |
| | NO: 104) | NO: 124) | NO: 54) | NO: 280) | NO: 13) | NO: 281) |
| r75 | QSSNRKT | TSSNRKT | DRSALSR | RSDHLSR | RSDNLAR | TSGNLVR |
| | (SEQ ID | (SEQ ID | (SEQ ID | (SEQ ID | (SEQ ID | (SEQ ID |
| | NO: 282) | NO: 283) | NO: 61) | NO: 16) | NO: 10) | NO: 213) |
| r78 | RSDHLSA | RNGNLTR | RSDALST | DRSALSR | RSDHLSR | RNDNRIT |
| | (SEQ ID | (SEQ ID | (SEQ ID | (SEQ ID | (SEQ ID | (SEQ ID |
| | NO: 36) | NO: 284) | NO: 285) | NO: 61) | NO: 16) | NO: 286) |
| r306 | RSDSLAR | QNGNLTR | DRSNLSR | QSGDLRR | QSGHLAR | QSGDLRT |
| | (SEQ ID | (SEQ ID | (SEQ ID | (SEQ ID | (SEQ ID | (SEQ ID |
| | NO: 21) | NO: 170) | NO: 13) | NO: 171) | NO: 78) | NO: 32) |
| r411 | DRGDLTR | TSGHLTR | TSGDLTR | RSGNL VR | RSGHLSR | RSGNLTR |
| | (SEQ ID | (SEQ ID | (SEQ ID | (SEQ ID | (SEQ ID | (SEQ ID |
| | NO: 8) | NO: 107) | NO: 287) | NO: 288) | NO: 121) | NO: 289) |
| r372 | RSDHLST | NSSTRIK | QSGNLAR | QNGNLSK | DSDVLNE | TSGNLTR |
| | (SEQ ID | (SEQ ID | (SEQ ID | (SEQ ID | (SEQ ID | (SEQ ID |
| | NO: 53) | NO: 290) | NO: 67) | NO: 291) | NO: 292) | NO: 269) |
| r76 | RSDNLRA | TSAHRIT | RSDSLSQ | RSDHLSR | RSDNLSA | RNSSRKN |
| | (SEQ ID | (SEQ ID | (SEQ ID | (SEQ ID | (SEQ ID | (SEQ ID |
| | NO: 250) | NO: 293) | NO: 227) | NO: 16) | NO: 43) | NO: 294) |
| r271 | DRGTLSR | QSSDLSR | RSDDLSR | DNQHLTS | QSSNLTR | RSDDLSK |
| | (SEQ ID | (SEQ ID | (SEQ ID | (SEQ ID | (SEQ ID | (SEQ ID |
| | NO: 295) | NO: 15) | NO: 296) | NO: 297) | NO: 147) | NO: 135) |
| r369 | DRGDLTR | QSAHLTR | QSGNLRA | RNDALTS | TSDTLSE | RNRDRTK |
| | (SEQ ID | (SEQ ID | (SEQ ID | (SEQ ID | (SEQ ID | (SEQ ID |
| | NO: 8) | NO: 111) | NO: 298) | NO: 299) | NO: 300) | NO: 301) |
| 11 | RSDALTR | DSATRKK | RSDSLST | DRATRKT | RSDNLAR | QSGHLSR |
| | (SEQ ID | (SEQ ID | (SEQ ID | (SEQ ID | (SEQ ID | (SEQ ID |
| | NO: 89) | NO: 183) | NO: 184) | NO: 185) | NO: 10) | NO: 79) |
| 311 | RSDALTR | RSDALAR | RSDNLSV | DNSNRTK | RSDSLLR | DRSHLTR |
| | (SEQ ID | (SEQ ID | (SEQ ID | (SEQ ID | (SEQ ID | (SEQ ID |
| | NO: 89) | NO: 11) | NO: 114) | NO: 302) | NO: 131) | NO: 105) |
| 437 | QSGNLSR | QSGNLRR | RSDALSR | DRANRKK | DRSYLSR | HRSYLTR |
| | (SEQ ID | (SEQ ID | (SEQ ID | (SEQ ID | (SEQ ID | (SEQ ID |
| | NO: 75) | NO: 93) | NO: 84) | NO: 303) | NO: 304) | NO: 305) |
| 359 | RSDNLRE | QSATRTK | RSDALSV | DRSSRTK | RSDTLSA | DNATRTK |
| | (SEQ ID | (SEQ ID | (SEQ ID | (SEQ ID | (SEQ ID | (SEQ ID |
| | NO: 306) | NO: 259) | NO: 104) | NO: 307) | NO: 308) | NO: 309) |
| 203 | ERGTLAR | DRSDLAR | QSGDLTR | LKQHLRS | DRGTLTR | QSGTLTR |
| | (SEQ ID | (SEQ ID | (SEQ ID | (SEQ ID | (SEQ ID | (SEQ ID |
| | NO: 190) | NO: 191) | NO: 19) | NO: 192) | NO: 155) | NO: 119) |
| 212 | ERGTLAR | DRSDLTR | DRSALRR | DRQHLSR | DRGTLSR | QSGDLAR |
| | (SEQ ID | (SEQ ID | (SEQ ID | (SEQ ID | (SEQ ID | (SEQ ID |
| | NO: 190) | NO: 117) | NO: 310) | NO: 311) | NO: 295) | NO: 312) |
| 353 | RSDALSA | DRRDRTK | RSDNLSV | DNSTRIK | RSDVLSQ | RNSSRKN |
| | (SEQ ID | (SEQ ID | (SEQ ID | (SEQ ID | (SEQ ID | (SEQ ID |
| | NO: 313) | NO: 314) | NO: 114) | NO: 315) | NO: 197) | NO: 294) |
| 320 | RSDSLSE | RSASRIN | RSDHLST | RSDALAR | RSDALSR | RSDNRIT |
| | (SEQ ID | (SEQ ID | (SEQ ID | (SEQ ID | (SEQ ID | (SEQ ID |
| | NO: 316) | NO: 317) | NO: 53) | NO: 11) | NO: 84) | NO: 82) |
| 59 | RSDDLSA | DRSHRTK | RSDDLSK | RSDARKA | QSGNRKA | DRSNRTK |
| | (SEQ ID | (SEQ ID | (SEQ ID | (SEQ ID | (SEQ ID | (SEQ ID |
| | NO: 318) | NO: 319) | NO: 135) | NO: 320) | NO: 321) | NO: 64) |
| 317 | RSDNLSR | HSGHLSR | RSDSLSR | RSDALAR | RSDVLSQ | DNHHRKT |
| | (SEQ ID | (SEQ ID | (SEQ ID | (SEQ ID | (SEQ ID | (SEQ ID |
| | NO: 51) | NO: 196) | NO: 62) | NO: 11) | NO: 197) | NO: 198) |

TABLE 6
hSDS CRISPR sgRNA for Knock Out,
Interference, & Activation

| | CRISPRko | CRISPRi | CRISPRa |
|---|---|---|---|
| 1 | GGAAGCAGATCAC | GTAGATAGCCCACGAA | GCCCAGGACCAAGTAG |
| | TTACCCG (SEQ | GAGA (SEQ ID NO: | CCCC (SEQ ID NO: |
| | ID NO: 322) | 323) | 324) |
| 2 | GCCCTAGCGAAGA | AGATAGCCCACGAAGA | ACGAAACTGAGGACAG |
| | ACAACCC (SEQ | GAGG (SEQ ID NO: | AAAG (SEQ ID NO: |
| | ID NO: 325) | 326) | 327) |
| 3 | GGGGAATGTAGAC | TAGATAGCCCACGAAG | CTCAGTGGCCTCAAGC |
| | CCAACCC (SEQ | AGAG (SEQ ID NO: | CAGG (SEQ ID NO: |
| | ID NO: 328) | 329) | 330) |
| 4 | AACTCCACATACC | AGTAGATAGCCCACGA | GTGCCAGAGGAATAAA |
| | AGATGAG (SEQ | AGAG (SEQ ID NO: | GCCC (SEQ ID NO: |
| | ID NO: 331) | 332) | 333) |
| 5 | CTCACCCACCACC | AGTCAGAGCCAAGCCA | AGTCTTCTGAATGGTC |
| | TTGACTG (SEQ | GCGA (SEQ ID NO: | CCAG (SEQ ID NO: |
| | ID NO: 334) | 335) | 336) |
| 6 | GCCGCCCACTGAC | GAGTCAGAGCCAAGCC | GGCCACTGAGGCTGTC |
| | AGCGCGA (SEQ | AGCG (SEQ ID NO: | AGAA (SEQ ID NO: |
| | ID NO: 337) | 338) | 339) |
| 7 | ACTGACAGCGCGA | AGAGCCAAGCCAGCGA | CCTGCCAGGACCCACC |
| | TGGCCCC (SEQ | GGGA (SEQ ID NO: | CCCC (SEQ ID NO: |
| | ID NO: 340) | 341) | 342) |
| 8 | GTCCATCTTGAGG | GCTAGCCTGTGGTTTC | AGGCCACTGAGGCTGT |
| | TAGACGC (SEQ | CCAG (SEQ ID NO: | CAGA (SEQ ID NO: |
| | ID NO: 343) | 344) | 345) |
| 9 | GGCAGCGGGCAAC | CAAAGGTGATGGGTCT | GCCTCAGTGGCCTCAA |
| | GCAGGCA (SEQ | GAGC (SEQ ID NO: | GCCA (SEQ ID NO: |
| | ID NO: 346) | 347) | 348) |
| 10 | TGCAGAAGTGCCC | GGCTAGCCTGTGGTTT | GCACCTCTGGGCAGCC |
| | AATGCCC (SEQ | CCCA (SEQ ID NO: | AATG (SEQ ID NO: |
| | ID NO: 349) | 350) | 351) |
| 11 | CAGGGAGACAAGT | TCAGTTGATCCCTCCC | GGACCCACCCCCCTGG |
| | TTGCCTG (SEQ | TCGC (SEQ ID NO: | CTTG (SEQ ID NO: |
| | ID NO: 352) | 353) | 354) |
| 12 | ATTGGATGAAGCC | CAGTCTTCTGAATGGT | TGAGGCTGTCAGAAGG |
| | TTCGAGC (SEQ | CCCA (SEQ ID NO: | GAGG (SEQ ID NO: |
| | ID NO: 355) | 356) | 357) |
| 13 | TAGCGAAGAACAA | AGAAAGAGTCTGTCAA | TCAGTGGCCTCAAGCC |
| | CCCGGGT (SEQ | GGCT (SEQ ID NO: | AGGG (SEQ ID NO: |
| | ID NO: 358) | 359) | 360) |
| 14 | TGAACTCCACATA | GACATAGAAAGAGTCT | AGCCTCAGTGGCCTCA |
| | CCAGATG (SEQ | GTCA (SEQ ID NO: | AGCC (SEQ ID NO: |
| | ID NO: 361) | 362) | 363) |
| 15 | GCCATCGCGCTGT | ACAGTCTTCTGAATGG | CCTCAGTGGCCTCAAG |
| | CAGTGGG (SEQ | TCCC (SEQ ID NO: | CCAG (SEQ ID NO: |
| | ID NO: 364) | 365) | 366) |
| 16 | ACCCTGGCAGCGG | CGGGTGGACAGTCTTC | GTGGGTCCTGGCAGGA |
| | GCAACGC (SEQ | TGAA (SEQ ID NO: | GGGA (SEQ ID NO: |
| | ID NO: 367) | 368) | 369) |
| 17 | GGGGCCATCGCGC | TCTATGTCTCCCTCTT | CACTGAGGCTGTCAGA |
| | TGTCAGT (SEQ | CCCC (SEQ ID NO: | AGGG (SEQ ID NO: |
| | ID NO: 370) | 371) | 372) |
| 18 | TCCGGCTCCTTCA | CCTCAGCCCCCTCTCT | CCCCTGGCTTGAGGCC |
| | AGATCCG (SEQ | TCGT (SEQ ID NO: | ACTG (SEQ ID NO: |
| | ID NO: 373) | 374) | 375) |
| 19 | ACTCCACATACCA | TGTGGTTTCCCAGGGG | TGGGTTGTGGGAAAGG |
| | GATGAGG (SEQ | AAGA (SEQ ID NO: | CAGG (SEQ ID NO: |
| | ID NO: 376) | 377) | 378) |
| 20 | GCTCTTTCACGAT | TCCTCAGCCCCCTCTC | CTCCCTTCTGACAGCC |
| | GGAAGCG (SEQ | TTCG (SEQ ID NO: | TCAG (SEQ ID NO: |
| | ID NO: 379) | 380) | 381) |
| 21 | TGCTGTGTGGAGT | GAGAGGGGGCTGAGGA | GTCAGAAGGGAGGTGG |
| | GGTCCAG (SEQ | CGGG (SEQ ID NO: | AATC (SEQ ID NO: |
| | ID NO: 382) | 383) | 384) |
| 22 | GGCCCTAGCGAAG | CAGAGCCAAGCCAGCG | AGAAAGGGGAAGTGGA |
| | AACAACC (SEQ | AGGG (SEQ ID NO: | AAGA (SEQ ID NO: |
| | ID NO: 385) | 386) | 387) |
| 23 | AGACACTGTGGGA | GAAGAGAGGGGGCTGA | GGGTCCTGGCAGGAGG |
| | AAAGCCG (SEQ | GGAC (SEQ ID NO: | GAGG (SEQ ID NO: |
| | ID NO: 388) | 389) | 390) |
| 24 | AAAGTCTCCATGG | CGAAGAGAGGGGGCTG | CAGAAAGGGGAAGTGG |
| | CGATGAC (SEQ | AGGA (SEQ ID NO: | AAAG (SEQ ID NO: |
| | ID NO: 391) | 392) | 393) |
| 25 | GGTGCTGGGCACC | CCCACGAAGAGAGGGG | AAGGGGAAGTGGAAAG |
| | ACGATGG (SEQ | GCTG (SEQ ID NO: | AGGG (SEQ ID NO: |
| | ID NO: 394) | 395) | 396) |

TABLE 7
mSds CRISPR sgRNA for Knock Out,
Interference, & Activation

| | CRISPRko | CRISPRi | CRISPRa |
|---|---|---|---|
| 1 | ACAGACAGCACGATAG | TCTAGATCAGGATGTC | ACTACGTGTCCAGC |
| | CCCC (SEQ ID NO: | GCTG (SEQ ID NO: | AACTGC (SEQ ID |
| | 397) | 398) | NO: 399) |
| 2 | GGTGTTGGGCACAACG | GATGTCGCTGGGGTGG | GTTATTCTGAGACT |
| | ATAG (SEQ ID NO: | CATG (SEQ ID NO: | GACCCC (SEQ ID |
| | 400) | 401) | NO: 402) |
| 3 | GCTCCAACTTACCAGA | GTCCCTGACTGTTAAC | TTATTCTGAGACTG |
| | TGAG (SEQ ID NO: | CTTG (SEQ ID NO: | ACCCCC (SEQ ID |
| | 403) | 404) | NO: 405) |
| 4 | GGGAAATGTACACCCA | TTCTAGATCAGGATGT | GTCCTGGGAGAGTA |
| | ACCC (SEQ ID NO: | CGCT (SEQ ID NO: | CAAAGT (SEQ ID |
| | 406) | 407) | NO: 408) |
| 5 | CTCTCCCACCACTTCA | AGATCAGGATGTCGCT | TATCGCTAAGAAAC |
| | ACTG (SEQ ID NO: | GGGG (SEQ ID NO: | AGCCAC (SEQ ID |
| | 409) | 410) | NO: 411) |
| 6 | TGGGCACAACGATAGT | GTAAAATGGGACGAAG | CAGGGGGCGGAGCT |
| | GGCT (SEQ ID NO: | TGGT (SEQ ID NO: | TGCTTG (SEQ ID |
| | 412) | 413) | NO: 414) |
| 7 | TCAGGCTCCTTCAAGA | CTGTCCCTGACTGTTA | GGGCGGAGCTTGCT |
| | TCCG (SEQ ID NO: | ACCT (SEQ ID NO: | TGGGGT (SEQ ID |
| | 415) | 416) | NO: 417) |
| 8 | TCCACTGACAGCTGGC | TGCCAACTTTGTACTC | GGGCCTCATTGGCT |
| | AACG (SEQ ID NO: | TCCC (SEQ ID NO: | ACCCGG (SEQ ID |
| | 418) | 419) | NO: 420) |
| 9 | ATTGAGCGGCTCAAGA | GGCCACATACCCGGCA | AAGAGCCCTTGCCT |
| | ATGA (SEQ ID NO: | CAGG (SEQ ID NO: | GGACCC (SEQ ID |
| | 421) | 422) | NO: 423) |
| 10 | CGACCGCCTACGCTGC | TGACTGTTAACCTTGG | CAGCCCTGAGAATA |
| | CAGG (SEQ ID NO: | GGAG (SEQ ID NO: | AAGCTC (SEQ ID |
| | 424) | 425) | NO: 426) |
| 11 | CACAGACAGCACGATA | TGTCCCTGACTGTTAA | GTGCCGCATGCCCT |
| | GCCC (SEQ ID NO: | CCTT (SEQ ID NO: | CCTCCG (SEQ ID |
| | 427) | 428) | NO: 429) |
| 12 | GATGAGAGGGTCATCA | CCAGCTTCAGCCTCTC | GAGCCCTTGCCTGG |
| | AAGG (SEQ ID NO: | CCCA (SEQ ID NO: | ACCCGG (SEQ ID |
| | 430) | 431) | NO: 432) |
| 13 | AGGCGCCACAGTTGAA | GGGGGCCACATACCCG | GAGCCTCCGGGTAG |
| | GTGG (SEQ ID NO: | GCAC (SEQ ID NO: | CCAATG (SEQ ID |
| | 433) | 434) | NO: 435) |
| 14 | GCCCAGCCTCCTGGCA | AGCTGGACAGAGCGAG | TGACCCCCGGGTCC |
| | GCGT (SEQ ID NO: | CGAG (SEQ ID NO: | AGGCAA (SEQ ID |
| | 436) | 437) | NO: 438) |
| 15 | CACCTGCCCTCACCAT | TGGGGAGAGGCTGAAG | CGGCCACGGAGGAG |
| | TGAG (SEQ ID NO: | CTGG (SEQ ID NO: | GGCATG (SEQ ID |
| | 439) | 440) | NO: 441) |
| 16 | GACGCTGAGCGCCAAG | TTGGGGAGAGGCTGAA | CAGCCCCGGCCTGG |
| | CCCG (SEQ ID NO: | GCTG (SEQ ID NO: | ACCAGG (SEQ ID |
| | 442) | 443) | NO: 444) |
| 17 | TGCTGTGCGGAGTGGT | GAAGCTGGGGGCCACA | GGCTGGGCGGCCAC |
| | CCAG (SEQ ID NO: | TACC (SEQ ID NO: | GGAGGA (SEQ ID |
| | 445) | 446) | NO: 447) |
| 18 | TGAAGGCGCCACAGTT | GTAGATTGTGTCTGTG | GAAGAGCCCTTGCC |
| | GAAG (SEQ ID NO: | TCCT (SEQ ID NO: | TGGACC (SEQ ID |
| | 448) | 449) | NO: 450) |
| 19 | CAGCCTCCTGGCAGCG | ATACCCGGCACAGGAG | TGGGAGAGTACAAA |
| | TAGG (SEQ ID NO: | GAGC (SEQ ID NO: | GTTGGC (SEQ ID |
| | 451) | 452) | NO: 453) |
| 20 | GCTATCGTGCTGTCTG | CTCCTCCTGTGCCGGG | AGAGCCCTTGCCTG |
| | TGGG (SEQ ID NO: | TATG (SEQ ID NO: | GACCCG (SEQ ID |
| | 454) | 455) | NO: 456) |
| 21 | GGGGCTATCGTGCTGT | CTGTCCAGCTCCTCCT | CCAGCCCCGGCCTG |
| | CTGT (SEQ ID NO: | GTGC (SEQ ID NO: | GACCAG (SEQ ID |
| | 457) | 458) | NO: 459) |
| 22 | GGCGCCACAGTTGAAG | TGTCCAGCTCCTCCTG | CGGGTAGCCAATGA |
| | TGGT (SEQ ID NO: | TGCC (SEQ ID NO: | GGCCCC (SEQ ID |
| | 460) | 461) | NO: 462) |
| 23 | AGATGAGAGGGTCATC | CTTGGGGAGAGGCTGA | GCTGCCATGTGGCT |
| | AAAG (SEQ ID NO: | AGCT (SEQ ID NO: | AGCCTG (SEQ ID |
| | 463) | 464) | NO: 465) |
| 24 | GCTCTTTCACAAGGGA | AGTAGATTGTGTCTGT | CAAGCTCCGCCCCC |
| | AGTG (SEQ ID NO: | GTCC (SEQ ID NO: | TGGTCC (SEQ ID |
| | 466) | 467) | NO: 468) |
| 25 | AAGGTCTCCATGGCGA | CCTTGGGGAGAGGCTG | TGGTCCAGGCCGGG |
| | TGAT (SEQ ID NO: | AAGC (SEQ ID NO: | GCTGGG (SEQ ID |
| | 469) | 470) | NO: 471) |

TABLE 8
rat-Sds CRISPR sgRNA for Knock Out,
Interference, & Activation

| | CRISPRko | CRISPRi | CRISPRa |
|---|---|---|---|
| 1 | GGGAGATGTACACCCA | GAGGGCCACATACC | AGGCCTATAGGACTCG |
| | ACCT (SEQ ID NO: | CGGCAC (SEQ ID | TGGG (SEQ ID NO: |
| | 472) | NO: 473) | 474) |
| 2 | GGTGCTTGGCACAACA | AGATCAGGACGTCG | AGTGGTGCAGCATGGA |
| | ATAG (SEQ ID NO: | CCGGGG (SEQ ID | CCAG (SEQ ID NO: |
| | 475) | NO: 476) | 477) |
| 3 | AGATGAGAGGGTCATC | GGCCAAGTCACACG | GCCCGGAGAATAAAGC |
| | GAAG (SEQ ID NO: | CCACCC (SEQ ID | CCTG (SEQ ID NO: |
| | 478) | NO: 479) | 480) |
| 4 | GCTCCAACTTACCAGA | TCTAGATCAGGACG | AAGTGGTGCAGCATGG |
| | TGAG (SEQ ID NO: | TCGCCG (SEQ ID | ACCA (SEQ ID NO: |
| | 403) | NO: 481) | 482) |
| 5 | GATGAGAGGGTCATCG | CCGGCGACGTCCTG | AAAGTTGGCAGGACAC |
| | AAGG (SEQ ID NO: | ATCTAG (SEQ ID | CCCA (SEQ ID NO: |
| | 483) | NO: 484) | 485) |
| 6 | CTCTCCCACCACTTCA | CCTCTAGATCAGGA | GTGGTGCAGCATGGAC |
| | ACTG (SEQ ID NO: | CGTCGC (SEQ ID | CAGG (SEQ ID NO: |
| | 409) | NO: 486) | 487) |
| 7 | TCCCCACAGAAGGCAA | ATGATAGGACCCTC | CGAAAGGCCTATAGGA |
| | AACA (SEQ ID NO: | TAGATC (SEQ ID | CTCG (SEQ ID NO: |
| | 488) | NO: 489) | 490) |
| 8 | ATTGAGCGGCTGAAGA | CGGCGACGTCCTGA | GAAAGGCCTATAGGAC |
| | ACGA (SEQ ID NO: | TCTAGA (SEQ ID | TCGT (SEQ ID NO: |
| | 491) | NO: 492) | 493) |
| 9 | TCCACTGACAGCGGGC | TGCTCTGGCTGTCA | CAAGTGGTGCAGCATG |
| | AACG (SEQ ID NO: | ATCTTG (SEQ ID | GACC (SEQ ID NO: |
| | 494) | NO: 495) | 496) |
| 10 | TGACGGCAGCGTGGAA | CTGGACAGAGCGAG | AGCCCGGAGAATAAAG |
| | GCTG (SEQ ID NO: | CGAGAA (SEQ ID | CCCT (SEQ ID NO: |
| | 497) | NO: 498) | 499) |
| 11 | TCTGGCTCCTTCAAGA | CTCTAGATCAGGAC | GAAAGTTGGCAGGACA |
| | TCCG (SEQ ID NO: | GTCGCC (SEQ ID | CCCC (SEQ ID NO: |
| | 500) | NO: 501) | 502) |
| 12 | GCTCTGGAAAAGAACA | GGCCACATACCCGG | AGAGAAGGTGAGGCGG |
| | ACCC (SEQ ID NO: | CACAGG (SEQ ID | ACCT (SEQ ID NO: |
| | 503) | NO: 422) | 504) |
| 13 | CACCTGCCCTCACCAT | TGGGGTGAGACTGA | GCAGAACAAGTGGTGC |
| | TGAG (SEQ ID NO: | GGCTGA (SEQ ID | AGCA (SEQ ID NO: |
| | 439) | NO: 505) | 506) |
| 14 | GACACTGAGCGCCAAG | TCAATCTTGGGGTG | AAGGTGAGGCGGACCT |
| | CCCG (SEQ ID NO: | AGACTG (SEQ ID | TGGA (SEQ ID NO: |
| | 507) | NO: 508) | 509) |
| 15 | TGCTGTGCGGAGTGGT | GCTGGACAGAGCGA | GAGCCTCCGGGCAGCC |
| | CCAG (SEQ ID NO: | GCGAGA (SEQ ID | AATG (SEQ ID NO: |
| | 445) | NO: 510) | 511) |
| 16 | CTTAAGGAACACACTA | CCTGCTCTGGCTGT | GGCCCTCATTGGCTGC |
| | GTGC (SEQ ID NO: | CAATCT (SEQ ID | CCGG (SEQ ID NO: |
| | 512) | NO: 513) | 514) |
| 17 | GGAAAAGAACAACCCA | ATTTGACTCATGCG | CAGGGGGCGGGGATTT |
| | GGTT (SEQ ID NO: | AGCACT (SEQ ID | CTGG (SEQ ID NO: |
| | 515) | NO: 516) | 517) |
| 18 | TTGGCACAACAATAGT | CTGCTCTGGCTGTC | ACCACTTGTTCTGCCC |
| | GGCT (SEQ ID NO: | AATCTT (SEQ ID | TCCA (SEQ ID NO: |
| | 518) | NO: 519) | 520) |
| 19 | TGAGCGGCTGAAGAAC | TGTCCAGCTCCTCC | CCAGGGGGCGGGGATT |
| | GAAG (SEQ ID NO: | TGTGCC (SEQ ID | TCTG (SEQ ID NO: |
| | 521) | NO: 461) | 522) |
| 20 | CTTCAGCCGCTCAATG | CCAAGATTGACAGC | ACCTTGGAGGGCAGAA |
| | GTGA (SEQ ID NO: | CAGAGC (SEQ ID | CAAG (SEQ ID NO: |
| | 523) | NO: 524) | 525) |
| 21 | GACGGCAGCGTGGAAG | CTCCTCCTGTGCCG | CCCCAGAAATCCCCGC |
| | CTGT (SEQ ID NO: | GGTATG (SEQ ID | CCCC (SEQ ID NO: |
| | 526) | NO: 455) | 527) |
| 22 | AGGCCACACTTCCCTT | ATACCCGGCACAGG | CAGCCCGGAGAATAAA |
| | GTGA (SEQ ID NO: | AGGAGC (SEQ ID | GCCC (SEQ ID NO: |
| | 528) | NO: 452) | 529) |
| 23 | AGACACTGAGCGCCAA | GAGGCTGAGGGCCA | GTCCTGGGAGAGTGGA |
| | GCCC (SEQ ID NO: | CATACC (SEQ ID | AAGT (SEQ ID NO: |
| | 530) | NO: 531) | 532) |
| 24 | CAGATGAGAGGGTCAT | CTGTCCAGCTCCTC | AAGAACGGGGAGAGAA |
| | CGAA (SEQ ID NO: | CTGTGC (SEQ ID | GGTG (SEQ ID NO: |
| | 533) | NO: 458) | 534) |
| 25 | TTCCACGCTGCCGTCA | TTGGGGTGAGACTG | AGCCTCCGGGCAGCCA |
| | AGGA (SEQ ID NO: | AGGCTG (SEQ ID | ATGA (SEQ ID NO: |
| | 535) | NO: 536) | 537) |

TABLE 9
canine-Sds CRISPR sgRNA for
Knock Out & Interference

| | CRISPRko | CRISPRi |
|---|---|---|
| 1 | GTGTTCCTGAAACAGCTTCA | TTGGACACCTCTGCAAGATG |
| | (SEQ ID NO: 538) | (SEQ ID NO: 539) |
| 2 | GCTGCCATTGAGAAGTTCGT | TGGACACCTCTGCAAGATGT |
| | (SEQ ID NO: 540) | (SEQ ID NO: 541) |
| 3 | CATACCCACGAACTTCTCAA | ATGGACAGTGCCCAACCTTC |
| | (SEQ ID NO: 542) | (SEQ ID NO: 543) |
| 4 | CACCACGCGGCTGTACACGG | ATAGATGGTCCTCTCACGGA |
| | (SEQ ID NO: 544) | (SEQ ID NO: 545) |
| 5 | CCCGCAGACAATGACCACGA | CTGACCAAGGCAGTGAAGGT |
| | (SEQ ID NO: 546) | (SEQ ID NO: 547) |
| 6 | GGTGGCAGCGTGGAAGCTGT | CTGGATAGATGGTCCTCTCA |
| | (SEQ ID NO: 548) | (SEQ ID NO: 549) |
| 7 | TGCCGGCAGTGGTGGCAGCG | ATGTCTGCTGGATCGTCCCA |
| | (SEQ ID NO: 550) | (SEQ ID NO: 551) |
| 8 | GACCAGCTTGCCGGCAGTGG | GGTGGTGCTGGGCACAACGA |
| | (SEQ ID NO: 552) | (SEQ ID NO: 553) |
| 9 | TCTCCCTGTCCCAGGTTACC | TGAGGGTGCCATAGTCAAGG |
| | (SEQ ID NO: 554) | (SEQ ID NO: 555) |
| 10 | CTCTGCTCACCTGGTAACCT | GCTGACCAAGGCAGTGAAGG |
| | (SEQ ID NO: 556) | (SEQ ID NO: 557) |
| 11 | CCTGTGCGTGAAGACTGTGG | GGGTGCCATAGTCAAGGTGG |
| | (SEQ ID NO: 558) | (SEQ ID NO: 559) |
| 12 | AGATGACTTCAGAGAAGATG | GGTGCCATAGTCAAGGTGGT |
| | (SEQ ID NO: 560) | (SEQ ID NO: 561) |
| 13 | CTGCACCACGCGGCTGTACA | AGCGACATGCTGTCACGGAT |
| | (SEQ ID NO: 562) | (SEQ ID NO: 563) |
| 14 | GCGTGGTGCAGAAGCTGCAA | CACCAGCGTCTACCTCAAGA |
| | (SEQ ID NO: 564) | (SEQ ID NO: 565) |
| 15 | GACCACGAGGGAGGACAGCG | GAATGAGGGTGCCATAGTCA |
| | (SEQ ID NO: 566) | (SEQ ID NO: 567) |
| 16 | ATGACCACGAGGGAGGACAG | GGTGCTGGGCACAACGATGG |
| | (SEQ ID NO: 568) | (SEQ ID NO: 569) |
| 17 | CATGCCCAGCTGTTCCTTGA | TGCAGAGGTGTCCAATGCCC |
| | (SEQ ID NO: 570) | (SEQ ID NO: 571) |
| 18 | GTGGGCTCCGGCGGTCTCCA | CTGGGCACAACGATGGTGGC |
| | (SEQ ID NO: 572) | (SEQ ID NO: 573) |
| 19 | GTGGAAGCTGTGGGCTCCGG | CGACATGCTGTCACGGATGG |
| | (SEQ ID NO: 574) | (SEQ ID NO: 575) |
| 20 | TCACCTGGTAACCTGGGACA | ATTGAGCGGCTCAAGAATGA |
| | (SEQ ID NO: 576) | (SEQ ID NO: 421) |
| 21 | GGCCCTGTGCGTGAAGACTG | CTTCAGGCTCCTTCAAGATC |
| | (SEQ ID NO: 577) | (SEQ ID NO: 578) |
| 22 | GCCCTGTGCGTGAAGACTGT | GATGGTCCTCTCACGGATGG |
| | (SEQ ID NO: 579) | (SEQ ID NO: 580) |
| 23 | CCCTGTGCGTGAAGACTGTG | CTTGAAGGAGCCTGAAGGTT |
| | (SEQ ID NO: 581) | (SEQ ID NO: 582) |
| 24 | GAGATGACTTCAGAGAAGAT | CATTGAGCGGCTCAAGAATG |
| | (SEQ ID NO: 583) | (SEQ ID NO: 584) |
| 25 | CGAGATGACTTCAGAGAAGA | TCTTGAAGGAGCCTGAAGGT |
| | (SEQ ID NO: 585) | (SEQ ID NO: 586) |

TABLE 10
equine-Sds CRISPR sgRNA for
Knock Out & Interference

| | CRISPRko | CRISPRi |
|---|---|---|
| 1 | ATCTGCCCAGTCTATCCATC | GTGATCACCATCGTAAGTCT |
| | (SEQ ID NO: 587) | (SEQ ID NO: 588) |
| 2 | AGATGGATAGACTGGGCAGA | CATGGATGCAACAAGCCAGG |
| | (SEQ ID NO: 589) | (SEQ ID NO: 590) |
| 3 | ATGGTCCAGATGGATAGACT | CCTGGCAAGATTGATTTCAT |
| | (SEQ ID NO: 591) | (SEQ ID NO: 592) |
| 4 | GATGGTCCAGATGGATAGAC | AAAAGATTTTGTTACTATGT |
| | (SEQ ID NO: 593) | (SEQ ID NO: 594) |
| 5 | ATGACTGGAGATGGTCCAGA | AGACATGGATGCAACAAGCC |
| | (SEQ ID NO: 595) | (SEQ ID NO: 596) |
| 6 | CATCTCCAGTCATCCCTCCT | GTACACTTGAAACTAGTATA |
| | (SEQ ID NO: 597) | (SEQ ID NO: 598) |
| 7 | CAAGGAGGGATGACTGGAGA | CATGACATTTATTTGATAAC |
| | (SEQ ID NO: 599) | (SEQ ID NO: 600) |
| 8 | GTAAGCCAAGGAGGGATGAC | AGATGTTCCTAGACTTACGA |
| | (SEQ ID NO: 601) | (SEQ ID NO: 602) |
| 9 | TAGAGCGAGTAAGCCAAGGA | AAAGAATTCTTTGCTTTTAG |
| | (SEQ ID NO: 603) | (SEQ ID NO: 604) |
| 10 | GTAGAGCGAGTAAGCCAAGG | AAAGATTTTGTTACTATGTA |
| | (SEQ ID NO: 605) | (SEQ ID NO: 606) |
| 11 | TGGGTAGAGCGAGTAAGCCA | ACAGGAGGAAAATTTAATAA |
| | (SEQ ID NO: 607) | (SEQ ID NO: 608) |
| 12 | TGCTGGCAAAGCGATGGAGC | GCAAAGAATTCTTTTTTTCC |
| | (SEQ ID NO: 609) | (SEQ ID NO: 610) |
| 13 | TTGCTGGCAAAGCGATGGAG | GTCTAAAAAGAGCTCTTTGT |
| | (SEQ ID NO: 611) | (SEQ ID NO: 612) |
| 14 | CGTCATTGCTGGCAAAGCGA | ATTTACAGATGAAATAATAC |
| | (SEQ ID NO: 613) | (SEQ ID NO: 614) |
| 15 | GGCTCTGTGGACGTCATTGC | GTTATCAAATAAATGTCATG |
| | (SEQ ID NO: 615) | (SEQ ID NO: 616) |
| 16 | ACCGGGGTCTGCACGTGCAG | TACAGGAGGAAAATTTAATA |
| | (SEQ ID NO: 617) | (SEQ ID NO: 618) |
| 17 | GACCGGGGTCTGCACGTGCA | AGTTATCAAATAAATGTCAT |
| | (SEQ ID NO: 619) | (SEQ ID NO: 620) |
| 18 | TGCACGTGCAGACCCCGGTC | AAAAGAATTCTTTGCTTTTA |
| | (SEQ ID NO: 621) | (SEQ ID NO: 622) |
| 19 | GGACCGGGGTCTGCACGTGC | CCAATGAAATCAATCTTGCC |
| | (SEQ ID NO: 623) | (SEQ ID NO: 624) |
| 20 | GCACGTGCAGACCCCGGTCC | CAGTTATCAAATAAATGTCA |
| | (SEQ ID NO: 625) | (SEQ ID NO: 626) |
| 21 | CAGCCCGTCCCTGTCCAAAG | AAAAAGAATTCTTTGCTTTT |
| | (SEQ ID NO: 627) | (SEQ ID NO: 628) |
| 22 | CCGTCCCTGTCCAAAGTGGC | GGAGGAAAATTTAATAAGGG |
| | (SEQ ID NO: 629) | (SEQ ID NO: 630) |
| 23 | CGGCCACTTTGGACAGGGAC | AAGGGAGGAAAAAATAACAG |
| | (SEQ ID NO: 631) | (SEQ ID NO: 632) |
| 24 | CCGGCCACTTTGGACAGGGA | AGGAAAAAATAACAGAGGAA |
| | (SEQ ID NO: 633) | (SEQ ID NO: 634) |
| 25 | GGTGCCGGCCACTTTGGACA | GAGGAAAAAATAACAGAGGA |
| | (SEQ ID NO: 635) | (SEQ ID NO: 636) |

TABLE 11
feline-Sds CRISPR sgRNA for
Knock Out & Interference

| | CRISPRko | CRISPRi |
|---|---|---|
| 1 | CTGGAGAGCCCCTGCACGTG | CATGTCCCTGTCCAAAGTGG |
| | (SEQ ID NO: 637) | (SEQ ID NO: 638) |
| 2 | GGACATGCTGTCACGGACGG | TTCCATCGTGGAAGAACTGA |
| | (SEQ ID NO: 639) | (SEQ ID NO: 640) |
| 3 | GGGACATGCTGTCACGGACG | CAAGGTGGTGGGTGAGATGT |
| | (SEQ ID NO: 641) | (SEQ ID NO: 642) |
| 4 | AGGGACATGCTGTCACGGAC | AGACGCTGAGTGCAAAGCCG |
| | (SEQ ID NO: 643) | (SEQ ID NO: 644) |
| 5 | CAGGGACATGCTGTCACGGA | GACGCTGAGTGCAAAGCCGG |
| | (SEQ ID NO: 645) | (SEQ ID NO: 646) |
| 6 | CAGCATGTCCCTGTCCAAAG | GTTGGATGAGGCCTTTGAGC |
| | (SEQ ID NO: 647) | (SEQ ID NO: 648) |
| 7 | CATGTCCCTGTCCAAAGTGG | ATGGACAGCGTCCAACCTTC |
| | (SEQ ID NO: 638) | (SEQ ID NO: 649) |
| 8 | ATGTCCCTGTCCAAAGTGGC | CTCCTTCAGTTCTTCCACGA |
| | (SEQ ID NO: 650) | (SEQ ID NO: 651) |
| 9 | GGTGCCCGCCACTTTGGACA | CTTTGAGCTGGCCAAGGCCC |
| | (SEQ ID NO: 652) | (SEQ ID NO: 653) |
| 10 | TGGTGCCCGCCACTTTGGAC | CACCTGCCCTCACCATCGAG |
| | (SEQ ID NO: 654) | (SEQ ID NO: 655) |
| 11 | CACCAGCGTCTATCTCAAGA | GAGACGCTGAGTGCAAAGCC |
| | (SEQ ID NO: 656) | (SEQ ID NO: 657) |
| 12 | GTCCATCTTGAGATAGACGC | GGAGACGCTGAGTGCAAAGC |
| | (SEQ ID NO: 658) | (SEQ ID NO: 659) |
| 13 | ATGGACAGCGTCCAACCTTC | CCTTTGACGACCCCCTCATC |
| | (SEQ ID NO: 649) | (SEQ ID NO: 660) |
| 14 | TCTTGAAGGAGCCTGAAGGT | CACCAGCGTCTATCTCAAGA |
| | (SEQ ID NO: 586) | (SEQ ID NO: 656) |
| 15 | CTTCAGGCTCCTTCAAGATC | CTTCAGGCTCCTTCAAGATC |
| | (SEQ ID NO: 578) | (SEQ ID NO: 578) |
| 16 | TTCAGGCTCCTTCAAGATCC | GACGACCCCCTCATCTGGGA |
| | (SEQ ID NO: 661) | (SEQ ID NO: 662) |
| 17 | CGGATCTTGAAGGAGCCTGA | CTTTGACGACCCCCTCATCT |
| | (SEQ ID NO: 663) | (SEQ ID NO: 664) |
| 18 | TCAGGCTCCTTCAAGATCCG | TGTGGCCTTCCCAGATGAGG |
| | (SEQ ID NO: 415) | (SEQ ID NO: 665) |
| 19 | TCCTTCAAGATCCGGGGCAT | TCTTGAAGGAGCCTGAAGGT |
| | (SEQ ID NO: 666) | (SEQ ID NO: 586) |
| 20 | GGGGCATCGGACATCTCTGC | CAGCATGTCCCTGTCCAAAG |
| | (SEQ ID NO: 667) | (SEQ ID NO: 647) |
| 21 | TCGGACATCTCTGCAGGATG | TCCTTCAAGATCCGGGGCAT |
| | (SEQ ID NO: 668) | (SEQ ID NO: 666) |
| 22 | TGAACATTTCGTCTGCTCCT | AGGGACATGCTGTCACGGAC |
| | (SEQ ID NO: 669) | (SEQ ID NO: 643) |
| 23 | ACATTTCGTCTGCTCCTCGG | TGAACATTTCGTCTGCTCCT |
| | (SEQ ID NO: 670) | (SEQ ID NO: 669) |
| 24 | CATTTCGTCTGCTCCTCGGC | GGAAGGCCACACTTCCATCG |
| | (SEQ ID NO: 671) | (SEQ ID NO: 672) |
| 25 | AGGGATGCCCAGCTTCCTGG | GAGGAATGTAGACCCAGCCT |
| | (SEQ ID NO: 673) | (SEQ ID NO: 674) |

TABLE 12
hSDS RNAi, shRNA, and Anti-Sense
Oligonucleotides Targets for Repression

| | |
|---|---|
| 1 | CAAGCAAGGCTGTGCACATTT |
| | (SEQ ID NO: 675) |
| 2 | TGCAGGTTAACTTCTTGTTAT |
| | (SEQ ID NO: 676) |
| 3 | CCACGCTTCCATCGTGAAAGA |
| | (SEQ ID NO: 677) |
| 4 | CGAGGCTATGAATTGGACCTT |
| | (SEQ ID NO: 678) |
| 5 | TCAGGCCCTGAAGCTGTTTCA |
| | (SEQ ID NO: 679) |
| 6 | CTCTGAAGTTATCTCGGACCA |
| | (SEQ ID NO: 680) |
| 7 | CATTGGGCACTTCTGCAAGAG |
| | (SEQ ID NO: 681) |
| 8 | CGCTGTCTATAGCCACGTGAT |
| | (SEQ ID NO: 682) |
| 9 | GCTGGGCATGACAAATAGGTT |
| | (SEQ ID NO: 683) |
| 10 | CAAGATCACCAGTGTTGCCAA |
| | (SEQ ID NO: 684) |
| 11 | GCCATTGAGAAGTTCGTGGAT |
| | (SEQ ID NO: 685) |
| 12 | TGAGTTATTGGATGAAGCCTT |
| | (SEQ ID NO: 686) |
| 13 | CACCGCAGGCAAACTTGTCTC |
| | (SEQ ID NO: 687) |

TABLE 13
mSds RNAi, shRNA, and Anti-Sense
Oligonucleotides Targets for Repression

| | |
|---|---|
| 1 | AGCATGGCGTTGTCCAAATTG |
| | (SEQ ID NO: 688) |
| 2 | GCCACACTTCCCTTGTGAAAG |
| | (SEQ ID NO: 689) |
| 3 | CATTGGGCATCTCTGCAAGAT |
| | (SEQ ID NO: 4) |
| 4 | CTTGAGAAGTTCGTGGACGAT |
| | (SEQ ID NO: 2) |
| 5 | TGTCTGTTCTTCAGCTGGCAA |
| | (SEQ ID NO: 6) |
| 6 | CTGGGAGGATGTGCCCATCAT |
| | (SEQ ID NO: 5) |
| 7 | CCTCACCATTGAGCGGCTCAA |
| | (SEQ ID NO: 3) |

In some embodiments, the methods and compositions of the present technology increase serine levels in one or more tissues in a subject in need thereof. In some embodiments, the one or more tissues is selected from blood tissue (e.g., plasma, serum), liver tissue, kidney tissue, skin tissue, or any combination thereof. In some embodiments, administration of a therapeutically effective amount of an agent that disrupts SDS activity according to the methods and compositions of the present technology increases tissue serine levels in the subject by about 1% to about 500%, or any value or range in between. For example, in some embodiments, administration of a therapeutically effective amount of an agent that disrupts SDS activity according to the methods and compositions of the present technology increases tissue serine levels in the subject by at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, or at least about 500%.

Additionally or alternatively, in some embodiments, the methods and compositions of the present technology increase glycine and/or threonine levels in one or more tissues in a subject in need thereof. In some embodiments, the one or more tissues is selected from blood tissue (e.g., plasma, serum), liver tissue, kidney tissue, skin tissue, or any combination thereof. In some embodiments, administration of a therapeutically effective amount of an agent that disrupts SDS activity according to the methods and compositions of the present technology increases tissue glycine and/or threonine levels in the subject by about 1% to about 600%, or any value or range in between. For example, in some embodiments, administration of a therapeutically effective amount of an agent that disrupts SDS activity according to the methods and compositions of the present technology increases tissue glycine and/or threonine levels in the subject by at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, or at least about 600%.

In some embodiments, the present technology relates to the unexpected discovery that the disruption of SDS activity according to the methods described herein also decreases the levels of cytotoxic long chain (LC) and very long chain (VLC) deoxydihydroceramides (doxDHCer), while increasing levels of beneficial ceramides in the subject. Long chain ceramides have an attached fatty acid with a chain length of more than 14 C-Atoms (C14) and very long chain ceramides with more than 22 C-Atoms (C22) and their accumulation is negatively associated with cell viability. For example, in some embodiments, administration of the agents of the present technology to a subject results in decreased levels of cytotoxic 1-deoxydihydroceramides (doxDHCer), including, but not limited to doxDHCer m18:0/22:0, doxDHCer m18:0/24:0, doxDHCer m18:0/24:1, and doxDHCer m18:0/26:0, in one or more tissues of the subject.

In some embodiments, administration of a therapeutically effective amount of an agent that disrupts SDS activity according to the methods and compositions of the present technology increases ceramide levels by about 1.1-fold to about 1.5-fold, and decreases deoxyceramide levels by about 1.1-fold to about 3.5-fold in the one or more tissues of the subject. In some embodiments, administration of a therapeutically effective amount of an agent that disrupts SDS activity according to the methods and compositions of the present technology increases liver tissue ceramide levels by about 1.3-fold to about 1.4-fold, and decreases liver deoxyceramide levels by about 2-fold to about 2.5-fold. In some embodiments, administration of a therapeutically effective amount of an agent that disrupts SDS activity according to the methods and compositions of the present technology decreases kidney deoxyceramide levels by about 3-fold to about 3.5-fold. In some embodiments, administration of a therapeutically effective amount of an agent that disrupts SDS activity according to the methods and compositions of the present technology increases skin ceramide levels by about 1.1-fold to about 1.2-fold, and decreases skin deoxyceramide levels by about 2.5-fold to about 3-fold.

### V. Methods of Treatment

In one aspect, the present disclosure provides methods of treating a subject for one or more diseases or injuries selected from the group consisting of diabetic peripheral neuropathy, macular telangiectasia (MacTel), one or more wounds, liver damage, nerve damage, and muscle damage comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system. In some embodiments, the subject is diagnosed with, suspected of having, or at an increased risk of, diabetic peripheral neuropathy, MacTel, one or more wounds, liver damage, nerve damage, or muscle damage. In some embodiments, the subject is diagnosed with diabetic peripheral neuropathy. In some embodiments, the subject is diagnosed with MacTel. In some embodiments, the subject is diagnosed with one or more wounds. In some embodiments, the subject is diagnosed with liver damage. In some embodiments, the subject is diagnosed with nerve damage. In some embodiments, the subject is diagnosed with muscle damage. In some embodiments, the treatment comprises reducing or ameliorating one or more signs and symptoms of diabetic peripheral neuropathy, MacTel, one or more wounds, liver damage, nerve damage, or muscle damage. Peripheral neuropathy and nerve damage can be measured using any appropriate methodology, including thermal hypoalgesia (Hargraeves assay in mice), allodynia (Von Frey assay in mice), intraepidermal nerve fiber density, and corneal nerve density. MacTel can be diagnosed and assessed using fundus photos, or any other appropriate method for assessing pathology. Liver damage can be assessed using any appropriate methodology, including assays for ALT and AST enzymes in circulation or hepatocyte proliferation (as performed in the examples below). Muscle damage can be assessed using any appropriate methodology, including assessing myofiber size and muscle stem cell frequency.

In some embodiments, the agent is administered orally, topically, systemically, intravenously, subcutaneously, transdermally, intraocularly, iontophoretically, intranasally, intraperitoneally, or intramuscularly. In some embodiments, the subject is human, canine, feline, murine, or equine. In some embodiments, the methods further comprise administering an additional therapy or therapeutic agent selected from the group consisting of serine or a Glucagon-like peptide-1 (GLP-1) agonist such as semaglutide to mitigate muscle loss due to prolonged fasting. In some embodiments, the agent is co-administered with a Glucagon-like peptide-1 (GLP-1) agonist such as semaglutide to mitigate muscle loss due to prolonged fasting. In some embodiments, the methods further comprise the administration of supplements such as serine, glycine, threonine, glucose, or glutamine (or during feeding in general) to augment nutrient intake and/or healing. In some embodiments, the administration of supplements mitigates the risk of hypoglycemia or low glutamine levels. In some embodiments, the additional therapy or therapeutic agent comprises the standard or care for the treatment of diabetic peripheral neuropathy, MacTel, one or more wounds, liver damage, nerve damage, or muscle damage. In some embodiments, the additional therapy or therapeutic agent is administered simultaneously, sequentially, or separately. In some embodiments, the method does not comprise administering serine to the subject.

The compositions of the present technology may optionally be administered as a single bolus to a subject in need thereof. Alternatively, the dosing regimen may comprise multiple administrations performed at various times after the appearance of a disease or disorder.

Administration of the compositions of the present technology can be carried out by any suitable route. Administration can be carried out by any suitable route, such as oral administration. Administration can be carried out orally. Administration can be carried out subcutaneously. Administration can be carried out intraperitoneally. Administration can be carried out topically. Administration can be carried out intraocularly. Administration can be carried out ophthalmically. Administration can be carried out systemically. Alternatively, administration may be carried out intravenously, intranasally, intradermally, transdermally, intrathecally, intracerebroventricularly, iontophoretically, transmucosally or intramuscularly.

In some embodiments, the compositions of the present technology comprise pharmaceutical formulations which may be administered to subjects in need thereof in one or more doses. The dosage and frequency of administration can vary depending on whether the treatment is prophylactic or therapeutic. In prophylactic applications, a relatively low dosage of compositions is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

The compositions of the present technology can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Oral compositions generally include an inert diluent or an edible carrier. They can be enclosed in gelatin capsules or compressed into tablets. For the purpose of oral therapeutic administration, the compositions of the present technology can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition.

The precise determination of what would be considered an effective dose may be based on factors individual to each subject, including their size, age, sex, weight, and condition of the particular subject. Dosages can be readily ascertained by those skilled in the art from this disclosure and the knowledge in the art.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Materials and Methods

Serine tolerance test in mice. Age-matched 8- to 12-week-old BKS (JAX 000662) and BKS-db db (JAX 000642) or Sds knockout mice (Taconic) mice were fasted overnight with water access provided ad libitum. For a STT, animals were weighed, and serine was administered via oral gavage at a dose of 400 mg kg-1 with tail tip blood samples collected into EDTA-coated microvette tubes (Sarstedt) before, and 15, 30, 60, 120 and 180 min after an oral gavage. EDTA microvettes were spun at 2,000 g at 4° C. for 5 min to obtain plasma, and samples stored at −80° C. until analysis. Blood glucose and serine concentrations were quantified using Contour Next glucometer (Bayer) and gas chromatography-mass spectrometry as described below, respectively. Plasma serine pharmacokinetics were determined for a 400 mg kg-1 dose using PK solver50. To qualify downstream fate of serine, wild-type mice were fasted overnight, weighed in the morning, and [U-13C3]serine administered via oral gavage at a dose of 400 mg kg-1, with tissues collected, using Wollenberger clamps pre-cooled to the temperature of liquid nitrogen, before, and 15, 30, 45, 60, and 120 min after oral gavage, and samples stored at −80° C. until analysis.

Serine dehydratase activity assay. Frozen liver and kidney samples were extracted in an ice-cold buffer containing 50 mM potassium phosphate, 1 mM ethylenediaminetetraacetic acid (EDTA), and 1 mM dithiothreitol (DTT), pH 8.0 using glass homogenizer. Maximal enzyme activity was determined using coupled-enzyme reaction with lactate dehydrogenase (Sigma 10127230001) in the presence of 300 mM serine, 0.75 mM reduced nicotinamide adenine dinucleotide (NADH), 0.25 mM pyridoxal phosphate (PLP), and 1.5 mM DTT at 340 nm. Tissue homogenate protein quantification was subsequently determined using BCA protein assay (Lambda Biotech, G1002), and maximal enzyme activity expressed in international units (U) per mg of protein.

Generation of cell lines with stable SDS overexpression. Cell lines overexpressing either human or mouse SDS or a non-target control sequence were produced by lentiviral transduction. Lentiviral particles were produced according to the Addgene pLKO vector protocol¹. In brief, lentiviral particles were packaged in HEK293T cells that were transfected with a pLKO transfer plasmid and the packaging plasmids psPAX2 (Addgene Plasmid #12260) and pMD2.G (Addgene Plasmid #12259) using FuGene 6 (FuGENE cat. no. F6-1000) as the transfection reagent. 3 mL of DMEM supplemented with 10% FBS and 1% PenStrep was added to the cells at 24 and 48 hours post transfection and collected at 48 and 72 hours post transfection. The lentiviral suspension was filtered through a 0.45 m filter to remove cellular debris, supplemented with polybrene to a concentration of 6 μg/mL, and stored at −80° C. until needed.

HCT116 and HUH7 cells were cultured in a 6 well plate and transduced with 500 μL of viral suspension. After 6 hours, they were supplemented with 2 mL of virus-free DMEM. The following day, DMEM containing puromycin was added to select for cells that were successfully transduced. HCT116 and H1UH7 cells were selected with 2 μg/mL and 3 μg/mL puromycin respectively.

TABLE A
Summary of SDS overexpressor cell lines generated

| Gene | HCT116 | HUH7 |
|---|---|---|
| Non-Target Control | X | X |
| Human SDS (hSDS) | X | X |
| Mouse Sds (mSds) | | X |

Validation of SDS overexpression. Protein was extracted by scraping cells with 1×RIPA Lysis and Extraction Buffer (Thermo Scientific cat. no. 89900) supplemented with 1×HALT protease inhibitor cocktail (Thermo Fisher Scientific, cat. no. 78430). Protein concentrations were determined by BCA assay. Equal amounts of protein were loaded onto a 4-20% SDS-PAGE gel and transferred to a nitrocellulose membrane. The membrane was blocked in 5% BSA in Tris-buffered saline for 2 hours and incubated with primary antibodies at 4° C. overnight. Anti-SDS antibody (rabbit polyclonal, Genetex, cat. no. GTX47143, lot 82203710, 1:1,000) or Vinculin (mouse monoclonal, Abcam, cat. no. ab18058, lot GR287850-1, 1:1,000). The immunoblots were incubated with secondary antibodies for 45 minutes at room temperature, polyclonal anti-rabbit IgG Alexa Fluor Plus 800 (Thermo Scientific, cat. no. A327325, lot XF349345, 1:10,000) and anti-mouse IgG Alexa Fluor 680 (Thermo Scientific, cat. no. A21058, lot 2478007, 1:10,000). Blots were imaged with a LICORbio Odyssey CLx Imaging System equipped with Image Studio software.

Activity levels of the SDS overexpressor cell lines were measured by stable-isotope tracing in combination with gas chromatography-mass spectrometry. Cells were cultured in 6 well plates in media that was reconstituted from glucose, glutamine, and amino acid free DMEM (Sigma Aldrich cat. No. D5030) supplemented with either 13C labeled (CLM-1574-H-PK and CLM-1017-PK) or 12C unlabeled serine and glycine, unlabeled constituents, and 10% FBS. Serine and glycine were each supplemented to a concentration of 400 μM. Cells were washed once with PBS before adding the respective labeled or unlabeled media. Media was collected after 6 hours of growth for analysis of analyte concentrations and isotope enrichment.

shRNA-Mediated Knockdown of mSds. Five shRNA candidate sequences targeting mSds and one non-target control (NTC) sequence were packaged into lentivirus particles using HEK293T cells as described earlier. shRNA was expressed using the U6 promoter. HUH7 mSds OE cells were cultured in a 6 well plate and transduced with 1 mL of fresh viral suspension. 2 mL of complete media were added to the cells after 6 hours. Cells were not selected with puromycin as they were already puromycin resistant from the previous transduction.

NTC 02-
(SEQ ID NO: 1)
CAACAAGATGAAGAGCACCAA
shRNA 71-
(SEQ ID NO: 2)
CTTGAGAAGTTCGTGGACGAT
shRNA 72-
(SEQ ID NO: 3)
CCTCACCATTGAGCGGCTCAA
shRNA 73-
(SEQ ID NO: 4
CATTGGGCATCTCTGCAAGAT
shRNA 74-
(SEQ ID NO: 5)
CTGGGAGGATGTGCCCATCAT
shRNA 75-
(SEQ ID NO: 6)
TGTCTGTTCTTCAGCTGGCAA

Quantifying mSds knockdown for selection of shRNAs. The degree of shRNA-mediated knockdown of mSds was validated by immunoblotting and stable isotope tracing. Immunoblotting and preparation of labeled 13C serine and glycine media were performed as described above. mSds OE cells were transduced with mSds shRNA lentivirus and treated with puromycin. After 72 hours, cells were passaged to a 6 cm plate. After 48 hours, cells were washed with PBS before 5 mL of labeled tracer media was added to the cells. After 24 hours, 1 mL of spent media was collected, and polar metabolites were extracted from 5 μL of the media as described earlier. 5 μL of a 100 μM a 100 μM 13C, 15N-labelled amino-acid mixture (Cambridge Isotope Laboratories, cat. no MSK-A2-1.2) was used as an internal standard for quantitation of amino acid abundances. Metabolites were derivatized and analyzed as described previously. The enrichment of serine as well as serine and threonine abundances in the media was used as a readout of knockdown efficiency of the shRNA sequences.

Metabolite extraction and quantification of abundance and isotope enrichment. Metabolite extractions were performed as described previously^{2,3,4}. Plasma and media polar metabolites were extracted from 3 μl of sample spiked with a norvaline (1 μg/200 μL) internal standard and/or a known amount of 13C- and 15N-labelled standards (Cambridge Isotope Laboratories, MSK-A2-1.2). Cell extraction was performed after washing once with saline (0.9% w/v NaCl) by adding 250 μL of −80° C. MeOH to quench metabolism. 100 μL of ice-cold water spiked with an unlabeled norvaline (1 μg/200 μL) internal standard was added before cells were scraped and transferred to a fresh 1.5 mL Eppendorf tube. For tissue metabolite extraction, ˜20 mg of tissue was homogenized for 5 min using Precellys beads with 500 μl −20° C. methanol, 400 μl ice-cold saline and 100 μl ice-cold water and spiked with 13C/15N polar metabolite standards (Cambridge Isotope Laboratories, MSK-A2-1.2). 250 μL of chloroform was added and the tube was vortexed for 5 min and centrifuged at 21,000 g for 5 min to allow for phase separation. For cell and tissue extraction, 10% of the total volume was transferred to a 96-well plate for total protein quantitation and normalization.

The upper polar phase was collected and dried under vacuum. Metabolite derivatization was performed as described previously^{2,3,4}. In brief, a Gerstel MultiPurposeSampler (MPS 2XL) was used with methoxylamine hydrochloride (MP Biomedicals, cat. no. 0215540525) in pyridine and N-tert-butyldimethylsily-N-methyltrifluoroacetamide (MTBSTFA) with 1% tert-butyldimethylchlorosilane (tBDMS) (Regis Technologies) to derivatize the metabolites. The derivatized metabolites were then analyzed by an Agilent 7890B gas chromatograph equipped with a DB-35MS column (30 m (length)×0.25 mm (inner diameter), Agilent J&W Scientific) connected to an Agilent 5977C mass spectrometer. Metabolite abundances and isotopologue distributions were determined by integrating mass fragments using an in-house python script with corrections applied for natural isotope abundances. Enrichment in the M+3 labeling of the pyruvate mass fragment m/z 174 as well as decreases in serine and threonine concentrations were used as the readouts of SDS overexpressor activity.

Targeted sphingolipid quantitation. For targeted sphingolipid analysis, 20 pmol of sphinganine-d7 (Avanti Polar Lipids, 860658), 2 pmol of deoxysphinganine-d3 (Avanti Polar Lipids, 860474), 100 pmol of 13C dihydroceramide-d7 (Avanti Polar Lipids, 330726), 200 pmol of C15-ceramide-d7 (Avanti Polar Lipids, 860681), 10 pmol of d18:1-d7 glucosylsphingosine (Avanti Polar Lipids, 860695), 100 pmol of d18:1-d7/15:0 glucosylceramide (Avanti Polar Lipids, 330729), 100 pmol of d18:1-d7/15:0 lactosylceramide (Avanti Polar Lipids, 330727), 200 pmol of sphingosine-d7 (Avanti Polar Lipids, 860657), and 200 pmol of d18:1/18:1-d9 sphingomyelin (Avanti Polar Lipids, 791649) were added to extracts before separation. The identification of 1 deoxydihydroceramides was confirmed via retention time matching and analysis of m18:0/24:1 deoxyDHCer (Avanti PolarLipids, 860464) and m18:0/16:0 deoxyDHCer (Avanti Polar Lipids, 860462) standards, and normalization for 1-deoxydihydroceramides was done with the 13C-dihydroceramide-d7 standard. Homogenate aliquot of 50 μl was taken to determine tissue protein content using BCA protein assay (Lambda Biotech, G1002). The remaining homogenate was transferred to a 2 ml Eppendorf tube and 1 ml of −20° C. chloroform was added. Samples were vortex-mixed for 5 min and spun down for 5 min at 4° C. at 15,000 g. The organic phase was collected and 2 μl of formic acid was added to the remaining polar phase which was re-extracted with 1 ml of −20° C. chloroform. Combined organic phases were dried and the pellet was resuspended in 100 μl of buffer containing 100% methanol, 1 mM ammonium formate and 0.2% formic acid.

Quantification of sphingolipid metabolites was determined using triple quadrupole liquid chromatography-mass spectrometry platform (Agilent 6460). Sphingolipid species were separated on a C8 column (Spectra 3 m C8SR 150×3 mm inner diameter, Peeke Scientific). Mobile phase A was composed of 100% HPLC-grade water containing 2 mM ammonium formate and 0.2% formic acid and mobile phase B consisted of 100% methanol containing 0.2% formic acid and 1 mM ammonium formate. The flow rate was 0.5 ml min-1. The gradient elution program consisted of the following profile: 0 min, 82% B; 3 min, 82% B; 4 min, 90% B, 18 min, 99% B; 25 min, 99%, 27 min, 82% B, 30 min, 82% B. Column re-equilibration followed each sample and lasted 10 min. The capillary voltage was set to 3.5 kV, the drying gas temperature was 350° C., the drying gas flow rate was 10 l min-1, and the nebulizer pressure was 60 psi. Sphingolipid species were analyzed by selective reaction monitoring (SRM) of the transition from precursor to product ions at associated optimized collision energies and fragmentor voltages. Calculation of sphingolipid abundances was performed using spiked-in deuterated standards.

Identification of potential targeting moieties for influencing SDS expression. As shown above, Tables 1, 2, 3, 4, and 5 display the predicted zinc finger variable binding domain amino acid sequences. Briefly, these sequences were returned from an internal computational tool that takes an input of 500 bp before the transcriptional start site of the SDS gene in each listed species (human, mouse, canine, equine, feline), resulting in the output of corresponding amino acid sequences of zinc fingers that can bind to that region. Tables 6, 7, and 8 present spacer sequences designed using CRISPick (Broad Institute; portals.broadinstitute.org/gppx/crispick/public). These were designed for human (Human GRCh38), mouse (Mouse GRCm38), and rat (Rat mRatBN7.2), respectively. The mechanisms selected were CRISPRko, CRISPRa, and CRISPRi, with SpyoCas9 Chen (2013) chosen as the enzyme. The gene target was serine dehydratase, and 25 sequences were selected for each quota. Tables 9, 10, and 11 also display spacer sequences, which were designed using IDT's CRISPR-Cas9 guide RNA design tool (www.idtdna.com/site/order/designtool/index/CRISPR_CUSTOM). For the CRISPRko spacer sequences, the full SDS gene from the specified organism was input with the species setting set to “other,” resulting in 24 sequences selected from the returned quota. Cor the CRISPRi spacer sequences, 500 bp upstream of the transcriptional start site of the SDS gene from the specified organism was input, with the species setting set to “other,” resulting in 25 sequences selected from the returned quota. Tables 12 and 13 show DNA sequences obtained using Millipore Sigma's predesigned shRNA tool (www.sigmaaldrich.com/US/en/semi-configurators/shrna?activeLink=productSearch). The gene input was SDS, and the returned sequences have the potential to be used as shRNA, RNAi, or anti-sense oligonucleotides. The human, mouse, rat, canine, equine, and feline SDS gene sequences are provided below. These sequences were retrieved from the UC Santa Cruz public genome browser (genome.ucsc.edu/cgi-bin/hgGateway). For each sequence, exons are shown in UPPER CASE, introns are shown in lower case, and promoter/upstream by 500 bases.

Human SDS
>hg38_knownGene_ENST00000257549.9
range = chr12: 113392445-113404387 5′pad = 0
3′pad = 0 strand = − repeatMasking = none
(SEQ ID NO: 690)
ccacacccggctgatttttgtatttttagtagagacgggatttcaccatg
ttggccaggctggtttcgaattcctgacctcaggtgatccacccgcctcg
gcctcccaaaatgcggagccaatatttaacctttcatctgaagctatctg
cttaggaactaaaggaaaggcagtttcttacatgactcagcttttagctt
aattctttttcttttggcatagtgagttgggggtcccaattttttatttt
cctttcacgaaactgaggacagaaaggggaagtggaaagagggaggagga
gtgggttgtgggaaaggcaggaggaagctgccaaagaagaaagcttggcc
agattccacctcccttctgacagcctcagtggcctcaagccaggggggtg
ggtcctggcaggagggaggggggggtgtagccagccccagggccccattg
gctgcccagaggtgccagaggaataaagcccaggaccaagtagcccctgg
GACCATTCAGAAGACTGTCCACCCGTCCTCAGCCCCCTCTCTTCGTGGGC
TATCTACTCAGTTGATCCCTCCCTCGCTGGCTTGGCTCTGACTCCTGCTC
AGACCCATCACCTTTGCCGGgtaagtggccccatcctcagtcccacctca
ggactggagtgtcccgggctagcctgtggtttcccaggggaagagggaga
catagaaagagtctgtcaaggctgggcatggtggctgatgcctgtaatcc
caacactttggaaggctgaggtgggagggttgcttgaggccacaagttca
agactgagctgggcaacagagtgagaccccccccaatctctacaaaaaaa
attttttttttttaagacagaggtctcactctgtcacccaggctgaagtg
caatggcacaatctgggctcactgcaacctccacctcctgggttcaagcg
attctcctgcctcagcctcccgagtagcttggactacaggcgtgtgccac
cacgcccggctaatttttgtatttttagtagagatggggtttcaccatgt
tggccaggctggtctcgaactcctgacctaaaaaaagaatttaattagta
tagtgtggtggtgcacacctgtagtcccagctactgggaaggctgagttg
ggaggatcgctggagcccaagaggtcgagtctgcagtgagcaattattgc
gcccctgcactccagcctgggtgacacagcaagaccttgtctcaaatttt
taaaaaacagccatttgggcctaaacactgagtttgtgtccagttctgtg
cctcagcttccacatctgtagaatggggatgagcatagcgccttcctcct
caggtggttgtgacggtgaaaggatgacctgcacttaaggtgtccagccc
agatttcataatagttcatatcgactgagcacttactgtgaaccaagtga
aagggaaggtggcttccgactgccggagtgggcagacagcggcttcaggg
ctccctccctcctcctccccgggcacaacctcttctgaagtcttgcgatc
cttggggcagggatccttggggcaggctggatgcagttaatggatgctca
cttcacggaaagccgcggcagagggagccctgtccttttacctgaaggca
tcccatggcaccgcctgggacaaacggcgagagaggcgccccaaacccac
ctgaaaagggtcttcgtggccggctcctctgactcccaccccgattgtcc
ctgaggccaggccttggtgacctacaccggcctttaatgtcaagtcagtc
ggtgtggtggctcacgcctgtaatcccagcactttgggaggccaaggcag
gcagatgacttgaggtcaggagttcgagaccagcctgcccaatatggtga
aaccttgtctccactaaaaatgcaaaaattattcgggcgtggtggtgcac
gtctgtgatcccagctacttgggaggctgaggcaggaaaatcgcttgaac
ccagaaggcggaggttgcagtgagccgagatcatgccgctgcactccagc
ctgggtgacagagggaggttctctaaaaataactaaataaaaaccgggat
cccattgtttggggattaacctccaggagctgaaagttcatgccgatttg
gggatgtcctgttttgaagtttgccccagtctctgatttttattcgagag
atccagcggagagagggtgtctgttcaagttcccctcccaggccccgaat
gtcagttaattcagatagacaaggggtgacctatttaggggatgctaagt
ctggcggggcagttggaatccccacccctcattctccctctgaccaaggg
atgtctggctcattctgggagcgggggatggatgtccaagctaggagtgc
ccaggacagggggctggacaaggtggcattggagatgggtctctggctag
ccacccttgcctggccttctgggatggtggagggagggggaaccagagag
ccccttggccatcgtcagctgatctcttctggcccggcccaagttgagga
gaatcaaatcctccctgagactctcagcagaaggcaggggagatgggaag
gctgggtgggtggacgtggctgtggctagaccccgggccaccccagctgc
ttcatcccccagagcccatccagaagcaggagcctgagccacacccatgg
gtcgcagagagagcctgagccctggagccagctgcccgagttcaaatcct
gcctccctgcctcactcacagcgtgactttgggcaaattatgttaatatt
ccccttcctgttgctgtgtaccaaactaccccaaaatgtgacttaaaaca
acagcaagccaggtgctgtggctcatgcctgtaatcccagcagtttggga
ggctgaggtgggcagaacgcttgaggccaggagttggagaccagcctggc
caacatagcaaaaccccatctctactggaatgaatgaatgaatgaatgaa
tgaatgaatgaataaaatttaaaagctgtgattagtcactgctataataa
ttatgacaaaattaataattatgaaagagcttttcctaactgaatattgg
gcttcccaaaagttaggttattttgtttttttttaaataaatatttattg
aacatctactatgagtcaggcattttgctgggtcctgggatcgtccattt
aagccaagatacttggaactgcatgtcagaagagccagctgccaggttgt
ttttgtttgtttgtttgtttgtttgagacagagtctcgatctgtagacga
ggctggagtgcagtggcgcgatctcggctcactacaagctccgcctcccg
ggttcacgccattctcctgcctcagcctcccaagtagcttgtatttttag
tagagacggggtttcaccatgttagccaggatggtctcgatctcctgacc
tcgtgatccgcccgcttcggcctcccaaagtgctgggattacaggtgtga
gtcaccgcgcccagcccaggttttttttaaatgtatgcatttaagtaagt
aatagaggcacatggtaaaacatcaaactgtgcaaaaggtgggtgctatg
atcccgagggccagctctcctcagaggccccaccctggccagcctgtcat
gcatgtttcctgaaatgctctgtgcacatggaaacatgtggaacaaaaca
aaaaaaaattcaaataaaaaaattaaaaaaatatataaaagaaacatgtg
gactatatgtgtgtgtggatggattgtgcgtgtgtgtggatggattgtgt
gtgggagtggatcgtgtgtgtgtggatggagtatgtgtgtgtggatggat
tatctgtgtgtgtgggtgtccctgaatttttcacatagatagatagacta
ttcttttttttttttgagacacagtctcactctgttgcccaggctggagt
gcagtggctcgatcttggcccattacaacctccacctcctcggttcaagt
gatgctcctccctcagcctcctgagtagctgagattacaggtgcctgcca
ccatgcctggctaattctctttcttcttcttttttttttttttctcaaga
cagagtcttgctctgtcacccaggcaggagtgcaatggcgcgatctcgct
cactgcaacctccgcctcctgggttcaagtgattctcctgcctcagcctc
ctaagtagctggggttacaggcgtgcgccaccatgcccagctaatttttt
gtatttttagtagagacggggtttcaccatgttggccaggctggtctcga
acttctgaccttgtgatccaccagccttggcctcccaaagtgctggtatt
acatgcataagccactgtgcccggcctcacctcatgcattttgaaagctc
caaggtttgagtgttctctcttcagcctcccagcaaatgtgtgagaggtc
aggagtggagggtgatccctgatagagctgaggccaaggcgtgattacct
gtctgatgtcacatggtgagtaggtggcccacttgcgattagagcccagg
tctccaggatgctctctcgtctgaggccttgcttggcttgctgggaagcg
aaggaagggatcactctggcagtgctggcaccggggctagctctcctaac
cctctgggtttccttcctttctcccagGAATGATGTCTGGAGAACCCCTG
CACGTGAAGACCCCCATCCGTGACAGCATGGCCCTGTCCAAAATGGCCGG
CACCAGCGTCTACCTCAAGATGGACAGTGCCCAGCCCTCCGGCTCCTTCA
AGATCCGGGGCATTGGGCACTTCTGCAAGAGGgtacgggaccgggtcagc
attatctgggcaggggtggcaggtcctccttcctcaacagggcagggcca
gtgctgtgtgaaattaaaggcgttgtaggataagcctactgactccccgc
aaggactgagcgtccaccattagggcttcctctgtttgaggggtgccctt
ccaggagggttccttgggactgatcccagagccatcagtcgcctattttc
ctccaccctttcctgagccagtctctttggagatgggaggtgcagtctca
gacaagggtagaacaaattctgagtgaaggaaacgtccgtgtccactgga
atcctggcaggtattccaggtggggaggaagagcacaggcaatgactggg
aggtgggcctgagtgcctctcctgttgtctccacagTGGGCCAAGCAAGG
CTGTGCACATTTTGTCTGCTCCTCGGgtaagtgatctgcttccccatcct
gtcctctgctgtgtgtcctgggatcagacactctgcttctctgagccctg
gtttccccagcaatgtgccactgaaaacaatggcagcagccaccaatttg
gaattccagggggaggccagcagtcgcccagggtggggaggtgagggggg
aactccagagcccccagtagagggtacgcagtcctgcctgggctcccaga
tgctcccgcactgacacgctctccacccccgaccctggcagCGGGCAACG
CAGGCATGGCGGCTGCATATGCGGCCAGGCAACTCGGCGTCCCCGCCACC
ATCGTGGTGCCCAGCACCACACCTGCTCTCACCATTGAGCGCCTCAAGAA
TGAAGGTGCCACAGTCAAGGTGGTGGGTGAGgtgagtgccgacccagggc
aaggaagagagagggcgcctggtggctggacagggtgccccctgtgcctc
accccctcctatctcccctgtggctttgcagTTATTGGATGAAGCCTTCG
AGCTGGCCAAGGCCCTAGCGAAGAACAACCCGGGTTGGGTCTACATTCCC
CCCTTTGATGACCCCCTCATCTGgtatgtggagttcaaggtcacttggtg
ggggtggcagccactgccctatatcactgggcaggcattgctatgatgca
catcatcacgtcctcatgcacagcagctgcacatgtgtgcgcacaccacc
atgtagaagcacacttaggcggggcacagtggctcacgcctgtaatccca
gcactttgggaggccaaggtgggaggatcacttgaggtcaggtgtttgag
accagtctggccaacatggggaaaccccatctctactaaaaatacaaaaa
ttacctgtacttggtgacacacgcctgtaatcccagctcctagggagact
aaggcaggagaatcgcttgaacccaggaggtggaagttgcagtgagccat
gatcacgccactgcactccagcctgggcaagagagtgagactccatctca
aaaaaaaaaacaaaaaaaaaaaaaagaaagaaacacacgttacacccagg
tgcagcagcaagtgcacacatgtcaccaaatgcccattatccacggctgc
acatgcaacacacccaaggctgaacatgcaagcacacagatgcccaagtg
tctgcagttgagtcctcctggaagcagacttggggacaaaggtttcaggg
aaaggagtttatttgggaggtgattcagtgggggagtggggaagtaagcc
agggaaggaagggggctttgctgtttcttttttctttttctatcatccag
tccaaaatgtcaggaagggggctttcaaaggcaggtggccgctgtgggtg
gctagagcttaatccctcggggaccctgggagccagtgcagaaggcgcac
agcagtttcccgccccctgggagaaggagcaggggagtttgtacaccgac
tcgtagccattggttgagggctgccttaaggggctgccatctcctgactc
tgtttgcaggtagctcaggtggccagagaaggtcctcaggcagagacaca
gatgctggcagctagaagccaaggttgggggtggggagggaactagctcc
aggccccatcagctgggataaggccggtgcaaacctgagctggtgtctcc
aggaagcctagccctgacctgccaccccctctccccaccctccagGGAAG
GCCACGCTTCCATCGTGAAAGAGCTGAAGGAGACACTGTGGGAAAAGCCG
GGGGCCATCGCGCTGTCAGTGGGCGGCGGGGGCCTGCTGTGTGGAGTGGT
CCAGGGGCTGCAGGAGGTGGGCTGGGGGGACGTGCCTGTCATCGCCATGG
AGACTTTTGGTGCCCACAGCTTCCACGCTGCCACCACCGCAGGCAAACTT
GTCTCCCTGCCCAAGATCACCAGgtgagcagctggggtgcctccctcggg
tgtccagcaagcactggggagtcccccttagggctggctttgagatagat
cctgatgtcagcctgtttcacagatgagacaatggaggctttgatcgctt
gcccagggccacgtccagtgagtggtacagcttggactcaaacccaagtc
tgtgtgactccaacatgtcacacttcctcacgtgaaaagcctcatatgag
gctgggcacagtgacttgtgcctgtaatcccagcactttgggtggctgag
gcaggaagatcacttgaggccaggagttcaagaccagcctgagcaacata
gagagattcccacctctaaaaaaagttttttgttttttttttaaattaac
tgggcgtggtagcacacacgcatagtcccaggctaaggtgggaaggattg
cttaagcccaggagactgaggctgtagtgagccatgatcatgccagtgca
ctccagcctgggtgacagagcaagaccctgtctagaaacaaagaaaaagg
aaggaagggagagagggagggagggaggaagggagggaaggaaggaagga
aggaaggaaggaaggaaggaaggaaggaaggaaggaaggaagggagagag
ggagggagggagggaaagaaggaaggaaggagaaaggaagaggaaagaaa
gagagagaaagaaagaaaaaaggagaaagagaaagagagaaagaaaaaga
aagagaaagaaagagagaaagaaagaaaaaagagaaagaaaggaagaaag
aaagaaaaagagagaaagagaaagaaaggaaaggaaaggaaaggaaaaag
ggagggagggaggaaggaaggaaggaaggaaggtctcataggaatcattt
actgagtcctctgtggcaaggttttacatacctcattactttgccaacca
tttactgagcccctattgtgtgtaagacactggctgatctggggagagag
tggtgcataaacacaagtcttgctcctaaggagttcatatccaagcccca
aacctttgatagtttgtttgtttttgtctttgttttttgagacagagttt
cgctcttgttgcccaagctggagtgcaatggtacaatctcagctcaccgc
aacctctgcctcccaagttcaagcgattctcctgcctcagcctcaggagt
aggcgtctgccccaacacttggctaattttgtatttttagtagaaatggg
gttttaccgtgttagccaggctagtctcgaactcctcacctcaggtgatc
tgcccacctcagcctcccaaagtgctgggattacaggcgtgagccaccgc
acccggctggtagatagtatttaattaggattaggttttgaccccattac
acagaaggcccaaataacagtgacttaaatgagattgaactttaccatga
aaagtcagggcgccaggtcccttgagtttgtttccccattatccttagca
tgtagtgtttcagtccaaaatggttgcctgagtgccagctatcacaccca
cattccatttggcagggatggggaagcaagggcagaaggctatgctccaa
ccttcaaagacagacacaacacttccacttacctttcgttggcctgaact
tagtcacatggccacatccagctgcaagggaggctgggaaatgtctttac
accaggtgcccataagtgcagctagaaaagacaccggggcagacaatagt
ctcccctacagatagggaagactgagcccagccaggtaagaacttcttcc
agattcctgagcctggcatctggagctgctgtgtcccacactgtagggac
tgaagacgtcatggctgagcgcctactgtaaaccacgtgctgctgagggc
tgaggtggctcaggccctcaagatgctcctggggaagggagagggggcag
ctgagctgggaatagagctagtggtcaccccaggaagctggggacagcgt
caacacattggttaccagtgcaggtcttgaagttatacagatcctgcttg
cagccccagctccagcactccctaggtgtgtgaccttgggcaagtgactg
tacactctctgcctcagtttcccctttccaaaatcaaaccaattaacaac
agcagaattcttgtgtgaattcaataatgcttgtgaagtgcttggcacct
aacactttctcaagaagtactcagggcgcttgttactagcagggaggact
atttgagaattccacatagggccgtgggactcagtactctcccaggagga
ctccttggaggaggtggccttaatcctgagccttgaaggacacagcagga
tgtgggcaggcagagtggtgctaagcttcatgagggttaaccaggtagcg
gttgtaacccggagcaaaagcagggagtcctagcagggaacaacaagggt
cctcttaggcatttgctcctgacttagtgttgatcagaatatgacgctgt
gccgaggcaggaggatcacttgagcccaggagttcaagaccagcctgggg
tcgggcatggtggctcatgcctgtaatcccagcactttggaaggccgagg
ggggtggatcacctaaggtcaggaattcgaggccagcctggtcaacatag
tgaaaccccatctctactaaaaatacaaaaattagccgggtgtggtggtg
ggcacctgtagtcccagctactcggtaggctgaggctggagaattgcttg
aacccgggaggcagagattgcagtgagctgagatcgcaccactgcactcc
agcctgggcaacagagcaagactccgtctaaaaaaaaaaaaaaacaaaaa
aacaacaaaaaaacaaacaaaacaaaaacccgcctgggcaacatagtgac
actcagtctcaaaaaaattaaaaaaatgatactaaagaatatactatatg
atgtcaattcaaacactctgcaccttattagaagaccccacttggtttgg
ggatccctgaggcttttcagggttttacagctgcagaggtgatacctgtc
cccctgcccccagatgcattgccttctctgtctcacatccatctccttct
ctccctccctgcccccatctctctcctgtttcccccactcccatcctctt
ctgggttcccacttctctcagTGTTGCCAAGGCCCTGGGCGTGAAGACTG
TGGGGGCTCAGGCCCTGAAGCTGTTTCAGGAACACCCCATTTTCTCTGAA
GTTATCTCGGACCAGGAGGCTGTGGCCGCCATTGAGAAGTTCGTGGgtat
gtgccaggtcctcccatgacctgacctgctgactcaggcgctaagtggct
gtccacttggtatgccactggggatcccactcaccccaagtttccttcac
agttctgtcttccacctctgcctagaattttccattaatagaagataata
caatgccgtgtagaaatagaaaagcaaaagccacacataatggaaaagta
aatagaaaagtaagcctgataaccatagcagcagaaaacaataagtgaat
atttaaaatgttgcttctgagtttcctggcagccagggtaaaaaaaggaa
aacatggggctcagagtttgattcattcagaggtgagtctgtaactaggc
tggactctttctgggttccgtctagggcagggatggcaaaaaagagagaa
aaaaatagtaccatttatgcttctgtacctgctgcacacttggctaatca
accatggaactcttgctcagatgatgcctcagaatccttctcaacacagt
gctcacgcaaccacactcattctatcagtgttagcactcatgatgaaacc
caggccctggctcaggcttggaagggccaattcaggccgatgacgggacg
tttggttcaggacctgcggctgcagccagggctgagagctgattggctca
gtattcccaggtcctgccaatggcctgtcagctcctgtgcaccctgggaa
actcaggccacgcccctgtcacgccccttctctggcagATGATGAGAAGA
TCCTGGTGGAGCCCGCCTGCGGGGCAGCCCTGGCCGCTGTCTATAGCCAC
GTGATCCAGAAGCTCCAACTGGAGGGGAATCTCCGAACCCCGCTGCCATC
CCTCGTGGTCATCGTCTGCGGGGGCAGCAACATCAGCCTGGCCCAGCTGC
GGGCGCTCAAGGAACAGCTGGGCATGACAAATAGGTTGCCCAAGTGAGGA
CGGACCCCTTACCGATCTGTGCTCTCCTAGCCCAAGAGACCCCTGGAGGG
GCTGGAGTTTTATCCAGCGCCTCGTCGTATGTTTGGCTGAGCACCTGTGG
CCTGGGTGCAGGTTAACTTCTTGTTATCAGGAGCCCACTATGCAGAGGCC
AAAGGTCGGCAGCCAGCGAGGCTATGAATTGGACCTTTTTGGTATCTGTG
TGACTGCTCTGTGCCCATCCTTAGCCAACTTGCTGGCGTGACAAGTGCCC
ACAAGTAACACACCAGGTACCCAGAGCAGGGTGGACAGGAGAGACCTGAA
TCACAGCAGTGAGGAATTCCTCAAAGCTGTGACCTCGCCCCTGAACTTGT
TCCCAGTGAAGGTCAACAGGGCCACCAACCAGGGTTGAGGAACCATCTCA
GCCATCCTCCCAGGAAGCCCACCCCTGACCCTTACTAATTTTTCTAATGT
GCAAACTTTTTCATTGAAAAATAAAATATATTTATGAAACAAA
Murine SDS
>mm10_knownGene_ENSMUST00000201684.3
range = chr5: 120476026-120483778 5′pad = 0
3′pad = 0 strand = + repeatMasking = none
(SEQ ID NO: 691)
ctgtgcctcaaccccaccccccgcaagtcccgcctctcactatctctgag
agcggtgcttcatttccactctgtacagacccattgtatggtctgacact
aatttgggtttaggtggctgagcctaagcccaatactccccacccctttt
ctttctattttaagacaggatctcactccttagttcaggctcaaacttgt
agtcctgcttcagcttccactgtgcggggatcacaggtgtgagctgccat
gtggctagcctgtggctgtttcttagcgatatactacgtgtccagcaact
gcaggcttttttgttattctgagactgacccccgggtccaggcaagggct
cttcagtgccgcatgccctcctccgtggccgcccagccccggcctggacc
agggggcggagcttgcttggggtggggggtggcctgcctggggcctcatt
ggctacccggaggctcagccctgagaataaagctctggagtgtcctgcca
ACTTTGTACTCTCCCAGGACACAGACACAATCTACTCCTCTCGCTCGCTC
TGTCCAGCTCCTCCTGTGCCGGgtatgtggcccccagcttcagcctctcc
ccaaggttaacagtcagggacagccagagcagggtacccgggaagtggag
aggaactgggactcatccagttcaagtctccgtgttcagatctgtaaaat
gggacgaagtggtaggaccttctagatcaggatgtcgctggggtggcatg
tggcaggctatctttgctgaatgctcgcatgagccaaatgacaaagaagg
gacttctgttctggaaccgatgtcgcactagccacctttgccaagacccc
cagtgctttttttctctcttgcaggtcatactatttatggtgccagatgg
gaccggcctctggggtgggaatcagaacagagtaccctgcccatttcccc
aaaggttcccaatgctgcagcctgggacagggactggtggggctccccaa
agccacctggaaggggtttgcgtggtggaagctatccactagccagaggc
tgcaacagctgggccttttaggtccagagaagacccactataccaggatg
ggactagctaggacaaagcaaacacatttggaggtactcttgatgtgtgc
cattcttcccggcagctgtctttcaagaggtcttctagaaagaggagtct
ggtgggctggcctgttatctcagcacttgagagactgagagcacttgaga
gactgaggcagggggaacacaaggagtttgagtctctctctctctctctc
tctctctctctctctctctctctctctctctctctctctctctctctccc
ctaaggcaggaggatcacaaggagtttgagtctctcctacactcacacac
atgcccttgaatacatgtggatgctcggaagagacaccatctgcctggat
ttgatcccacttggctctgaatctctctcagggcttctcacagggccccg
aggtgacagtagctgagccagtgttagcccaaaggcttcctcactgccat
ggctaccgaagggaccttggggtgtcattagccagacttgaacttgtcat
gcagtctctcctgtgtccaggttccctctcagtgtggcagctgtgtgatg
gggtgggccagggttctctgttgcctcttccccatgccagaaaaaaaaat
ctataactctatctttttaaaagaacaagggcctttcagagctcgatact
ggacttccaggtcttttgggtttttttttgtttttgaaacagggtcctac
gtagcccgggatagcctcaaactccctacatagccaaggttatgaatgac
taattctcttgccttcgcctgccaagtgatgggattacaggcctgcccca
aaccccaaaggctcttggtttttgggtttttttcccccgaaatatgtatt
tgttgaacgccagACATAGCAGGGAACACCTGTGATCCCAGCAATTGGGA
GACTGAGATAGAAGGACCCAACGTTCAAAACTAGCCTAGgtcacatggta
agaacgcactgagttttttcctttttaaaagcatttgtgggtttgggatg
tggctccgtctgcgaagggcttcccatgaaagtgcgaggacctatgttca
tattcccagaatggagcacataaaaatgctgagctcagcaataaatgcct
gccatccattcactagggagcaagagacaggaggacctcagggaacctct
ggaggacctcagggaacctctggaggacctcagggaacctctggggacct
caggggacctctggaggacctcagggaacctctggggacctcaggggacc
tccagggagcctccagaaaaatttaggggacctccagagatctcagtgta
ccttgggagagctcagtgaacctcaggggacctcaagggacctcagggag
tctctaaggttccctggtcagccagttgaactgaatcagggagctcccag
ctcaatgcaagaccctgtctcaaaaaataaactggagggcaactgaggga
gatactgacctatgttgacctctggcctctgcacacatacacacacatat
gcgcacacacacacacgtgcacactgctgagcatggagggatgctgtgga
cccgagaaccactcttcacaggtactccacttggcccatttcttttgtgt
ctgctgacgtgctctgagctcacgggaataggtcagctggggttggttcc
tggggactttgcaggaacagaccacagctgactctgaccttgcttggtcc
ccatcttgtggctcattctgagacagtttaaacaagccccttcccctgaa
cttgcccaaggatgtttttgacctcgtgaccttctaactttgggggtcaa
agttgagggcttgacctcctatttggccactaggtagtcagagctgagat
ttgattcggtgtcctttctttatcattatgggtatgaaagaggagtcata
gagcccaggagggcagcctctcttgaccctctgtatctcctccctgcccc
cagCCATGGCTGCCCAGGAGTCCCTGCATGTGAAGACCCCACTTCGTGAC
AGCATGGCGTTGTCCAAATTGGCCGGCACCAGCGTGTTCCTAAAAATGGA
CAGCTCCCAGCCCTCAGGCTCCTTCAAGATCCGAGGCATTGGGCATCTCT
GCAAGATGgtacaaggcagggtggcatcctggcaggaggggcagtccctc
ccacctgacagttgtagaggcaggtgtgcagacagacacccaggattcca
aaccgagtcaggaaggtagaaagtgaacggtatcccagagtgtgtagcaa
gaccctgtctctacggaaataataattttagggcctggggtttagacagc
tcagcgcttaagagcaccggctgcttttgcagaggacctgagctcagttc
ccagcacccacatgggggctcacaaccacctataactccagttccaggag
atctgacaccctcttctgagttccaggagcaactgcacttccgtgtatat
aatccaaaataagtaaacattgggctggggagatagctcagctgttagga
atgcttggggctctttcagaagaaccatgctcaccctcagcatccaagtc
agtgagtggctccagcccaaagatttatttagtaagcacatacattttat
gtgctgtctttagacatcccagaagagggcgccagatctcattatgggtg
gtttgaactcaggacctttggaagaacaatcagtgctcctacccgctgag
ccatctcaccagctcaatgaaacaaatctcttttattattattatttctt
atatatatgatgagtacactgtcactgtcttcagacgcaccagaagaagg
cattacagaaggttgtgagccaccacgtgggtgccaggaattgaactcag
gacacctggaagagcagtcagtgctcttaactgctgagccgtctctccag
ccccaacaaatcttaaataaaaaaaaattttttaagaaaatcattttaga
ataggttacacatatcagggctctctctggctaaggggtgtccacccata
caggtccctagagactgacacctgcctccttgccccagttaatctccttg
gggaagatatacctgagagagaatggttcagatactaagtgggtaagctc
agcttaagatagaagatgttccagtgggagagatgaacctaggctgggac
tggggggtggcctgaccctccctcttgtctccacagAAAGCAAAACAAGG
CTGTAGACATTTTGTCTGTTCTTCAGgtaagtgcccagttagctttcttt
cccttcctaccctggtctgtgcgtcctgtggcccagtaccaggcactgtt
tctctctgggccccgggactccgcaccagtgtatccattcgtcatcaccc
agatttgaggttccagaatgaagcagaattccctctggagatgagcagtc
ccacctggggtcccagaccctcctccctgacacgccctctccactgacag
CTGGCAACGCGGGCATGGCGACCGCCTACGCTGCCAGGAGGCTGGGCATC
CCAGCCACTATCGTTGTGCCCAACACCACACCTGCCCTCACCATTGAGCG
GCTCAAGAATGAAGGCGCCACAGTTGAAGTGGTGGGAGAGgtgagtgcgg
atccctcgtgggcaggagggctgataactagatggagatcctcatcatct
cagtctccccgccccgccccgccccttgcagATGCTGGATGAGGCCATCC
AAGTGGCCAAGGCTCTGGAAAAGAACAACCCGGGTTGGGTGTACATTTCC
CCCTTTGATGACCCTCTCATCTGgtaagttggagccaccccactccgatc
tgccatgtgtggcttctgctcctgccctctccggcacacccaatgtgtgc
aggagagaacacacaaacgcacagatgtacacatcagcgatgaggggtcc
ccagatgcagagacttggggagatcagctcatctgggggtgactaggaac
aaacgggctagggaagcatccgtggaggtggctctcgagtgccagcatct
gtctgccatgagcaactgaagcagggtccctgggaaccctgggagttggt
ggggagtaagcatccaaggatgtatccactggtgggcaggaaatggagat
ttatccaccaacccccacagccatctcctctctccatgagtgggggaggg
gggcttgtctcctacacaatcagtgtcctgtgaggcaggctggacagcag
gaaggtcagaagccaggttcatcattgcctgggatagaaggaaataaaga
ccttagtcagcatctttggccagccattgtgtcttcgtgaagccctggcc
tgaccctgctacccttctcctgcccagGGAAGGCCACACTTCCCTTGTGA
AAGAGCTGAAGGAGACGCTGAGCGCCAAGCCCGGGGCTATCGTGCTGTCT
GTGGGCGGTGGAGGCCTGCTGTGCGGAGTGGTCCAGGGGCTGCGGGAGGT
GGGCTGGGAGGATGTGCCCATCATCGCCATGGAGACCTTTGGCGCCCACA
GCTTCCATGCTGCCATCAAGGAAGGAAAGCTGGTCACCCTGCCCAAGATC
ACCAGgtaaacccggggcatctctccatcacatgagcagtgggattgagg
cttcgggagctgcgtgcctatgtgtagcccgtctaacacagcgtgggggg
ggggggaaacgccctctttgcctgcaaagaatttataaccaacccccaca
ccagtagcttaaaccaaattgaactctagttctattagtggcaactcctt
agagcaggaagctgagttcctggagtatcttctcctatcactctgagtat
gctgacctagggtccaaaatggctgcttttgtgccagccaccacacccac
actccatctgagaagagggagagcagaaggatgctcctaaactttaaaga
ctaagtggagctgctccccagctgacagcggggtcacacccagcaaacca
atcattagagctgagagtgttgtgaacgagttgaacctggtctgctgggc
accttacctaagctgtttggcacacagtatggaactggccgtttcttgct
gacaggctgatggagagctgctacccagcatcccgagagtgaactgtacc
acaaacctatagttggggtgtgtgtgggggggtcacagttcaactctcaa
agtctatcttgtaccaagtgtagatggtttgttgtgccaccacaatgttg
aactctcatagacgatccatcagaattcagagctatctatacttgctcct
atcatgcattgggcagagctgggtcgcatggtcacacctagctgcaaggg
aggctaggtaaggtttgccgtagacatccacaagtacggctagaagggat
ttcaagggagaagacagcttctgtcataggtagggaacactgagattctt
tctggatttctgggcaacatgtccctattagactgatgctggtctgctac
cactattctttcctgccagatggaatacgggtgcaatagttggcactcca
gcagccattttggaccctaagttaatataaccctggagcagctattgcct
aatgtttcccattctgtacaactgagcttggaaagagcatgacacctggc
attttaggaaggactagctctccctgttcattagggtgtgggagggatgt
ctgagaacctcaactaatccatgggaccaaggagtccctctggggggctt
cccggaagaggaggcaagtgagcaaagcaggcagaggttacaagctgggg
cggggggcagcagggtcttcaggcatttgagtcaggccagtttgatgccc
tagaccactgtgcaggatcccagggctgggcaggggttgccactcccacc
actcaccttaagtcctcacctctcacatgcacccttctaccttgtctctc
cccctgtgcccacccccctcctcatccccacctcccagTGTCGCCAAGGC
CTTGGGCGTGAACACTGTCGGGGCGCAGACCCTGAAGCTGTTTTACGAAC
ACCCCATTTTCTCCGAGGTCATCTCAGACCAGGAGGCTGTGTCTGCTCTT
GAGAAGTTCGTGGgtacgttctgtggaatctttttgccccttgactttct
ccctcctcagcctagactctcatgccagtccctggtaaatgcaaaagcca
ctcacaaaaatgtgagtctggtaatcatgatagcagtagctgggatcaaa
ggggagaggaccaagatgagctggacttggagatgatctttagcagaact
ctctggggccagccttgaagatagccaaagagaacaagagtaagaacacg
cactgctgttaatcatctcagcttgctgccacacaggagtgaccttctca
ggtgcctgagtgcccttagcacaggctgtaggaacaagcagttccagcca
tagggagatgttgagagcttaccagccgcctccccctccccccgtagACG
ATGAGAAGATTCTGGTGGAGCCCGCGTGCGGCGCGGCGCTGGCTGCTGTG
TACAGCCGCGTGGTGTGCAGGCTGCAGGATGAAGGCCGGCTGCAAACCCC
ACTGGCCTCGCTGGTTGTCATCGTGTGTGGCGGCAGCAACATAAGCCTGG
CGCAGCTGCAGGCACTCAAGGTGCAGCTGGGCCTGAACGGGCTGCCCGAG
TGA
Rat SDS
>rn7_refGene_NM_053962
range = chr12: 36083385-36091040
5′pad = 0 3′pad = 0
strand = − repeatMasking = none
(SEQ ID NO: 692)
gggttcatgaagctgagccccatactccccgccctttttctttctatttt
gagacaggctctcactccttagtccaggctgtccttctgcctcagcttct
attgttctggcttgaaggtgtgagttgccatatggctggcctgtggctgt
ttcttggccataaactacgtgtccagcaactgtccggcttttctgttatt
ctgggattgacccctgggactctggcaggagctcctcagtgctgcgctgc
ccgcccacgagtcctataggcctttcggagatgaatgtcttcatttgcaa
cggaaaaaaaaaaaaaaaaaaaaaaaaagaacggggagagaaggtgaggc
ggaccttggagggcagaacaagtggtgcagcatggaccagggggcgggga
tttctgggggcgtggcctgcctgggccctcattggctgcccggaggctca
gcccggagaataaagccctggggtgtcctgccaactttccactctcccag
GACACAGACAAAATCTACCCTTCTCGCTCGCTCTGTCCAGCTCCTCCTGT
GCCGGgtatgtggccctcagcctcagtctcaccccaagattgacagccag
agcagggtaccggggaagggaagagggactgggactcatccagttcaagt
ctccatgtactgatctgtaaaatgggacgatgataggaccctctagatca
ggacgtcgccgggtgggcgtgtgacttggccaagtgctcgcatgagtcaa
atgacaaggaagagacttctgccgtggaacccatgccgcaccggccacct
ttgccaagaccgcctgtgcctttttctctcgcaggtcatatcacctatag
aggcggatgcgactggcctctggggtgggaatcagtatggagtacactgc
ccattttcccaaaggtttccaatgctacagcctgggacaggaaccaggca
gggttcgccaaagccacctggaaggggtctgcgtggcggaaactatccac
tagccaaaggctgcatcggctgggccttttgggtccagagaagacctagt
atgtcaggatggggctagctaggacaaagcaaagaaatttggagttactc
ttgtgtgccattgtttccgactgcctttcaagaggtcttctagaaagagg
agtctggtgggcttgcctataatctcagcacttgagagactgaggcagga
ggatcacaagtctgtctctgtctgtctgtctcttgactgaggcaggagaa
tcacaaggagtttgaatctctcctacactcacacacatgcactcgaattc
gtgtagatgctgggaagagacaccacctgcctaggtttggtcccacttga
ctctgtatctctcttggggcttctcacagggctccaaggtgaaagtggct
gagccggtgttaacccaaaggcttcctcactgtcctggctcccgagggga
cctcagggtgccattagccaaacttgccgtgcagcctctatctgcatcga
ggttccctctcattgtggacgctgtatgacgagggcaagccagaggtctc
tgttgcctcttccccatgccagaaaaagcaattccataattctatctttt
taaaagaatgagagcctttcagagctcgatgctggatttccaggtctttt
gggggttttgttttgttttgaaacagactcctatacagcccagggtggcc
tcaaactccctacatagccaaggttgaccttgaatggctagtcctcttgc
ccttgcccccaagcaatgggattacaggcctcctgcaccaagcccatttt
tttcttcaaaatatgtatttgtcgaacaccagGTGCGGCGGGGCATACCT
GTGATCCCAGCAATTGGGAGACTGAGACAGGAGGATCCAACCTTCAAAGC
TACATGgtaagaacatactgagtttgttttaaagcatgtgtggggttggg
atatggctcagtctatgaagggcttcctgggaaagcatgagggcctgtgt
tcatattcccagaatgcacctcaaaaatgctgagctcaacaataaatggc
tatcatccctgcactagggagcaagagacagaaggacctcaagggacatc
caggggcctctggaacctcaggggacttctgggaacctctggggacctca
gagcctccggggttctctggccagccagttggactgaatcagggtgctcc
tggttcagtgcaagaccctgcctcaaaaatagtgggcagctgaggaggct
actgactgatgttgacctctggtttctgcacacacacacacacacacaca
cacacacacgcacgcacgcacgcgcgcgcgcgcgcgcgcacgcacacaca
cacacatgtttaagtaatatataatagggcaaaatgctgagcacggaggg
gtgctgtggcccggagggtcactcttcacaggtaccccatttggcccatt
ttgtgtgtgtgtgtgtgtgtgtgtgtgtgtgtgtgtgtgtgtgtgtgtgt
gtgtgtgctgacatactctgagctcatgggaacaggtcagcccgggttgg
ttcctgaggactttgcagaaacagaccctggctgaccctggctgtgcttt
gttcccatcttgtggctcattctgagccagtttgaacaagcccctccctc
tgaacttgcccaacgctgcagcctaaagatgtttctgagcttgtgacctt
ctaactttgggccactgggtagttggagctgagctttgatttggtgtcct
ttccttatcattatgggtatgaaagagtggtcctagagcccaggagggca
gcctctcttgaccctctgtatctcctctctgcccccagCCATGGCTGCCC
AGGAGTCCCTGCACGTGAAGACCCCACTACGTGACAGCATGGCATTGTCC
AAAGTGGCCGGCACTAGTGTGTTCCTTAAGATGGACAGCTCTCAGCCCTC
TGGCTCCTTCAAGATCCGAGGCATTGGGCATCTCTGCAAGATGgtacaag
gcagggtggcatactggcaggagaggcagtccctcctacctgacagctgt
agaggcaggtgtgcagatacacacccaggattccaaacggaggcaggaag
gtggaaagtgaatggtatcccagagtgcatagcaagaacctgtctctaag
gaagtaataataattttagggactgcagttgagacagctcggcggctgag
agcactggctgcttttgcaggggacttgagctcagttcccgcaaccacat
ggtggctcacgatcaactataactccagctccaggagatccaacactctc
ttctgatttcccggagcaactgcactcacatgcatataattcaaaataag
taaatattgggctggagcgataattcagtggttaggaatacttggggctc
tttcagaacagccatgctcaccctcagcatccaagtgagcgaggggctgt
aactccagctgcagggcatcaaatgctctcctctgccctctacaggcaat
acgtacacacagacacaagtgcacataaagaaaacacatctttttttttt
ctttttaagaatttttatttattttacttatatgagcacactgttgctgt
cttcagaaacaccagaagagggcaccagatcccatcgcagatggttgtga
gccaccatgtggttgctggggattgaactcaggacctctggaagagcagt
cagtgctcttaacccctaagccatctctccagcccaagaaagcacatcct
aaataaataaataaaatatgttttaaagaaaataataattttagaatagg
tttcacacatcagggctctctctggctaaggggtgcccaccctacagact
gacacctgcctccttaccccagttaatcaccttgggggagatgagcctga
gagagtggttcagataccaagtgggcaagctcagatgttccagtggtgag
gacgtgcctaggcagggacttgggggtggcctgatccttcctcttgtccc
cacagAAGGCAAAACAAGGCTGTAAACATTTCGTCTGCTCTTCAGgtgag
tgcccagttcgttttctttcccttcctaccctgttctgtgtgtcctgtgg
cccagcaccaggcattctccctggactctgggctctgcaacaacacatcc
attcgtagtcgtccagatttggggttccagaatgaggcagaagtcactct
ggagatgagcagccccacgtgaggtcccaggccctccttcctgatacacc
ctctccactgacagCGGGCAACGCGGGCATGGCGACTGCCTATGCTGCCA
GGAGGCTGGGCCTCCCAGCCACTATTGTTGTGCCAAGCACCACACCTGCC
CTCACCATTGAGCGGCTGAAGAACGAAGGGGCCACAGTTGAAGTGGTGGG
AGAGgtgagtgcagacctattgtgggcagcagagctggcgggtggaggtg
gggggctgataactggatagagccctcgttcatctcaatctcttcctgcc
cccacccctgcagATGCTGGATGAGGCCATCCAACTGGCCAAGGCTCTGG
AAAAGAACAACCCAGGTTGGGTGTACATCTCCCCCTTCGATGACCCTCTC
ATCTGgtaagttggagccaccccactccctactgcatgtgtggtctctgc
tcatgttctcgccggcacaccagccatgaggggtccccagatgcagagat
tcggagagatcaactcatcttggggtggctaggagcaaactaactaggga
acgcatcggtggaggtggctttcatgtgccagcatctgccctgaacaact
gaagcttggcccctgggaaccctgggagtcggtggggaatgagcatctaa
ggatgtgtccactggtgggcaggaaatgggggtttatctaccacacccac
agccatctcctctctgggagtgcgtggggtgagggggtggggaggtgggg
tcttgtctcctaccctgtgccctgagcagcagggaggtcagaggtcaggt
tcatcatggcctggcatagagggaaaaacagaccttagctggtatctttg
accagcccttgtgtcttcaggaagccctgacctgacctgccacccctctc
ttgcccagGGAAGGCCACACTTCCCTTGTGAAGGAGCTGAAGGAGACACT
GAGCGCCAAGCCCGGGGCCATTGTGCTGTCTGTGGGCGGTGGAGGCCTGC
TGTGCGGAGTGGTCCAGGGGCTGCGGGAGGTGGGCTGGGAGGATGTGCCC
ATCATCGCCATGGAGACCTTCGGCGCCCACAGCTTCCACGCTGCCGTCAA
GGAAGGAAAGCTGGTCACCCTGCCCAAGATCACCAGgtgagcccggggca
cctctccagcacacaggcggtggagctgagcctttggtcctttgcccgaa
gccacactctcgcgcggcccgtctgggccttgaacgtgagtccgttcttt
atgtgacagtgcttaagtgacgtccttcgcttcttgaggtcctcacgcct
catttcttttgctgctgtttgatctggcctccgaaatgtgccagaccctg
gctggtcctggactcagcctgggagaaaacaatctctggcctgcaaagaa
tttataaccaaaccccaccgcggcggctcaaaccaaactgagctctagtt
ctatttaagactattaggggcaactcttcgaaccaggggctcagttcctt
tagtctcttgtcccgccactgagagtgttgacctagggtccaaagtggct
gctttggtgccagccaccgcacacacactccctctgaaaagaggaagcaa
gagcagaagttgtttctaaactttaaagaccaagtagagctgctccccag
ctgacaatggggtcacacccaatctttaaccattcgttaaagattgctat
gaatgagttgaacctggtctgctggacgccttacctaagctttttggcac
acggtacagaactggctgcttcttgctgataggctgatgggagaggtctg
gctcaaggctgccactgggcaacccaagagcggattgtactgcaaaccca
tagtctgagaaaaggtctgagttcaactctcaaagtttggtttgtaccaa
atgtggattttttttttttttgacaccacaacgttgaactctcatagacc
aaccctcagaaatcagagctgtctgtacttccctcttatcatgcactggg
cagtgcttggtcacatgaccatacctagctgcaagggaggcagggcaagg
ttcacactaggcatccacaagaacagctagaagggatttctagagagagg
atggcttctgtcataggtagggaacaccgaatcagttaagagactcttcc
atatttttgggcaccatggtgagactgatgccatcctgctaccactattc
tttcctgccagatggaatacagatgcagtagctggcactcaagcagccat
tttggaccctaagttaataggaccctggggcagatattgcctcatctgtt
tcccactctatacaactgagcttggaaagtgtgtgacagacacctgatac
tttaggaaggactagttctccctgtttgttagagtgtgggagggctgtct
gagagcctcaactaatccatgagaccaaggagtccctctgggagtctccc
tggaagaggtagcaagtgagcaaagcaggcggaggttacaagctggcagg
ctgggggtgggggtggggcagcagagtcttctggtatctgaatcaggcca
gtttgatatcctagaccactgtacaggttcctggggctgggcagggttgc
ccactcccactgttcacctgtagtccccgcatctcacgtgcacccttcta
ccttgtctctccccatgtccacccccctctcattcccacctcccagTGTT
GCCAAGGCCTTGGGTGTGAACACTGTGGGGGCACAGACCCTGAAGCTGTT
TTACGAACACCCCATTTTCTCTGAGGTCATCTCAGACCAGGAGGCTGTGA
CTGCTATCGAGAAGTTCGTAGgtatgttctgtgggatctttttgtccttg
attttctccctcctcagcctagaccatctcatgaaaggaaactaaagcca
gtcagtggtaaacgcaaaagctgctcgtgaaaacatgaacctggttaacc
atgatagcagggaggacaaactagttttataagtgccagttttgagcttt
ctggtagccagagtcaaaagggaaagggccaagatgaggtgatctctctg
ggcttagcctgggagatggtcaaaggaacgagagtagtgcacactgccat
taataacttcagcttgctacaccactagagagcgaccttctcaggggccc
aggaaccccaagcacagactgtagaaaccgagactgggttcagaacaaac
agtcctggccacagtgactgacgagagctgacgagccccctcccctacag
ACGATGAGAAGATCCTGGTGGAGCCCGCGTGTGGCGCTGCCCTGGCTGCA
GTGTACAGCGGTGTGGTGTGCAGGCTGCAGGCTGAGGGCCGACTGCAAAC
CCCACTGGCCTCGCTGGTTGTCATTGTGTGTGGTGGCAGCAACATCAGCC
TGGCACAGCTGCAGGCACTCAAGGCACAGCTGGGCCTGAATGAGCTACTC
AAGTGA
Equine SDS
>equCab3_ncbiRefSeqPredicted_XM_001490154.5
range = chr8: 21618657-21627483
(SEQ ID NO: 693)
5′pad = 0 3′pad = 0 strand = + repeatMasking = none
aaagagtcctaaaagatgacaggggaaggcaataaggaactgcgttcgga
ttgcaggtttgggagtccaactgctggattcaagtgtcggctcatccctc
tcactagctgtgtgcctcagtttccccttctgtaagatggggataatgat
agtacccacttcctcctggggtttgttccttgggtgcctactatgtgcca
tgctgacgactttaagtgccttctcctggccctcctcctgtaagctcctc
acagcccttgggaagtaggtatgggccccattcgcaagtaaggaagctga
ggaccgggaagaggaagtggccatgcggagaggagagggagggagagatg
ggaggaagctgccggagggaaatgcacagccagcctccctgccccctcct
ggaagcccccagcggcttcaggccagggggcggggcctggcgggggcggg
gccagccctggggccccattggctgcgcggaggcacccgctgaataaagc
CCGGATGGGCAGCCCCTGGGACAGTTCAGAAGACAGTCCACCTGTCCACC
TACCCTCCATCTGTCCATGGGCCATCTGCCCAGTCTATCCATCTGGACCA
TCTCCAGTCATCCCTCCTTGGCTTACTCGCTCTACCCAGCGCCCCGCTCC
ATCGCTTTGCCAGgtgagcggccccatcctcagccccaccccaggaccgg
agtgcctgcgccagccggggcttcccaggggaagaagggcaggtagaaag
agtctgttgggcataaaaacccatgctttggagccagactgcctgggttc
acatccagctctgtgcctcagtttcctcatctataaaatggggacggtca
taggaccttcctcataaggtttctgtgagggtgaaatgagatagtgcatt
taaagtgtccggtccagaagtcatcatatttcgtttttactgagctctga
ctgtgagccgagtgagaggggagtgacttctgacggtggggcgcctggct
agcgtccctttctgaagtctccccttcaggggcgagagtagcagggtggg
aatggggtgggggcagtttacaggtgctggcttcacacagggcgaggggt
gatgcccgcgaggggtccctgttgcctcactgaaggcatctgatgctgca
acagaggcaggtggtgggaggggcttcccaaaccagcggtaaggggtctt
cgtggttgacacttcccacctccaaccgggagagatccctgaggccaggg
cttggtgacttcacaagacccagggctttaaggtccagtccgccagaaca
gggggtgggggggtggctgggggggaggccatggcttggggaaagccccc
aggagctgagagctattcagaccgatgggggaagccccttgtggagtgcc
tccagtctctagctttcattcacaagatctagaggggaggcggtgtctgt
ctgggccccctcccaggccctgaatgtcggttaatttggacagacatggg
ataccaacatttggggggttctaaggccggggggtgtcaggaatcccacc
gcctactttccttctgacccaggcatatctggcccaatctgggagaggcg
atgcctgagccaggagggccagggcagggggctggaccagacagtgtcac
tggaaacgcggtgggggcttggccttccgggatggtacagggcggggggg
ggaccagagaggccgctcacccaactacccgctgatcaattctggctcag
cccaagtcggggggcatcagcctcccccatgattctcagcaggagagagg
acaagatggaaaggctgggccacctcaacttctccgtctcccagggggcc
tgagccacccccacgtgtcctagagggagcctcggccctggaaccggact
gctgggttcagatcctgcccccccccaaccccccgctgtgtgatcttggg
caaggcacattaatgttccagtccccatcgctgggtgacaaactactgca
agatgtagcggcttcaaatgacagccatcatcacgctgttacctgtcgtg
gttctgggctcagccaggcagcccccactgggctctcatatggttgcagt
gtgacgggggctgggctggagccatctcaaagtggtgtcccctatctggc
agttgacattggccatcagtggtcactgggacgcccatatgtggcctccg
cacgtggcctgggcttactcccatctcggtggctgcgccttgagagggag
cttcccggagagatggccaggctgtgtggcctctgtgaccccgcctgggg
gcggtggggccctggagctggcttacactggctctgggagtggattgtgc
ctgtccgtgttcattgcatcatgttggcctcggcgagggacgtgcgtcac
ggaagggcacgccctgcagtcggggcctcgctttccttcagagatcccct
tgttaaccattcaccagcacaccactgctcggacgtcacaccgtcacctc
cattgcagccacaggccccccgggggccaggggaggacacatagacccct
cctcccgcagagagctcgtcagtggcgcctcgaaagcggtgcctgtggga
tgggaggaggttatgtagccatcctggaaaacatgatgggagcctgtctc
ctgatcagaagaatggggcggtagcggcatctctagggttgtccaaagga
tgcaatgaattaatatctgccaaactgaaaacagtgcctggcacatggtg
atcactttataagtgatcattaagtactatagtgattattattaaaacat
gataagaagaagaagctggataataataaataataataaaataataaata
taaataaaataatttaaaacagggaacagggctttccaaaagtttgtttt
aaaatacacacatatttaaataaggaatatgtgcccgcggccaatcgaac
agcacaaaagggaactcggagcgcccgctctccccagaggccccgcccgg
actagttcctcgcgtgtgtgtcctgggatgctccatgcacgtggaagcaa
gtgaactgtgtgtgtccacatttttcgcacacatagcccagacctgatgt
cccgcccctgctttacccactgagccatcctgccattccttctgagtcag
cacctgcaccgccctcccccccacgcccctccaaagctgtggggtctgag
tgttcttgctgagggtcgagcctcccagcagggtgggtgttgggggggtc
cttatccccattttacagatgaggcagctgaggccctggggagatgactt
gtctgatgtcacccggtgaggaggaggccgagttgagatttgaccccgtc
tccaggacattgtgatccgggccctgtcccagcctcctgggaagggaagg
aacagggtcactcacagccacaccgagaggctgcgctggcaccagggcca
ccctccaaccctccgggtttccttccttcctccagCAATGACGTCCACAG
AGCCCCTGCACGTGCAGACCCCGGTCCGGGACAGCCCGTCCCTGTCCAAA
GTGGCCGGCACCAGGGTCTACCTCAAGATGGACAGCGCCCAGCCGTCCGG
CTCCTTCAAGATCCGGGGCATCGGGCACCTCTGCAGGACGgtacaggacg
ggctccgtcagcagggcgggggcgccaggtcctctctccttgacagggca
gggcgagtgctgcgtgaagtctcgggcttccaggctgggcctctaactcc
ccacaatgcctgagcatccaccattagcacctcctcgggttgaggggtgc
ccgtccgtgaggtcccctcctgggctgaccccagagccaccaggtgccgg
cctctccctcacccgtccctgaaccagcctcttcggggagggaggatgtc
agaggccagggcagtacaattctgtgtgcaggaaggagaggtcagcataa
cttggaaacagtaggaggtattccaggtgggaggggaaagccctgggaca
gactttgaggtgggcctgacccctccccacttacctccacagTGGGCTGA
GCGAGGCTGTAAACATTTCATCTGCTGCTCGGgtaggtggcctgcatgcc
tcttcctgtcctctgctgtgtgtcccggggccaagtgctccacctctctg
ggccctggtttccccagcaatgagctgtggcaaacatctatagcagtccc
aacttggattccagggggaggccggcggtcacccaggggagttcagagcc
ctgaggttgggttgaatggtcccagccaggctcccggttcctcccacact
gacgctctctccaccccctacccctggcagCGGGCAACGCAGGCATGGCA
GCTGCCTACGCCGCCAGGAAGCTGGGCGTTCCCGCCACCATCATTGTGCC
CAGCACCACCCCTGCCGTCACCATCGAGCGGCTCAAGAATGAGGGTGCCA
TGGTCAAGGTGGCGGGTGAGgtgagtgctgaccggggcgggggccacaga
gggcacctggtggcagggcagggtcctccggcgcctcaccccatgccacc
tcccctgtggtcttgcagATGCTGGATGACACCTTCGAGCTGGCCAAGGC
CCTGGCGAGGAACAACCCCAGCTGGGTCTACATCTCTCCCTTCGACGACC
CCCTCATCTGgtacgcagagcccggggccacccctccacccctgggcacc
taccacaaagcacaccatcatgtgcggcagttgtacacgcatgtgcacgg
catcgtgtagaagcacacgcacaagcagatagaaaacgcacacgcacgtg
ccccaacgcccattcgccgtggccacacccaacactcccatgagccaaca
cacaagctcagagacacaggaatctgccactgggtcccccgggaagcaga
cccgagacaaagatttcacagaaagcagtttgcgtgggaggtgattcgtt
agcggcgtggggaaggaaggaagccactaaggctcaactgggcaggcggc
tgctgggggcagctggagccgaaaccctcggggggcccctgggagccagt
gtggaacatgaaccagagttatcccacccgtgcgagaggatgctggagtt
tagacagcaacacccgcaactcttcattctcatcatcactggctgggggc
tattctcagggggtgtccttgggggctgtcgtctgctggcaccgccggtt
gtcacattcataggcagctgcggtggcagagagagccctcgggggagagg
cagctgctggcagctggagatctgggcggagtcggggaggaggggactag
ccccgaggcccgtcagctgcctgggataagggcaagtggcaccgccagga
gccccgcctgctgtccccaagaagcccaaccctgacctgccaccctccca
ccccctgcagGGAAGGCCACCGTTCCATCGTGAGAGAGCTGAAGGAGACC
CTGAGCACAAAGCCGGGGGCCATCGCGCTGTCGGTGGGCGGTGGGGGCCT
GCTGTGCGGAGTGGTCCAGGGGCTGCAGGAGGTGGGCTGGGGGGATGTGC
CCGTCATCGCCATGGAGACCCTCGGAGCCCACAGCTTCCACGCCGCCACC
ACTGCCGGCAAGCTCGTCTCCCTGCCCCAGATTACCAGgtgagcagccga
gggagggccccgtcgggtggtcagccagcctgggggctgccgggcaggtc
ccgacaccagtaccatttcacaggtgatgacaccaaggcttcggtcacct
gcccagagtcacagtccatgggtggtactgctgggacccaacccaagtct
atgtggctcagcaccgtgaacgtcacacttctgcacatgaaaagtctaac
actagtcattcattctgatccttgggggagggctccacacctcattaatt
tgccagccatttaccgagcccctaatctgtgcaagacgctggctgatcct
gggggacagtgaggaataaacacagtgcccgggctcatgtcccagcccca
cacctttgatggtgtttcattaggatcaggtttggccgcattatacagga
gcccaaaataacagtggctcaaacaagattgaacttcatttcctctttta
tattaaaaaaaaaaagtctggggggcgggcgatgggcgaaatgagtcaag
ggggtcaaaaggtacaaactcccagttaaaaaataaataagccctggggc
tgcaatgtacagactggtgactatcgtcaatgataccgtattgcatattc
gaaagttgctaacagaatagatcttaaaagttctcatcgcaagaaaaaaa
ttgtaactatgtgaggtgacagaagttacctagacgtattgtggtgatca
tttcacaatatatacaaatagtgaatcattatgttgcacacctgaaactc
catataatgttatatgtcaattatacctcaatttaaaaaaagtctagagg
caggcaggcagtcccagggcagcaccctgaaggcagcggcccagattcct
tgagttttttgccccatcatcctcatcatgtagcctatggcccaaaatgg
ctgcttaagtgccaactatcacacccatactccatgtggcaggaaggtgg
aagcaagggcagaagctatgcccctcctttaaagacagacacaatacttc
cacttacctctcattggccagaacttggtcacatggccacacctactgca
ggggtggctgcgaaatgtcttcccaccaggcctccatgtgtgcagctgga
atagatatggggccgacatgtctgccacagataggggccactgagtctga
gtaagaacaacttctggattgccatgcccggtgccccggagccaccgtgc
tggcctctgatcccacactgttgggactgaacaaataattgttgagcgcc
aagtctgagccacacgctgggctaagggctaaggtggtccagaccccggg
atgcacatgggcgagtcggggagatgggagcagccagggatgactcgggg
aggtgggggatggaacaggctggttacacacgcaggtcctagtcacagga
ccctgccggcagccacagcaccactgctccctccacgcgggacctcgggc
aagtcactgcgtgctctctgcctcagtttctccttctataaaatcgaacc
aactaacagcaggaactgttgcgagagttaaatgacacctgtgaagtgct
cagcacccagtacttctcaagaagtcagcgctatgcttgtgttcaggggg
aggaccgttggagaattccacgtggggccgcgggactgagtgctccccct
ggaggactcctgggaggaggtggccttaatcctgagccttgaagggcggg
caggccttgggccagcagagcggtgctaagctcagtgtgggtgaagcagg
tggcggtgacagcccggagcaaaggctgggagtccctctgggggaccgca
agggggccttccaggcacttgctcctgagctgatgttgaacagaaaatga
caccgtttgccccactgaagcaacctgtgcctcattagaggaccccactc
gctccggggagcccccgggacctctcagtgcttcacggctgcggagggta
cgccccgtgcccccgcctgcggagccccagttgcctcctccctctcacac
ccatccccgtctccgtctctcccccccatcccgacctctgcctccctcct
ctctctgtttcccccattcctgtcccctcctccatcactcccctgccagT
GTCGCCAAGGCCCTGGGTGTGAAGACTGTGGCGGCTGAGGCCCTGAAGCT
GTTTTGGGAACACCCCGTTTTCTCTGAAGTTGTCTCGGACCAGGAGGCAG
TGGCCGCCGTGGAGAAGTTCCTGGgtacgtgccaagtcctttcaagacct
gacccactggtccaggaactaatttgctgtccacttgaggccacaggatt
gcattcaccacaaacttccctcacagttctgtctcctgtcccttcctaga
acttcccgttaatgaagagaatacagtgcagtggtaaaaatgggagggcc
aaagccacaaatgaagatgtttgataaccatgacagtaggggacagtaac
tgagtattttaaaaaggttgcttctgagcttcctggtagccagggtgaaa
aaggaaagcatgggcctcagctgtgggctcaggggtgggtctggaagtac
gtgggactctttctaggttcagcctaggacagggatggcaaacagacaca
cacacgcacacacacacctgcgtgcacacacatgcatacagggtcgcaga
tatgctgccacctaattctcctgcactggcagtcagtttggctaatcaac
cacggctctcctactcaggcgatgcctcagaatccttctcaacacagtgc
tcaggcgactatcaccattttatcaatgttagcatcaggatgaaacctag
ccctggctcaggttgggaggtccgtctagggtaacgatggggctggtcca
ggacctgagtctgtggccaggccaagagtggactggctcataaaccccac
gctttacccagggcccgacagtccccttgcaccctgtggaactcgggccc
cgccctcacctccccctcctcgggcagATGACGAAAAGATCCTGGTGGAG
GCCGCCTGCGGGGCAGCCCTGGCCGCCGTCTACAGCCACGTGGTGCAGAA
GCTGCAGGGCGAGGGGAAGCTCCGTGCCCCGCTGTCCTCGCTCGTGGTCA
TCGTCTGTGGGGGCAGCAACATCAGCCTGGCCCAGCTGAGGGAGCAGCTG
GGCATGAAGGACGGGCTGCCCCAGTGA
Feline SDS
>felCat9 ncbiRefSeqPredicted XM_003994691.4
range = chrD3: 11018073-11027171
5′pad = 0 3′pad = 0 strand = − repeatMasking = none
(SEQ ID NO: 694)
ggaggcaaacggacctgcccagggcaacacagctagtaaccggcagagcc
aggacttgaaccccgacccgccagggtggtccccagaaggccatctgccc
cttcgagacgaaagcgtcaccgagagcgccagaggaaggcaacggagacc
tgtgctcacactgcagcccactggggtccggggccggctcatctctgtca
ccacgtgggtctccgcttcccttgtcgtcaaatgagggtagtgacagtac
ccactttcccctgggggcccactaggtgccaagcacctgctagtgactaa
gtgcaccgtctcacgccatcctcctgcaggccctggggggggggggggtg
gggaagggaggtggccaccgaggacagagacccgagggagccgcaaggca
ggacaagcccggccagcttccccctcccctcccggaagcccccgggccag
cggccaggggcggggcctggcggggggcgtggccagccccggggctccat
TGGCTGCCGGGAGGCGCCCGGGGAATAAAGCCTGGGACCGAAGGCCCAGA
CCGGTAGTCCCCGGGACAAATCGGGAGACCCTCTACCTGCCCCGCTAGCC
TCCATCTGTCCGAGGCCCATCTACCCAGTCCATCCATCTAGATCACCTCC
TGGCCGTCCCTTCCCGGCTTGCTCGCTCTGTCCAGCCGCCTTGGCCAGgt
aagtacccctcctcagccccaccccagactggagtgcctgcagcccagcg
agggggcaggttccaggccaaggcgaggaggtagagtccgtggagcgtaa
aaacccatgctctgaagccagaccggctgcgttggcatctagctctgggc
ctcagtggtctcttctctaaaatgggggtcatgacggacccttcctcatc
aggcagtgggaagggtgagatagtacactcaaaatgagagttcgttttta
ttgagctcttgctatgagagggggagtgacttcttcttctccttcttctt
aatttatttttgagagacggaatgcaagtgggttacgggcagagagagag
ggagacacagaatccgaagcgaggtccaggctctgagctgtcagcacaga
cgcagggcttgaacccacgaactgggagatcatgacctgagccgaagtca
tgatgctcagccgactgagccgcccaggcgccccgagggggagtgacttc
tgagcgagaggggacacatgacagcagcttccagccccctccctcctccg
tgtttggcaccccccacccaggatgttgtgctggagggagcagtgcccat
agggggtccctgcccctcatctgaaggcatccgtggtgcaaccagaccca
ggtgggggcagaggcttcccaaatccgcccgaaagcagtcttgggggtat
gccccccactgccaacccggagagctccctgaggccagagattggccaca
cacacacggggtcccgtcagccagaagcgggggtgccgttgtttcaggaa
taacctccaggagggtaataatcaaatagatatggggacgcttctgtaat
ggggtgcatctagtctccagcttttgttcaggagacctaggggagaagtg
gtatccgttcagggcctcccgctcccaggccctggatgttagttaattca
cacagacatcagttgtccgtatgtgggggagctaaggctggggaggtggt
caggaaccccatccctcgcttttcttctgagccaggcatatatggccctc
tctgggagccagaggcctgagctggaggccgtggccagggcaggctgcac
cgggtgggtcactggaaatggggcaggggtgggcggggaggggcagccag
ccttgtgtgggacggagaaggacatggggagggccagacaggctccccac
gcaaccacccgctgatcagttctggctcagcctgagttggggagactcca
ggaggagaagggacatgtgtggtggccagactctgggccacttcagcttc
tcaatcacccagagcccgtgcagaaggggagccctgagccgcgcccacag
gtgctagactgtctcatccagtcctgcctcccccactaactagctgtgtg
gccttgggcaagtcacattaatattccggtccctgttgctgtgtgaccaa
ccgccccaaggtcaacagcgttcatcgtattgttgtctgtcacggccctg
gggcttatcggggcagttcgcactggggtctctcagatggctgcagggtg
acatctccaggtcaccccgccatgtctggccatcagctgtcaggtagggc
acctacatgtggcctctgcatgcggcctgggcttactcccatcctggcag
ccgggtttcgagaggggagctgccccagagagacggccaggcggaggctg
tgtccccttctgtgacttggcctggggagcagggctgcactggagccagc
agattgtgccgtctcttcccagctccacaggcaatgacgtcatgttggta
actcgagagtggccttggagaaggttacgtacagaacagaagtgcgcccg
ctacaaatcggggctttgtcttgggagagtccattgctgaacgtttacaa
gcacaccaccgcttggaaatcgcatcgcgtcttttccagctgcggccacc
gaccacccccctcctccagggttcaaggggaggaaacacagagcccacct
ctcactgagaaaagcatctgtgtcccatcatgaagggatgggaagggctt
gcagggaccatccatcctgggaaacacaatccgagcctgtgttctcatcg
ggagaatgggataatgatggcacctcccaggattggtcaaaggaataagt
gaattgatattggtaaagcttaggggcgcccggatggctcagtcggttaa
gcgtctgactcttgattttggctcaggtcatgatctcaccgctcatgaga
ctgagccccgcattgggctctgggctgacagcccagagcctgcttgggat
tctctctctccttctctctctctgcccctctcctgctcatgttctctctc
tctctcaaaataaataggtaaacttaaactatatatatctgtcaagttta
aaatagtgcctggcacatggtaatcgcttcagaagcaattaataaacaat
atagtaattattataaaataataataattggggcgcctgggtggcgcagt
cggttaagcgtccgacttcagccaggtcacgatctcgcggtccgtgagtt
cgagccccgcgtcaggctctgggctgatggcttggagcctggagcctgtt
tccgattctgtgtctccctctctttctgcccctcccccgttcatgctctg
tctctctgtgtcccaaaaataaataaacgttaaaaaaaaaatttaaaata
ataataataataataagttttccctggttgggtacagggctttccaaagg
ttagtataaaaaatacacgtgtttaaataaagaggcaatacctcttcgta
gcaaaatagttaactagcacaaaaggggaggccgagtgccggctctcccc
agagagcgtttctcaccctagcgagtttcttgtgtgtttcctgcgatgtt
ctatgcccgcagaagcctatggattgtgtgtgtgtgtgtgtgtgtgtgtg
tgtgtgtgcgcgcgcgtgtccctgtgtcctcgcacagactatacacaccc
gccgtcctgccccttctttagcacgtaaccgtctcgcagctcgctctgca
tccgcccctgcgaaaccatcggagtcacgccccttgacaggtgccaggtc
ccaatgctatttctgagttcacagcttcccagcacagtgtgccctggggg
ggggggggggcttcccccccgttttacagacgaggcagctggagctgagg
gggcatcgctcgtctgatgtcacctggtgagtaggtggctcagttgagat
gggaatccaggcgtccgggacgcttctcatgtgggcgtttccctcggcct
cgtgggacgggagggcagggctcactggcaggcgcgccgagggccccggg
gccggctctcccgaccatctgggtttccttcccttctcccagCAATGATG
TCTGGAGAGCCCCTGCACGTGCGGACCCCCGTCCGTGACAGCATGTCCCT
GTCCAAAGTGGCGGGCACCAGCGTCTATCTCAAGATGGACAGCGTCCAAC
CTTCAGGCTCCTTCAAGATCCGGGGCATCGGACATCTCTGCAGGATGgta
cgggcgggatgggctccacgtcgtctgagggagggggccagtcccacgac
gcatccgcggttagcactccccccaaagaggcggtgcccgcccgggaggg
tctgcccgtggactcatcccagagccgtcatttgcttgcctttcccccgc
acccttccctgagccaggccctttggggaggggcgatgtccgcggagagg
caagggcaatacaaagtcctactccctgggggaggggagagcccggggac
agcctgggacgtgggcctgaccctccccccttgtctccacagTGGGCTGA
GCAAGGCTGTGAACATTTCGTCTGCTCCTCGGgtcagtggcggtgtgcct
cctcctgtgtggcccagggccgggcgctctacctctctgggccctggttt
ccccagcgacgagccattacaaacgcctgcggcagccgccgttcagaatt
ccagggggggaggcgggaaggcactcgtaggggggggtgttcggagcccc
gaggctggggacgacagtcccagctgggctcccaggccatccttcactga
cacgctctccaccccatccccggcagCGGGCAACGCAGGCATGGCAGCCG
CCTATGCCGCCAGGAAGCTGGGCATCCCTGCCACCATCGTCGTGCCCAGC
ACCACACCTGCCCTCACCATCGAGCGGCTCAAGAATGAGGGTGCCGTGGT
CAAGGTGGTGGGTGAGgtgagcaccaacccgggggcggggaccacggggg
gggggcacctggtggcagggcagggtccccccagcccctcaccctctcca
acctcccctgagcaccaacccgggggcggggaccacggaggggggggcaa
cctggtggcaggcagggtccccccagcccctcaccctctccaacctcccc
tgagcaccaacccgggggcggggaccacggaggggggggcacctggtggc
agggcagggtccccccagcccctcaccctctccaacctcccctgagcacc
aacccgggggcggggaccacggaggggggggcacctggtggcagggcagg
gtccccccagcccctcaccctctccaacctcccctgagcaccaacccggg
gcagggaccacggggggcggcacctggtggcagggcagggtccccccagc
ccctcaccctctccaacctcccctgagcaccaaccgggggcggggaccac
ggaggggggggcacctggtggcagggcagggtccccccagcccctcaccc
tctccaacctcccctgagtaccaacccggggcagggaccacggggagggg
ggggcacctggtggcagggcagggtccccccagcccctcaccctctccaa
cctcccctgagcaccaacccggggcagggaccacggggggcggcacctgg
tggcagggcagggtccccccagcccctcaccctctccaacctccccagag
caccaacccggggcagggaccacgggggggggcacctggtggcagggcag
ggtccccccagcccctcaccctctccaacttcccctgtggtcttgcagAT
GTTGGATGAGGCCTTTGAGCTGGCCAAGGCCCTGGCAAAGAACAACCCAG
GCTGGGTCTACATTCCTCCCTTTGACGACCCCCTCATCTGgtatgtggag
ccccaaggcacccggtgcagggggcgacagagggctctcgtgggcaggtg
actgatgcgggcagccggaaacccgggtggaggtggggaggaggcgactg
gcccaaagcccgtcagccgcctgggttgaggacgagcgtctccaggatcg
cccccagcccccgtggggtctccagggagcccagccctgacccgcccccc
acccccacccccgtccctaactctccagGGAAGGCCACACTTCCATCGTG
GAAGAACTGAAGGAGACGCTGAGTGCAAAGCCGGGGGCCATCGCGCTGTC
GGTGGGTGGCGGGGGCCTGCTGTGCGGAGTGGTCCAGGGGCTGCAGAAGG
TGGGCTGGGGGGACGTGCCCGTCATCGCCATGGAGACCGCCGGAGCCCAC
AGCTTCCACGCCGCCACCACCGCGGGCAAGCTCGTCTCCCTGCCCCAGGT
TACCAGgtgagcagcggggcccctctggtggatgttcagcaggccttggg
aagcccacctcggtgcccgctgcgaggcagagccccgccacctgtcccat
tttacagacgaggaaactgaggctttccacttgcccagagtcacacggcc
agagagtgggtctgctgggaccgagcccaggtctgtgtgactctgtagga
ggaacgtcacatttcctcacgtgaagagttcactgttaatcactgaccga
ggcccctgccacagggttttacacccctcgttaatctgccaaatggttgg
cccctaacaggggccagtctgagcccctaacaggtgcaagcattgctgat
cccggggacagggtggtgaacaaacacagtccctgctcctaaggagttca
tattccagccccgaacctttgacagatggtgtgtaattaggattaggttt
ggcacattacacaggaggcccaagtaacagtgacttaaacaagatggaac
tttacttcctctctgtaaagtgaaaaaaatgtctagaggtacacagtctc
ggggcagggagctccctgaagccaggggtccaggttcctcgagtttgttc
tccctcatcctcatcatgtagccttacggcccaaaagggctgcctaagtg
ccaactgtcacacccacagtccatctggccaggaagagccatgccccctg
cctttaaagatacagtacttccactgacctcttactggccagcatttagt
cacgtggccacaactactgcaggggaggctgggaaatgtcttcctatcgg
gtctccacgtgtgcagctaggatagatattgggtagacaggtccctacct
cagatagggaagactgagtccaggtgagacctccttgtggattcctgagc
ctggtacccagcagccaccatgttggcctctgatcctgcgccgtaggcat
tgaaataatggcggagtgcctaccacgagtcacgtgccagggctcagggc
tcaggtggttcaggctgcaggtgctcatgggagatgtgagctgggaataa
ggccggcgatgactccagggagggcggggagaaagacaaggcaccgatta
cgcgtgcaggtcccagggttacgtggatcctgctccgggtgcccatgcca
ccactccctgaacacataaccgtgggccagtgacccgcgtgctctctgcc
tgggtttcctcttccacggaatcgaaccagtggaacggcaaacccagccg
tgaggatttaaaaatgcgcacaaaacgggtggtacctcagactccctcga
gaaggggtcgctatgtctgtttatgggggaggaccgaccgttcgagagtt
ccgtccggagacacaggcactggagtccctgggggactccttggaggagg
tggccttaatcctgagccctgaaggacagggcgggacttggacgggcaga
gtggtgcccagctgaggggagggtggagcaggtggcgtcctttcagggaa
ccgcaagggggcttctccgtccgcgtggatcaggataggctatttacccc
actcacgcctccacgtcttatgagacctcactctggcggggagcctctgg
ggcctttcagtgctttacagccacagagggcatgcccgcgcccctgcccg
tggaccccacttctctcctccctctcaaatccatttttctctttctatcc
ctcctcgtcccgatctctgccgccttcctccctcctgttttcccccgttc
cggccttcttctccatcgccacctctcccagCGTGGCCAAGGCCCTGTGC
GTGAAGACCGTGGGGGCTCAGGCCCTGAAGCTGTTTCAGGAACACcCCCA
TTTTCTCTGAGGTGATCTCGGACCAGGAGGCTGTGGCCGCCATTGAGAAG
TTTGTGGgtatgtgccaagtccttccaagccctgctggctcagggactaa
ctggtgtcctaacagatcccattcacgacaaattttcctcacagttctgt
ctcccaccccttgctagaacttcccgttaatgaaagataatacaaggcag
cggtataactagaaaggccagagccacaagtgaagaggactgataaccat
gatagcagggagcttcctggtagccagggtgaaaaaggaaagccaacccc
agggttggcttcagaagtgggtctgtgatgccgttggactcttctgggtt
cagcctaggacagacaccgcacacacacacacacacacacacgaaaggga
gcacttaatcctcctgtaccagccgtcagtttggctaatcacccatggcg
gtcttgctcaagcgacggcccaagccttctcaacacagtgctcgggcagt
taacatgcatcggtgggagcgcgctccgggtgaaacctagccctgcctca
gggctccgaaggtcaactaggatagcgatgggaggtttggtgcccagact
cctagccccgagtctttcccagggacccaacagtccccttgcccccccgc
ggaagccaggccccaccctcatcaccccctcctctggcagATGACGAGAA
GATCCTGGTGGAGCCCGCCTGCGGGGCGGCCCTGGCCGCCGTGTACAGCC
GCGTGGTTCAGAAGCTTCAAGCGGAGGGGAAGCTCCGTGCCCCGCTGTCC
TCCCTCGTGGTCATCGTCTGTGGCGGCAGCAACATCAGTCTGGGCCAGCT
GCGGGCCCTCAAGGAACAGCTGGGCATGAAGAACGGGCTGCCCGAGTGA
Canine SDS
>canFam4 ncbiRefSeqPredicted XM_038575178.1
range = chr26: 11055244-11063163
5′pad = 0 3′pad = 0 strand = − repeatMasking = none
(SEQ ID NO: 695)
attaagccatctgccccttcaagacaaaagaatcataaaggatgacagga
gaaggtaattcacattgaggcaggcccactgggttcaaatacctgctcac
gtctcttactctctgggtctcagcctccctttctgtaaaacaaggatgat
cataggacccacttcctcccatggtttgtttcctgggtgcctactatgtg
ccaagcacatgctagtgacatgaagtgcaccgtctcccaccctgcaaact
cttgggaacaatcctggggaagtcagtattattcccacttgccagtgaag
acgctgagaatggggaagggaaagggtgaatgaggggagagaggagagga
agctgccagcaggggaagcacagccagcttccctctctccttctggaagc
cccagggccacaggccagggggcgggcctggcgggggtgtggccagccct
gggtccccattggctgctggacggcgccctgggaataaagctgggaacca
AAGACCCAGACCCAGACCCATAGGCCCTGGGACGATCCAGCAGACATTCT
AACTGTCCATCTACCTCCATCCGTGAGAGGACCATCTATCCAGTCTATAC
ATCTAGACCACCTCCCGTCTATCCCTCCTCGGCTTGCTCCCTCTGCCTGG
GCGGCCCACCTTCACTGCCTTGGTCAGgtaagtacccccacctcagcccc
accccagactggagtgcctgtggaatgccaggagattccaggagaagagg
aggggatagagtcagttgagcataaaaacccatgctttgaagccagactg
gctgggtcggcatctagctctgggcctcagtttcccatttccctaaaatg
ggaacaataactgacccttcctcatcgagctgttgtaaagatgaaatgag
atcatgcatttaaaatgtccagtccatggacagcccgggtggctcaggag
ttaagcgcctgcctacagcccagggagtgatcctggagtcccaggatcga
gtcccacatcaggctctctgcatggagcctgcttctccctctgcttgtgt
ctctgcctctctctcatgcataaatttttaaaatcttaaaaaaaaaataa
aatgtccagcccagatataatgatagttcatttttattgagctcttgcta
tataagccaagtgagagggggagtgacttctgaccaggagggaacattag
gtagatagcagcttctagtaccctctgtcctgtgtttggcaaaacctcag
gatgttatgctggagggagtggtgaccacagggggtccctacccctcccc
tgaaggcattgcatggtgcaaccagaggtggaggaggcttcccaaatcca
cccaaaaggggtcttgtgggggttgcctcccacatccagccaggacagct
ctttgaggctggggctgggcaacgtgcacaaggtccactcagccagaatg
agggtgcccttgtttcagggttaacctccagaaggttcattaaacttaat
tcagacagatatgggatgctcccgtaatagagtgcctctagtccctggct
tttgctcaagagatcctaggggagagatggtatctgttgagcacctctct
taggccccgaatgtcagttaattcagacagacgagcgatccatatttggg
ggtgctaagggtggtggagtggtcaggaagcctacccctcactttgcttc
tgaccaaggcatatctagcccagtctgggagtggcaatggcccagggcta
gggcggcaccaggtagtgtcattggaaatagggtgggaggtagaaagagg
gagccagtcttgcttggccttgtgggatgggggaggtggtagtggtgggg
gaccagagaggtgacttacccaaccatctgtggatcagttctggttcagc
ccaagttggggagaatcagaaaaagaagagaaaaaaaggctgcatgtatg
tggccagactccggaccatggcagcttcttcatcactcagagtcagtcca
gaccgggaggcctgagccatgcccatgtgtcctggagacagcttagttca
ggtgctagactgtctgaattcaaatcctgcctcccccactcactagctgt
gtgaccttgggcaagtcacattaatattccgatccctattgctatgtgac
aaactgcccgaaggtatagtgaccaaaaacaacagcaatcattgtattgt
tgtctcatggctctggggcttaattgggctctgcaggcagttctcgctgg
actctctcaggcggctgcagtgtgacagtggctggtgatgtcccaaggtc
acacacacacacagacacctacccgtctcacaagtgacgctggccatcag
ctgtcaggtaggggcacctccatatggcctctgcatgaggcctacgctta
cccgcctcacggcagctgggttttggaagagagttgctcaagagaaatag
ccaggtggaggctgtgtccccttctgtgacccagccctgggtgaggggct
gccctgaagccgactcacacagactctggaaagcggactgtgccatctct
tcccagctccacactcatgacatcgtgttggtagtgtgaacgcggccttg
gtgaagcttatgtacagcacagaggtgcacacactacaaatcagggcttt
atcttggagagtaaacatttttaccagcactttgctgcttggaagccaca
tagtgtcactgcagccacagacaccctctgcccctccagggctcatgtgg
aggaagcacagaccccacctctcactgagatgatcactcataagagatgc
gtgtgggatggggagagcttgcagcggccatcccgggaaacacaatccga
gcctttttcctcatcaggagaatgggatgataaggcacctcctaggaagg
ctaaaaggattaaatgaattaatatctgtaaggcttaaaacattgtgtgg
cacgtggtgatcactttagaagtgattatcacataatataatgactataa
aaagtctctctaactgggtgcggggctttccaaaggttagtataaaaaaa
atgcatgtatctaaacagataatccctaaacacgacaaaacattcaaaca
gtacaaaagggatgctgagtgccagctctccccagaagccccttgcccta
acaagtttcttgtgtgtatcctgagacggcctatgcacacagaagcttat
ggattatgtacgtggtgggggtgtccccatattttttcacaaatagactc
tctctgctatcctgccccttctttaccactaccacttaagcatcttgaag
ctcactctgaatcagcacctgcaaatccatctccacctcctgcccctgga
aaggtgcaaggtctgaacgttatttctaatgttcacagcttcccagcatg
ctgtgtgcatggggtgggtggcggtgctggtgatggtaagaatccttgtc
cccattttacagatgaggcagctgaggcagagggggggattggttgtgtg
atgtcacctggtacgtaggtggctcagttgagatttgaattcaggtctcc
aggatgctcctcatctgggctctttcctcggcttcgtgggcagtgagggg
aggagtcactcagaggcacactgagggctgtatggcagcggctggctctc
ctgaccatctgggtgtccttccttcctcccagCGATGACGCCTGGAGAGC
CCCTGCACGTGAAGACCCCCATCCGTGACAGCATGTCGCTGTCCAAAGTG
GCAGGCACCAGCGTCTACCTCAAGATGGACAGTGCCCAACCTTCAGGCTC
CTTCAAGATCCGGGGCATTGGACACCTCTGCAAGATGgtacaggagggct
ccgcagcatctgagtgagggtggcatgtcctttatcttcaaaggacaagg
ccagtgttgtgtgctcactctcacaaagcccttgtatctgccattagcac
tccctctaggaaggggggccacccaagggggtctccttatggactgagcc
cagagccatcatttgcctgcttcccttcaccccaacccttccctgagcca
gtctccggggcggggcgatgtcatctgagaggctagggcaatacagagtc
tttgtgaaggaaagagggcctggtgcatattggaatcactgggaagcact
ccggacaggagagctgtctgggcggtggcctgacctcccctcttgtctcc
acagTGGGCTGAGCAAGGCTGTGAACATTTCGTCTGCTCCTCGGgtgagt
ggcctgcatgcttcctcctgtgccctgctgtgtgtcccaggccaggcacc
cccccggcgcaccccccctcccggggccttggtttccgtagcaatgagcc
attgcaaacatctgcagcggccccaatttggaactccggggcgggggggg
gggggggggggggaggcgagctgtcactcagggaggtgtgcagagcccca
gggttgggggcgagcaggcccagctgggctcccaggcctttctgcactga
catactctccacccccatccctggcagCGGGCAATGCAGGCATGGCAGCC
GCCTATGCTGCCAGGAAGCTGGGCATCCCTGCCACCATCGTTGTGCCCAG
CACCACCCCTGCCCTCACCATTGAGCGGCTCAAGAATGAGGGTGCCATAG
TCAAGGTGGTGGGTGAGgtgagtaccaacaacccagggcaggggccgcgg
agggcacccggtggctgggcagggtctccagcccctcaccccctccaacc
tccctgtggtcttgcagATGTTGGATGAGGCCTTCGAGCTGGCCAAGGCC
CTGGCCAAGAACAACCCTGGCTGGGTGTACATTCCTCCCTTTGACGACCC
CCTCATCTGgtatgtggagcccagtcccctggtacaggtggcgttagaga
aaggggaggtgagccgaagccggaagggagccagtaaagggctctcatgg
gcaggtgaccgctatgggcccactcggggcccctgggagccagtgtggat
cccaaaccagaattattccacctgcgggagagcgtgctggggtttataca
ccaactgccaccgttctggactcccgcagacattggttgagctgctttcc
gggggtgtcctctcctgccacagttgtaagcagctccagtggccagagaa
agcccttgggtagtcacgcagatgttggcagctggaagtcggggtggagt
tggggatcaggcaactagcccaaagcccatcagctgcctgggaggacagg
tggcacctccaggacccctgcgtggtgtctccagggagcccagccctgac
ctgtgccccctccccccctccccagGGAAGGCCACACTTCCATCGTGAAG
GAGCTGAAGGAGACCCTGAGTGCAAAGCCAGGGGCCATCGCGCTGTCAGT
GGGCGGCGGGGGCCTGCTGTGCGGAGTGGTCCAGGGGCTGCAAGAGGTGG
GCTGGGGGGACGTGCCTGTCATCGCCATGGAGACCGCCGGAGCCCACAGC
TTCCACGCTGCCACCACTGCCGGCAAGCTGGTCTCCCTGTCCCAGGTTAC
CAGgtgagcagagggcctgcctctgtcggtgttcggtaagccttggggag
cccgcctcggtgttggctgcaaggtagacagaccacctccatgtcagtcc
cattttacagatgaggaagctgaggctttggtcacttgcccagagtcaca
agccagcaagtggtactgctgagactgaatgcgaccgagtctacgtgact
cagtaccatgaacattacactcctcacatgaagaggccaatattacgcat
ttattgagtttcctgcagcagggttttacacaccttgttgatttgccaac
catttactgggcccctaaaatgtgcaagcggtgctgatcctggggacaga
gtggtgaataaacacagcccccggtccccaggagctcatatcccagcccc
aaacctttgagagatgatacttaagattaggtttggcacattatacaagt
accccaaatagcagtgacttaaaccagattcaactttactttctcttata
taagtggaaaaaaaaaaaaaaaaaaagcctagaggtaggcggtccagggc
agctccatgaagccagaggcccaggttcgttgcgtttgttctccaccgtc
cttatcatgtagccctatggcccaaaatagctgctcatgtgccgactatc
acacccatgctcgttctggcaggaggagccccctccctttaaagacaggc
acaggggatgcctgggtggctcagcggttgagcgtctgccttccactgag
ggcgtgatcctggtcctgggatcgagtcccgcctcagggtccttgcgggg
agcctgcttcttcctctgcctgtgtctctgcctctctctgtgtctctcat
gaataaataaatctttaaaaaaaaataaagacaggcacagtacttccact
tacctctcattggccagaacttagtcatgtggccacgactactgcaaggg
aggctgggaaatatcttcctaccacgtctccacatgcagctgggttggat
actgggtagacaagtccttaccgcagatagggaagactgagcccaggtaa
gaactcctgggctcctgaacctggcacctgggagctaccatgttgggctc
tgatcctgcagggttgggattgaggataattgtcgcgtgcctacatgtgc
cacatgctgggcttagggctaaggtggttcaggccccaggggctccccag
tgagctgggaatagaaccggtgatgactccagggagaggggggagaaagt
gaatgcactgaataggagtgcaggtcctggagttacatggatcctgctta
aggatctttctcctgcctcctgtttgccccattcccgtcctcttctctat
ccccacctctcccagCGTGGCCAAGGCCCTGTGCGTGAAGACTGTGGGGG
CTCAAGCCCTGAAGCTGTTTCAGGAACACCCCATCTTCTCTGAAGTCATC
TCGGACCAGGAGGCTGTGGCTGCCATTGAGAAGTTCGTGGgtatgtttca
agacctgaccccctagcttggggattaactggctgtctgctcagtggcct
aagagatcccattcgctgtaaattttcttcatcgtcctatctccccatcc
cttcctagaacctcccactatgaaagataatacaatgcagcagtaaaaat
agaaaggccaaagccacaagtgaagaaacctgataaccatgacagcaagg
aataataactgtttaaaatgctgtgagcttcctggtagccagggtgaaaa
aggaaaatacaggcctcagggttggcttcagaaataggtctataactagg
ctggactcctaggttcagtctaggacaggggtggggtgcatgcatatgtg
cgcatgcacacacgtgcacacacacacatacacacacgcaaaggtagcac
taacactgccagttaactcctgtgccagcagtcagttgggctaaccgacc
acagcagtcttgctcaaatgatgactcaagccttctcaacccagtgcttg
ggcagttacaatcgtttcatcagtgcgagcactcgagatgagacctcgcc
tcggaaggtcaatctagcatagtgacgggaggtgtggcccggacccagtc
tgcggccctgccgttcccttgcagcctgcacccaggcctcaccctcacca
cccttccccggcagATGACGAGAAGATCCTGGTGGAGCCCGCCTGCGGGG
CAGCCCTGGCCGCCGTGTACAGCCGCGTGGTGCAGAAGCTGCAAGGGGAG
GGGAAGCTCCAGGCCCCGCTGTCCTCCCTCGTGGTCATTGTCTGCGGGGG
CAGCAACATCAGTCTGAGCCAGCTGCGGGCCCTCAAGGAACAGCTGGGCA
TGAATGGGCTGCCCAAGTGA

### Example 1: Serine Metabolism Disrupted in Diabetes Model Mice and SDS Overexpression Mice

This example shows that diabetes model mice and SDS-overexpression mice have disrupted serine metabolic homeostasis.

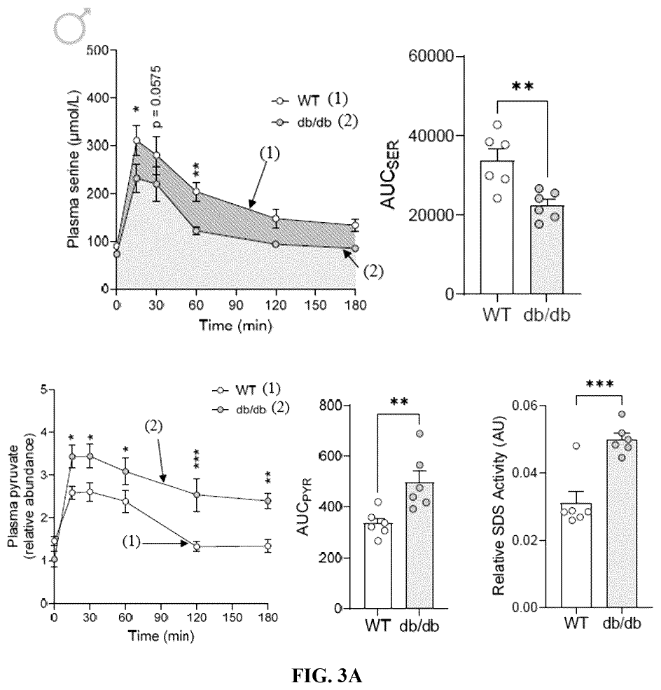

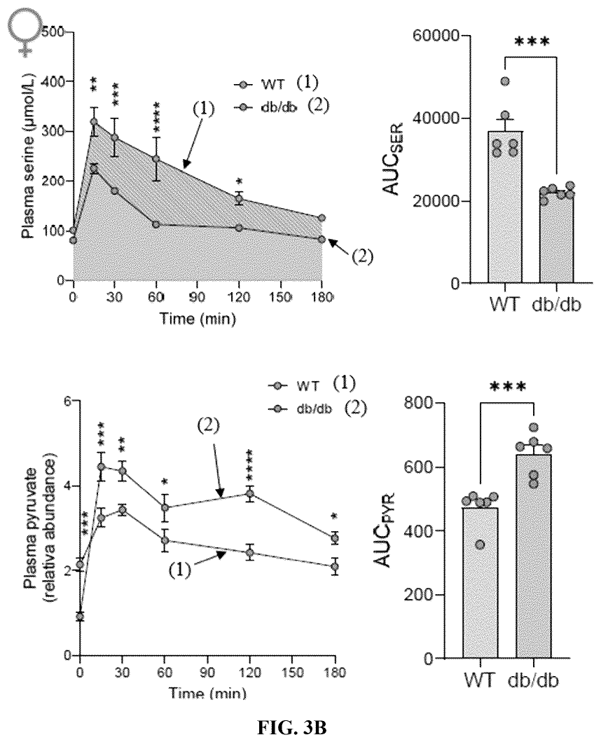

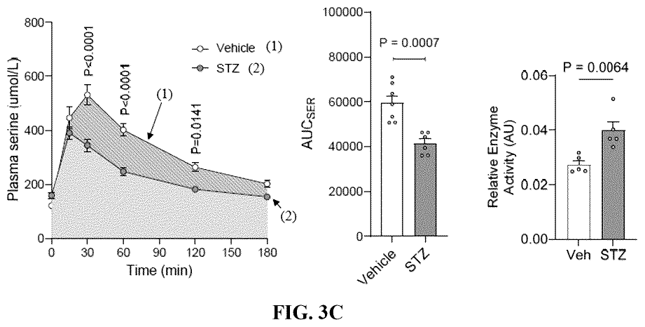

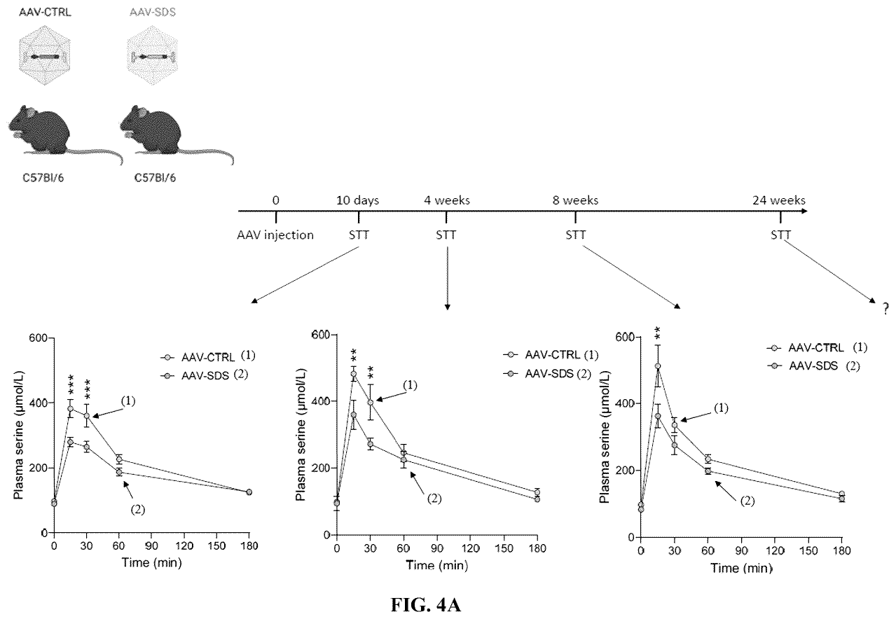

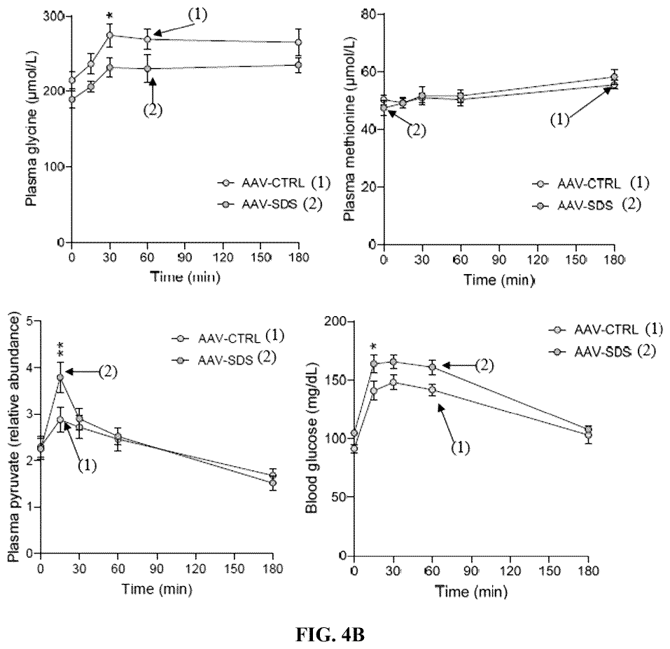

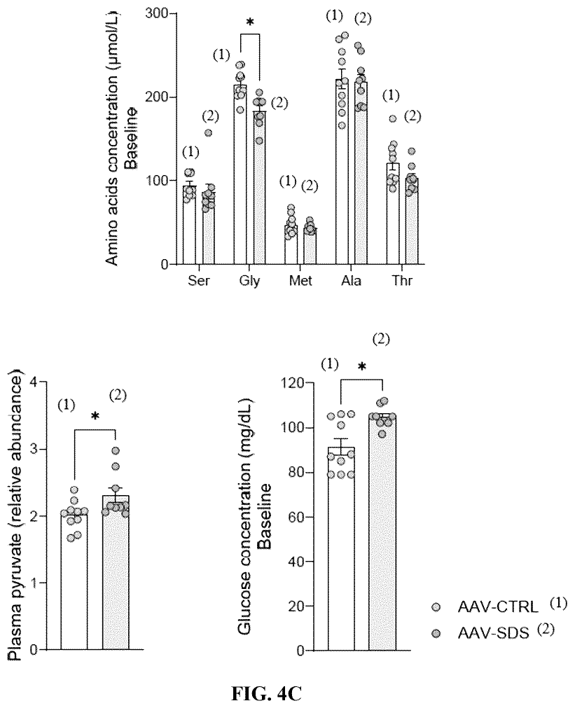

As discussed above, SDS is involved in serine conversion to pyruvate. FIG. 3A shows that male diabetic mice (db/db) had substantially lower plasma serine levels and higher plasma pyruvate levels after a serine tolerance test compared to wild type mice (WT). The same phenotype was observed in female db/db mice (FIG. 3B) and in streptozotocin (STZ)-treated mice (FIG. 3C), a model of type 1 diabetes. Accordingly, these data demonstrate that diabetes results in a dysregulation of serine metabolism and homeostasis. Overexpressing SDS in wild-type mice recapitulates the phenotype observed in diabetic mice, such as decreased plasma serine levels and increased pyruvate levels upon a serine tolerance test (FIGS. 4A-4B).

These results show that diabetes and overexpression of the SDS gene both disrupt serine metabolic regulation in animals and result in decreased serine availability. Accordingly, these results demonstrate that serine metabolic homeostasis is disrupted in diabetes and that SDS expression alters systemic serine metabolic regulation.

### Example 2: SDS Knockout Systemically Alters Serine Metabolism

This example shows that knocking out SDS in an animal model changes the metabolism of serine and important downstream metabolites in multiple tissues.

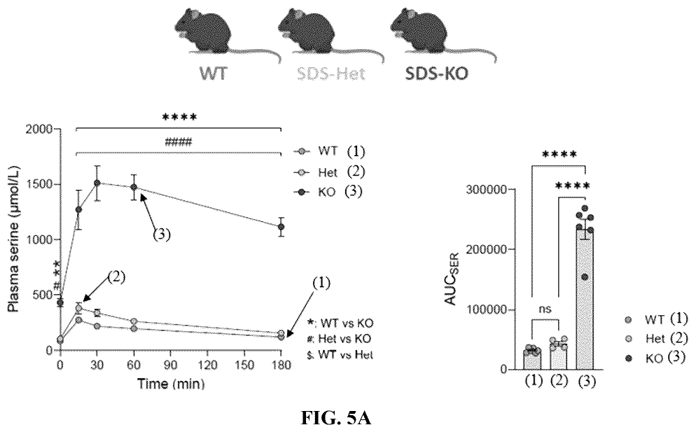

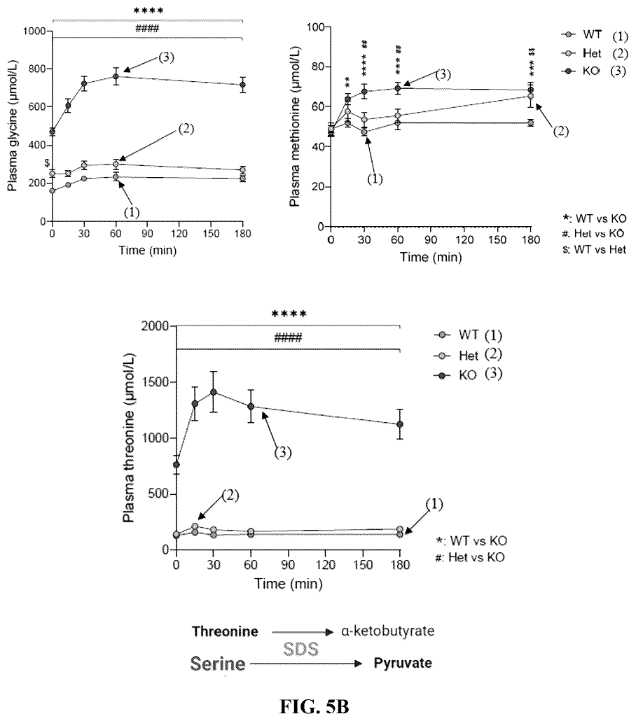

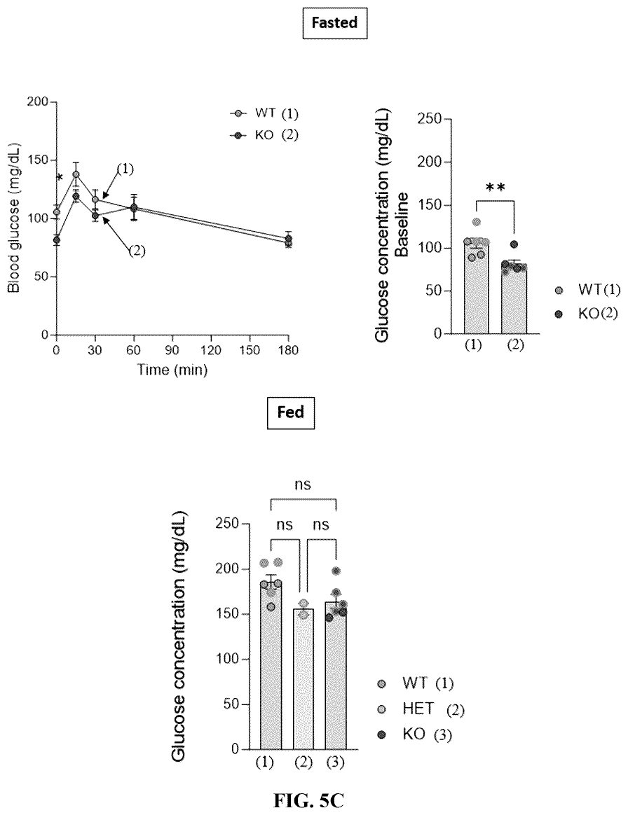

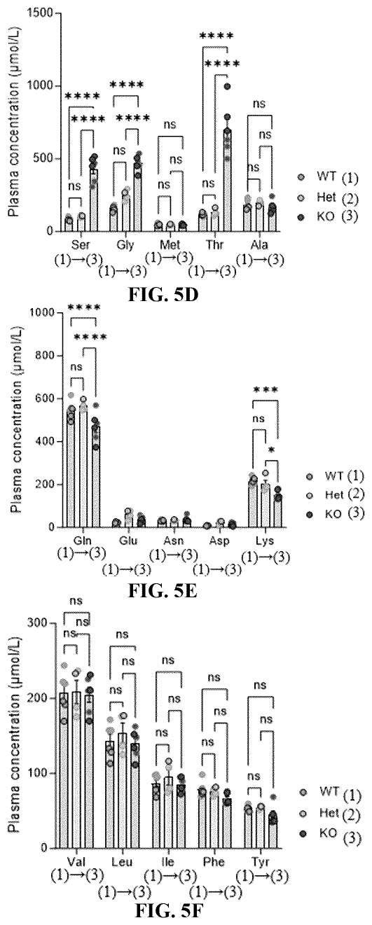

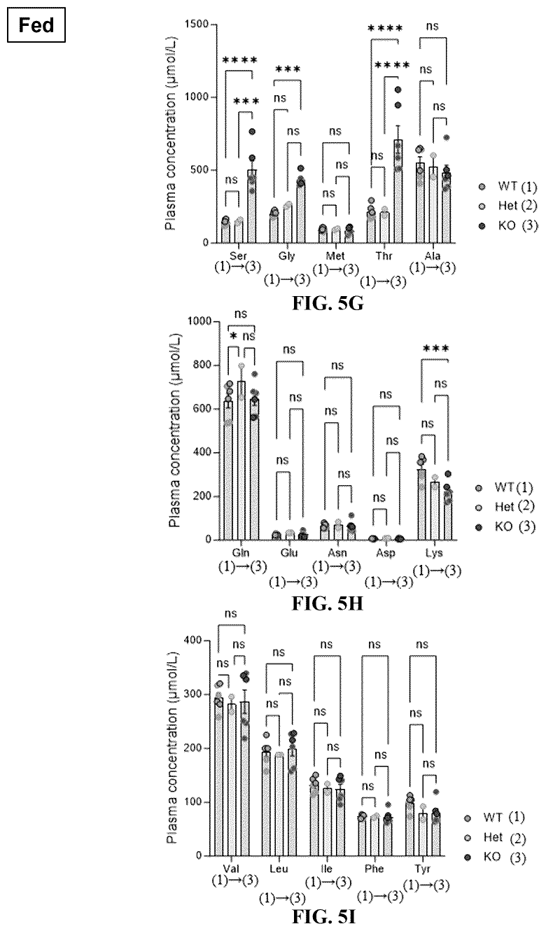

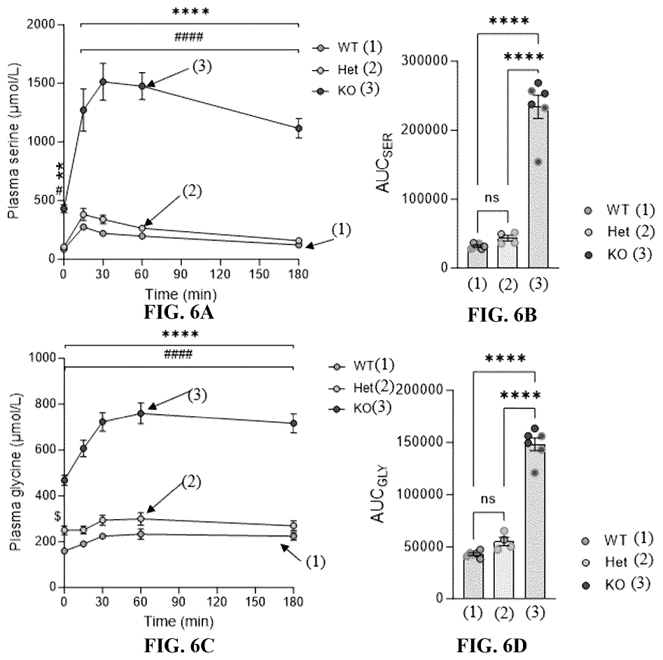

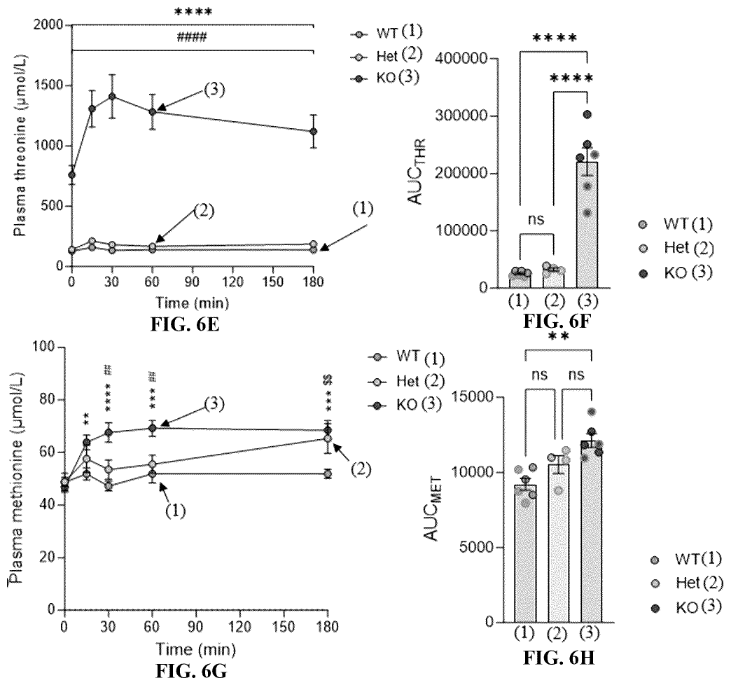

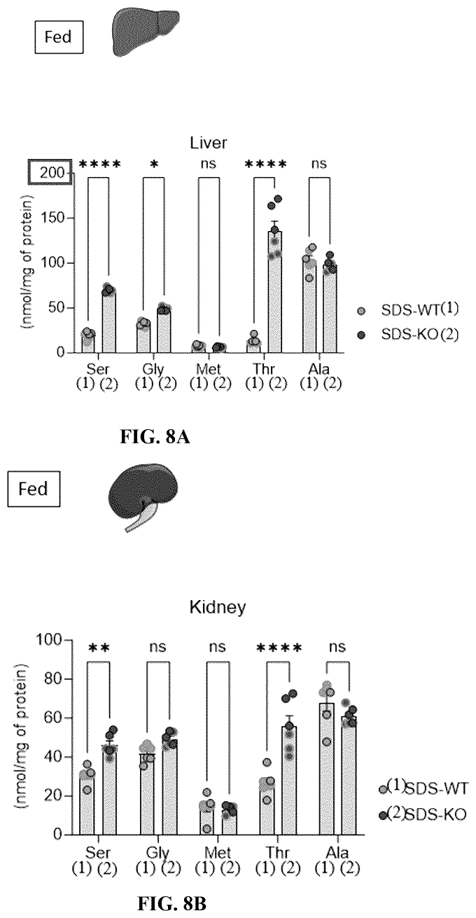

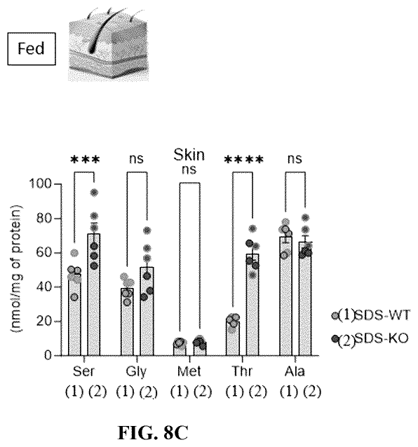

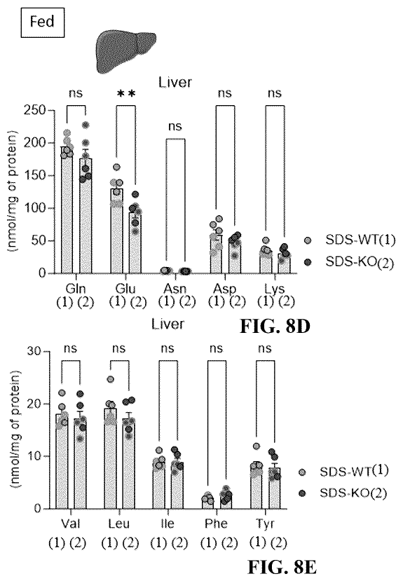

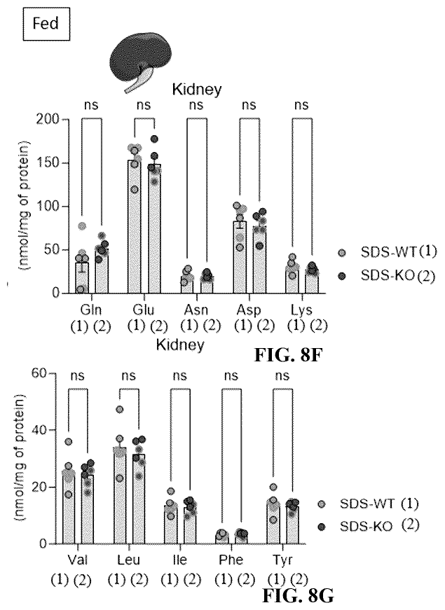

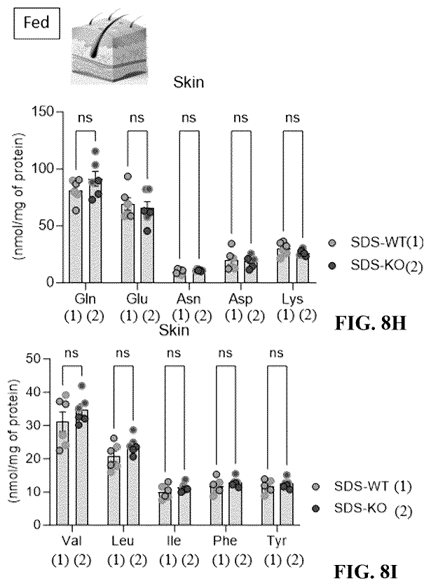

SDS knockout mice were assayed as described above for plasma and tissue metabolite abundance. Plasma and tissue serine, glycine and threonine levels are highly increased in SDS-KO mice (FIGS. 5A-5B, 6A-6H, and 8A-8C). Other amino acid levels in tissues were not substantially different between SDS-KO mice and WT mice (FIGS. 8D-8I). Blood glucose concentration is also increased in SDS KO mice (FIG. 5C). Sphingolipid metabolism was also altered, with decreased abundance of multiple doxDHCer metabolites (FIGS. 7A-7M, 9G-9I, 9P-9R, and 9Y-9AA) and increased abundance of multiple ceramides (FIGS. 9A-9F, 9J-9O, and 9S-9X). Abundance of doxSA and doxDHCer species are known to correlate with peripheral sensory neuropathy and macular disease.

These results demonstrate that disrupting the activity of SDS in an animal model increases the levels of serine and several beneficial ceramides while reducing the abundance of disadvantageous deoxyceramides in multiple tissues. Accordingly, the methods and compositions of the present disclosure are useful for increasing serine and ceramide levels and decreasing deoxyceramides levels in a subject in need thereof.

### Example 3: SDS Disruption Systemically Alters Serine Metabolism

This example shows that shRNA-disruption of SDS activity leads to systemic changes in metabolism.

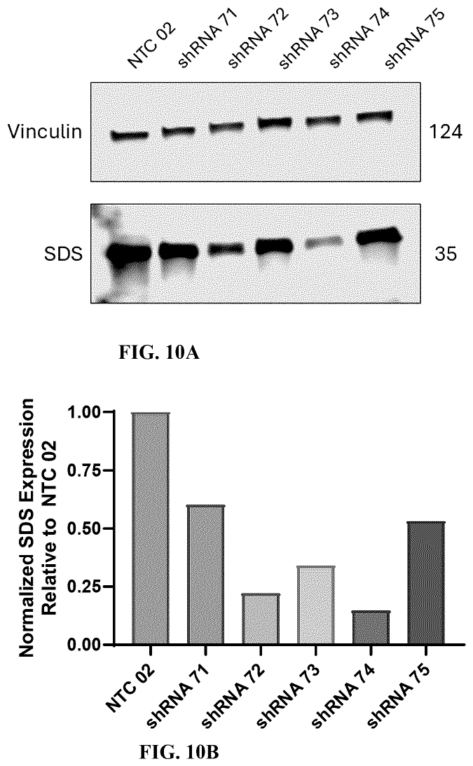

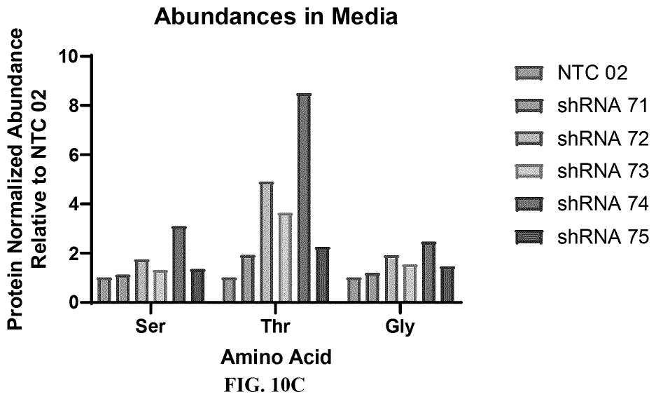

SDS was disrupted using shRNA as described above in a human cell line. As shown in FIGS. 10A-10C, shRNA 74 and 72 were the most effective for decreasing SDS protein levels in cells and caused the greatest relative increase in serine, threonine, and glycine in the media of treated cells. These results confirm that SDS is an important regulatory enzyme for the metabolism of serine, threonine, and glycine in human cells, and that disruption of SDS activity leads to increased abundance of serine, threonine, and glycine.

Accordingly, the methods and compositions of the present disclosure are useful for increasing serine levels in a subject in need thereof.

### Example 4: SDS Disruption Treats Diabetic Peripheral Neuropathy

This example shows that disruption of SDS is therapeutically effective in an animal model for diabetic peripheral neuropathy.

Diabetic mice (db/db) were treated with either a control shRNA or SDS-targeted shRNA using the above described methods to assess how SDS inhibition affects serine and sphingolipid metabolism. Plasma samples were taken from mice at various timepoints, and were assayed for metabolite abundance using the techniques described herein. Thermal latency was also measured as an indicator of peripheral neuropathy. Thermal latency was quantified by behavioral responses to heat using a thermal nociception test device (UARD). In brief, the apparatus surface was warmed up to 30° C., and animals were placed in individual testing chambers for 20-30 min prior to testing. Four separate measurements were performed, and the mean of the last triplicate was taken to represent response latency for each animal. All measurements were made on coded animals by an observer unaware of the treatment groups.

As shown in, FIGS. 11A-11D, serine dehydratase knock-down in BKS-db/db mice decreased serine catabolism and mitigated peripheral neuropathy. A serine tolerance test in BKS-db/db mice 4 weeks after shRNA injection showed elevated serine levels in Sds shRNA treated mice as compared to control (FIG. 11A). Plasma from Sds shRNA treated mice had elevated levels of serine, glycine, methionine, and threonine (FIG. 11B) and an increased serine to alanine ratio (FIG. 11C) as compared to control mice. Sds shRNA treated mice exhibited a reduced thermal latency (FIG. 11D), indicating that Sds shRNA treatment mitigated peripheral neuropathy in the mice.

These results demonstrate that disrupting the activity of SDS in an animal model of diabetic peripheral neuropathy decreases disease symptoms and improves therapeutic outcomes. Accordingly, the methods and compositions of the present disclosure are useful for methods of treating diabetic peripheral neuropathy.

### Example 5: SDS Disruption Promotes Wound Healing

This example shows that disruption of SDS is therapeutically effective in an animal model for wound healing.

SDS knockout diabetic mice (db/db) were generated using the above-described methods, and control SDS WT db/db mice were also maintained. Plasma amino acid abundance, liver serine to alanine ratio, and skin deoxydihydroceramide abundances were quantified in db/db-Sds-WT and db/db-Sds-KO mice using previously described methods. Wound healing was also assayed as follows. After anesthesia, a donut-shaped splint made from a 0.5 mm-thick silicone sheet was placed and sewn on the mouse shaved back. A 4 mm biopsy was performed so the wound was centered within the splint. Mice were single housed and monitored every other day for 22 days for wound closing. Wound pictures were analyzed using QuPath software.

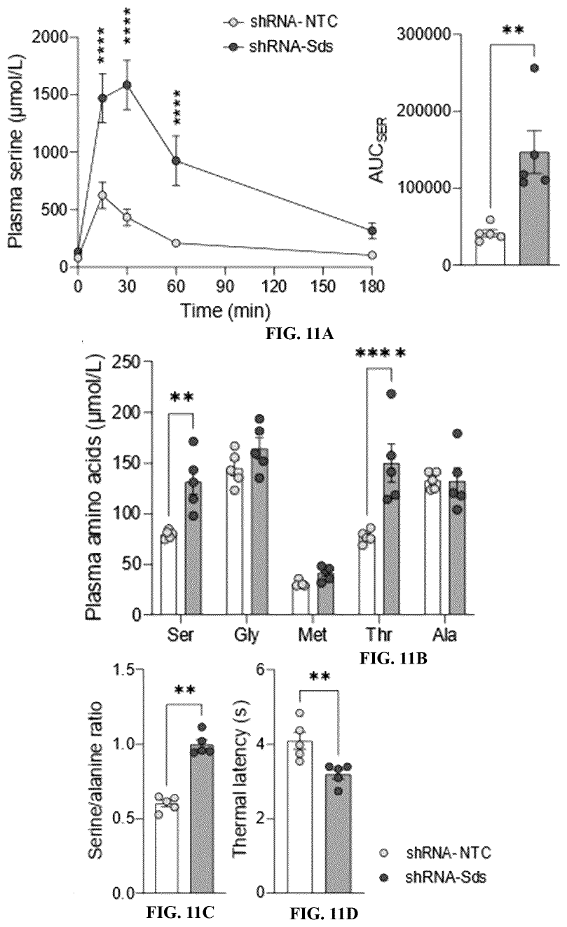

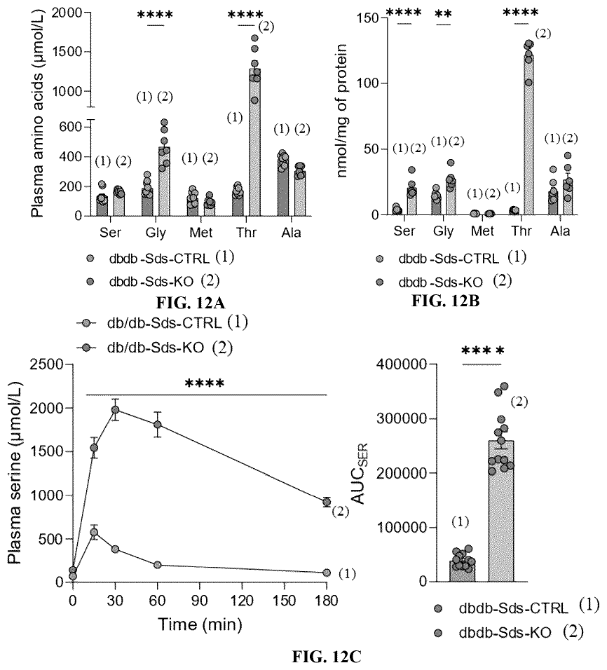

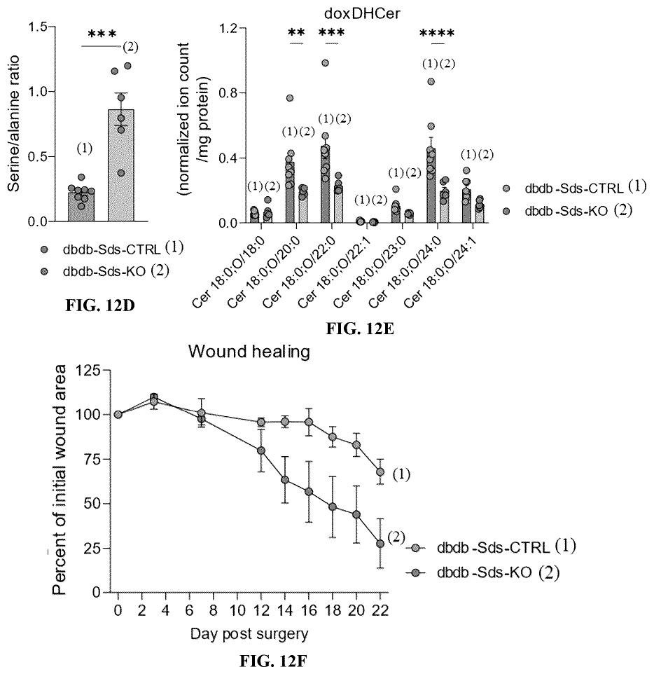

As shown in FIGS. 12A-12F serine dehydratase deletion in db/db mice decreased serine catabolism and improved wound healing. The plasmatic (FIG. 12A) and hepatic (FIG. 12B) amino acid concentration in db/db-Sds-KO mice display similar patterns, including increased serine, glycine, and threonine abundance. A serine tolerance test performed on 12-14 week old female and male mice showed increased serine levels in Sds-KO mice relative to control mice. The serine and alanine ratio in the liver was elevated in Sds-KO mice as compared to control mice (FIG. 12D) and skin deoxydihydroceramide abundances were largely decreased in Sds-KO mice compared to control (FIG. 12E). As shown in FIG. 12E, db/db-Sds-KO female mice healed faster from a wound than control mice.

db/db-SDS-CTRL and db/db-SDS-KO mice were also monitored for weight gain and blood glucose levels until 7 weeks of age. SDS-KO mice exhibited decreased weight and blood glucose compared to control mice at 7 weeks (data not shown). A glucose tolerance test was also performed in db/db-SDS-CTRL and db/db-SDS-KO mice, and db/db-SDS-KO mice showed a minor decrease in blood glucose during the test as compared to control mice (data not shown).

These results demonstrate that disrupting the activity of SDS in an animal model of diabetes and wound healing decreases symptoms and improves therapeutic outcomes. Accordingly, the methods and compositions of the present disclosure are useful for methods of treating one or more wounds and for treating diabetes.

### Example 6: SDS Disruption Treats Obesity

This example shows that disruption of SDS is therapeutically effective in an animal model for obesity.

Sds-KO mice were generated as described previously, and control SDS-WT mice were also maintained. Mice were put on a high fat diet for 16 weeks as described above and assessed over time for weight, fat composition, histology, and metabolite abundance. For histology, liver and inguinal white adipose tissue (iWAT) sections were fixed overnight in 10% neutral buffered formalin to perform. Fixed tissues were washed with PBS and stored in 70% ethanol until sectioning. Liver and iWAT sections were stained with H&E (hematoxylin & eosin) to visualize liver steatosis and adipocyte size in iWAT.

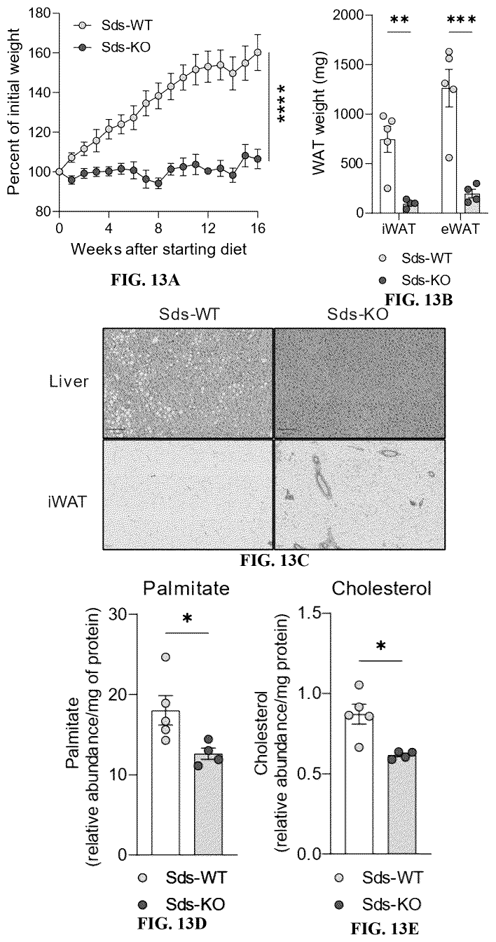

As shown in FIGS. 13A-13E, serine dehydratase deletion protected against High Fat Diet-induced obesity. While Sds-WT mice exhibited a steady and substantial increase in the percent of initial body weight, there was dramatically reduced weight gain in Sds-KO mice (FIG. 13A). Sds-WT mice had dramatically increased inguinal and epididymal white adipose tissue (iWAT and eWAT) weights as compared to Sds-KO mice (FIG. 13B). H&E staining on liver and iWAT slices from Sds-WT and KO mice fed with HFD for 16 weeks confirmed that SDS deletion led to compositional changes in these tissues (FIG. 13C). Liver palmitate (FIG. 13D) and cholesterol (FIG. 13E) levels were reduced in Sds-KO mice as compared to Sds-WT mice fed with HFD for 16 weeks.

These results demonstrate that disrupting the activity of SDS in an animal model of diet induced obesity decreased or prevented one or more symptoms and improved one or more therapeutic outcomes. Accordingly, the methods and compositions of the present disclosure are useful for methods of treating obesity (e.g., diet induced obesity).

### Example 7: SDS Disruption Treats Liver Damage

This example will show that disruption of SDS is therapeutically effective in an animal model for liver damage.

To analyze the effect of SDS disruption on liver damage, liver damage will be induced, either by using a chemical agent (e.g., CCl4) or by directly inducing a partial hepatectomy, in SDS KO mice, optionally with WT control mice. Liver damage recovery will be assessed by measuring hepatic enzyme levels, which are a readout of liver injury, in the plasma and analyzing cell proliferation in the liver at several time points post liver injury. Corresponding assays will be performed using other methods of SDS disruption, including, but not limited to, CRISPR (clustered regularly interspaced short palindromic repeats)/Cas (CRISPR-associated) system, meganucleases, zinc finger nucleases (ZFNs), RNAi, anti-sense oligonucleotides, and TAL effector nucleases (TALENs).

These results are expected to demonstrate that disrupting the activity of SDS in an animal model of liver damage decreases one or more symptoms and/or improves one or more therapeutic outcomes. Accordingly, the methods and compositions of the present disclosure are useful for methods of treating liver damage.

### Example 8: SDS Disruption Treats Nerve Damage

This example will show that disruption of SDS is therapeutically effective in an animal model for nerve damage.

To analyze the effect of SDS disruption on nerve damage and recovery, sciatic nerve ligation will be induced in SDS KO mice and optionally in WT control mice. Blood collection will be performed before and after nerve injury to assess amino acid and sphingolipid profiles in plasma. Pain sensitivity will be assayed to determine nerve regeneration. Thermal sensitivity and mechanical allodynia will be measured using Hargreave's and Von Frey assays respectively. Corresponding assays will be performed using other methods of SDS disruption, including, but not limited to, CRISPR (clustered regularly interspaced short palindromic repeats)/Cas (CRISPR-associated) system, meganucleases, zinc finger nucleases (ZFNs), RNAi, anti-sense oligonucleotides, and TAL effector nucleases (TALENs).

These results are expected to demonstrate that disrupting the activity of SDS in an animal model of nerve damage decreases one or more symptoms and/or improves one or more therapeutic outcomes. Accordingly, the methods and compositions of the present disclosure are useful for methods of treating nerve damage.

### Example 9: SDS Disruption Treats Muscle Damage

This example will show that disruption of SDS is therapeutically effective in an animal model for muscle damage.

To analyze the effect of SDS disruption on muscle damage, notexin will be administered intramuscularly and a muscle injury will be induced in the SDS KO mice and optionally in WT control mice. Blood collection will be performed before and after muscle injury to assess amino acid and sphingolipid profiles in plasma. To assess muscle regeneration, muscle-specific stem cell frequency will be assayed using Pax7 staining in the muscle and myofiber size will be measured in during muscle regeneration phase. Corresponding assays will be performed using other methods of SDS disruption, including, but not limited to, CRISPR (clustered regularly interspaced short palindromic repeats)/Cas (CRISPR-associated) system, meganucleases, zinc finger nucleases (ZFNs), RNAi, anti-sense oligonucleotides, and TAL effector nucleases (TALENs).

These results are expected to demonstrate that disrupting the activity of SDS in an animal model of muscle damage decreases one or more symptoms and/or improves one or more therapeutic outcomes. Accordingly, the methods and compositions of the present disclosure are useful for methods of treating muscle damage.

### Example 10: SDS Disruption Treats Macular Telangiectasia (MacTel)

This example will show that disruption of SDS is therapeutically effective in an in vitro model for MacTel.

It has been shown that dietary serine/glycine restriction alters retinal function measured by electroretinogram (ERG) in mice. Gantner et al. NEJM 2019 and impending/accepted publication Lim et al. Cell Metabolism. Mice will be fed a serine/glycine-restricted diet and administered an agent to disrupt SDS, such as shRNA CRISPR (clustered regularly interspaced short palindromic repeats)/Cas (CRISPR-associated) system, meganucleases, zinc finger nucleases (ZFNs), RNAi, anti-sense oligonucleotides, and TAL effector nucleases (TALENs). ERG defects will be measured before and after treatment using standard protocols. It is anticipated that SDS disruption will prevent, ameliorate, or restore ERG defects.

These results are expected to demonstrate that disrupting the activity of SDS in a model of MacTel disease progression decreases one or more symptoms and/or improves one or more therapeutic outcomes. Accordingly, the methods and compositions of the present disclosure are useful for methods of treating MacTel.

### REFERENCES

1. www.addgene.org/protocols/plko/#top
2. Muthusamy, T., Cordes, T., Handzlik, M. K., You, L., Lim, E. W., Gengatharan, J., . . . & Metallo, C. M. (2020). Serine restriction alters sphingolipid diversity to constrain tumour growth. Nature, 586(7831), 790-795.
3. Cordes, T., & Metallo, C. M. (2019). Quantifying intermediary metabolism and lipogenesis in cultured mammalian cells using stable isotope tracing and mass spectrometry. High-throughput metabolomics: methods and protocols, 219-241.
4. Handzlik M, Gengatharan J M, Frizzi K E, McGregor G H, Martino C, Rahman G, Gonzalez A, Moreno A M, Green C R, Guernsey L S, lin T, Tseng P, Ideguchi Y, Fallon R J, Chaix A, Panda S, Mali P, Wallace M, Knight R, Gantner M, Calcutt N A, and Metallo C M.† Insulin-regulated serine and lipid metabolism drive. Nature 614:118-124 (2023).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for increasing serine levels in one or more tissues in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system.

2. The method of claim 1, wherein the agent that disrupts SDS activity is delivered via a vector selected from the group consisting of: an adeno-associated virus, a virus-like particle, and a lipid nanoparticle, optionally wherein the vector is targeted to the liver and/or the kidney.

3. (canceled)

4. The method of claim 1, wherein administration of the agent to the subject results in one or more of inhibiting the transcription of the SDS gene or translation of mRNA transcribed from the SDS gene.

5. The method of claim 1, wherein:
the one or more tissues is selected from blood tissue, liver tissue, kidney tissue, ocular tissue, muscle tissue, and skin tissue, or any combination thereof; and/or
the one or more tissues is blood tissue, and administration of the agent to the subject increases plasma serine levels in the subject in both fed and fasting states; and/or
the one or more tissues is blood tissue, and administration of the agent to the subject increases plasma serine levels in the subject by at least about 10% to at least about 300%.

6.-7. (canceled)

8. The method of claim 1, wherein administration of the agent to the subject increases glycine and/or threonine levels in the one or more tissues.

9. The method of claim 8, wherein:
the one or more tissues is blood tissue, and administration of the agent to the subject increases plasma glycine and/or threonine levels in the subject in either fed or fasting states, and/or
the one or more tissues is blood tissue, and the administration of the agent to the subject increases plasma glycine levels by at least about 10% to at least about 300% and/or plasma threonine levels by at least about 10% to at least about 600%.

10. (canceled)

11. The method of claim 1, wherein:
administration of the agent to the subject decreases 1-deoxydihydroceramide (doxDHCer) levels in the one or more tissues, and/or
the one or more tissues is blood, and the 1-deoxydihydroceramide is selected from the group consisting of: doxDHCer m18:0/22:0, doxDHCer m18:0/24:0, doxDHCer m18:0/24:1, and doxDHCer m18:0/26:0.

12. (canceled)

13. The method of claim 1, wherein the subject is diagnosed with, suspected of having, or at an increased risk of, diabetic peripheral neuropathy, macular telangiectasia (MacTel), one or more wounds, liver damage, nerve damage, and muscle damage.

14. The method of claim 1, wherein the agent is administered orally, topically, systemically, intravenously, subcutaneously, transdermally, iontophoretically, intranasally, intraperitoneally, or intramuscularly.

15. The method of claim 1, wherein the subject is human, canine, feline, murine, or equine.

16. The method of claim 1, further comprising administering an additional therapeutic agent selected from the group consisting of serine, glycine, threonine, glucose, a Glucagon-like peptide-1 (GLP-1) agonist, or glutamine to the subject.

17. The method of claim 1, wherein the method does not comprise administering serine to the subject.

18. The method of claim 17, wherein the subject is at risk for or has been diagnosed with Type 1 diabetes or Type 2 diabetes.

19. The method of claim 1, further comprising administering a Glucagon-like peptide-1 (GLP-1) agonist to support maintenance of muscle mass.

20. The method of claim 1, wherein the agent is:
(i) a shRNA, RNAi, or anti-sense oligonucleotide selected from any one or more of the oligonucleotides as set forth in SEQ ID NOs: 2-6, Table 12, or Table 13;
(ii) a zinc finger nuclease comprising a zinc finger having the amino acid sequence as set forth in Table 1, Table 2, Table 3, Table 4, or Table 5; or
(iii) a Clustered Regularly Interspaced Short Palindromic Repeats (CRISPR)-associated (CRISPR-Cas) system comprising a guide RNA comprising an oligonucleotide selected from any one or more of the oligonucleotides as set forth in Table 6, Table 7, Table 8, Table 9, Table 10, or Table 11.

21.-22. (canceled)

23. The method of claim 1, wherein the agent is conjugated to at least one sugar moiety, optionally N-acetyl galactosamine (GalNAc), in particular triantennary GalNAc.

24. A composition comprising an agent the disrupts serine dehydratase (SDS) activity, wherein the agent is selected from the group consisting of:
(a) an antisense oligonucleotide, or a nucleic acid encoding the same, comprising a nucleotide sequence selected from any one of the oligonucleotides as set forth in Table 12 or Table 13;
(b) a siRNA, or a nucleic acid encoding the same, comprising a nucleotide sequence selected from any one of the oligonucleotides as set forth in Table 12 or Table 13;
(c) a shRNA, or a nucleic acid encoding the same, comprising a nucleotide sequence selected from any one of the oligonucleotides as set forth in SEQ ID NOs: 2-6, Table 12, or Table 13;
(d) a zinc finger peptide, or a nucleic acid encoding the same, as set forth in Table 1, Table, 2, Table 3, Table 4, or Table 5; and
(e) a nucleic acid encoding a CRISPR-Cas system, wherein the nucleic acid encodes a guide RNA comprising the oligonucleotide selected from any one of the oligonucleotides as set forth in Table 6, Table 7, Table 8, Table 9, Table 10, or Table 11.

25.-31. (canceled)

32. A method for increasing ceramide levels and/or decreasing deoxyceramide levels in one or more tissues in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger, and a nucleic acid encoding a CRISPR-Cas system.

33.-40. (canceled)

41. A method for selecting a subject for treatment with a therapeutically effective amount of an agent that disrupts serine dehydratase (SDS) activity, the method comprising:
(a) detecting levels of serine in a blood sample from the subject;
(b) selecting the subject for treatment with the agent that disrupts SDS activity where the level of serine and/or glycine in the blood sample from the subject is reduced by about 20% compared to the normal control sample; and
(c) administering to the subject a therapeutically effective amount of the agent that disrupts SDS activity, wherein the agent is selected from the group consisting of: an antisense oligonucleotide, a shRNA, a siRNA, a zinc finger nuclease, and CRISPR-Cas system.

42.-45. (canceled)

46. A method for treating diabetes, treating diabetic peripheral neuropathy, treating obesity, or treating one or more wounds in a subject in need thereof, the method comprising administering to the subject a therapeutically effective amount of an agent that disrupts serine dehydratase (SDS) activity, wherein the agent is selected from the group consisting of: an antisense oligonucleotide, a shRNA, a siRNA, a zinc finger nuclease, and CRISPR-Cas system.

47.-64. (canceled)
